(19) United States
(12) Patent Application Publication
(10) Pub. No.: US 2025/0188464 A1
(43) Pub. Date: Jun. 12, 2025

(54) POLYNUCLEIC ACID MOLECULES FOR INHIBITING EXPRESSION OF LP(A), PHARMACEUTICAL COMPOSITIONS, AND USES THEREOF

(71) Applicant: Sirius Therapeutics, Inc., San Diego, CA (US)

(72) Inventors: Curt BRADSHAW, San Diego, CA (US); Anthony NICHOLAS, Oregon, WI (US)

(21) Appl. No.: 19/048,756

(22) Filed: Feb. 7, 2025

Related U.S. Application Data
(63) Continuation of application No. PCT/US2023/030019, filed on Aug. 10, 2023.
(60) Provisional application No. 63/449,914, filed on Mar. 3, 2023.
(60) Provisional application No. 63/371,167, filed on Aug. 11, 2022.

(51) Int. Cl. C12N 15/113; A61P 3/06

(52) U.S. Cl. CPC C12N 15/113; A61P 3/06; C12N 2310/11; C12N 2310/14; C12N 2310/315; C12N 2310/321; C12N 2310/322; C12N 2310/351

(57) ABSTRACT

Disclosed herein are polynucleic acid molecules, pharmaceutical compositions, and methods for suppressing the expression of Lipoprotein(a) (Lp(a)) gene.

### CROSS-REFERENCES

This application is a continuation of International Application No. PCT/US2023/030019, filed Aug. 10, 2023, which claims the benefit of U.S. Provisional Application No. 63/371,167, filed on Aug. 11, 2022, and U.S. Provisional Application No. 63/449,914, filed on Mar. 3, 2023, each of which is incorporated herein by reference in its entirety.

### SEQUENCE LISTING

The instant application contains a Sequence Listing which has been submitted electronically in XML format and is hereby incorporated by reference in its entirety. Said XML copy, created on Feb. 6, 2025, is named 61382-712.301_SL.xml and is 2,341,181 bytes in size.

### BACKGROUND OF THE DISCLOSURE

The discovery of RNA interference (RNAi) as a cellular mechanism that selectively degrades mRNAs allows for both the targeted manipulation of cellular phenotypes in cell culture and the potential for development of directed therapeutics (Behlke, 2006, Mol. Ther. 13, 644-670; Xie et al., 2006, Drug Discov. Today 11, 67-73).

Lipoprotein(a)(Lp(a)) is a lipoprotein composed of an LDL (low-density lipoprotein) lipid core, apolipoprotein B (apo B), and a unique apolipoprotein, apo(a). Lp(a) is considered one of the most common independent genetically inherited causal risk factors for cardiovascular disease (CVD). Accordingly, there is a need for developing an effective Lp(a) inhibitor. The polynucleic acid molecules, conjugates thereof, and methods described herein satisfy this need and provide related advantages.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### SUMMARY OF THE DISCLOSURE

Disclosed herein, in certain aspects, is a polynucleic acid molecule for modulating expression of lipoprotein(a) (Lp(a)) gene, comprising a sense strand and an antisense strand, wherein the antisense strand comprises a nucleic acid sequence that is at least 80%, at least 85%, at least 90%, at least 95%, at least 96%, at least 97%, at least 98%, at least 99% identical to a nucleic acid sequence selected from SEQ ID NOs: 1-154. In some cases, the sense strand comprises comprises a nucleic acid sequence that is at least 80%, at least 85%, at least 90%, at least 95%, at least 96%, at least 97%, at least 98%, at least 99% identical to a nucleic acid sequence selected from SEQ ID NOs: 309-462. In some cases, the antisense strand comprises a nucleic acid sequence comprising at least 14, 15, 16, 17, 18, 19, 20, 21, or 22 consecutive sequences of a nucleic acid sequence selected from SEQ ID NOs: 1-154, with no more than 1, 2, 3, or 4 mismatches. In some cases, the sense strand comprises a nucleic acid sequence comprising at least 14, 15, 16, 17, 18, 19, or 20 consecutive sequences from a nucleic acid sequence selected from SEQ ID NOs: 309-462, with no more than 1, 2, 3, or 4 mismatches. In some cases, the sense strand comprises one of SEQ ID NOs: 309-462 and the antisense strand comprises a nucleic acid sequence that is selected from SEQ ID NOs: 1-154

In some cases, the sense strand comprises at least two consecutive modified internucleotide linkages at the 5′ end. In some cases, the antisense strand comprises at least two consecutive modified internucleotide linkages at the 5′ end and/or 3′ end. In some cases, the modified internucleotide linkage is a phosphorothioate linkage. In some cases, the modified internucleotide linkage comprises a stereochemically enriched phosphorothioate internucleotide linkage. In some cases, the modified internucleotide linkage is an S_{P} chiral internucleotide phosphorothioate linkage. In some cases, the polynucleic acid comprises a plurality of modified internucleotide linkages, and at least 1, 2, 3, or 4 of the plurality of modified internucleotide linkages are stereochemically enriched phosphorothioate internucleotide linkages. In some cases, at least one stereochemically enriched phosphorothioate is disposed between two consecutive nucleosides that are two of six 5′ or 3′-terminal nucleosides of the sense strand or the antisense strand. In some cases, the sense strand comprises 5′-nnnnnnNfnNfnNfnnnnnnnnnn-3′, wherein the antisense strand comprises 5′-nNfnnnnNfnnnnNfnNfnNfnnnnnnn-3′, wherein n stands for a 2′-O-methyl modified nucleotide, and wherein Nf stands for a 2′-fluoro modified nucleotide.

In some cases, the sense strand or antisense strand is about 19-25, or about 21-23 nucleotides in length.

In some cases, the sense strand comprises a nucleic acid sequence that is at least 80%, at least 85%, at least 90%, at least 95% identical to a nucleic acid sequence selected from SEQ ID NOs: 463-616. In some cases, the antisense strand comprises a nucleic acid sequence that is at least 80%, at least 85%, at least 90%, at least 95% identical to a nucleic acid sequence selected from SEQ ID NOs: 155-308. In some cases, the sense strand comprises a sequence selected from a nucleic acid sequence of SEQ ID NOs: 463-616, and the antisense strand comprises a sequence selected from a nucleic acid sequence of SEQ ID NOs: 155-308.

In another aspect, provided herein is polynucleic acid molecule for modulating expression of Lipoprotein(a) (Lp(a)) gene, wherein polynucleic acid molecule comprises:
(a) an antisense strand comprising the nucleotide sequence of UAGAUGACCAAGCUUGGCAGGUC (SEQ ID NO: 4) and a sense strand comprising the nucleotide sequence of CCUGCCAAGCUUGGUCAUCUA (SEQ ID NO: 312); (b) an antisense strand comprising the nucleotide sequence of UAUAGAUGACCAAGCUUGGCAGG (SEQ ID NO: 5) and a sense strand comprising the nucleotide sequence of UGCCAAGCUUGGUCAUCUAUA (SEQ ID NO:313); (c) an antisense strand comprising the nucleotide sequence of UCAUAGAUGACCAAGCUUGGCAG (SEQ ID NO: 6) and a sense strand comprising the nucleotide sequence of GCCAAGCUUGGUCAUCUAUGA (SEQ ID NO: 314); (d) an antisense strand comprising the nucleotide sequence of UCGACGGCAGUCCCUUCUGCGUC (SEQ ID NO: 11) and a sense strand comprising the nucleotide sequence of CGCAGAAGGGACUGCCGUCGA (SEQ ID NO: 319); (e) an antisense strand comprising the nucleotide sequence of UUCUAGGCUUGGAACCGGGGUAA (SEQ ID NO: 18) and a sense strand comprising the nucleotide sequence of ACCCCGGUUCCAAGCCUAGAA (SEQ ID NO: 326); (f) an antisense strand comprising the nucleotide sequence of UAGCCUCUAGGCUUGGAACCGGG (SEQ ID NO: 21) and a sense strand comprising the nucleotide sequence of CGGUUCCAAGCCUAGAGGCUA (SEQ ID NO: 329); (g) an antisense strand comprising the nucleotide sequence of UUUACCGUGGUAGCACUCCUGCA (SEQ ID NO: 44) and a sense strand comprising the nucleotide sequence of CAGGAGUGCUACCACGGUAAA (SEQ ID NO: 352): (h) an antisense strand comprising the nucleotide sequence of UUGUCCAUUACCGUGGUAGCACU (SEQ ID NO: 47) and a sense strand comprising the nucleotide sequence of UGCUACCACGGUAAUGGACAA (SEQ ID NO: 355); (i) an antisense strand comprising the nucleotide sequence of UCUCUGUCCAUUACCGUGGUAGC (SEQ ID NO: 49) and a sense strand comprising the nucleotide sequence of UACCACGGUAAUGGACAGAGA (SEQ ID NO: 357); (j) an antisense strand comprising the nucleotide sequence of UAUUGUGUCAGGUUGCAGUACUC (SEQ ID NO: 60) and a sense strand comprising the nucleotide sequence of GUACUGCAACCUGACACAAUA (SEQ ID NO: 368); (k) an antisense strand comprising the nucleotide sequence of UUGCGUCUGAGCAUUGUGUCAGG (SEQ ID NO: 64) and a sense strand comprising the nucleotide sequence of UGACACAAUGCUCAGACGCAA (SEQ ID NO: 372). (l) an antisense strand comprising the nucleotide sequence of UUAACUCUGUCCAUAAUGGUAGU (SEQ ID NO: 88) and a sense strand comprising the nucleotide sequence of UACCAUUAUGGACAGAGUUAA (SEQ ID NO: 396); (m) an antisense strand comprising the nucleotide sequence of UCCAAGCUUGGCAAGUUCUUCCU (SEQ ID NO: 89) and a sense strand comprising the nucleotide sequence of GAAGAACUUGCCAAGCUUGGA (SEQ ID NO: 397); (n) an antisense strand comprising the nucleotide sequence of UAGAUGACCAAGCUUGGCAAGUU (SEQ ID NO: 90) and a sense strand comprising the nucleotide sequence of CUUGCCAAGCUUGGUCAUCUA (SEQ ID NO: 398); or (o) an antisense strand comprising the nucleotide sequence of UGGUCCGACUAUGCUGGUGUGGU (SEQ ID NO: 98) and a sense strand comprising the nucleotide sequence of CACACCAGCAUAGUCGGACCA (SEQ ID NO: 406).

In another aspect, provided herein is a polynucleic acid molecule for modulating expression of Lipoprotein(a) (Lp(a)) gene, wherein polynucleic acid molecule comprises: (a) an antisense strand comprising the nucleotide sequence of usAfsgaugAfccaaGfcUfuGfgcaggsusc (SEQ ID NO: 158) and a sense strand comprising the nucleotide sequence of cscsugccAfaGfcUfuggucaucua (SEQ ID NO: 466); (b) an antisense strand comprising the nucleotide sequence of usAfsuagaUfgaccAfaGfcUfuggcasgsg (SEQ ID NO: 159) and a sense strand comprising the nucleotide sequence of usgsccaaGfcUfuGfgucaucuaua (SEQ ID NO: 467); (c) an antisense strand comprising the nucleotide sequence of usCfsauagAfugacCfaAfgCfuuggcsasg (SEQ ID NO: 160) and a sense strand comprising the nucleotide sequence of gscscaagCfuUfgGfucaucuauga (SEQ ID NO: 468); (d) an antisense strand comprising the nucleotide sequence of usCfsgacgGfcaguCfcCfuUfcugcgsusc (SEQ ID NO: 165) and a sense strand comprising the nucleotide sequence of csgscagaAfgGfgAfcugccgucga (SEQ ID NO: 473); (e) an antisense strand comprising the nucleotide sequence of usUfscuagGfcuugGfaAfcCfggggusasa (SEQ ID NO: 172) and a sense strand comprising the nucleotide sequence of ascscccgGfuUfcCfaagccuagaa (SEQ ID NO: 480); (f) an antisense strand comprising the nucleotide sequence of usAfsgccuCfuaggCfuUfgGfaaccgsgsg (SEQ ID NO: 175) and a sense strand comprising the nucleotide sequence of csgsguucCfaAfgCfcuagaggcua (SEQ ID NO: 483); (g) an antisense strand comprising the nucleotide sequence of usUfsuaccGfugguAfgCfaCfuccugscsa (SEQ ID NO: 198) and a sense strand comprising the nucleotide sequence of csasggagUfgCfuAfccacgguaaa (SEQ ID NO: 506); (h) an antisense strand comprising the nucleotide sequence of usUfsguccAfuuacCfgUfgGfuagcascsu (SEQ ID NO: 201) and a sense strand comprising the nucleotide sequence of usgscuacCfaCfgGfuaauggacaa (SEQ ID NO: 509); (i) an antisense strand comprising the nucleotide sequence of usCfsucugUfccauUfaCfcGfugguasgsc (SEQ ID NO: 203) and a sense strand comprising the nucleotide sequence of usasccacGfgUfaAfuggacagaga (SEQ ID NO: 511); (j) an antisense strand comprising the nucleotide sequence of usAfsuuguGfucagGfuUfgCfaguacsusc (SEQ ID NO: 214) and a sense strand comprising the nucleotide sequence of gsusacugCfaAfcCfugacacaaua (SEQ ID NO: 522); (k) an antisense strand comprising the nucleotide sequence of usUfsgcguCfugagCfaUfuGfugucasgsg (SEQ ID NO: 218) and a sense strand comprising the nucleotide sequence of usgsacacAfaUfgCfucagacgcaa (SEQ ID NO: 526); (l) an antisense strand comprising the nucleotide sequence of usUfsaacuCfugucCfaUfaAfugguasgsu (SEQ ID NO: 242) and a sense strand comprising the nucleotide sequence of usasccauUfaUfgGfacagaguuaa (SEQ ID NO: 550); (m) an antisense strand comprising the nucleotide sequence of usCfscaagCfuuggCfaAfgUfucuucscsu (SEQ ID NO: 243) and a sense strand comprising the nucleotide sequence of gsasagaaCfuUfgCfcaagcuugga (SEQ ID NO: 551); (n) an antisense strand comprising the nucleotide sequence of usAfsgaugAfccaaGfcUfuGfgcaagsusu (SEQ ID NO: 244) and a sense strand comprising the nucleotide sequence of csusugccAfaGfcUfuggucaucua (SEQ ID NO: 552); or (o) an antisense strand comprising the nucleotide sequence of usGfsguccGfacuaUfgCfuGfgugugsgsu (SEQ ID NO: 252) and a sense strand comprising the nucleotide sequence of csascaccAfgCfaUfagucggacca (SEQ ID NO: 560),
wherein “A” refers to adenosine-3′-phosphate; “a” refers to 2′-O-methyladenosine-3′-phosphate; “Af” refers to 2′-fluoroadenosine-3′-phosphate; “C” refers to cytidine-3′-phosphate; “c” refers to 2′-O-methylcytidine-3′-phosphate; “Cf” refers to 2′-fluorocytidine-3′-phosphate; “G” refers to guanosine-3′-phosphate; “g” refers to 2′-O-methylguanosine-3′-phosphate; “Gf” refers to 2′-fluoroguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “s” refers to 3′-phosphorothioate.

In another aspect, provided herein is a polynucleic acid molecule conjugate for modulating expression of lipoprotein(a) gene (Lp(a)), wherein the polynucleic acid molecule conjugate comprises a polynucleic acid molecule described herein and an asialoglycoprotein receptor targeting moiety. In some cases, the asialoglycoprotein receptor targeting moiety comprises N-Acetylgalactosamine (GalNAc) or galactose. In some cases, the GalNAc comprises an anomeric carbon bonded to trivalent, tetravalent linker, pentavalent, or hexavalent linker, wherein the anomeric carbon is part of a hemiaminal group.

In some cases, the polynucleic acid molecule and the asialoglycoprotein receptor targeting moiety is coupled via a linker. In some cases, the linker is a cleavable linker. In some cases, the linker comprises formula (IV) below,

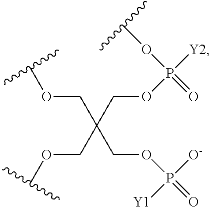

wherein at least one of Y1 and Y2 is a nucleotide in the polynucleic acid molecule. In some cases, the Y1 is the last nucleotide on the 3′-terminus of the sense strand of the polynucleic acid molecule.

In some cases, the linker and the asialoglycoprotein receptor targeting moiety with the last nucleotide on the 3′-terminus of the sense strand of the polynucleic acid molecule are shown in Formula (V′), Formula (V″″), Formula (V′″″), or Formula (V″″″):

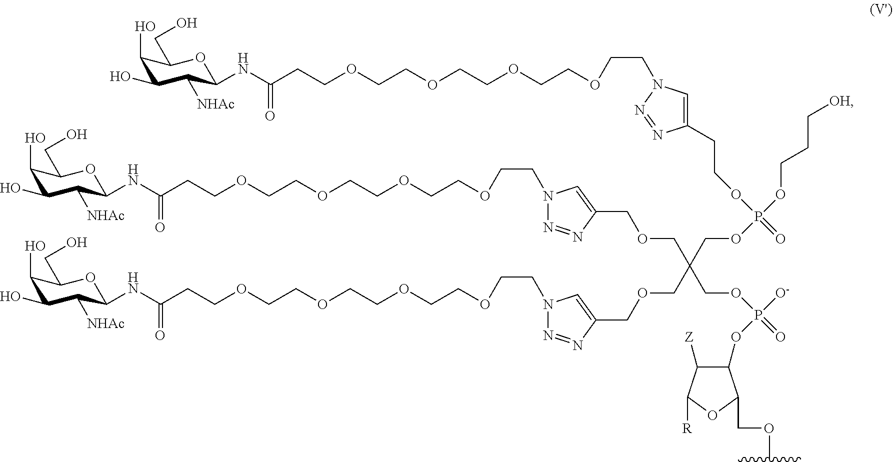

wherein Z in formula (V′) is —H, —OH, —O-Methyl, —F, or —O-methoxyethyl and R in formula (V′) is adenine, uracil, guanine, cytosine, thymine, abasic, or others;

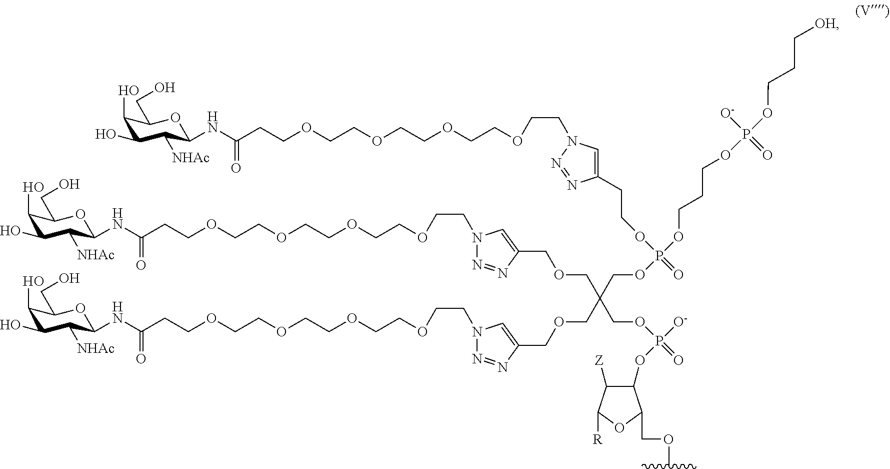

wherein Z in formula (V″″) is a moiety that corresponds to one of the sugar modifications described herein (e.g., —H, —OH, —O-Methyl, —F, or —O-methoxyethyl) and R in formula (V″″) is adenine, uracil, guanine, cytosine, thymine, abasic, or others.

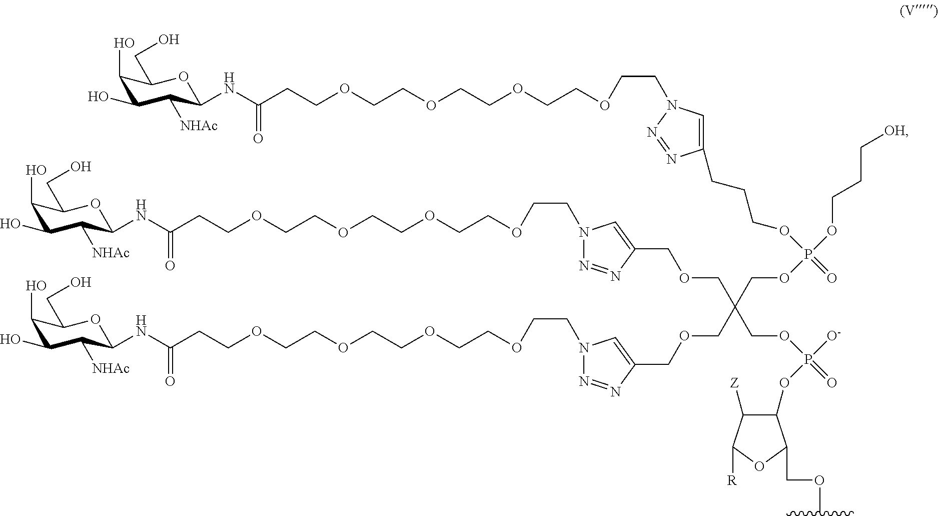

wherein Z in formula (V′″″) is a moiety that corresponds to one of the sugar modifications described herein (e.g., —H, —OH, —O-Methyl, —F, or —O-methoxyethyl) and R in formula (V′″″) is adenine, uracil, guanine, cytosine, thymine, abasic, or others; or

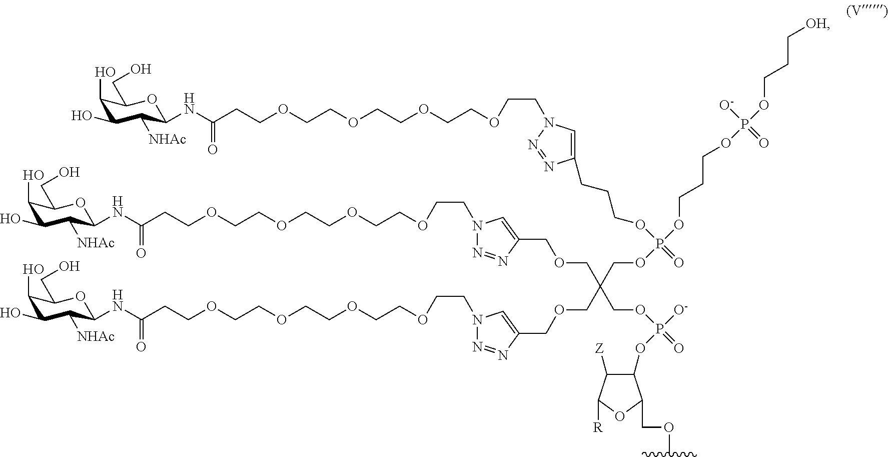

wherein Z in formula (V″″″) is a moiety that corresponds to one of the sugar modifications described herein (e.g., —H, —OH, —O-Methyl, —F, or —O-methoxyethyl) and R in formula (V″″″) is adenine, uracil, guanine, cytosine, thymine, abasic, or others.

In some cases, the linker and the asialoglycoprotein receptor targeting moiety with the last nucleotide on the 3′-terminus of the sense strand of the polynucleic acid molecule are shown in Formula (V′):

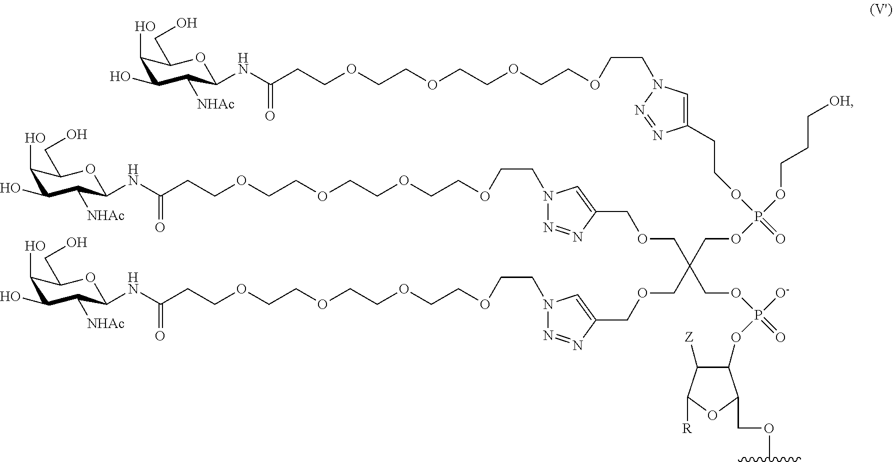

wherein Z in formula (V′) is —H, —OH, —O-Methyl, —F, or —O-methoxyethyl and R in formula (V′) is adenine, uracil, guanine, cytosine, thymine, abasic, or others.

In another aspect, provided herein is a pharmaceutical composition comprising a polynucleic acid molecule described herein or a polynucleic acid molecule conjugate described herein, and a pharmaceutically acceptable excipient. In some cases, the pharmaceutical composition is formulated for parenteral, oral, intranasal, buccal, rectal, transdermal, intravenous, subcutaneous, or intrathecal administration.

In another aspect, provided herein is method of modulating expression of lipoprotein(a) (Lp(a)) gene in a subject in need thereof, comprising: administering to the subject a polynucleic acid molecule described herein, a polynucleic acid molecule conjugate described herein, or a pharmaceutical composition described herein, thereby modulating the expression of Lp(a) gene in the subject in need thereof.

In another aspect, provided herein is a method for treating or preventing a cardiovascular disease or a lipid disorder, comprising: administering to the subject a polynucleic acid molecule described herein, a polynucleic acid molecule conjugate described herein, or a pharmaceutical composition described herein, thereby modulating the expression of Lp(a) gene in the subject in need thereof. In some cases, the cardiovascular disease is coronary artery disease, acute myocardial infarction, asymptomatic carotid atherosclerosis, stroke, atrial fibrillation, hypercholesterolemia, or peripheral artery occlusive disease. In some cases, the lipid disorder is hyperlipidemia or hypercholesterolemia.

In some cases, the polynucleic acid molecule is administered at a dose sufficient to decrease the expression of Lp(a) gene in a cell of said subject or to decrease plasma Lp(a) level of said subject by at least about 10%, 20%, 30%, 40%, 50%, 60%, 70%, 80%, or 90% compared to a control.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative aspects, in which the principles of the disclosure are utilized, and the accompanying drawings below.

FIGS. 1A-1VV show dose responses of siRNA compositions on Lp(a) expression levels in primary human hepatocytes (PHH).

FIG. 2 shows % change in serum Lp(a) level from base line (Day −1) in cynomolgus monkeys.

FIG. 3 shows % change in serum Lp(a) level from base line (Day −1) in cynomolgus monkeys.

### DETAILED DESCRIPTION OF THE DISCLOSURE

Lp(a) gene (LPA) is located on chromosome 6q26-q27 is the major gene locus for Lp(a) concentrations in all populations (F. Kronenberg et al., J Intern Med, 273 (1) (2013)). LPA gene is one of the strongest monogenic risk factors for CVD (S. Tsimikas, J Am Coll Cardiol, 69 (6) (2017)). The LPA gene is highly expressed in liver with dramatic decrease as well as increase in Lp(a) observed after liver transplant (A. A. Damluji et al., Journal of clinical lipidology, 10 (2) (2016)).

Lipoprotein (a)(Lp(a)) is a low-density lipoprotein-like particle formed by the association of apolipoprotein (a) (apo(a)) with apolipoprotein B (apo B). The apo(a) protein is covalently linked to apo B in the assembled Lp(a) particle via a disulfide bond. Lp(a) carries atherosclerosis-causing cholesterol and binds atherogenic pro-inflammatory oxidized phospholipids as a preferential carrier of oxidized phospholipids in human plasma, which facilitate inflammation reactions.

The plasma level of Lp(a) is primarily determined by the LPA gene encoding apo(a) (S. Tsimikas, J Am Coll Cardiol, 69 (6) (2017). The Lp(a) level varies between individuals and is directly proportional to CVD risk. Elevated plasma levels of Lp(a) are associated with increased risk for atherosclerosis and its manifestations, which may include hypercholesterolemia (Seed et al., N. Engl. J. Med., 1990, 322, 1494-1499), myocardial infarction (Sandkamp et al., Clin. Chem., 1990, 36, 20-23), and thrombosis (Nowak-Gottl et al., Pediatrics, 1997, 99, E11). Therefore, inhibition of LPA is viewed as a potential therapeutic strategy to treat CVD.

Described herein is a polynucleic acid molecule for modulating expression of Lp(a) gene, comprising a nucleic acid sequence selected from Table 1 or Table 2. In some instances, the polynucleic acid molecule is a single-stranded nucleic acid molecule. In some instances, the polynucleic acid molecule is a double-stranded nucleic acid molecule comprising a sense strand and an antisense strand. Accordingly, provided herein are various target regions of human Lp(a) mRNA the polynucleic acid molecule described herein hybridizes to. The polynucleic acid molecule described herein can be a modified polynucleic acid molecule. For example, the modified polynucleic acid molecule can comprise a 2′-fluoro modified nucleotide, a 2′-O-methyl modified nucleotide, or a modified internucleotide linkage such as a phosphorothioate linkage. In some aspects, provided herein is the polynucleic acid conjugated with a targeting moiety described herein.

Also described herein is a method of modulating expression of Lp(a) mRNA or protein in a subject. Described further herein is a method of modulating LDL and/or cholesterol in a subject in need thereof.

### Definition

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

“Percent (%) sequence identity” or “Percent (%) identity” with respect to the nucleic acid sequences identified herein is defined as the percentage of nucleic acid in a candidate sequence that are identical with the nucleic acid sequence being compared, after aligning the sequences considering any conservative substitutions as part of the sequence identity.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and the like. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Unless defined otherwise, all technical and scientific terms used herein have the same meaning as commonly understood by one of ordinary skill in the art to which the polynucleic acid molecules, the polynucleic acid molecule conjugates, the pharmaceutical compositions, the methods and other aspects belong.

As used herein, the term “complementary” indicates a sufficient degree of complementarity between two nucleic acid molecules that bind stably and specifically to avoid nonspecific binding.

As used herein, the term “polynucleic acid” and the term “polynucleotide” are interchangeably used to refer a chain of nucleotides. The term “nucleotide” includes a sequence “G,” “C,” “A,” “T” and “U” each generally stand for a nucleotide that contains guanine, cytosine, adenine, thymidine and uracil as a base. In some instances, the “nucleotide” can refer to a modified nucleotide (e.g., with modified sugar moiety, modified base, modified internucleotide linkage, or combination thereof, including, but not limited to 2′-modified nucleotide, LNA, ENA, BNA, UNA, GNA etc.) In some instances, the “nucleotide” can refer to a modified nucleotide with a non-canonical base (e.g. including, but not limited to, 2-thiouridine, 2-thiothymidine, inosine, 2-aminopurine, 2,6-diaminopurine, dihydrouridine, 4-thiouridine, 4-thiothymidine, 2-thiocytidine).

As used herein, a “subject” can be any mammal, including a human and a non-human primate.

A “subject in need thereof”, refers to a subject having a Lp(a) related disorder or symptoms thereof, including but not limited to, CVD or a lipid disorder, or a subject having an increased risk of developing Lp(a) related disorder or symptoms thereof, including but not limited to, a CVD or lipid disorder relative to the population at large. In some instances, a subject in need thereof has coronary heart disease or atherosclerosis, or symptoms thereof. In some instances, a subject in need thereof has hyperlipidemia or hypercholesterolemia, or symptoms thereof. In some instances, a subject in need thereof is being administered or has been administered a drug different from the polynucleic acid molecules or conjugates disclosed herein, for treating or preventing a CVD or lipid disorder. For example, a subject in need thereof is being administered or has been administered atorvastatin.

The term “condition,” as used herein, includes diseases, disorders, and susceptibilities. In some cases, the condition is an Lp(a) related disorder, atherosclerotic vascular disease, or symptoms thereof. In some cases, the condition is a hypertriglyceridemia or symptoms thereof.

The term “atherosclerosis” or “atherosclerotic vascular disease,” as used herein, refers to a disease in which the inside of an artery narrows due to the buildup of plaque. In some instances, it may result in coronary artery disease, stroke, peripheral artery disease, or kidney problems.

The term “cardiovascular disease” or “CVD” refers to all types of diseases that affect the heart or blood vessels, including coronary heart disease (clogged arteries), which can cause heart attacks, stroke, congenital heart defects and peripheral artery disease. A CVD or symptoms thereof can include myocardial infarction, stroke, atrial fibrillation, or calcific aortic valve stenosis. A CVD or symptoms thereof include cardiac arrest or peripheral arterial disease.

The term “lipid disorder” refers to a disorder or a condition that increase levels of LDLs, triglycerides, or both. For example, the lipid disorder can be hyperlipidemia or hypercholesterolemia.

The term “low-density lipoprotein (LDL),” as used herein, refers to a microscopic blob made up of an outer rim of lipoprotein and a cholesterol center. LDL can have a highly hydrophobic core composed of a polyunsaturated fatty acid known as linoleate and hundreds to thousands esterified and unesterified cholesterol molecules. The core of LDL can also carry triglycerides and other fats and can be surrounded by a shell of phospholipids and unesterified cholesterol.

As used herein, the term “treat,” “treating” or “treatment” of any disease or disorder refers, in one instance, to ameliorating the disease or disorder (i.e., slowing or arresting or reducing the development of the disease or at least one of the clinical symptoms thereof). In another instance, “treat”, “treating” or “treatment” refers to alleviating or ameliorating at least one physical parameter including those which may not be discernible by the patient. In yet another instance, “treat”, “treating” or “treatment” refers to modulating the disease or disorder, either physically, (e.g., stabilization of a discernible symptom), physiologically, (e.g., stabilization of a physical parameter), or both.

The terms “prevent,” “preventing,” and “prevention,” as used herein, refer to a decrease in the occurrence of pathology of a condition in a subject, who does not have, but is at risk of or susceptible to developing a disease or condition. The prevention may be complete, e.g., the total absence of pathology of a condition in a subject. The prevention may also be partial, such that the occurrence of pathology of a condition in a subject is less than that which would have occurred without the present disclosure.

“Administering” and its grammatical equivalents as used herein can refer to providing pharmaceutical compositions described herein to a subject or a patient. Conventional methods, known to those of ordinary skill in the art of medicine, can be used to administer the composition to the subject, depending upon the type of disease to be treated or the site of the disease. For example, the composition can be administered, e.g., orally, parenterally, by inhalation spray, topically, rectally, nasally, buccally, vaginally, via an implanted reservoir, or via infusion. One or more such routes can be employed.

The terms “pharmaceutical composition” and its grammatical equivalents as used herein can refer to a mixture or solution comprising a therapeutically effective amount of an active pharmaceutical ingredient together with one or more pharmaceutically acceptable excipients, carriers, and/or a therapeutic agent to be administered to a subject, e.g., a human in need thereof.

The term “pharmaceutically acceptable” and its grammatical equivalents as used herein can refer to an attribute of a material which is useful in preparing a pharmaceutical composition that is generally safe, non-toxic, and neither biologically nor otherwise undesirable and is acceptable for veterinary as well as human pharmaceutical use. “Pharmaceutically acceptable” can refer a material, such as a carrier or diluent, which does not abrogate the biological activity or properties of the compound, and is relatively nontoxic, i.e., the material may be administered to a subject without causing undesirable biological effects or interacting in a deleterious manner with any of the components of the pharmaceutical composition in which it is contained.

A “pharmaceutically acceptable excipient” refers to an excipient that can be administered to a subject, together with an agent, and which does not destroy the pharmacological activity thereof and is nontoxic when administered in doses sufficient to deliver a therapeutic amount of the agent.

The term “therapeutic agent” can refer to any agent that, when administered to a subject, has a therapeutic, diagnostic, and/or prophylactic effect and/or elicits a desired biological and/or pharmacological effect. Therapeutic agents can also be referred to as “actives” or “active agents.” Such agents include, but are not limited to, cytotoxins, radioactive ions, chemotherapeutic agents, small molecule drugs, proteins, and nucleic acids.

It is appreciated that certain features of the polynucleic acid molecules, and/or polynucleic acid molecule conjugates, pharmaceutical composition comprising the polynucleic acid molecules or the polynucleic acid molecule conjugates, methods and other aspects, which are, for clarity, described in the context of separate embodiments, may also be provided in combination in a single embodiment. Conversely, various features of the polynucleic acid molecules, and/or polynucleic acid molecule conjugates, pharmaceutical composition comprising the polynucleic acid molecules or the polynucleic acid molecule conjugates, methods and other aspects, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present polynucleic acid molecules, and/or polynucleic acid molecule conjugates, pharmaceutical composition comprising the polynucleic acid molecules or the polynucleic acid molecule conjugates, methods and other aspects and are disclosed herein just as if each and every such sub-combination was individually and explicitly disclosed herein.

### Polynucleic Acid Molecules

### Target Regions of Polynucleic Acid Molecules

Described herein is a polynucleic acid molecule for modulating expression of Lp(a) gene. In some instances, the polynucleic acid molecule is a single-stranded nucleic acid molecule. In some instances, the polynucleic acid molecule is a double-stranded nucleic acid molecule.

In some aspects, the polynucleic acid molecule described herein hybridizes to certain regions of human Lp(a) mRNA. In some instances, the polynucleic acid molecule comprises a sense strand and an antisense strand, and wherein the antisense strand hybridizes to a certain region of Lp(a) mRNA. As used herein, the term “sense strand” can be interchangeably used with the term “passenger strand”, and the term “antisense strand” can be interchangeably used with the term “guide strand”.

In some aspects, the polynucleic acid molecule described herein hybridizes to the 5′ UTR region of human Lp(a) mRNA. In some aspects, the polynucleic acid molecule described herein hybridizes to the coding region of human Lp(a) mRNA. In some aspects, the polynucleic acid molecule described herein hybridizes to the 3′ UTR region of human Lp(a) mRNA. In some aspects, the polynucleic acid molecule described herein hybridizes to a portion of exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 of human Lp(a) mRNA. In some aspects, the polynucleic acid molecule described herein hybridizes to a coding region of human Lp(a) m RNA (NCBI Reference Sequence: NM_005577.4).

In some aspects, the starting position of the binding site of the polynucleic acid molecule described herein on human Lp(a) mRNA (NCBI Reference Sequence: NM_005577.4) is between position 190-210, 250-270; 360-390; 400-450; 460-520, 530-550, 1100-1200, 2600-2700, 2700-2800,2800-2900, 3000-3300; 3300-3600, 3600-4000, 40004200, 4400-4700, 4700-5000, 5000-5200, or 5500-6000.

The starting position of the binding site of the polynucleic acid molecule described herein on human Lp(a) mRNA (NCBI Reference Sequence: NM_005577.4) can be between position 200-210, 380-390, 410-420, 1100-1200, 2700-2800, 3200-3300, or 3300-3400.

The starting position of the binding site of the polynucleic acid molecule described herein on human Lp(a) mRNA (NCBI Reference Sequence: NM_005577.4) can be between position 200-210,250-260, 380-390, 410-430, 430-440, 460-470, 490-500, 500-510, 540-550, 1150-1180, 2650-2700, 2700-2750, 2760-2770, 2850-2900, 3200-3250, 3250-3300, 3300-3310, 3700-3750, 3750-3800, 3950-4000, 4800-4850, 5100-5150, 5500-5550, 5850-5900, or 2750-2800.

### Structure of Polynucleic Acid Molecules

Described herein is a polynucleic acid molecule for modulating expression of Lp(a) gene. In some instances, the polynucleic acid molecule is a single-stranded nucleic acid molecule, wherein the single-stranded nucleic acid molecule is reverse complementary to a target region of Lp(a) mRNA.

In some aspects, the polynucleic acid molecule described herein is not 100% complementary to the target region of Lp(a) mRNA. Accordingly, in some instances, the polynucleic acid molecule described herein is about 95% complementary to the target region of Lp(a) mRNA. In some instances, the polynucleic acid molecule described herein is about 90% complementary to the target region of Lp(a) mRNA. In some instances, the polynucleic acid molecule described herein is about 85% complementary to the target region of Lp(a) mRNA. In some instances, the polynucleic acid molecule described herein is about 80% complementary to the target region of Lp(a) mRNA. In some instances, the polynucleic acid molecule described herein is about 75% complementary to the target region of Lp(a) mRNA. In some instances, the polynucleic acid molecule described herein is about 70% complementary to the target region of Lp(a) mRNA.

In some aspects, the polynucleic acid molecule described herein comprises a nucleic acid sequence in Table 1, Table 2 or Table 5. In some instances, the polynucleic acid molecule described herein comprises a nucleic acid sequence that is at least 80%, at least 85%, at least 90%, or at least 95% complementary to a sequence in Table 1, Table 2, or Table 5. In some instances, the polynucleic acid molecule described herein comprises a nucleic acid sequence that is at least 80%, at least 85%, at least 90%, or at least 95% complementary to a nucleic acid sequence selected from SEQ ID NOs: 309-462. In some instances, the polynucleic acid molecule described herein comprises a nucleic acid sequence that is at least 80%, at least 85%, at least 90%, or at least 95% complementary to a nucleic acid sequence selected from SEQ ID NOs: 1-154.

In some instances, the polynucleic acid molecule described herein comprises a nucleic acid sequence that is at least 80%, at least 85%, at least 90%, or at least 95% complementary to a sequence in Table 1, Table 2, and Table 5, excluding overhangs. In some instances, the polynucleic acid molecule described herein comprises a nucleic acid sequence that is at least 80%, at least 85%, at least 90%, or at least 95% complementary to a nucleic acid sequence selected from SEQ ID NOs: 309-462, excluding overhangs. In some instances, the polynucleic acid molecule described herein comprises a nucleic acid sequence that is at least 80%, at least 85%, at least 90%, or at least 95% complementary to a nucleic acid sequence selected from SEQ ID NOs: 1-154, excluding overhangs.

In yet other aspects, the polynucleic acid molecule described herein comprises a nucleic acid sequence comprising 14 consecutive nucleotides that are complementary to a sequence in Table 1, Table 2, and Table 5 with no more than 1, 2, 3, or 4 mismatches. In some aspects, the polynucleic acid molecule described herein comprises a nucleic acid sequence comprising 14 consecutive nucleotides that are complementary to a nucleic acid sequence of SEQ ID NO: 309-462 with no more than 1, 2, 3, or 4 mismatches. In yet other aspects, the polynucleic acid molecule described herein comprises a nucleic acid sequence comprising 15 consecutive nucleotides that are complementary to a sequence in Table 1, Table 2, and Table 5 with no more than 1, 2, 3, or 4 mismatches. In some aspects, the polynucleic acid molecule described herein comprises a nucleic acid sequence comprising 15 consecutive nucleotides that are complementary to a nucleic acid sequence of SEQ ID NO: 309-462 with no more than 1, 2, 3, or 4 mismatches. In yet other aspects, the polynucleic acid molecule described herein comprises a nucleic acid sequence comprising 16 consecutive nucleotides that are complementary to a sequence in Table 1, Table 2, and Table 5 with no more than 1, 2, 3, or 4 mismatches. In some aspects, the polynucleic acid molecule described herein comprises a nucleic acid sequence comprising 16 consecutive nucleotides that are complementary to a nucleic acid sequence of SEQ ID NO: 309-462 with no more than 1, 2, 3, or 4 mismatches. In yet other aspects, the polynucleic acid molecule described herein comprises a nucleic acid sequence comprising 17 consecutive nucleotides that are complementary to a sequence in Table 1, Table 2, and Table 5 with no more than 1, 2, 3, or 4 mismatches. In some aspects, the polynucleic acid molecule described herein comprises a nucleic acid sequence comprising 17 consecutive nucleotides that are complementary to a nucleic acid sequence of SEQ ID NO: 309-462 with no more than 1, 2, 3, or 4 mismatches. In yet other aspects, the polynucleic acid molecule described herein comprises a nucleic acid sequence comprising 18 consecutive nucleotides that are complementary to a sequence in Table 1, Table 2, and Table 5 with no more than 1, 2, 3, or 4 mismatches. In some aspects, the polynucleic acid molecule described herein comprises a nucleic acid sequence comprising 18 consecutive nucleotides that are complementary to a nucleic acid sequence of SEQ ID NO: 309-462 with no more than 1, 2, 3, or 4 mismatches. In yet other aspects, the polynucleic acid molecule described herein comprises a nucleic acid sequence comprising 19 consecutive nucleotides that are complementary to a sequence in Table 1, Table 2, and Table 5 with no more than 1, 2, 3, or 4 mismatches. In some aspects, the polynucleic acid molecule described herein comprises a nucleic acid sequence comprising 19 consecutive nucleotides that are complementary to a nucleic acid sequence of SEQ ID NO: 309-462 with no more than 1, 2, 3, or 4 mismatches. In yet other aspects, the polynucleic acid molecule described herein comprises a nucleic acid sequence comprising 20 consecutive nucleotides that are complementary to a sequence in Table 1, Table 2, and Table 5 with no more than 1, 2, 3, or 4 mismatches. In some aspects, the polynucleic acid molecule described herein comprises a nucleic acid sequence comprising 20 consecutive nucleotides that are complementary to a nucleic acid sequence of SEQ ID NO: 309-462 with no more than 1, 2, 3, or 4 mismatches. In yet other aspects, the polynucleic acid molecule described herein comprises a nucleic acid sequence comprising 21 consecutive nucleotides that are complementary to a sequence in Table 1, Table 2, and Table 5 with no more than 1, 2, 3, or 4 mismatches. In some aspects, the polynucleic acid molecule described herein comprises a nucleic acid sequence comprising 21 consecutive nucleotides that are complementary to a nucleic acid sequence of SEQ ID NO: 309-462 with no more than 1, 2, 3, or 4 mismatches. In yet other aspects, the polynucleic acid molecule described herein comprises a nucleic acid sequence comprising 22 consecutive nucleotides that are complementary to a sequence in Table 1, Table 2, and Table 5 with no more than 1, 2, 3, or 4 mismatches. In some aspects, the polynucleic acid molecule described herein comprises a nucleic acid sequence comprising 22 consecutive nucleotides that are complementary to a nucleic acid sequence of SEQ ID NO: 309-462 with no more than 1, 2, 3, or 4 mismatches.

In some aspects, the polynucleic acid molecule described herein comprises a nucleic acid sequence comprising 14 consecutive nucleotides that are complementary to a nucleic acid sequence of SEQ ID NO: 1-154 with no more than 1, 2, 3, or 4 mismatches. In some aspects, the polynucleic acid molecule described herein comprises a nucleic acid sequence comprising 15 consecutive nucleotides that are complementary to a nucleic acid sequence of SEQ ID NO: 1-154 with no more than 1, 2, 3, or 4 mismatches. In some aspects, the polynucleic acid molecule described herein comprises a nucleic acid sequence comprising 16 consecutive nucleotides that are complementary to a nucleic acid sequence of SEQ ID NO: 1-154 with no more than 1, 2, 3, or 4 mismatches. In some aspects, the polynucleic acid molecule described herein comprises a nucleic acid sequence comprising 17 consecutive nucleotides that are complementary to a nucleic acid sequence of SEQ ID NO: 1-154 with no more than 1, 2, 3, or 4 mismatches. In some aspects, the polynucleic acid molecule described herein comprises a nucleic acid sequence comprising 18 consecutive nucleotides that are complementary to a nucleic acid sequence of SEQ ID NO: 1-154 with no more than 1, 2, 3, or 4 mismatches. In some aspects, the polynucleic acid molecule described herein comprises a nucleic acid sequence comprising 19 consecutive nucleotides that are complementary to a nucleic acid sequence of SEQ ID NO: 1-154 with no more than 1, 2, 3, or 4 mismatches. In some aspects, the polynucleic acid molecule described herein comprises a nucleic acid sequence comprising 20 consecutive nucleotides that are complementary to a nucleic acid sequence of SEQ ID NO: 1-154 with no more than 1, 2, 3, or 4 mismatches. In some aspects, the polynucleic acid molecule described herein comprises a nucleic acid sequence comprising 21 consecutive nucleotides that are complementary to a nucleic acid sequence of SEQ ID NO: 1-154 with no more than 1, 2, 3, or 4 mismatches. In some aspects, the polynucleic acid molecule described herein comprises a nucleic acid sequence comprising 22 consecutive nucleotides that are complementary to a nucleic acid sequence of SEQ ID NO: 1-154 with no more than 1, 2, 3, or 4 mismatches.

In yet other aspects, the polynucleic acid molecule described herein comprises a nucleic acid sequence comprising 14, 15, 16, 17, 18, 19, 20, 21, or 22 consecutive nucleotides that are complementary to a sequence in Table 1, Table 2, and Table 5 without overhangs with no more than 1, 2, 3, or 4 mismatches. In some aspects, the polynucleic acid molecule described herein comprises a nucleic acid sequence comprising 14, 15, 16, 17, 18, 19, 20, 21, or 22 consecutive nucleotides that are complementary to a nucleic acid sequence of SEQ ID NO: 309-462 without overhangs with no more than 1, 2, 3, or 4 mismatches. In some aspects, the polynucleic acid molecule described herein comprises a nucleic acid sequence comprising 14, 15, 16, 17, 18, 19, 20, 21, or 22 consecutive nucleotides that are complementary to a nucleic acid sequence of SEQ ID NO: 1-154 without overhangs with no more than 1, 2, 3, or 4 mismatches.

In some aspects, the polynucleic acid molecule described herein comprises a strand of at least 10, 11, 12, 13, 14, or 15 nucleotides in length. In some aspects, the polynucleic acid molecule described herein comprises a strand of about 15-40, 16-30, 17-30, 18-30, 18-27, 18-25, 18-23, 19-23, 20-23, or 21-23 nucleotides in length. In some aspects, the polynucleic acid molecule described herein comprises a strand of about 15, 16, 17, 18, 19, 20 nucleotides long. In some aspects, the polynucleic acid molecule described herein comprises a strand of about 21, 22, 23, 24, 25 nucleotides long. In some aspects, the polynucleic acid molecule described herein comprises a strand of about 26, 27, 28, 29, 30 nucleotides long.

In some aspects, the polynucleic acid molecule described herein comprises a single-stranded nucleic acid of at least 10, 11, 12, 13, 14, or 15 nucleotides in length. In some aspects, the polynucleic acid molecule described herein comprises a single-stranded nucleic acid of about 15-30, 16-30, 17-30, 18-30, 18-27, 18-25, 18-23, 19-23, 20-23, or 21-23 nucleotides in length. In some aspects, the polynucleic acid molecule described herein comprises a single-stranded nucleic acid of about 15, 16, 17, 18, 19, 20 nucleotides long. In some aspects, the polynucleic acid molecule described herein comprises a single-stranded nucleic acid of about 21, 22, 23, 24, 25 nucleotides long. In some aspects, the polynucleic acid molecule described herein comprises a single-stranded nucleic acid of about 26, 27, 28, 29, 30 nucleotides long. In some aspects, the polynucleic acid molecule described herein comprises a single-stranded nucleic acid of about 21 nucleotides long. In some aspects, the polynucleic acid molecule described herein comprises a single-stranded nucleic acid of about 23 nucleotides long.

In some instances, the polynucleic acid molecule is a double-stranded nucleic acid molecule comprising a sense strand and an antisense strand, and wherein the antisense strand is at least partially reverse complementary to a target region of Lp(a) mRNA.

In some aspects, the antisense strand described herein is not 100% complementary to a target region of Lp(a) mRNA. Accordingly, in some instances, the antisense strand described herein is 100% complementary to a target region of Lp(a) mRNA. In some aspects, the antisense strand described herein is about 95% complementary to a target region of Lp(a) mRNA. In some aspects, the antisense strand described herein is about 90% complementary to a target region of Lp(a) mRNA. In some aspects, the antisense strand described herein is about 85% complementary to a target region of Lp(a) mRNA. In some aspects, the antisense strand described herein is about 80% complementary to a target region of Lp(a) mRNA. In some aspects, the antisense strand described herein is about 75% complementary to a target region of Lp(a) mRNA. In some aspects, the antisense strand described herein is about 70% complementary to a target region of Lp(a) mRNA.

In some aspects, the polynucleic acid molecule described herein comprises a nucleic acid sequence in Table 1 or Table 2. In other aspects, the polynucleic acid molecule described herein comprises a nucleic acid sequence that is at least 80%, at least 85%, at least 90%, or at least 95% identical to a sequence in Table 1 or Table 2. In some instances, the sense strand described herein comprises a nucleic acid sequence that is at least 80%, at least 85%, at least 90%, or at least 95% identical to a nucleic acid sequence selected from SEQ ID NOs:309-462. In some instances, the antisense strand described herein comprises a nucleic acid sequences that is at least 80%, at least 85%, at least 90%, or at least 95% identical to a nucleic acid sequence selected from SEQ ID NOs:1-154. In some instances, the sense strand described herein comprises a nucleic acid sequence that is at least 80%, at least 85%, at least 90%, at least 95% identical to a nucleic acid sequence selected from SEQ ID NOs: 312-314, 319, 326, 329, 352, 355, 357, 368, 372, 396-398, and 406. In some instances, the antisense strand described herein comprises a nucleic acid sequence that is at least 80%, at least 85%, at least 90%, at least 95%, identical to a nucleic acid sequence selected from SEQ ID NOs: 4-6, 11, 18, 21, 44, 47, 49, 60, 64, 88-90, and 98.

In yet other aspects, the polynucleic acid molecule described herein comprises a nucleic acid sequence comprising 15 consecutive sequences of the sequences in Table 1 or Table 2 with no more than 1, 2, 3, or 4 mismatches. In specific aspects, the sense strand described herein comprises a nucleic acid sequence comprising at least 15 consecutive sequences from SEQ ID NOs: 309-462 with no more than 1, 2, 3, or 4 mismatches. In specific aspects, the antisense strand described herein comprises a nucleic acid sequence comprising at least 15 consecutive sequences from SEQ ID NOs: 1-154 with no more than 1, 2, 3, or 4 mismatches. In yet still other aspects, the polynucleic acid molecule described herein comprises a nucleic acid sequence comprising at least 16 consecutive sequences out of the sequences in Table 1 or Table 2 with no more than 1, 2, 3, or 4 mismatches. In specific aspects, the sense strand described herein comprises a nucleic acid sequence comprising at least 16 consecutive sequences from SEQ ID NOs: 309-462 with no more than 1, 2, 3, or 4 mismatches. In specific aspects, the antisense strand described herein comprises a nucleic acid sequence comprising at least 16 consecutive sequences from SEQ ID NOs: 1-154 with no more than 1, 2, 3, or 4 mismatches. In yet still other aspects, the polynucleic acid molecule described herein comprises a nucleic acid sequence comprising at least 17 consecutive sequences from the sequences in Table 1 or Table 2 with no more than 1, 2, 3, or 4 mismatches. In specific aspects, the sense strand described herein comprises a nucleic acid sequence comprising at least 17 consecutive sequences from SEQ ID NOs: 309-462 with no more than 1, 2, 3, or 4 mismatches. In specific aspects, the antisense strand described herein comprises a nucleic acid sequence comprising at least 17 consecutive sequences from SEQ ID NOs: 1-154 with no more than 1, 2, 3, or 4 mismatches. In yet other aspects, the polynucleic acid molecule described herein comprises a nucleic acid sequence comprising at least 18 consecutive sequences from the sequences in Table 1 or Table 2 with no more than 1, 2, 3, or 4 mismatches. In specific aspects, the sense strand described herein comprises a nucleic acid sequence comprising at least 18 consecutive sequences from SEQ ID NOs: 309-462 with no more than 1, 2, 3, or 4 mismatches. In specific aspects, the antisense strand described herein comprises a nucleic acid sequence comprising at least 18 consecutive sequences from SEQ ID NOs: 1-154 with no more than 1, 2, 3, or 4 mismatches. In yet other aspects, the polynucleic acid molecule described herein comprises a nucleic acid sequence comprising at least 19 consecutive sequences from the sequences in Table 1 or Table 2 with no more than 1, 2, 3, or 4 mismatches. In specific aspects, the sense strand described herein comprises a nucleic acid sequence comprising at least 19 consecutive sequences from SEQ ID NOs: 309-462 with no more than 1, 2, 3, or 4 mismatches. In specific aspects, the antisense strand described herein comprises a nucleic acid sequence comprising at least 19 consecutive sequences from SEQ ID NOs: 1-154 with no more than 1, 2, 3, or 4 mismatches. In yet other aspects, the polynucleic acid molecule described herein comprises a nucleic acid sequence comprising at least 20 consecutive sequences from the sequences in Table 1 or Table 2 with no more than 1, 2, 3, or 4 mismatches. In specific aspects, the sense strand described herein comprises a nucleic acid sequence comprising at least 20 consecutive sequences from SEQ ID NOs: 309-462 with no more than 1, 2, 3, or 4 mismatches. In specific aspects, the antisense strand described herein comprises a nucleic acid sequence comprising at least 20 consecutive sequences from SEQ ID NOs: 1-154 with no more than 1, 2, 3, or 4 mismatches. In yet other aspects, the polynucleic acid molecule described herein comprises a nucleic acid sequence comprising at least 21 consecutive sequences from the sequences in Table 1 or Table 2 with no more than 1, 2, 3, or 4 mismatches. In specific aspects, the sense strand described herein comprises a nucleic acid sequence comprising at least 21 consecutive sequences from SEQ ID NOs: 309-462 with no more than 1, 2, 3, or 4 mismatches. In specific aspects, the antisense strand described herein comprises a nucleic acid sequence comprising at least 21 consecutive sequences from SEQ ID NOs: 1-154 with no more than 1, 2, 3, or 4 mismatches. In yet other aspects, the polynucleic acid molecule described herein comprises a nucleic acid sequence comprising at least 22 consecutive sequences from the sequences in Table 1 or Table 2 with no more than 1, 2, 3, or 4 mismatches. In specific aspects, the antisense strand described herein comprises a nucleic acid sequence comprising at least 22 consecutive sequences from SEQ ID NOs: 1-154 with no more than 1, 2, 3, or 4 mismatches. In specific aspects, the antisense strand described herein comprises a nucleic acid sequence comprising at least 23 consecutive sequences from SEQ ID NOs: 1-154 with no more than 1, 2, 3, or 4 mismatches. In yet other aspects, the polynucleic acid molecule described herein comprises a nucleic acid sequence comprising at least 23 consecutive sequences from the sequences in Table 1 or Table 2 with no more than 1, 2, 3, or 4 mismatches. As used herein, the term “consecutive sequence” refers to a sequence contains a number of consecutive nucleotides from a reference sequence. For example, if a reference sequence is N₁N₂N₃N₄N₅N₆N₇, a consecutive sequence can be N₁N₂N₃N₄ or N₃N₄N₅N₆, but a sequence of N₁N₃N₄N₅ or N₃N₄N₇ cannot be a consecutive sequence.

In some aspects, the sense strand described herein comprises a nucleic acid sequence comprising at least 15 consecutive sequences of SEQ ID NO: 312-314, 319, 326, 329, 352, 355, 357, 368, 372, 396-398, and 406 with no more than 1, 2, 3, or 4 mismatches. In some aspects, the antisense strand described herein comprises a nucleic acid sequence comprising at least 15 consecutive sequences of SEQ ID NO: 4-6, 11, 18, 21, 44, 47, 49, 60, 64, 88-90, and 98 with no more than 1, 2, 3, or 4 mismatches. In some aspects, the sense strand described herein comprises a nucleic acid sequence comprising at least 16 consecutive sequences of SEQ ID NO: 312-314, 319, 326, 329, 352, 355, 357, 368, 372, 396-398, and 406 with no more than 1, 2, 3 or 4 mismatches. In some aspects, the antisense strand described herein comprises a nucleic acid sequence comprising at least 16 consecutive sequences of SEQ ID NO: 4-6, 11, 18, 21, 44, 47, 49, 60, 64, 88-90, and 98 with no more than 1, 2, 3, or 4 mismatches. In some aspects, the sense strand described herein comprises a nucleic acid sequence comprising at least 17 consecutive sequences of SEQ ID NO: 312-314, 319, 326, 329, 352, 355, 357, 368, 372, 396-398, and 406 with no more than 1, 2, 3, or 4 mismatches. In some aspects, the antisense strand described herein comprises a nucleic acid sequence comprising at least 17 consecutive sequences of SEQ ID NO: 4-6, 11, 18, 21, 44, 47, 49, 60, 64, 88-90, and 98 with no more than 1, 2, 3, or 4 mismatches. In some aspects, the sense strand described herein comprises a nucleic acid sequence comprising at least 18 consecutive sequences of SEQ ID NO: 312-314, 319, 326, 329, 352, 355, 357, 368, 372, 396-398, and 406 with no more than 1, 2 or 3 mismatches. In some aspects, the antisense strand described herein comprises a nucleic acid sequence comprising at least 18 consecutive sequences of SEQ ID NO: 4-6, 11, 18, 21, 44, 47, 49, 60, 64, 88-90, and 98 with no more than 1, 2, 3, or 4 mismatches. In some aspects, the sense strand described herein comprises a nucleic acid sequence comprising at least 19 consecutive sequences of SEQ ID NO: 312-314, 319, 326, 329, 352, 355, 357, 368, 372, 396-398, and 406 with no more than 1, 2 or 3 mismatches. In some aspects, the antisense strand described herein comprises a nucleic acid sequence comprising at least 19 consecutive sequences of SEQ ID NO: 4-6, 11, 18, 21, 44, 47, 49, 60, 64, 88-90, and 98 with no more than 1, 2 or 3 mismatches. In some aspects, the sense strand described herein comprises a nucleic acid sequence comprising at least 20 consecutive sequences from SEQ ID NO: 312-314, 319, 326, 329, 352, 355, 357, 368, 372, 396-398, and 406 with no more than 1, 2 or 3 mismatches. In some aspects, the antisense strand described herein comprises a nucleic acid sequence comprising at least 20 consecutive sequences of SEQ ID NO: 4-6, 11, 18, 21, 44, 47, 49, 60, 64, 88-90, and 98 with no more than 1, 2 or 3 mismatches. In some aspects, the sense strand described herein comprises a nucleic acid sequence comprising at least 21 consecutive sequences of SEQ ID NO: 312-314, 319, 326, 329, 352, 355, 357, 368, 372, 396-398, and 406 with no more than 1, 2 or 3 mismatches. In some aspects, the antisense strand described herein comprises a nucleic acid sequence comprising at least 21 consecutive sequences of SEQ ID NO: 4-6, 11, 18, 21, 44, 47, 49, 60, 64, 88-90, and 98 with no more than 1, 2 or 3 mismatches. In some aspects, the antisense strand described herein comprises a nucleic acid sequence comprising at least 22 consecutive sequences of SEQ ID NO: 4-6, 11, 18, 21, 44, 47, 49, 60, 64, 88-90, and 98 with no more than 1, 2 or 3 mismatches. In some aspects, the antisense strand described herein comprises a nucleic acid sequence comprising at least 23 consecutive sequences of SEQ ID NO: 4-6, 11, 18, 21, 44, 47, 49, 60, 64, 88-90, and 98 with no more than 1, 2 or 3 mismatches.

In some aspects, the polynucleic acid molecule described herein comprises a sense and an antisense strand of at least 10, 11, 12, 13, 14, or 15 nucleotides in length. In some aspects, the polynucleic acid molecule described herein comprises a sense and an antisense strand of about 15-30, 16-30, 17-30, 18-30, 18-27, 18-25, 18-23, 19-23, 20-23, or 21-23 nucleotides in length. In some aspects, the polynucleic acid molecule described herein comprises a sense and an antisense strand of about 15, 16, 17, 18, 19, 20 nucleotides long. In some aspects, the polynucleic acid molecule described herein comprises a sense and an antisense strand of about 21, 22, 23, 24, 25 nucleotides long. In some aspects, the polynucleic acid molecule described herein comprises a sense and an antisense strand of about 26, 27, 28, 29, 30 nucleotides long. In some aspects, the polynucleic acid molecule described herein comprises a sense strand of 19 nucleotides long, and an antisense strand of about 21 nucleotides long. In some aspects, the polynucleic acid molecule described herein comprises a sense strand of 21 nucleotides long, and an antisense strand of about 23 nucleotides long.

In some aspects, the sense strand and the antisense strand described herein are reverse complementary to each other and form a duplex with a 3′ overhang on the antisense strand. In some aspects, the sense strand and the antisense strand described herein are reverse complementary to each other and form a duplex with a 5′ overhang on the antisense strand. In some aspects, the sense strand and the antisense strand described herein are reverse complementary to each other and form a duplex with a 3′ overhang on the sense strand. In some aspects, the sense strand and the antisense strand described herein are reverse complementary to each other and form a duplex with a 5′ overhang on the sense strand.

### Modifications of Polynucleic Acid Molecules

In some aspects, described herein is the polynucleic acid molecule described herein with modifications. In some aspects, the modifications described herein occurs one or more different structures of the polynucleic acid molecule described herein (e.g., modifications on sugar ring(s), backbone(s), base(s)). In some aspects, the modifications described herein comprise substitutions of one or more nucleotide in the polynucleic acid molecule described herein. In some aspects, different percentages of the polynucleic acid molecule described herein comprise the modifications described herein. In some aspects, different positions of the polynucleic acid molecule described herein comprise the modifications described herein. WO/2018/035380 is herein incorporated by reference in its entirety.

### Types of Modifications

In some aspects, the polynucleic acid molecule described herein comprises one or more sugar-modified nucleotide. In some instances, the sugar-modified nucleotide includes a modification at a 2′ hydroxyl group of the ribose moiety. In some instances, the sugar-modified nucleotide includes modification with an H, OR, R, halo, SH, SR, NH2, NHR, NR2, or CN, wherein R is an alkyl moiety. In some aspects, the sugar-modified nucleotide is a 2′-fluoro modified nucleotide. In some aspects, the sugar-modified nucleotide is a 2′-O-methyl modified nucleotide or a 2′-alkoxy modified nucleotide (e.g., 2′-methoxy modified nucleotide). In some instances, 2′ hydroxyl group modification includes 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In some instances, the alkyl moiety comprises a hetero substitution. In some instances, the carbon of the heterocyclic group is substituted by a nitrogen, oxygen or sulfur. In some instances, the heterocyclic substitution includes morpholino, imidazole, and pyrrolidino.

In some instances, 2′ hydroxyl group of the ribose moiety includes a locked or bridged ribose modification (e.g., LNA), an unlocked ribose modification (e.g., UNA), or ethylene nucleic acids (ENA).

In some aspects, the sugar-modified nucleotide is a 2′-amino modified nucleotide. In some aspects, the sugar-modified nucleotide is a 2′-azido modified nucleotide. In some aspects, the sugar-modified nucleotide is a 2′-deoxy modified nucleotide. In some aspects, the sugar-modified nucleotide is a 2′-O-methoxythyl (2′-MOE). In some aspects, the sugar-modified nucleotide is a locked nucleic acid (LNA). In some aspects, the sugar-modified nucleotide is an ethylene-bridged nucleic acid (ENA). In some aspects, the sugar-modified nucleotide is a (S)-constrained ethyl (cEt). In some aspects, the sugar-modified nucleotide is a tricyclo-DNA (tcDNA). In some aspects, the sugar-modified nucleotide is a 2′-NH2 nucleic acid.

In some aspects, the polynucleotide acid molecule described herein comprises one or more sugarphosphate-modified nucleotide. In some aspects, the modified sugarphosphate is phosphorodiamidate morpholino (PMO). In some aspects, the modified sugarphosphate is phosphoramidate. In some aspects, the modified sugarphosphate is thiophosphoramidate. In some aspects, the modified sugarphosphate is peptide nucleic acid (PNA).

In some aspects, the polynucleic acid molecule described herein comprises one or more backbone-modified nucleotide. In some aspects, the modified backbone is phosphorothioate. In some aspects, the modified backbone is methylphosphonate. In some aspects, the modified backbone is guanidinopropyl phosphoramidate. In some aspects, the modified backbone is a mesyl-phosphoramidate (MsPA) linkages. In some instances, the modified backbone comprises one or more of phosphorodithioates, methylphosphonates, 5′-alkylenephosphonates, 5′-methylphosphonate, 3′-alkylene phosphonates, borontrifluoridates, borano phosphate esters and selenophosphates of 3′-5′ linkage or 2′-5′ linkage, phosphotriesters, thionoalkylphosphotriesters, hydrogen phosphonate linkages, alkyl phosphonates, alkylphosphonothioates, arylphosphonothioates, phosphoroselenoates and phosphoramidates.

In some aspects, the modified nucleotide comprises a modified guanine (e.g., inosine) or one or more of any types of unnatural nucleic acids.

In some aspects, the modified backbone is phosphorothioate, and the phosphorothioate is a stereochemically enriched phosphorothioate. In certain aspects, the strand contains at least one stereochemically enriched phosphorothioate. In some aspects, the strand comprises at least 1, 2, 3 stereochemically enriched phosphorothioates. In some aspects, the strand comprises only 1, 2, 3, or 4 stereochemically enriched phosphorothioates. In further aspects, at least one (e.g., one or two) stereochemically enriched phosphorothioate is disposed between two consecutive nucleosides that are two of six 5′-terminal nucleosides of the strand. In yet further aspects, at least one (e.g., one or two) stereochemically enriched phosphorothioate is disposed between two consecutive nucleosides that are two of six 3′-terminal nucleosides of the strand. In still further aspects, one stereochemically enriched phosphorothioate is covalently bonded to the first nucleoside and the second nucleoside from the 5′-end within the strand. In some aspects, one stereochemically enriched phosphorothioate is covalently bonded to the twenty first nucleoside and the twenty second nucleoside from the 5′-end within the strand. In certain aspects, one stereochemically enriched phosphorothioate is covalently bonded to the twenty second nucleoside and the twenty third nucleoside from the 5′-end within the strand. In particular aspects, the stereochemically enriched phosphorothioate has R_{P} stereochemical identity. In certain aspects, the stereochemically enriched phosphorothioate has S_{P} stereochemical identity.

In some aspects, the polynucleotide molecules described herein comprises one or more (e.g., from 1 to 20, from 1 to 10, or from 1 to 5) stereochemically enriched (e.g., internucleoside) phosphorothioates (e.g., having diastereomeric excess of at least 10%, at least 50%, at least 60%, at least 70%, at least 80%, or at least 90%, e.g., up to about 99%, for the P-stereogenic center). The polynucleotide molecules described herein comprises one or more (e.g., from 1 to 20, from 1 to 10, or from 1 to 5; e.g., internucleoside) phosphorodithioates. The phosphorodithioates may be non-P-stereogenic in the polynucleotide molecules described herein. Phosphorothioates and phosphorodithioates may enhance the stability of the polynucleotide molecules described herein to exonuclease activity of serum. Non-P-stereogenic phosphorodithioates may simplify the synthesis of the polynucleotide molecule described herein by reducing the number of possible diastereomers. Typically, the phosphorothioate or phosphorodithioate may connect two contiguous nucleosides within the six 3′-terminal nucleosides and the six 5′-terminal nucleosides of the polynucleotide molecules described herein. In some aspects, the stereochemically enriched phosphorothioate (e.g., R_{P}-enriched phosphorothioate) may be covalently bonded to the first nucleoside(e.g., the 3′-carbon atom of the first nucleoside) and the second nucleoside (e.g., the 5′-carbon atom of the second nucleoside) from the 5′-end of the antisense strand. Additionally or alternatively, the stereochemically enriched phosphorothioate (e.g., S_{P}-enriched phosphorothioate) may be covalently bonded to the 21^{st} nucleoside (e.g., the 3′-carbon atom of the 21^{st} nucleoside) from the 5′-end and the 22^{nd} nucleoside (e.g., the 5′-carbon atom of the 22^{nd} nucleoside) of the antisense strand. Further, additionally or alternatively, the stereochemically enriched phosphorothioate (e.g., S_{P}-enriched phosphorothioate or Rp-enriched phosphorothioate) may be covalently bonded to the 22^{nd} nucleoside (e.g., the 3′-carbon atom of the 22^{nd} nucleoside) and the 23^{rd} nucleoside (e.g., the 5′-carbon atom of the 23^{rd} nucleoside) from the 5′-end of the antisense strand.

Combinations of a 5′ R_{P}-enriched phosphorothioate (e.g., R_{P}-enriched phosphorothioate covalently bonded to the first nucleoside (e.g., the 3′-carbon atom of the first nucleoside) and the second nucleoside (e.g., the 5′-carbon atom of the second nucleoside) from the 5′-end and a 3′ S_{P}-enriched phosphorothioate (e.g., S_{P}-enriched phosphorothioate covalently bonded to the 21^{st} nucleoside (e.g., the 3′-carbon atom of the 21^{st} nucleoside) and the 22^{nd} nucleoside (e.g., the 5′-carbon atom of the 22^{nd} nucleoside) from the 5′-end in an antisense strand can produce superior efficacy and/or duration of action, e.g., as measured by the reduction in the activity of the target relative to a reference guide strand that lacks the combination of a 5′ R_{P}-enriched phosphorothioate and a 3′ S_{P}-enriched phosphorothioate, or a 5′ Rp-enriched phosphorothioate and a 3′ Sp and Rp-enriched phosphorothioate. In some embodiments, the stereochemically enriched phosphorothioate may comprise RₚRₚSₚSₚ (RₚRₚ at the positions 1 and 2 of the guide strand and SₚSₚ at the positions 21 and 22 of the guide strand) or RₚRₚSₚRₚ (RₚRₚ at the positions 1 and 2 of the guide strand and SₚRₚ at the positions 21 and 22 of the guide strand). In some aspects, the polynucleotide molecules described herein comprises four stereochemically enriched phosphorothioates: (1) a Rp-enriched phosphorothioate covalently bonded to the 1^{st} nucleoside (e.g., the 3′-carbon atom of the 1^{st} nucleoside) and the 2^{nd} nucleoside (e.g., the 5′-carbon atom of the 2^{nd} nucleoside) from the 5′-end of the antisense strand; (2) a Rp-enriched phosphorothioate covalently bonded to the 2^{nd} nucleoside (e.g., the 3′-carbon atom of the 2^{nd} nucleoside) and the 3^{rd} nucleoside (e.g., the 5′-carbon atom of the 3^{rd} nucleoside) from the 5′-end of the antisense strand; (3) a Sp-enriched phosphorothioate covalently bonded to the 21^{st} nucleoside (e.g., the 3′-carbon atom of the 21^{st} nucleoside) and the 22^{th} nucleoside (e.g., the 5′-carbon atom of the 22^{th} nucleoside) from the 5′-end of the antisense strand; and (4) a Sp-enriched phosphorothioate covalently bonded to the 22^{th} nucleoside (e.g., the 3′-carbon atom of the 22^{th} nucleoside) and the 23^{rd} nucleoside (e.g., the 5′-carbon atom of the 23^{rd} nucleoside) from the 5′-end of the antisense strand. In some aspects, the polynucleotide molecules described herein comprises four stereochemically enriched phosphorothioates: (1) a Rp-enriched phosphorothioate covalently bonded to the 1^{st} nucleoside (e.g., the 3′-carbon atom of the 1^{st} nucleoside) and the 2^{nd} nucleoside (e.g., the 5′-carbon atom of the 2^{nd} nucleoside) from the 5′-end of the antisense strand; (2) a Rp-enriched phosphorothioate covalently bonded to the 2^{nd} nucleoside (e.g., the 3′-carbon atom of the 2^{nd} nucleoside) and the 3^{rd} nucleoside (e.g., the 5′-carbon atom of the 3^{rd} nucleoside) from the 5′-end of the antisense strand; (3) a Sp-enriched phosphorothioate covalently bonded to the 21^{st} nucleoside (e.g., the 3′-carbon atom of the 21^{st} nucleoside) and the 22^{th} nucleoside (e.g., the 5′-carbon atom of the 22^{th} nucleoside) from the 5′-end of the antisense strand; and (4) a Rp-enriched phosphorothioate covalently bonded to the 22^{th} nucleoside (e.g., the 3′-carbon atom of the 22^{th} nucleoside) and the 23^{rd} nucleoside (e.g., the 5′-carbon atom of the 23^{rd} nucleoside) from the 5′-end of the antisense strand.

In some aspects, the polynucleotide molecule described herein comprises one or more purine modification. In some aspects, the purine modification described herein is 2,6-diaminopurine. In some aspects, the purine modification described herein is 3-deaza-adenine. In some aspects, the purine modification described herein is 7-deaza-guanine. In some aspects, the purine modification described herein is 8-azido-adenine.

In some aspects, the polynucleotide molecule described herein comprises one or more pyrimidine modification. In some aspects, the pyrimidine modification described herein is 2-thiothymidine. In some aspects, the pyrimidine modification described herein is 5-carboxamide-uracil. In some aspects, the pyrimidine modification described herein is 5-methyl-cytosine. In some aspects, the pyrimidine modification described herein is 5-ethynyl uracil.

In some cases, the polynucleic acid molecule described herein comprises an abasic substitution. In those cases where a hybridized polynucleotide construct is contemplated for use as siRNA, a reduction of miRNA-like off-target effects is desirable. The inclusion of one or more (e.g., one or two) abasic substitutions in the hybridized polynucleotide constructs may reduce or even eliminate miRNA-like off-target effects, as the abasic substitutions lack nucleobases that are capable of engaging in base-pairing interactions and alleviate steric hindrance. Thus, the polynucleotide molecule disclosed herein may include one or more (e.g., one or two) abasic substitutions. In some aspects, abasic substitution is at the 5^{th} nucleotide from the 5′ end of the antisense strand described herein. In some aspects, abasic substitution is at the 7^{th} nucleotide from the 5′ end of the antisense strand described herein.

When the polynucleotide molecule disclosed herein includes two or more of the abasic substitutions, their structures may be same or different. In certain aspects, a sense strand contains one abasic substitution (e.g., an antisense strand may be free of abasic substitutions). In other aspects, an antisense strand contains one abasic substitution (e.g., a sense strand may be free of abasic substitutions). In yet other aspects, an antisense strand contains one abasic substitution, and a sense strand contains one abasic substitution. In further aspects, a sense strand includes an abasic substitution between a nucleoside number (x) and a nucleoside number (x+1), where x is an integer from 2 to 7. In yet further aspects, an antisense strand includes an abasic substitution between a nucleoside number (x) and a nucleoside number (x+1), where x is an integer from 2 to 7.

The abasic substitution may be of formula (III):

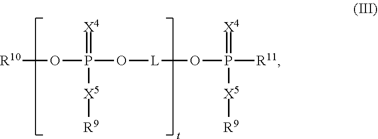

where
L is a sugar analogue, or is substituted with a heteroacyl from A, U, C, G, or is any other substituted nucleic acid (e.g., locked or unlocked nucleic acid, glycol nucleic acid, etc.);
each X⁴ is independently O or S;
each X⁵ is independently O, S, NH, or a bond;
each R⁹ is independently H, optionally substituted C₁₋₆ alkyl, optionally substituted C₂₋₆ alkenyl, optionally substituted C₂₋₆ alkynyl, optionally substituted (C₁₋₉ heterocyclyl)-C₁₋₆-alkyl, optionally substituted (C₆₋₁₀ aryl)-C₁₋₆-alkyl, optionally substituted (C₃₋₈ cycloalkyl)-C₁₋₆-alkyl, -LinkA(-T)ₚ, or a conjugation moiety;
each LinkA is independently a multivalent linker (e.g., including —C(O)—N(H)—);
each T is independently an auxiliary moiety;
R¹⁰ is a bond to a 3′-carbon atom of a nucleoside (x) in the strand;
R¹¹ is a bond to a 5′-oxygen atom of a nucleoside (x+1) in the strand;
p is an integer from 1 to 6; and
t is an integer from 1 to 6.

In some aspects, the abasic substitution described herein is attached to the antisense strand of the polynucleic acid molecule described herein. In particular aspects, an abasic substitution (e.g., an internucleotide, abasic spacer of formula (III) in which t is 1) may be included in the antisense strand described herein (e.g., within the seed region of the guide strand). In some aspects, an abasic substitution (e.g., an internucleotide, abasic spacer of formula (III) in which t is 1) may be bonded to the 3′ carbon atom of the second, third, fourth, or fifth nucleoside from the 5′-end of the antisense strand described herein. In certain aspects, an abasic substitution (e.g., an internucleotide, abasic spacer of formula (III) in which t is 1) may be bonded to the 3′ carbon atom of the thirteenth, fourteenth, fifteenth, or sixteenth nucleoside from the 5′-end of the antisense strand described herein. In some aspects, an abasic substitution fourth, fifth, sixth, seventh, eighth, and/or ninth nucleoside from the 5′-end of the antisense strand described herein.

The polynucleotide molecule described herein may contain a strand including a seed region including a hypoxanthine nucleobase-containing nucleoside (e.g., inosine).

In certain aspects, the hypoxanthine nucleobase-containing nucleoside is a second nucleoside from the 5′-end in the strand. In further aspects, the hypoxanthine nucleobase-containing nucleoside is a third nucleoside from the 5′-end in the strand. In yet further aspects, the hypoxanthine nucleobase-containing nucleoside is a fourth nucleoside from the 5′-end in the strand. In still further aspects, the hypoxanthine nucleobase-containing nucleoside is a fifth nucleoside from the 5′-end in the strand. In particular aspects, the hypoxanthine nucleobase-containing nucleoside is a sixth nucleoside in the strand. In particular aspects, the hypoxanthine nucleobase-containing nucleoside is a seventh nucleoside in the strand.

### The Amount and Location of Modifications

In some aspects, the polynucleotide molecule described herein comprises one or more type of modifications as described above. Accordingly, in some aspects, about 10% of the nucleotides from the polynucleotide molecule described herein are modified with one or more type of modifications as described above. In other aspects, about 20% of the nucleotides from the polynucleotide molecule described herein are modified with one or more type of modifications as described above. In other aspects, about 30% of the nucleotides from the polynucleotide molecule described herein are modified with one or more type of modifications as described above. In other aspects, about 40% of the nucleotides from the polynucleotide molecule described herein are modified with one or more type of modifications as described above. In other aspects, about 50% of the nucleotides from the polynucleotide molecule described herein are modified with one or more type of modifications as described above. In other aspects, about 60% of the nucleotides from the polynucleotide molecule described herein are modified with one or more type of modifications as described above. In other aspects, about 70% of the nucleotides from the polynucleotide molecule described herein are modified with one or more type of modifications as described above. In other aspects, about 80% of the nucleotides from the polynucleotide molecule described herein are modified with one or more type of modifications as described above. In other aspects, about 90% of the nucleotides from the polynucleotide molecule described herein are modified with one or more type of modifications as described above. In other aspects, 100% of the nucleotides from the polynucleotide molecule described herein are modified with one or more type of modifications as described above.

In some aspects, the one or more types of modifications described herein occurs in the seed region within the the polynucleotide molecule described herein. In some aspects, the one or more types of modifications described herein occurs at different positions within the polynucleotide molecule described herein. In specific aspects, the one or more types of modifications described herein occurs at 3′ terminal of the polynucleotide molecule described herein. In specific aspects, the one or more types of modifications described herein occurs at 5′ terminal of the polynucleotide molecule described herein. In specific aspects, the one or more types of modifications described herein occurs dispersedly within the polynucleotide molecule described herein. In specific aspects, the one or more types of modifications described herein occurs in clusters within the polynucleotide molecule described herein.

### Specific Modification Patterns

In some aspects, described herein is a specific modification pattern, wherein the sense strand comprises about three 2′-fluoro modified nucleotides and about eighteen 2′-O-methyl modified nucleotides, and wherein the antisense strand comprises about five 2′-fluoro modified nucleotides and about eighteen 2′-O-methyl modified nucleotides.

In some aspects, described herein is a polynucleic acid modified with a specific modification pattern, wherein the sense strand comprises 5′-nnnnnnNfnNfnNfnnnnnnnnnn-3′, wherein the antisense strand comprises 5′-nNfnnnnNfnnnnNfnNfnNfnnnnnnn-3′, wherein n stands for a 2′-O-methyl modified nucleotide, and wherein Nf stands for a 2′-fluoro modified nucleotide. In some aspects, the sense strand comprises ‘5-NfnNfnNfnNfnNfNfNfnNfnNfnNfnNfnNf-3’, wherein the antisense strand comprises ‘5-nNfnNfnNfnNfnNfnnnNfnNfnNfnNfnnn-3’, wherein Nf stands for a 2′-fluoro modified nucleotide, and wherein n stands for a 2′-O-methyl modified nucleotide. In some aspects, the sense strand comprises ‘5-nnnnnnNfnNfNfNfnnnnnnnnnn-3’, wherein the antisense strand comprises ‘5-nNfnnnNfnNfNfnnnnNfnNfnnnnnnn-3’, wherein Nf stands for a 2′-fluoro modified nucleotide, and wherein n stands for a 2′-O-methyl modified nucleotide. In some aspects, the sense strand comprises ‘5-nnnnnnnnNfnNfnnnnnnnnnn-3’, wherein the antisense strand comprises ‘5-nNfnnnnnnnnnNfnNfnnnnnnnnn-3’, wherein Nf stands for a 2′-fluoro modified nucleotide, and wherein n stands for a 2′-O-methyl modified nucleotide. In some aspects, the sense strand comprises ‘5-nnnnnnNfnNfnNfnnnnnnnnnn-3’, wherein the antisense strand comprises ‘5-nNfnnnnnnnnnNfnNfnNfnnnnnnn-3’, wherein Nf stands for a 2′-fluoro modified nucleotide, and wherein n stands for a 2′-O-methyl modified nucleotide. In some instances, each modification pattern includes one or more phosphorothioate internucleotide linkages in locations such as between 1^{st} and 2^{nd} nucleotides on 5′ end, between 2^{nd} and 3^{rd} nucleotides on 5′ end, between 1^{st} and 2^{nd} nucleotides on 3′ end, and/or between 2^{nd} and 3^{rd} nucleotides on 3′ end.

In some aspects, the antisense strand comprises 5′-nNfnnnNfnNfNfnnnnNfnNfnnnnnnn-3′, wherein Nf stands for a 2′-fluoro modified nucleotide, and wherein n stands for a 2′-O-methyl modified nucleotide. In some aspects, the antisense strand comprises 5′-nNfnnnNfnnnnnnnNfnNfnnnnnnn-3′, wherein Nf stands for a 2′-fluoro modified nucleotide, and wherein n stands for a 2′-O-methyl modified nucleotide. In some aspects, the antisense strand comprises 5′-nNfnnnnNfnnnnNfnNfnnnnnnnnn-3′, wherein Nf stands for a 2′-fluoro modified nucleotide, and wherein n stands for a 2′-O-methyl modified nucleotide. In some aspects, the antisense strand comprises or consists of two 2′-fluoro modified nucleotides at positions 2 and 14, respectively. In some aspects, the antisense strand comprises or consists of three 2′-fluoro modified nucleotides at positions 2, 12, and 14, respectively. In some aspects, the antisense strand comprises or consists of three 2′-fluoro modified nucleotides at positions 2, 14, and 16, respectively. In some aspects, the antisense strand comprises or consists of four 2′-fluoro modified nucleotides at positions 2, 12, 14, and 16, respectively. In some aspects, the antisense strand comprises a 2′-fluoro modified nucleotide at position 6, 7, 8, or 9. In some aspects, the antisense strand comprises a 2′-fluoro modified nucleotide at position 4. In some aspects, the antisense strand does not contain a 2′-fluoro modified nucleotide at position 4.

In some aspects, the sense strand comprises 5′-nnnnnnNfnNfnNfnnnnnnnnnn-3′, wherein Nf stands for a 2′-fluoro modified nucleotide, and wherein n stands for a 2′-O-methyl modified nucleotide. In some aspects, the sense strand comprises 5′-nnnnnnNfnNfNfNfnnnnnnnnnn-3′, wherein Nf stands for a 2′-fluoro modified nucleotide, and wherein n stands for a 2′-O-methyl modified nucleotide. In some aspects, the sense strand comprises 5′-nnnnnnnnNfNfNfnnnnnnnnnn-3′, wherein Nf stands for a 2′-fluoro modified nucleotide, and wherein n stands for a 2′-O-methyl modified nucleotide. In some aspects, the sense strand comprises or consists of two 2′-fluoro modified nucleotides at positions 9 and 11, respectively. In some aspects, the sense strand comprises or consists of three 2′-fluoro modified nucleotides at positions 7, 9 and 11, respectively. In some aspects, the sense strand comprises or consists of three 2′-fluoro modified nucleotides at positions 9, 10 and 11, respectively. In some aspects, the sense strand comprises or consists of four 2′-fluoro modified nucleotides at positions 7, 9, 10 and 11, respectively. In some aspects, the sense strand comprises a 2′-fluoro modified nucleotide at position 8, 12, or 16. In some aspects, the sense strand does not contain a 2′-fluoro modified nucleotide at position 8, 12, or 16.

In some aspects, described herein is a specific modification pattern for the polynucleic acid molecule which is a double-stranded nucleic acid molecule comprising a sense strand and an antisense strand. In some aspects, the antisense strand comprises a 2′-fluoro modified nucleotide in position 2 from the 5′ end. In some aspects, the antisense strand comprises a 2′-fluoro modified nucleotide in position 14 from the 5′ end. In some aspects, the antisense strand comprises 2′-fluoro modified nucleotides in positions 2 and 14 from the 5′ end. In some aspects, the antisense strand comprises a 2′-fluoro modified nucleotide in position 12 from the 5′ end. In some aspects, the antisense strand comprises a 2′-fluoro modified nucleotide in position 16 from the 5′ end. In other aspects, the antisense strand comprises a 2′-fluoro modified nucleotide in position 6 from the 5′ end. In other aspects, the antisense strand comprises a 2′-fluoro modified nucleotide in position 7 from the 5′ end. In other aspects, the antisense strand comprises a 2′-fluoro modified nucleotide in position 8 from the 5′ end. In other aspects, the antisense strand comprises a 2′-fluoro modified nucleotide in position 9 from the 5′ end. In other aspects, the antisense strand comprises a 2′-fluoro modified nucleotide in position 4 from the 5′ end.

In some aspects, described herein is a specific modification pattern for the polynucleic acid molecule which is a double-stranded nucleic acid molecule comprising a sense strand and an antisense strand. In some aspects, the sense strand comprises a 2′-fluoro modified nucleotide in position 9 from the 5′ end. In some aspects, the sense strand comprises a 2′-fluoro modified nucleotide in position 11 from the 5′ end. In some aspects, the sense strand comprises 2′-fluoro modified nucleotides in positions 9 and 11 from the 5′ end. In some aspects, the sense strand comprises a 2′-fluoro modified nucleotide in position 7 from the 5′ end. In some aspects, the sense strand comprises a 2′-fluoro modified nucleotide in position 10 from the 5′ end. In some aspects, the sense strand comprises 2′-fluoro modified nucleotides in positions 9, 11, and 7 from the 5′ end. the sense strand comprises 2′-fluoro modified nucleotides in positions 9 and 11, and 10 from the 5′ end. the sense strand comprises 2′-fluoro modified nucleotides in positions 9 and 7 from the 5′ end. the sense strand comprises 2′-fluoro modified nucleotides in positions 9 and 10 from the 5′ end. the sense strand comprises 2′-fluoro modified nucleotides in positions 9, 11, 7, and 10 from the 5′ end. In other aspects, the sense strand comprises a 2′-fluoro modified nucleotide in position 8 from the 5′ end. In other aspects, the sense strand comprises a 2′-fluoro modified nucleotide in position 12 from the 5′ end. In other aspects, the sense strand comprises a 2′-fluoro modified nucleotide in position 16 from the 5′ end.

In some aspects, the sense and antisense strand of the polynucleic acid molecule comprises any combination of two or more 2′-fluoro modified nucleotides at the positions described in the above two paragraphs.

In some aspects, the antisense strand comprises 5′-nNfnnnNfnNfNfnnnnNfnNfnnnnnnn-3′. In some aspects, the antisense strand comprises 5′-nNfnnnNfnnnnnnnNfnNfnnnnnnn-3′. In some aspects, the antisense strand comprises 5′-nNfnnnnNfnnnnNfnNfnnnnnnnnn-3′. In the modification patterns described above, “Nf” stands for a 2′-fluoro modified nucleotide, and wherein “n” stands for a 2′-O-methyl modified nucleotide.

In some aspects, the sense strand comprises 5′-nnnnnnNfnNfnNfnnnnnnnnnn-3′. In some aspects, the sense strand comprises 5′-nnnnnnNfnNfNfNfnnnnnnnnnn-3′. In some aspects, the sense strand comprises 5′-nnnnnnnnNfNfNfnnnnnnnnnn-3′. In the modification patterns described above, “Nf” stands for a 2′-fluoro modified nucleotide, and wherein “n” stands for a 2′-O-methyl modified nucleotide.

In some aspects, described herein is a specific modification pattern for the polynucleic acid molecule which is a double-stranded nucleic acid molecule comprising a sense strand and an antisense strand, wherein the sense strand comprises about twelve 2′-fluoro modified nucleotides and about nine 2′-O-methyl modified nucleotides, and wherein the antisense strand comprises about nine 2′-fluoro modified nucleotides and about fourteen 2′-O-methyl modified nucleotides.

In some aspects, described herein is a specific modification pattern, wherein the sense strand is fully modified and comprises twelve 2′-fluoro modified nucleotides, nine 2′-O-methyl modified nucleotides, and wherein the antisense strand is fully modified and comprises nine 2′-fluoro modified nucleotides and fourteen 2′-O-methyl modified nucleotides.

In some aspects, described herein is a specific modification pattern, wherein the sense strand comprises 5′-NfnNfnNfnNfnNfNfNfnNfnNfnNfnNfnNf-3′, wherein the antisense strand comprises 5′-nNfnNfnNfnNfnNfnnnNfnNfnNfnNfnnn-3′, wherein “Nf” stands for a 2′-fluoro modified nucleotide, and wherein “n” stands for a 2′-O-methyl modified nucleotide.

In some aspects, described herein is a specific modification pattern, wherein the sense strand comprises 5′-NfnNfnNfnNfnNfNfNfnNfnNfnNfnNfnNf-3′, wherein the antisense strand comprises 5′-nNfnNfnNfnNfnNfnnnNfnNfnNfnNfnnn-3′, wherein the sense and/or antisense strand comprises one or more phosphorothioate linkage, wherein “Nf” stands for a 2′-fluoro modified nucleotide, and wherein “n” stands for a 2′-O-methyl modified nucleotide. In other aspects, described herein is a specific modification pattern, wherein the sense strand comprises 5′-NfnNfnNfnNfnNfNfNfnNfnNfnNfnNfnNf-3′, wherein the antisense strand comprises 5′-nNfnNfnNfnNfnNfnnnNfnNfnNfnNfnnn-3′, wherein the sense comprises two phosphorothioate linkages, wherein the antisense comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the sense strand and/or antisense strand is modified as Type I in Table 8.

TABLE 8
Nucleotide Modification Patterns

| Pattern Name | Pattern |
|---|---|
| Type I for sense strand | 5′-NfsnsNfnNfnNfnNfNfNfnNfnNfnNfnNfnNf-3′ |
| Type I for antisense strand | 5′-nsNfsnNfnNfnNfnNfnnnNfnNfnNfnNfnsnsn-3′ |
| Type II for sense strand | 5′-nsnsnnnnNfnNfNfNfnnnnnnnnnn-3′ |
| Type II for antisense strand | 5′-nsNfsnnnNfnNfNfnnnnNfnNfnnnnnsnsn-3′ |
| Type III for sense strand | 5′-nsnsnnnnnnNfnNfnnnnnnnnnn-3′ |
| Type III for antisense strand | 5′-nsNfsnnnnnnnnnNfnNfnnnnnnnsnsn-3′ |
| Type IV for sense strand | 5′-nsnsnnnnNfnNfnNfnnnnnnnnnn-3′ |
| Type IV for antisense strand | 5′-nsNfsnnnnnnnnnNfnNfnNfnnnnnsnsn-3′ |
| Type V for sense strand | 5′-nsnsnnnnNfnNfnNfnnnnnnnnnn-3′ |
| Type V for antisense strand | 5′-_nsNfsnnnnNfnnnnNfnNfnNfnnnnnsnsn-3′ |
| Type VI for sense strand | 5′-nnnnnnNfnNfnNfnnnnnnnnnn-invdN-invdN-3′ |

Note:
“Nf” stands for a 2′-fluoro modified nucleotide, “n” stands for a 2′-O-methyl modified nucleotide, “s” stands for a 3′-phosphorothioate, “invdN” stands for an inverted deoxy-nucleotide.

In some aspects, the polynucleotide molecule provided herein comprises a sense strand comprising a nucleic acid sequence of SEQ ID NOs: 309-462, an antisense strand comprises a nucleic acid sequence of SEQ ID NOs: 1-154, and wherein the sense and/or antisense strand is modified in Type I modification pattern specified in Table 8.

In some aspects, described herein is a specific modification pattern, wherein the sense strand comprises about four 2′-fluoro modified nucleotides and about seventeen 2′-O-methyl modified nucleotides, and wherein the antisense strand comprises about six 2′-fluoro modified nucleotides and about seventeen 2′-O-methyl modified nucleotides.

In some aspects, described herein is a specific modification pattern, wherein the sense strand is fully modified and comprises four 2′-fluoro modified nucleotides, seventeen 2′-O-methyl modified nucleotides, and wherein the antisense strand is fully modified and comprises six 2′-fluoro modified nucleotides and seventeen 2′-O-methyl modified nucleotides.

In some aspects, described herein is a specific modification pattern, wherein the sense strand comprises 5′-nnnnnnNfnNfNfNfnnnnnnnnnn-3′, wherein the antisense strand comprises 5′-nNfnnnNfnNfNfnnnnNfnNfnnnnnnn-3′, wherein “Nf” stands for a 2′-fluoro modified nucleotide, and wherein “n” stands for a 2′-O-methyl modified nucleotide.

In some aspects, described herein is a specific modification pattern, wherein the sense strand comprises 5′-nnnnnnNfnNfNfNfnnnnnnnnnn-3′, wherein the antisense strand comprises 5′-nNfnnnNfnNfNfnnnnNfnNfnnnnnnn-3′, wherein the sense and/or antisense strand comprises one or more phosphorothioate linkage, wherein “Nf” stands for a 2′-fluoro modified nucleotide, and wherein “n” stands for a 2′-O-methyl modified nucleotide. In other aspects, described herein is a specific modification pattern, wherein the sense strand comprises 5′-nnnnnnNfnNfNfNfnnnnnnnnnn-3′, wherein the antisense strand comprises 5′-nNfnnnNfnNfNfnnnnNfnNfnnnnnnn-3′, wherein the sense comprises two phosphorothioate linkages, wherein the antisense comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the sense strand and/or antisense strand is modified as Type II in Table 8.

In some aspects, the polynucleotide molecule provided herein comprises a sense strand comprising a nucleic acid sequence of SEQ ID NOs: 309-462, and/or an antisense strand comprising a nucleic acid sequence of SEQ ID NOs: 1-154, and wherein the sense and/or antisense strand is modified in Type II modification pattern specified in Table 8.

In some aspects, described herein is a specific modification pattern, wherein the sense strand comprises about two 2′-fluoro modified nucleotides and about nineteen 2′-O-methyl modified nucleotides, and wherein the antisense strand comprises about three 2′-fluoro modified nucleotides and about twenty 2′-O-methyl modified nucleotides.

In some aspects, described herein is a specific modification pattern, wherein the sense strand is fully modified and comprises two 2′-fluoro modified nucleotides and nineteen 2′-O-methyl modified nucleotides, and wherein the antisense strand is fully modified and comprises three 2′-fluoro modified nucleotides and twenty 2′-O-methyl modified nucleotides.

In some aspects, described herein is a specific modification pattern, wherein the sense strand comprises 5′-nnnnnnnnNfnNfnnnnnnnnnn-3′, wherein the antisense strand comprises 5′-nNfnnnnnnnnnNfnNfnnnnnnnnn-3′, wherein “Nf” stands for a 2′-fluoro modified nucleotide, and wherein “n” stands for a 2′-O-methyl modified nucleotide.

In some aspects, described herein is a specific modification pattern, wherein the sense strand comprises 5′-nnnnnnnnNfnNfnnnnnnnnnn-3′, wherein the antisense strand comprises 5′-nNfnnnnnnnnnNfnNfnnnnnnnnn-3′, wherein the sense and/or antisense strand comprises one or more phosphorothioate linkage, wherein “Nf” stands for a 2′-fluoro modified nucleotide, and wherein “n” stands for a 2′-O-methyl modified nucleotide. In other aspects, described herein is a specific modification pattern, wherein the sense strand comprises 5′-nnnnnnnnNfnNfnnnnnnnnnn-3′, wherein the antisense strand comprises 5′-nNfnnnnnnnnnNfnNfnnnnnnnnn-3′, wherein the sense comprises two phosphorothioate linkages, wherein the antisense comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the sense strand and/or antisense strand is modified as Type III in Table 8.

In some aspects, the polynucleotide molecule provided herein comprises a sense strand comprising a nucleic acid sequence of SEQ ID NOs: 309-462, and/or an antisense strand comprising a nucleic acid sequence of SEQ ID NOs: 1-154, and wherein the sense and/or antisense strand is modified in Type III modification pattern specified in Table 8.

In some aspects, described herein is a specific modification pattern, wherein the sense strand comprises about three 2′-fluoro modified nucleotides and about eighteen 2′-O-methyl modified nucleotides, and wherein the antisense strand comprises about four 2′-fluoro modified nucleotides and about nineteen 2′-O-methyl modified nucleotides.

In some aspects, described herein is a specific modification pattern, wherein the sense strand is fully modified and comprises three 2′-fluoro modified nucleotides, eighteen 2′-O-methyl modified nucleotides, and wherein the antisense strand is fully modified and comprises four 2′-fluoro modified nucleotides, nineteen 2′-O-methyl modified nucleotides.

In some aspects, described herein is a specific modification pattern, wherein the sense strand comprises 5′-nnnnnnNfnNfnNfnnnnnnnnnn-3′, wherein the antisense strand comprises 5′-nNfnnnnnnnnnNfnNfnNfnnnnnnn-3′, wherein “Nf” stands for a 2′-fluoro modified nucleotide, and wherein “n” stands for a 2′-O-methyl modified nucleotide.

In some aspects, described herein is a specific modification pattern, wherein the sense strand comprises 5′-nnnnnnNfnNfnNfnnnnnnnnnn-3′, wherein the antisense strand comprises 5′-nNfnnnnnnnnnNfnNfnNfnnnnnnn-3′, wherein the sense and/or antisense strand comprises one or more phosphorothioate linkage, wherein “Nf” stands for a 2′-fluoro modified nucleotide, and wherein “n” stands fora 2′-O-methyl modified nucleotide. In other aspects, described herein is a specific modification pattern, wherein the sense strand comprises 5′-nnnnnnNfnNfnNfnnnnnnnnnn-3′, wherein the antisense strand comprises 5′-nNfnnnnnnnnnNfnNfnNfnnnnnnn-3′, wherein the sense comprises two phosphorothioate linkages, wherein the antisense comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the sense strand and/or antisense strand is modified as Type IV in Table 8.

In some aspects, the polynucleotide molecule provided herein comprises a sense strand comprising a nucleic acid sequence of SEQ ID NOs: 309-462, and/or an antisense strand comprising a nucleic acid sequence of SEQ ID NOs: 1-154, and wherein the sense and/or antisense strand is modified in Type IV modification pattern specified in Table 8.

In some aspects, described herein is a specific modification pattern, wherein the sense strand comprises about three 2′-fluoro modified nucleotides and about eighteen 2′-O-methyl modified nucleotides, and wherein the antisense strand comprises about five 2′-fluoro modified nucleotides and about eighteen 2′-O-methyl modified nucleotides.

In some aspects, described herein is a specific modification pattern, wherein the sense strand is fully modified and comprises three 2′-fluoro modified nucleotides and eighteen 2′-O-methyl modified nucleotides, and wherein the antisense strand is fully modified and comprises five 2′-fluoro modified nucleotides, eighteen 2′-O-methyl modified nucleotides.

In some aspects, described herein is a specific modification pattern, wherein the sense strand comprises 5′-nnnnnnNfnNfnNfnnnnnnnnnn-3′, wherein the antisense strand comprises 5′-nNfnnnnNfnnnnNfnNfnNfnnnnnnn-3′, wherein “Nf” stands for a 2′-fluoro modified nucleotide, and wherein “n” stands for a 2′-O-methyl modified nucleotide.

In some aspects, described herein is a specific modification pattern, wherein the sense strand comprises 5′-nnnnnnNfnNfnNfnnnnnnnnnn-3′, wherein the antisense strand comprises 5′-nNfnnnnNfnnnnNfnNfnNfnnnnnnn-3′, wherein the sense and/or antisense strand comprises one or more phosphorothioate linkage, wherein “Nf” stands for a 2′-fluoro modified nucleotide, and wherein “n” stands for a 2′-O-methyl modified nucleotide. In other aspects, described herein is a specific modification pattern, wherein the sense strand comprises 5′-nnnnnnNfnNfnNfnnnnnnnnnn-3′, wherein the antisense strand comprises 5′-_nNfnnnnNfnnnnNfnNfnNfnnnnnnn-3′, wherein the sense comprises two phosphorothioate linkages, wherein the antisense comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the sense strand and/or antisense strand is modified as Type V in Table 8.

In some aspects, the polynucleotide molecule provided herein comprises a sense strand comprising a nucleic acid sequence of SEQ ID NOs: 309-462, and/or an antisense strand comprising a nucleic acid sequence of SEQ ID NOs: 1-154, and wherein the sense and/or antisense strand is modified in Type V modification pattern specified in Table 8.

In some aspects, described herein is a specific modification pattern, wherein the sense strand comprises about three 2′-fluoro modified nucleotides and about eighteen 2′-O-methyl modified nucleotides, with one or more inverted deoxy-nucleotides on the 3′ end as an overhang.

In some aspects, described herein is a specific modification pattern, wherein the sense strand is fully modified and comprises three 2′-fluoro modified nucleotides and eighteen 2′-O-methyl modified nucleotides, with two inverted deoxy-nucleotides on the 3′ end as an overhang.

In some aspects, described herein is a specific modification pattern, wherein the sense strand comprises 5′-nnnnnnNfnNfnNfnnnnnnnnnn-invdN-invdN-3′, wherein “Nf” stands fora 2′-fluoro modified nucleotide, wherein “n” stands for a 2′-O-methyl modified nucleotide, and “invdN” stands for an inverted deoxy-nucleotide. In some instances, the invdN is an inverted deoxyl-thymine. In some aspects, the linker conjugated with one or more targeting moieties as shown in Formula (IV″ or IV′″) is added to the first nucleic acid on the 5′ end. In some aspects, the linker conjugated with one or more GalNAc as shown in Formula (V″) or (V′″) is added to the first nucleic acid on the 5′ end. In some aspects, the modification pattern comprises one or more phosphorothioate linkages. In some aspects, the modification pattern is shown in Formula (VII). In some aspects, the 5′ end modification known in the art is applied to the one or more inverted nucleotides.

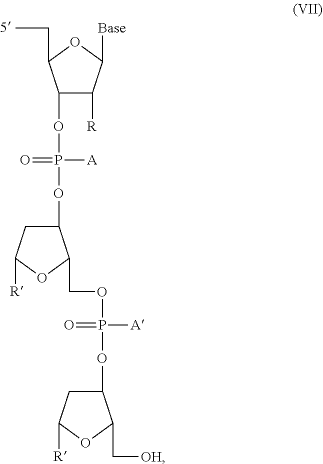

wherein R is a moiety that corresponds to the sugar modification described herein, in some instances, R is —O-methyl; wherein R′ is thymine, abasic, or others; wherein A is —O or —S; and wherein A′ is —O or —S.

In some aspects, the polynucleotide molecule provided herein comprises a sense strand comprising a nucleic acid sequence of SEQ ID NOs: 309-462, and/or an antisense strand comprising a nucleic acid sequence of SEQ ID NOs: 1-154, and wherein the sense and/or antisense strand is modified in Type VI modification pattern specified in Table 8.

In some aspects, the polynucleotide molecule provided herein comprises a single-stranded nucleic acid comprising a nucleic acid sequence selected from one of SEQ ID NOs: 1-154. In some aspects, the polynucleotide molecule provided herein comprises a single-stranded nucleic acid that is complementary to a nucleic acid sequence selected from SEQ ID NOs: 309-462.

In some aspects, the polynucleotide molecule provided herein comprises a sense strand comprising a nucleic acid sequence selected from one of SEQ ID NOs: 309-462 and an antisense strand comprising a nucleic acid sequence selected from one of SEQ ID NOs: 1-154. In other aspects, the polynucleotide molecule provided herein comprises a sense strand comprising a nucleic acid sequence selected from one of SEQ ID NOs: 309-462, an antisense strand comprising a nucleic acid sequence selected from one of SEQ ID NOs: 1-154, and wherein the sense and/or antisense strand is modified in the modification pattern as described herein.

In some aspects, the polynucleotide molecule provided herein comprises a sense strand comprising a nucleic acid sequence selected from one of SEQ ID NOs: 463-616, and an antisense strand comprising a nucleic acid sequence selected from SEQ ID NOs: 155-308.

Further provided herein is a polynucleic acid molecule for modulating expression of Lipoprotein(a) (Lp(a)) gene, wherein polynucleic acid molecule comprises an antisense strand comprising the nucleotide sequence of usAfsgaugAfccaaGfcUfuGfgcaggsusc (SEQ ID NO: 158) and a sense strand comprising the nucleotide sequence of cscsugccAfaGfcUfuggucaucua (SEQ ID NO: 466), wherein “A” refers to adenosine-3′-phosphate; “a” refers to 2′-O-methyladenosine-3′-phosphate; “Af” refers to 2′-fluoroadenosine-3′-phosphate; “C” refers to cytidine-3′-phosphate; “c” refers to 2′-O-methylcytidine-3′-phosphate; “Cf” refers to 2′-fluorocytidine-3′-phosphate; “G” refers to guanosine-3′-phosphate; “g” refers to 2′-O-methylguanosine-3′-phosphate; “Gf” refers to 2′-fluoroguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “s” refers to 3′-phosphorothioate.

Further provided herein is a polynucleic acid molecule for modulating expression of Lipoprotein(a) (Lp(a)) gene, wherein polynucleic acid molecule comprises an antisense strand comprising the nucleotide sequence of usAfsuagaUfgaccAfaGfcUfuggcasgsg (SEQ ID NO: 159) and a sense strand comprising the nucleotide sequence of usgsccaaGfcUfuGfgucaucuaua (SEQ ID NO: 467), wherein “A” refers to adenosine-3′-phosphate; “a” refers to 2′-O-methyladenosine-3′-phosphate; “Af” refers to 2′-fluoroadenosine-3′-phosphate; “C” refers to cytidine-3′-phosphate; “c” refers to 2′-O-methylcytidine-3′-phosphate; “Cf” refers to 2′-fluorocytidine-3′-phosphate; “G” refers to guanosine-3′-phosphate; “g” refers to 2′-O-methylguanosine-3′-phosphate; “Gf” refers to 2′-fluoroguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “s” refers to 3′-phosphorothioate.

Further provided herein is a polynucleic acid molecule for modulating expression of Lipoprotein(a) (Lp(a)) gene, wherein polynucleic acid molecule comprises an antisense strand comprising the nucleotide sequence of usCfsauagAfugacCfaAfgCfuuggcsasg (SEQ ID NO. 160) and a sense strand comprising the nucleotide sequence of gscscaagCfuUfgGfucaucuauga (SEQ ID NO: 468), wherein “A” refers to adenosine-3′-phosphate; “a” refers to 2′-O-methyladenosine-3′-phosphate; “Af” refers to 2′-fluoroadenosine-3′-phosphate; “C” refers to cytidine-3′-phosphate; “c” refers to 2′-O-methylcytidine-3′-phosphate; “Cf” refers to 2′-fluorocytidine-3′-phosphate; “G” refers to guanosine-3′-phosphate; “g” refers to 2′-O-methylguanosine-3′-phosphate; “Gf” refers to 2′-fluoroguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “s” refers to 3′-phosphorothioate.

Further provided herein is a polynucleic acid molecule for modulating expression of Lipoprotein(a) (Lp(a)) gene, wherein polynucleic acid molecule comprises an antisense strand comprising the nucleotide sequence of usCfsgacgGfcaguCfcCfuUfcugcgsusc (SEQ ID NO: 165) and a sense strand comprising the nucleotide sequence of csgscagaAfgGfgAfcugccgucga (SEQ ID NO: 473), wherein “A” refers to adenosine-3′-phosphate; “a” refers to 2′-O-methyladenosine-3′-phosphate; “Af” refers to 2′-fluoroadenosine-3′-phosphate; “C” refers to cytidine-3′-phosphate; “c” refers to 2′-O-methylcytidine-3′-phosphate; “Cf” refers to 2′-fluorocytidine-3′-phosphate; “G” refers to guanosine-3′-phosphate; “g” refers to 2′-O-methylguanosine-3′-phosphate; “Gf” refers to 2′-fluoroguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “s” refers to 3′-phosphorothioate.

Further provided herein is a polynucleic acid molecule for modulating expression of Lipoprotein(a) (Lp(a)) gene, wherein polynucleic acid molecule comprises an antisense strand comprising the nucleotide sequence of usUfscuagGfcuugGfaAfcCfggggusasa (SEQ ID NO: 172) and a sense strand comprising the nucleotide sequence of ascscccgGfuUfcCfaagccuagaa (SEQ ID NO: 480), wherein “A” refers to adenosine-3′-phosphate; “a” refers to 2′-O-methyladenosine-3′-phosphate; “Af” refers to 2′-fluoroadenosine-3′-phosphate; “C” refers to cytidine-3′-phosphate; “c” refers to 2′-O-methylcytidine-3′-phosphate; “Cf” refers to 2′-fluorocytidine-3′-phosphate; “G” refers to guanosine-3′-phosphate; “g” refers to 2′-O-methylguanosine-3′-phosphate; “Gf” refers to 2′-fluoroguanosine-3′-phosphate; “U” refers to uridine-3′-phosphate; “u” refers to 2′-O-methyluridine-3′-phosphate; “Uf” refers to 2′-fluorouridine-3′-phosphate; “1” refers to 5-methyluridine-3′-phosphate; “t” refers to 2′-O-methyl-5-methyluridine-3′-phosphate; “Tf” refers to 2′-fluoro-5-methyluridine-3′-phosphate; “s” refers to 3′-phosphorothioate.

Further provided herein is a polynucleic acid molecule for modulating expression of Lipoprotein(a) (Lp(a)) gene, wherein polynucleic acid molecule comprises an antisense strand comprising the nucleotide sequence of usAfsgccuCfuaggCfuUfgGfaaccgsgsg (SEQ ID NO: 175) and a sense strand comprising the nucleotide sequence of csgsguucCfaAfgCfcuagaggcua (SEQ ID NO: 483), wherein “A” refers to adenosine-3′-phosphate; “a” refers to 2′-O-methyladenosine-3′-phosphate; “Af” refers to 2′-fluoroadenosine-3′-phosphate; “C” refers to cytidine-3′-phosphate; “c” refers to 2′-O-methylcytidine-3′-phosphate; “Cf” refers to 2′-fluorocytidine-3′-phosphate; “G” refers to guanosine-3′-phosphate; “g” refers to 2′-O-methylguanosine-3′-phosphate; “Gf” refers to 2′-fluoroguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “s” refers to 3′-phosphorothioate.

Further provided herein is a polynucleic acid molecule for modulating expression of Lipoprotein(a) (Lp(a)) gene, wherein polynucleic acid molecule comprises an antisense strand comprising the nucleotide sequence of usUfsuaccGfugguAfgCfaCfuccugscsa (SEQ ID NO: 198) and a sense strand comprising the nucleotide sequence of csasggagUfgCfuAfccacgguaaa (SEQ ID NO: 506), wherein “A” refers to adenosine-3′-phosphate; “a” refers to 2′-O-methyladenosine-3′-phosphate; “Af” refers to 2′-fluoroadenosine-3′-phosphate; “C” refers to cytidine-3′-phosphate; “c” refers to 2′-O-methylcytidine-3′-phosphate; “Cf” refers to 2′-fluorocytidine-3′-phosphate; “G” refers to guanosine-3′-phosphate; “g” refers to 2′-O-methylguanosine-3′-phosphate; “Gf” refers to 2′-fluoroguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “s” refers to 3′-phosphorothioate.

Further provided herein is a polynucleic acid molecule for modulating expression of Lipoprotein(a) (Lp(a)) gene, wherein polynucleic acid molecule comprises an antisense strand comprising the nucleotide sequence of usUfsguccAfuuacCfgUfgGfuagcascsu (SEQ ID NO: 201) and a sense strand comprising the nucleotide sequence of usgscuacCfaCfgGfuaauggacaa (SEQ ID NO: 509), wherein “A” refers to adenosine-3′-phosphate; “a” refers to 2′-O-methyladenosine-3′-phosphate; “Af” refers to 2′-fluoroadenosine-3′-phosphate; “C” refers to cytidine-3′-phosphate; “c” refers to 2′-O-methylcytidine-3′-phosphate; “Cf” refers to 2′-fluorocytidine-3′-phosphate; “G” refers to guanosine-3′-phosphate; “g” refers to 2′-O-methylguanosine-3′-phosphate; “Gf” refers to 2′-fluoroguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “s” refers to 3′-phosphorothioate.

Further provided herein is a polynucleic acid molecule for modulating expression of Lipoprotein(a) (Lp(a)) gene, wherein polynucleic acid molecule comprises an antisense strand comprising the nucleotide sequence of usCfsucugUfccauUfaCfcGfugguasgsc (SEQ ID NO: 203) and a sense strand comprising the nucleotide sequence of usasccacGfgUfaAfuggacagaga (SEQ ID NO: 511), wherein “A” refers to adenosine-3′-phosphate; “a” refers to 2′-O-methyladenosine-3′-phosphate; “Af” refers to 2′-fluoroadenosine-3′-phosphate; “C” refers to cytidine-3′-phosphate; “c” refers to 2′-O-methylcytidine-3′-phosphate; “Cf” refers to 2′-fluorocytidine-3′-phosphate; “G” refers to guanosine-3′-phosphate; “g” refers to 2′-O-methylguanosine-3′-phosphate; “Gf” refers to 2′-fluoroguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “T” refers to 2′-fluoro-5-methyluridine-3′-phosphate; “s” refers to 3′-phosphorothioate.

Further provided herein is a polynucleic acid molecule for modulating expression of Lipoprotein(a) (Lp(a)) gene, wherein polynucleic acid molecule comprises an antisense strand comprising the nucleotide sequence of usAfsuuguGfucagGfuUfgCfaguacsusc (SEQ ID NO: 214) and a sense strand comprising the nucleotide sequence of gsusacugCfaAfcCfugacacaaua (SEQ ID NO: 522), wherein “A” refers to adenosine-3′-phosphate; “a” refers to 2′-O-methyladenosine-3′-phosphate; “Af” refers to 2′-fluoroadenosine-3′-phosphate; “C” refers to cytidine-3′-phosphate; “c” refers to 2′-O-methylcytidine-3′-phosphate; “Cf” refers to 2′-fluorocytidine-3′-phosphate; “G” refers to guanosine-3′-phosphate; “g” refers to 2′-O-methylguanosine-3′-phosphate; “Gf” refers to 2′-fluoroguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “s” refers to 3′-phosphorothioate.

Further provided herein is a polynucleic acid molecule for modulating expression of Lipoprotein(a) (Lp(a)) gene, wherein polynucleic acid molecule comprises an antisense strand comprising the nucleotide sequence of usUfsgcguCfugagCfaUfuGfugucasgsg (SEQ ID NO: 218) and a sense strand comprising the nucleotide sequence of usgsacacAfaUfgCfucagacgcaa (SEQ ID NO: 526), wherein “A” refers to adenosine-3′-phosphate; “a” refers to 2′-O-methyladenosine-3′-phosphate; “Af” refers to 2′-fluoroadenosine-3′-phosphate; “C” refers to cytidine-3′-phosphate; “c” refers to 2′-O-methylcytidine-3′-phosphate; “Cf” refers to 2′-fluorocytidine-3′-phosphate; “G” refers to guanosine-3′-phosphate; “g” refers to 2′-O-methylguanosine-3′-phosphate; “Gf” refers to 2′-fluoroguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “s” refers to 3′-phosphorothioate.

Further provided herein is a polynucleic acid molecule for modulating expression of Lipoprotein(a) (Lp(a)) gene, wherein polynucleic acid molecule comprises an antisense strand comprising the nucleotide sequence of usUfsaacuCfugucCfaUfaAfugguasgsu (SEQ ID NO: 242) and a sense strand comprising the nucleotide sequence of usasccauUfaUfgGfacagaguuaa (SEQ ID NO: 550), wherein “A” refers to adenosine-3′-phosphate; “a” refers to 2′-O-methyladenosine-3′-phosphate; “Af” refers to 2′-fluoroadenosine-3′-phosphate; “C” refers to cytidine-3′-phosphate; “c” refers to 2′-O-methylcytidine-3′-phosphate; “Cf” refers to 2′-fluorocytidine-3′-phosphate; “G” refers to guanosine-3′-phosphate; “g” refers to 2′-O-methylguanosine-3′-phosphate; “Gf” refers to 2′-fluoroguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “s” refers to 3′-phosphorothioate.

Further provided herein is a polynucleic acid molecule for modulating expression of Lipoprotein(a) (Lp(a)) gene, wherein polynucleic acid molecule comprises an antisense strand comprising the nucleotide sequence of usCfscaagCfuuggCfaAfgUfucuucscsu (SEQ ID NO: 243) and a sense strand comprising the nucleotide sequence of gsasagaaCfuUfgCfcaagcuugga (SEQ ID NO: 551), wherein “A” refers to adenosine-3′-phosphate; “a” refers to 2′-O-methyladenosine-3′-phosphate; “Af” refers to 2′-fluoroadenosine-3′-phosphate; “C” refers to cytidine-3′-phosphate; “C” refers to 2′-O-methylcytidine-3′-phosphate; “Cf” refers to 2′-fluorocytidine-3′-phosphate; “G” refers to guanosine-3′-phosphate; “g” refers to 2′-O-methylguanosine-3′-phosphate; “Gf” refers to 2′-fluoroguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “T” refers to 2′-fluoro-5-methyluridine-3′-phosphate; “s” refers to 3′-phosphorothioate.

Further provided herein is a polynucleic acid molecule for modulating expression of Lipoprotein(a) (Lp(a)) gene, wherein polynucleic acid molecule comprises an antisense strand comprising the nucleotide sequence of usAfsgaugAfccaaGfcUfuGfgcaagsusu (SEQ ID NO: 244) and a sense strand comprising the nucleotide sequence of csusugccAfaGfcUfuggucaucua (SEQ ID NO: 552), wherein “A” refers to adenosine-3′-phosphate; “a” refers to 2′-O-methyladenosine-3′-phosphate; “Af” refers to 2′-fluoroadenosine-3′-phosphate; “C” refers to cytidine-3′-phosphate; “c” refers to 2′-O-methylcytidine-3′-phosphate; “Cf” refers to 2′-fluorocytidine-3′-phosphate; “G” refers to guanosine-3′-phosphate; “g” refers to 2′-O-methylguanosine-3′-phosphate; “Gf” refers to 2′-fluoroguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “s” refers to 3′-phosphorothioate.

Further provided herein is a polynucleic acid molecule for modulating expression of Lipoprotein(a)(Lp(a)) gene, wherein polynucleic acid molecule an antisense strand comprising the nucleotide sequence of usGfsguccGfacuaUfgCfuGfgugugsgsu (SEQ ID NO: 252) and a sense strand comprising the nucleotide sequence of csascaccAfgCfaUfagucggacca (SEQ ID NO: 560), wherein “A” refers to adenosine-3′-phosphate; “a” refers to 2′-O-methyladenosine-3′-phosphate; “Af” refers to 2′-fluoroadenosine-3′-phosphate; “C” refers to cytidine-3′-phosphate; “c” refers to 2′-O-methylcytidine-3′-phosphate; “Cf” refers to 2′-fluorocytidine-3′-phosphate; “G” refers to guanosine-3′-phosphate; “g” refers to 2′-O-methylguanosine-3′-phosphate; “Gf” refers to 2′-fluoroguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “s” refers to 3′-phosphorothioate.

Further provided herein is a polynucleic acid molecule for modulating expression of Lipoprotein(a)(Lp(a)) gene, wherein polynucleic acid molecule an antisense strand comprising the nucleotide sequence of usAfsgccuCfuaggCfuUfgGfaaccgsgsg (SEQ ID NO: 175) and a sense strand comprising the nucleotide sequence of csgsguucCfaAfgCfcuagaggcua (SEQ ID NO: 483), wherein “A” refers to adenosine-3′-phosphate; “a” refers to 2′-O-methyladenosine-3′-phosphate; “Af” refers to 2′-fluoroadenosine-3′-phosphate; “C” refers to cytidine-3′-phosphate; “c” refers to 2′-O-methylcytidine-3′-phosphate; “Cf” refers to 2′-fluorocytidine-3′-phosphate; “G” refers to guanosine-3′-phosphate; “g” refers to 2′-O-methylguanosine-3′-phosphate; “Gf” refers to 2′-fluoroguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “s” refers to 3′-phosphorothioate.

Further provided herein is a polynucleic acid molecule for modulating expression of Lipoprotein(a) (Lp(a)) gene, wherein polynucleic acid molecule an antisense strand comprising the nucleotide sequence of usAfsuuguGfucagGfuUfgCfaguacsusc (SEQ ID NO: 214) and a sense strand comprising the nucleotide sequence of gsusacugCfaAfcCfugacacaaua (SEQ ID NO: 522), wherein “A” refers to adenosine-3′-phosphate; “a” refers to 2′-O-methyladenosine-3′-phosphate; “Af” refers to 2′-fluoroadenosine-3′-phosphate; “C” refers to cytidine-3′-phosphate; “c” refers to 2′-O-methylcytidine-3′-phosphate; “Cf” refers to 2′-fluorocytidine-3′-phosphate; “G” refers to guanosine-3′-phosphate; “g” refers to 2′-O-methylguanosine-3′-phosphate; “Gf” refers to 2′-fluoroguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “s” refers to 3′-phosphorothioate.

TABLE 1

| | | | | Modified | | | | | Modified | | Position |
|---|---|---|---|---|---|---|---|---|---|---|---|
| | | Unmodified | SEQ | Antisense | SEQ | | Unmodified | SEQ | Sense | SEQ | at |
| Compd | siRNA | Antisense Sequence | ID | Sequence | ID | siRNA | Sense Sequence | ID | Sequence | ID | Human |
| ID | ID | (5′→3′) | NO. | (5′→3′) | NO. | ID | (5′→3′) | NO. | (5′→3′) | NO. | Transcript |
| SRS- | G- | UCCAAGCUUGGCAGGUCCUUCCU | 1 | [symbol] | 155 | P- | GAAGGACCUGCCAAGCUUGGA | 309 | [symbol] | 463 | 198 |
| 000023 | 000023 | | | | | 000023 | | | | | |
| SRS- | G- | UACCAAGCUUGGCAGGUCCUUCC | 2 | [symbol] | 156 | P- | AAGGACCUGCCAAGCUUGGUA | 310 | [symbol] | 464 | 199 |
| 000024 | 000024 | | | | | 000024 | | | | | |
| SRS- | G- | UAUGACCAAGCUUGGCAGGUCCU | 3 | [symbol] | 157 | P- | GACCUGCCAAGCUUGGUCAUA | 311 | [symbol] | 465 | 202 |
| 000025 | 000025 | | | | | 000025 | | | | | |
| SRS- | G- | UAGAUGACCAAGCUUGGCAGGUC | 4 | [symbol] | 158 | P- | CCUGCCAAGCUUGGUCAUCUA | 312 | [symbol] | 466 | 204 |
| 000026 | 000008 | | | | | 000026 | | | | | |
| SRS- | G | UAUAGAUGACCAAGCUUGGCAGG | 5 | [symbol] | 159 | P- | UGCCAAGCUUGGUCAUCUAUA | 313 | [symbol] | 467 | 206 |
| 000027 | 000009 | | | | | 000027 | | | | | |
| SRS- | G- | UCAUAGAUGACCAAGCUUGGCAG | 6 | [symbol] | 160 | P- | GCCAAGCUUGGUCAUCUAUGA | 314 | [symbol] | 468 | 207 |
| 000028 | 000010 | | | | | 000028 | | | | | |
| SRS- | G- | UAUCAAGCCAGCAUUUGGGUAGU | 7 | [symbol] | 161 | P- | UACCCAAAUGCUGGCUUGAUA | 315 | [symbol] | 469 | 260 |
| 000029 | 000026 | | | | | 000029 | | | | | |
| SRS- | G- | UGUCCCUUCUGCGUCUGAGCAUU | 8 | [symbol] | 162 | P- | UGCUCAGACGCAGAAGGGACA | 316 | [symbol] | 470 | 374 |
| 000030 | 000027 | | | | | 000030 | | | | | |
| SRS- | G- | UCGGCAGUCCCCUCUGCGUCUGA | 9 | [symbol] | 163 | P- | AGACGCAGAAGGGACUGCCGA | 317 | [symbol] | 471 | 379 |
| 000031 | 000028 | | | | | 000031 | | | | | |
| SRS- | G- | UGACGGCAGUCCCUUCUGCGUCU | 10 | [symbol] | 164 | P- | ACGCAGAAGGGACUGCCGUCA | 318 | [symbol] | 472 | 381 |
| 000032 | 000029 | | | | | 000032 | | | | | |
| SRS- | G- | UCGACGGCAGUCCCUUCUGCGUC | 11 | [symbol] | 165 | P- | CGCAGAAGGGACUGCCGUCGA | 319 | [symbol] | 473 | 382 |
| 000033 | 000011 | | | | | 000033 | | | | | |
| SRS- | G- | UGCGACGGCAGUCCCUUCUGCGU | 12 | [symbol] | 166 | P- | GCAGAAGGGACUGCCGUCGCA | 320 | [symbol] | 474 | 383 |
| 000034 | 000030 | | | | | 000034 | | | | | |
| SRS- | G- | UGAACCGGGGUAACAGUCGGAGG | 13 | [symbol] | 167 | P- | UCCGACUGUUACCCCGGUUCA | 321 | [symbol] | 475 | 406 |
| 000035 | 000031 | | | | | 000035 | | | | | |
| SRS- | G- | UGGAACCGGGGUAACAGUCGGAG | 14 | [symbol] | 168 | P- | CCGACUGUUACCCCGGUUCCA | 322 | [symbol] | 476 | 407 |
| 000036 | 000032 | | | | | 000036 | | | | | |
| SRS- | G- | UUGGAACCGGGGUAACAGUCGGA | 15 | [symbol] | 169 | P- | CGACUGUUACCCCGGUUCCAA | 323 | [symbol] | 477 | 408 |
| 000037 | 000033 | | | | | 000037 | | | | | |
| SRS- | G- | UGCUUGGAACCGGGGUAACAGUC | 16 | [symbol] | 170 | P- | CUGUUACCCCGGUUCCAAGCA | 324 | [symbol] | 478 | 411 |
| 000038 | 000034 | | | | | 000038 | | | | | |
| SRS- | G- | UGGCUUGGAACCGGGGUAACAGU | 17 | [symbol] | 171 | P- | UGUUACCCCGGUUCCAAGCCA | 325 | [symbol] | 479 | 412 |
| 000039 | 000035 | | | | | 000039 | | | | | |
| SRS- | G- | UUCUAGGCUUGGAACCGGGGUAA | 18 | [symbol] | 172 | P- | ACCCCGGUUCCAAGCCUAGAA | 326 | [symbol] | 480 | 416 |
| 000040 | 000012 | | | | | 000040 | | | | | |
| SRS- | G- | UCUCUAGGCUUGGAACCGGGGUA | 19 | [symbol] | 173 | P- | CCCCGGUUCCAAGCCUAGAGA | 327 | [symbol] | 481 | 417 |
| 000041 | 000036 | | | | | 000041 | | | | | |
| SRS- | G- | UGCCUCUAGGCUUGGAACCGGGG | 20 | [symbol] | 174 | P- | CCGGUUCCAAGCCUAGAGGCA | 328 | [symbol] | 482 | 419 |
| 000042 | 000037 | | | | | 000042 | | | | | |
| SRS- | G- | UAGCCUCUAGGCUUGGAACCGGG | 21 | [symbol] | 175 | P- | CGGUUCCAAGCCUAGAGGCUA | 329 | [symbol] | 483 | 420 |
| 000043 | 000013 | | | | | 000043 | | | | | |
| SRS- | G- | UGAGCCUCUAGGCUUGGAACCGG | 22 | [symbol] | 176 | P- | GGUUCCAAGCCUAGAGGCUCA | 330 | [symbol] | 484 | 423 |
| 000044 | 000038 | | | | | 000044 | | | | | |
| SRS- | G- | UGAAGGAGCCUCUAGGCUUGGAA | 23 | [symbol] | 177 | P- | CCAAGCCUAGAGGCUCCUUCA | 331 | [symbol] | 485 | 425 |
| 000045 | 000039 | | | | | 000045 | | | | | |
| SRS- | G- | UCGGAAGGAGCCUCUAGGCUUGG | 24 | [symbol] | 178 | P- | AAGCCUAGAGGCUCCUUCCGA | 332 | [symbol] | 486 | 427 |
| 000046 | 000040 | | | | | 000046 | | | | | |
| SRS- | G- | UGUUCGGAAGGAGCCUCUAGGCU | 25 | [symbol] | 179 | P- | CCUAGAGGCUCCUUCCGAACA | 333 | [symbol] | 487 | 430 |
| 000047 | 000041 | | | | | 000047 | | | | | |
| SRS- | G- | UUGCUUGUUCGGAAGGAGCCUCU | 26 | [symbol] | 180 | P- | AGGCUCCUUCCGAACAAGCAA | 334 | [symbol] | 488 | 435 |
| 000048 | 000042 | | | | | 000048 | | | | | |
| SRS- | G- | UGUGCUUGUUCGGAAGGAGCCUC | 27 | [symbol] | 181 | P- | GGCUCCUUCCGAACAAGCACA | 335 | [symbol] | 489 | 436 |
| 000049 | 000043 | | | | | 000049 | | | | | |
| SRS- | G- | UCGGUGCUUGUUCGGAAGGAGCC | 28 | [symbol] | 182 | P- | CUCCUUCCGAACAAGCACCGA | 336 | [symbol] | 490 | 438 |
| 000050 | 000044 | | | | | 000050 | | | | | |
| SRS- | G- | UUCGGUGCUUGUUCGGAAGGAGC | 29 | [symbol] | 183 | P- | UCCUUCCGAACAAGCACCGAA | 337 | [symbol] | 491 | 439 |
| 000051 | 000045 | | | | | 000051 | | | | | |
| SRS- | G- | UCAGUCGGUGCUUGUUCGGAAGG | 30 | [symbol] | 184 | P- | UUCCGAACAAGCACCGACUGA | 338 | [symbol] | 492 | 442 |
| 000052 | 000046 | | | | | 000052 | | | | | |
| SRS- | G- | UUGCUCAGUCGGUGCUUGUUCGG | 31 | [symbol] | 185 | P- | GAACAAGCACCGACUGAGCAA | 339 | [symbol] | 493 | 446 |
| 000053 | 000047 | | | | | 000053 | | | | | |
| SRS- | G- | UUAGCACUCCUGCACCCCAGGCC | 32 | [symbol] | 186 | P- | CCUGGGGUGCAGGAGUGCUAA | 340 | [symbol] | 494 | 470 |
| 000054 | 000048 | | | | | 000054 | | | | | |
| SRS- | G- | UGUAGCACUCCUGCACCCCAGGC | 33 | [symbol] | 187 | P- | CUGGGGUGCAGGAGUGCUACA | 341 | [symbol] | 495 | 471 |
| 000055 | 000049 | | | | | 000055 | | | | | |
| SRS- | G- | UGAUAACUCUGUCCAUUACCAUG | 34 | [symbol] | 188 | P- | UGGUAAUGGACAGAGUUAUCA | 342 | [symbol] | 496 | 493 |
| 000056 | 000050 | | | | | 000056 | | | | | |
| SRS- | G- | UCGAUAACUCUGUCCAUUACCAU | 35 | [symbol] | 189 | P- | GGUAAUGGACAGAGUUAUCGA | 343 | [symbol] | 497 | 494 |
| 000057 | 000051 | | | | | 000057 | | | | | |
| SRS- | G- | UUAUGUGCCUCGAUAACUCUGUC | 36 | [symbol] | 190 | P- | CAGAGUUAUCGAGGCACAUAA | 344 | [symbol] | 498 | 503 |
| 000058 | 000052 | | | | | 000058 | | | | | |
| SRS- | G- | UGUAUGUGCCUCGAUAACUCUGU | 37 | [symbol] | 191 | P- | AGAGUUAUCGAGGCACAUACA | 345 | [symbol] | 499 | 504 |
| 000059 | 000053 | | | | | 000059 | | | | | |
| SRS- | G- | UAGUAUGUGCCUCGAUAACUCUG | 38 | [symbol] | 192 | P- | GAGUUAUCGAGGCACAUACUA | 346 | [symbol] | 500 | 505 |
| 000060 | 000054 | | | | | 000060 | | | | | |
| SRS- | G- | UACAGUGGUGGAGUAUGUGCCUC | 39 | [symbol] | 193 | P- | GGCACAUACUCCACCACUGUA | 347 | [symbol] | 501 | 515 |
| 000061 | 000055 | | | | | 000061 | | | | | |
| SRS- | G- | UACCAAGCUUGGCAGGUUCUUCC | 40 | [symbol] | 194 | P- | AAGAACCUGCCAAGCUUGGUA | 348 | [symbol] | 502 | 541 |
| 000062 | 000056 | | | | | 000062 | | | | | |
| SRS- | G- | UAUGACCAAGCUUGGCAGGUUCU | 41 | [symbol] | 195 | P- | AACCUGCCAAGCUUGGUCAUA | 349 | [symbol] | 503 | 544 |
| 000063 | 000057 | | | | | 000063 | | | | | |
| SRS- | G- | UAGAUGACCAAGCUUGGCAGGUU | 42 | [symbol] | 196 | P- | CCUGCCAAGCUUGGUCAUCUA | 350 | [symbol] | 504 | 546 |
| 000064 | 000058 | | | | | 000064 | | | | | |
| SRS- | G- | UUACCGUGGUAGCACUCCUGCAC | 43 | [symbol] | 197 | P- | GCAGGAGUGCUACCACGGUAA | 351 | [symbol] | 505 | 1162 |
| 000065 | 000059 | | | | | 000065 | | | | | |
| SRS- | G- | UUUACCGUGGUAGCACUCCUGCA | 44 | [symbol] | 198 | P- | CAGGAGUGCUACCACGGUAAA | 352 | [symbol] | 506 | 1163 |
| 000066 | 000014 | | | | | 000066 | | | | | |
| SRS- | G- | UCAUUACCGUGGUAGCACUCCUG | 45 | [symbol] | 199 | P- | GGAGUGCUACCACGGUAAUGA | 353 | [symbol] | 507 | 1165 |
| 000067 | 000060 | | | | | 000067 | | | | | |
| SRS- | G- | UGUCCAUUACCGUGGUAGCACUC | 46 | [symbol] | 200 | P- | GUGCUACCACGGUAAUGGACA | 354 | [symbol] | 508 | 1168 |
| 000068 | 000061 | | | | | 000068 | | | | | |
| SRS- | G- | UUGUCCAUUACCGUGGUAGCACU | 47 | [symbol] | 201 | P- | UGCUACCACGGUAAUGGACAA | 355 | [symbol] | 509 | 1169 |
| 000069 | 000015 | | | | | 000069 | | | | | |
| SRS- | G- | UUCUGUCCAUUACCGUGGUAGCA | 48 | [symbol] | 202 | P- | CUACCACGGUAAUGGACAGAA | 356 | [symbol] | 510 | 1171 |
| 000070 | 000062 | | | | | 000070 | | | | | |
| SRS- | G- | UCUCUGUCCAUUACCGUGGUAGC | 49 | [symbol] | 203 | P- | UACCACGGUAAUGGACAGAGA | 357 | [symbol] | 511 | 1172 |
| 000071 | 000016 | | | | | 000071 | | | | | |
| SRS- | G- | UAACUCUGUCCAUUACCGUGGUA | 50 | [symbol] | 204 | P- | CCACGGUAAUGGACAGAGUUA | 358 | [symbol] | 512 | 1174 |
| 000072 | 000063 | | | | | 000072 | | | | | |
| SRS- | G- | UGAUAACUCUGUCCAUUACCGUG | 51 | [symbol] | 205 | P- | CGGUAAUGGACAGAGUUAUCA | 359 | [symbol] | 513 | 1177 |
| 000073 | 000064 | | | | | 000073 | | | | | |
| SRS- | G- | UCGAUAACUCUGUCCAUUACCGU | 52 | [symbol] | 206 | P- | GGUAAUGGACAGAGUUAUCGA | 360 | [symbol] | 514 | 1178 |
| 000074 | 000065 | | | | | 000074 | | | | | |
| SRS- | G- | UACAGGAUCUGGAUUCCUGCAGU | 53 | [symbol] | 207 | P- | UGCAGGAAUCCAGAUCCUGUA | 361 | [symbol] | 515 | 2684 |
| 000075 | 000066 | | | | | 000075 | | | | | |
| SRS- | G- | UAAUAAGGGGCUGCCACAGGAUC | 54 | [symbol] | 208 | P- | UCCUGUGGCAGCCCCUUAUUA | 362 | [symbol] | 516 | 2698 |
| 000076 | 000067 | | | | | 000076 | | | | | |
| SRS- | G- | UAACAAUAAGGGGCUGCCACAGG | 55 | [symbol] | 209 | P- | UGUGGCAGCCCCUUAUUGUUA | 363 | [symbol] | 517 | 2701 |
| 000077 | 000068 | | | | | 000077 | | | | | |
| SRS- | G- | UGUAUAACAAUAAGGGGCUGCCA | 56 | [symbol] | 210 | P- | GCAGCCCCUUAUUGUUAUACA | 364 | [symbol] | 518 | 2705 |
| 000078 | 000069 | | | | | 000078 | | | | | |
| SRS- | G- | UCGUAUAACAAUAAGGGGCUGCC | 57 | [symbol] | 211 | P- | CAGCCCCUUAUUGUUAUACGA | 365 | [symbol] | 519 | 2706 |
| 000079 | 000070 | | | | | 000079 | | | | | |
| SRS- | G- | UUCGUAUAACAAUAAGGGGCUGC | 58 | [symbol] | 212 | P- | AGCCCCUUAUUGUUAUACGAA | 366 | [symbol] | 520 | 2707 |
| 000080 | 000071 | | | | | 000080 | | | | | |
| SRS- | G- | UUGUGUCAGGUUGCAGUACUCCC | 59 | [symbol] | 213 | P- | GAGUACUGCAACCUGACACAA | 367 | [symbol] | 521 | 2747 |
| 000081 | 000072 | | | | | 000081 | | | | | |
| SRS- | G- | UAUUGUGUCAGGUUGCAGUACUC | 60 | [symbol] | 214 | P- | GUACUGCAACCUGACACAAUA | 368 | [symbol] | 522 | 2749 |
| 000082 | 000017 | | | | | 000082 | | | | | |
| SRS- | G- | UGCAUUGUGUCAGGUUGCAGUAC | 61 | [symbol] | 215 | P- | ACUGCAACCUGACACAAUGCA | 369 | [symbol] | 523 | 2751 |
| 000083 | 000073 | | | | | 000083 | | | | | |
| SRS- | G- | UGAGCAUUGUGUCAGGUUGCAGU | 62 | [symbol] | 216 | P- | UGCAACCUGACACAAUGCUCA | 370 | [symbol] | 524 | 2753 |
| 000084 | 000074 | | | | | 000084 | | | | | |
| SRS- | G- | UUGAGCAUUGUGUCAGGUUGCAG | 63 | [symbol] | 217 | P- | GCAACCUGACACAAUGCUCAA | 371 | [symbol] | 525 | 2754 |
| 000085 | 000075 | | | | | 000085 | | | | | |
| SRS- | G | UUGCGUCUGAGCAUUGUGUCAGG | 64 | [symbol] | 218 | P- | UGACACAAUGCUCAGACGCAA | 372 | [symbol] | 526 | 2760 |
| 000086 | 000018 | | | | | 000086 | | | | | |
| SRS- | G- | UUCUGCGUCUGAGCAUUGUGUCA | 65 | [symbol] | 219 | P- | ACACAAUGCUCAGACGCAGAA | 373 | [symbol] | 527 | 2762 |
| 000087 | 000076 | | | | | 000087 | | | | | |
| SRS- | G- | UCUUCUGCGUCUGAGCAUUGUGU | 66 | [symbol] | 220 | P- | ACAAUGCUCAGACGCAGAAGA | 374 | [symbol] | 528 | 2764 |
| 000088 | 000077 | | | | | 000088 | | | | | |
| SRS- | G- | UGAGCCUCUAGGCUUGGAAUCGG | 67 | [symbol] | 221 | P- | GAUUCCAAGCCUAGAGGCUCA | 375 | [symbol] | 529 | 2815 |
| 000089 | 000078 | | | | | 000089 | | | | | |
| SRS- | G- | UUUCAGAAGGAGCCUCUAGGCUU | 68 | [symbol] | 222 | P- | GCCUAGAGGCUCCUUCUGAAA | 376 | [symbol] | 530 | 2823 |
| 000090 | 000079 | | | | | 000090 | | | | | |
| SRS- | G- | UUUGCUCAGUUGGUGCUUGUUCA | 69 | [symbol] | 223 | P- | AACAAGCACCAACUGAGCAAA | 377 | [symbol] | 531 | 2841 |
| 000091 | 000080 | | | | | 000091 | | | | | |
| SRS- | G- | UCUUUGCUCAGUUGGUGCUUGUU | 70 | [symbol] | 224 | P- | CAAGCACCAACUGAGCAAAGA | 378 | [symbol] | 532 | 2843 |
| 000092 | 000081 | | | | | 000092 | | | | | |
| SRS- | G- | UGUGCCCUGAUAACUCUGUCCAU | 71 | [symbol] | 225 | P- | GGACAGAGUUAUCAAGGCACA | 379 | [symbol] | 533 | 2894 |
| 000093 | 000082 | | | | | 000093 | | | | | |
| SRS- | G- | UGGAUCUGGAUUUCGGCAGUAGU | 72 | [symbol] | 226 | P- | UACUGCCGAAAUCCAGAUCCA | 380 | [symbol] | 534 | 3023 |
| 000094 | 000083 | | | | | 000094 | | | | | |
| SRS- | G- | UUAACACCAAGGGGCUGCCACAG | 73 | [symbol] | 227 | P- | GUGGCAGCCCCUUGGUGUUAA | 381 | [symbol] | 535 | 3044 |
| 000095 | 000084 | | | | | 000095 | | | | | |
| SRS- | G- | UGUAUAACACCAAGGGGCUGCCA | 74 | [symbol] | 228 | P- | GCAGCCCCUUGGUGUUAUACA | 382 | [symbol] | 536 | 3047 |
| 000096 | 000085 | | | | | 000096 | | | | | |
| SRS- | G- | UUUGUAUAACACCAAGGGGCUGC | 75 | [symbol] | 229 | P- | AGCCCCUUGGUGUUAUACAAA | 383 | [symbol] | 537 | 3049 |
| 000097 | 000086 | | | | | 000097 | | | | | |
| SRS- | G- | UGUUGUAUAACACCAAGGGGCUG | 76 | [symbol] | 230 | P- | GCCCCUUGGUGUUAUACAACA | 384 | [symbol] | 538 | 3050 |
| 000098 | 000087 | | | | | 000098 | | | | | |
| SRS | G | UUCGUGUCAGGUUGCAGUACUCC | 77 | [symbol] | 231 | P- | AGUACUGCAACCUGACACGAA | 385 | [symbol] | 539 | 3090 |
| 000099 | 000088 | | | | | 000099 | | | | | |
| SRS- | G- | UAGCAUCGUGUCAGGUUGCAGUA | 78 | [symbol] | 232 | P- | CUGCAACCUGACACGAUGCUA | 386 | [symbol] | 540 | 3094 |
| 000100 | 000089 | | | | | 000100 | | | | | |
| SRS- | G- | UGAGCAUCGUGUCAGGUUGCAGU | 79 | [symbol] | 233 | P- | UGCAACCUGACACGAUGCUCA | 387 | [symbol] | 541 | 3095 |
| 000101 | 000090 | | | | | 000101 | | | | | |
| SRS- | G- | UUGAGCAUCGUGUCAGGUUGCAG | 80 | [symbol] | 234 | P- | GCAACCUGACACGAUGCUCAA | 388 | [symbol] | 542 | 3096 |
| 000102 | 000091 | | | | | 000102 | | | | | |
| SRS- | G- | UUCUGAGCAUCGUGUCAGGUUGC | 81 | [symbol] | 235 | P- | AACCUGACACGAUGCUCAGAA | 389 | [symbol] | 543 | 3098 |
| 000103 | 000092 | | | | | 000103 | | | | | |
| SRS- | G | UAAAGCCUCUAGGCUUGGAGCCA | 82 | [symbol] | 236 | P- | GCUCCAAGCCUAGAGGCUUUA | 390 | [symbol] | 544 | 3158 |
| 000104 | 000093 | | | | | 000104 | | | | | |
| SRS- | G- | UCCAUAAUGGUAGUAGCAGUCCU | 83 | [symbol] | 237 | P- | GACUGCUACUACCAUUAUGGA | 391 | [symbol] | 545 | 3218 |
| 000105 | 000094 | | | | | 000105 | | | | | |
| SRS- | G- | UUCCAUAAUGGUAGUAGCAGUCC | 84 | [symbol] | 238 | P- | ACUGCUACUACCAUUAUGGAA | 392 | [symbol] | 546 | 3219 |
| 000106 | 000095 | | | | | 000106 | | | | | |
| SRS- | G- | UGUCCAUAAUGGUAGUAGCAGUC | 85 | [symbol] | 239 | P- | CUGCUACUACCAUUAUGGACA | 393 | [symbol] | 547 | 3220 |
| 000107 | 000096 | | | | | 000107 | | | | | |
| SRS- | G- | UUCUGUCCAUAAUGGUAGUAGCA | 86 | [symbol] | 240 | P- | CUACUACCAUUAUGGACAGAA | 394 | [symbol] | 548 | 3223 |
| 000108 | 000097 | | | | | 000108 | | | | | |
| SRS- | G- | UAACUCUGUCCAUAAUGGUAGUA | 87 | [symbol] | 241 | P- | CUACCAUUAUGGACAGAGUUA | 395 | [symbol] | 549 | 3226 |
| 000109 | 000098 | | | | | 000109 | | | | | |
| SRS- | G- | UUAACUCUGUCCAUAAUGGUAGU | 88 | [symbol] | 242 | P- | UACCAUUAUGGACAGAGUUAA | 396 | [symbol] | 550 | 3227 |
| 000110 | 000019 | | | | | 000110 | | | | | |
| SRS- | G- | UCCAAGCUUGGCAAGUUCUUCCU | 89 | [symbol] | 243 | P- | GAAGAACUUGCCAAGCUUGGA | 397 | [symbol] | 551 | 3276 |
| 000111 | 000020 | | | | | 000111 | | | | | |
| SRS- | G- | UAGAUGACCAAGCUUGGCAAGUU | 90 | [symbol] | 244 | P- | CUUGCCAAGCUUGGUCAUCUA | 398 | [symbol] | 552 | 3282 |
| 000112 | 000021 | | | | | 000112 | | | | | |
| SRS- | G- | UAUAGAUGACCAAGCUUGGCAAG | 91 | [symbol] | 245 | P- | UGCCAAGCUUGGUCAUCUAUA | 399 | [symbol] | 553 | 3284 |
| 000113 | 000099 | | | | | 000113 | | | | | |
| SRS- | G- | UCAUAGAUGACCAAGCUUGGCAA | 92 | [symbol] | 246 | P- | GCCAAGCUUGGUCAUCUAUGA | 400 | [symbol] | 554 | 3285 |
| 000114 | 000100 | | | | | 000114 | | | | | |
| SRS- | G- | UACUAUGCUGGUGUGGUGUCAUA | 93 | [symbol] | 247 | P- | UGACACCACACCAGCAUAGUA | 401 | [symbol] | 555 | 3303 |
| 000115 | 000101 | | | | | 000115 | | | | | |
| SRS- | G- | UGACUAUGCUGGUGUGGUGUCAU | 94 | [symbol] | 248 | P- | GACACCACACCAGCAUAGUCA | 402 | [symbol] | 556 | 3304 |
| 000116 | 000102 | | | | | 000116 | | | | | |
| SRS- | G- | UCGACUAUGCUGGUGUGGUGUCA | 95 | [symbol] | 249 | P- | ACACCACACCAGCAUAGUCGA | 403 | [symbol] | 557 | 3305 |
| 000117 | 000103 | | | | | 000117 | | | | | |
| SRS- | G- | UUCCGACUAUGCUGGUGUGGUGU | 96 | [symbol] | 250 | P- | ACCACACCAGCAUAGUCGGAA | 404 | [symbol] | 558 | 3307 |
| 000118 | 000104 | | | | | 000118 | | | | | |
| SRS- | G- | UGUCCGACUAUGCUGGUGUGGUG | 97 | [symbol] | 251 | P- | CCACACCAGCAUAGUCGGACA | 405 | [symbol] | 559 | 3308 |
| 000119 | 000105 | | | | | 000119 | | | | | |
| SRS- | G- | UGGUCCGACUAUGCUGGUGUGGU | 98 | [symbol] | 252 | P- | CACACCAGCAUAGUCGGACCA | 406 | [symbol] | 560 | 3309 |
| 000120 | 000022 | | | | | 000120 | | | | | |
| SRS- | G- | UUUUCUGGGGUCCGACUAUGCUG | 99 | [symbol] | 253 | P- | GCAUAGUCGGACCCCAGAAAA | 407 | [symbol] | 561 | 3316 |
| 000121 | 000106 | | | | | 000121 | | | | | |
| SRS- | G- | UUUUUCUGGGGUCCGACUAUGCU | 100 | [symbol] | 254 | P- | CAUAGUCGGACCCCAGAAAAA | 408 | [symbol] | 562 | 3317 |
| 000122 | 000107 | | | | | 000122 | | | | | |
| SRS- | G- | UUAGUUUUCUGGGGUCCGACUAU | 101 | [symbol] | 255 | P- | AGUCGGACCCCAGAAAACUAA | 409 | [symbol] | 563 | 3320 |
| 000123 | 000108 | | | | | 000123 | | | | | |
| SRS- | G- | UAUCCAUGGUGUAACACCAAGGG | 102 | [symbol] | 256 | P- | CUUGGUGUUACACCAUGGAUA | 410 | [symbol] | 564 | 3396 |
| 000124 | 000109 | | | | | 000124 | | | | | |
| SRS- | G- | UGAUCCAUGGUGUAACACCAAGG | 103 | [symbol] | 257 | P- | UUGGUGUUACACCAUGGAUCA | 411 | [symbol] | 565 | 3397 |
| 000125 | 000110 | | | | | 000125 | | | | | |
| SRS- | G- | UGGAUCCAUGGUGUAACACCAAG | 104 | [symbol] | 258 | P- | UGGUGUUACACCAUGGAUCCA | 412 | [symbol] | 566 | 3398 |
| 000126 | 000111 | | | | | 000126 | | | | | |
| SRS- | G- | UGGGAUCCAUGGUGUAACACCAA | 105 | [symbol] | 259 | P- | GGUGUUACACCAUGGAUCCCA | 413 | [symbol] | 567 | 3399 |
| 000127 | 000112 | | | | | 000127 | | | | | |
| SRS- | G- | UUGACACUGGGAUCCAUGGUGUA | 106 | [symbol] | 260 | P- | CACCAUGGAUCCCAGUGUCAA | 414 | [symbol] | 568 | 3406 |
| 000128 | 000113 | | | | | 000128 | | | | | |
| SRS- | G- | UCUGACACUGGGAUCCAUGGUGU | 107 | [symbol] | 261 | P- | ACCAUGGAUCCCAGUGUCAGA | 415 | [symbol] | 569 | 3407 |
| 000129 | 000114 | | | | | 000129 | | | | | |
| SRS- | G- | UGUUGCAAGGACACUUGAUUCUG | 108 | [symbol] | 262 | P- | GAAUCAAGUGUCCUUGCAACA | 416 | [symbol] | 570 | 3464 |
| 000130 | 000115 | | | | | 000130 | | | | | |
| SRS- | G- | UCAUGGUAUAACACCAAGGACUA | 109 | [symbol] | 263 | P- | GUCCUUGGUGUUAUACCAUGA | 417 | [symbol] | 571 | 3735 |
| 000131 | 000116 | | | | | 000131 | | | | | |
| SRS- | G- | UUCCAUGGUAUAACACCAAGGAC | 110 | [symbol] | 264 | P- | CCUUGGUGUUAUACCAUGGAA | 418 | [symbol] | 572 | 3737 |
| 000132 | 000117 | | | | | 000132 | | | | | |
| SRS- | G- | UAUCCAUGGUAUAACACCAAGGA | 111 | [symbol] | 265 | P- | CUUGGUGUUAUACCAUGGAUA | 419 | [symbol] | 573 | 3738 |
| 000133 | 000118 | | | | | 000133 | | | | | |
| SRS- | G- | UGGGAUCCAUGGUAUAACACCAA | 112 | [symbol] | 266 | P- | GGUGUUAUACCAUGGAUCCCA | 420 | [symbol] | 574 | 3741 |
| 000134 | 000119 | | | | | 000134 | | | | | |
| SRS- | G- | UGACAUUGUGUCAGGUUGCAGUA | 113 | [symbol] | 267 | P- | CUGCAACCUGACACAAUGUCA | 421 | [symbol] | 575 | 3778 |
| 000135 | 000120 | | | | | 000135 | | | | | |
| SRS- | G- | UGGACAUUGUGUCAGGUUGCAGU | 114 | [symbol] | 268 | P- | UGCAACCUGACACAAUGUCCA | 422 | [symbol] | 576 | 3779 |
| 000136 | 000121 | | | | | 000136 | | | | | |
| SRS- | G- | UUGGACAUUGUGUCAGGUUGCAG | 115 | [symbol] | 269 | P- | GCAACCUGACACAAUGUCCAA | 423 | [symbol] | 577 | 3780 |
| 000137 | 000122 | | | | | 000137 | | | | | |
| SRS- | G- | UCACUGGACAUUGUGUCAGGUUG | 116 | [symbol] | 270 | P- | ACCUGACACAAUGUCCAGUGA | 424 | [symbol] | 578 | 3783 |
| 000138 | 000123 | | | | | 000138 | | | | | |
| SRS- | G- | UAUUCUGUCACUGGACAUUGUGU | 117 | [symbol] | 271 | P- | ACAAUGUCCAGUGACAGAAUA | 425 | [symbol] | 579 | 3790 |
| 000139 | 000124 | | | | | 000139 | | | | | |
| SRS- | G- | UCACCAUUUGGGUAGUAUUCUGU | 118 | [symbol] | 272 | P- | AGAAUACUACCCAAAUGGUGA | 426 | [symbol] | 580 | 3991 |
| 000140 | 000125 | | | | | 000140 | | | | | |
| SRS- | G- | UGUAUAACACCAAGGGCGAAUCU | 119 | [symbol] | 273 | P- | AUUCGCCCUUGGUGUUAUACA | 427 | [symbol] | 581 | 4049 |
| 000141 | 000126 | | | | | 000141 | | | | | |
| SRS- | G- | UCAUGGUAUAACACCAAGGGCGA | 120 | [symbol] | 274 | P- | GCCCUUGGUGUUAUACCAUGA | 428 | [symbol] | 582 | 4053 |
| 000142 | 000127 | | | | | 000142 | | | | | |
| SRS- | G- | UUCCAUGGUAUAACACCAAGGGC | 121 | [symbol] | 275 | P- | CCUUGGUGUUAUACCAUGGAA | 429 | [symbol] | 583 | 4055 |
| 000143 | 000128 | | | | | 000143 | | | | | |
| SRS- | G- | UAUCCAUGGUAUAACACCAAGGG | 122 | [symbol] | 276 | P- | CUUGGUGUUAUACCAUGGAUA | 430 | [symbol] | 584 | 4056 |
| 000144 | 000129 | | | | | 000144 | | | | | |
| SRS- | G- | UUGACACUGGGAUCCAUGGUAUA | 123 | [symbol] | 277 | P- | UACCAUGGAUCCCAGUGUCAA | 431 | [symbol] | 585 | 4066 |
| 000145 | 000130 | | | | | 000145 | | | | | |
| SRS- | G- | UCUGACACUGGGAUCCAUGGUAU | 124 | [symbol] | 278 | P- | ACCAUGGAUCCCAGUGUCAGA | 432 | [symbol] | 586 | 4067 |
| 000146 | 000131 | | | | | 000146 | | | | | |
| SRS- | G- | UGGACCACCGUGGGAGUUGUGAG | 125 | [symbol] | 279 | P- | CACAACUCCCACGGUGGUCCA | 433 | [symbol] | 587 | 4138 |
| 000147 | 000132 | | | | | 000147 | | | | | |
| SRS- | G- | UGGGACCACCGUGGGAGUUGUGA | 126 | [symbol] | 280 | P- | ACAACUCCCACGGUGGUCCCA | 434 | [symbol] | 588 | 4139 |
| 000148 | 000133 | | | | | 000148 | | | | | |
| SRS- | G- | UUGCUUGGAACUGGGACCACCGU | 127 | [symbol] | 281 | P- | GGUGGUCCCAGUUCCAAGCAA | 435 | [symbol] | 589 | 4150 |
| 000149 | 000134 | | | | | 000149 | | | | | |
| SRS- | G- | UAUUCUGUCACUGGACAUCGUGU | 128 | [symbol] | 282 | P- | ACGAUGUCCAGUGACAGAAUA | 436 | [symbol] | 590 | 4450 |
| 000150 | 000135 | | | | | 000150 | | | | | |
| SRS- | G- | UGAUGCCAGUGUGGUAUCAUAGA | 129 | [symbol] | 283 | P- | UAUGAUACCACACUGGCAUCA | 437 | [symbol] | 591 | 4645 |
| 000151 | 000136 | | | | | 000151 | | | | | |
| SRS- | G- | UUGAUGCCAGUGUGGUAUCAUAG | 130 | [symbol] | 284 | P- | AUGAUACCACACUGGCAUCAA | 438 | [symbol] | 592 | 4646 |
| 000152 | 000137 | | | | | 000152 | | | | | |
| SRS- | G- | UGUGUAACACCAGGGUUGUUUCC | 131 | [symbol] | 285 | P- | AAACAACCCUGGUGUUACACA | 439 | [symbol] | 593 | 4733 |
| 000153 | 000138 | | | | | 000153 | | | | | |
| SRS- | G- | UAGGACACCUGAUUCUGUUUCUG | 132 | [symbol] | 286 | P- | GAAACAGAAUCAGGUGUCCUA | 440 | [symbol] | 594 | 4802 |
| 000154 | 000139 | | | | | 000154 | | | | | |
| SRS- | G- | UUAGGACACCUGAUUCUGUUUCU | 133 | [symbol] | 287 | P- | AAACAGAAUCAGGUGUCCUAA | 441 | [symbol] | 595 | 4803 |
| 000155 | 000140 | | | | | 000155 | | | | | |
| SRS- | G- | UUCUAGGACACCUGAUUCUGUUU | 134 | [symbol] | 288 | P- | ACAGAAUCAGGUGUCCUAGAA | 442 | [symbol] | 596 | 4805 |
| 000156 | 000141 | | | | | 000156 | | | | | |
| SRS- | G- | UCUCUAGGACACCUGAUUCUGUU | 135 | [symbol] | 289 | P- | CAGAAUCAGGUGUCCUAGAGA | 443 | [symbol] | 597 | 4806 |
| 000157 | 000142 | | | | | 000157 | | | | | |
| SRS- | G- | UGUCUCUAGGACACCUGAUUCUG | 136 | [symbol] | 290 | P- | GAAUCAGGUGUCCUAGAGACA | 444 | [symbol] | 598 | 4808 |
| 000158 | 000143 | | | | | 000158 | | | | | |
| SRS- | G- | UGGGAGUCUCUAGGACACCUGAU | 137 | [symbol] | 291 | P- | CAGGUGUCCUAGAGACUCCCA | 445 | [symbol] | 599 | 4812 |
| 000159 | 000144 | | | | | 000159 | | | | | |
| SRS- | G- | UGGAACAACAGUGGGAGUCUCUA | 138 | [symbol] | 292 | P- | GAGACUCCCACUGUUGUUCCA | 446 | [symbol] | 600 | 4823 |
| 000160 | 000145 | | | | | 000160 | | | | | |
| SRS- | G- | UGAAUGUGCCUCGAUAACUCUGG | 139 | [symbol] | 293 | P- | AGAGUUAUCGAGGCACAUUCA | 447 | [symbol] | 601 | 4926 |
| 000161 | 000146 | | | | | 000161 | | | | | |
| SRS- | G- | UUGGAUGACCAAGAUUGACAUGU | 140 | [symbol] | 294 | P- | AUGUCAAUCUUGGUCAUCCAA | 448 | [symbol] | 602 | 4969 |
| 000162 | 000147 | | | | | 000162 | | | | | |
| SRS- | G- | UAUGGAUGACCAAGAUUGACAUG | 141 | [symbol] | 295 | P- | UGUCAAUCUUGGUCAUCCAUA | 449 | [symbol] | 603 | 4970 |
| 000163 | 000148 | | | | | 000163 | | | | | |
| SRS- | G- | UAAAACACCAAGGGOCUGUAUCG | 142 | [symbol] | 296 | P- | AUACAGGCCCUUGGUGUUUUA | 450 | [symbol] | 604 | 5073 |
| 000164 | 000149 | | | | | 000164 | | | | | |
| SRS- | G- | UGCAUCGCGUCAGGUUGCAGUAC | 143 | [symbol] | 297 | P- | ACUGCAACCUGACGCGAUGCA | 451 | [symbol] | 605 | 5121 |
| 000165 | 000150 | | | | | 000165 | | | | | |
| SRS- | G- | UAGCAUCGCGUCAGGUUGCAGUA | 144 | [symbol] | 298 | P- | CUGCAACCUGACGCGAUGCUA | 452 | [symbol] | 606 | 5122 |
| 000166 | 000151 | | | | | 000166 | | | | | |
| SRS- | G- | UGAGCAUCGCGUCAGGUUGCAGU | 145 | [symbol] | 299 | P- | UGCAACCUGACGCGAUGCUCA | 453 | [symbol] | 607 | 5123 |
| 000167 | 000152 | | | | | 000167 | | | | | |
| SRS- | G | UUGAGCAUCGCGUCAGGUUGCAG | 146 | [symbol] | 300 | P- | GCAACCUGACGCGAUGCUCAA | 454 | [symbol] | 608 | 5124 |
| 000168 | 000153 | | | | | 000168 | | | | | |
| SRS- | G- | UUCUGAGCAUCGCGUCAGGUUGC | 147 | [symbol] | 301 | P- | AACCUGACGCGAUGCUCAGAA | 455 | [symbol] | 609 | 5126 |
| 000169 | 000154 | | | | | 000169 | | | | | |
| SRS- | G- | UGUCUGAGCAUCGCGUCAGGUUG | 148 | [symbol] | 302 | P- | ACCUGACGCGAUGCUCAGACA | 456 | [symbol] | 610 | 5127 |
| 000170 | 000155 | | | | | 000170 | | | | | |
| SRS- | G- | UUGUCUGAGCAUCGCGUCAGGUU | 149 | [symbol] | 303 | P- | CCUGACGCGAUGCUCAGACAA | 457 | [symbol] | 611 | 5128 |
| 000171 | 000156 | | | | | 000171 | | | | | |
| SRS- | G- | UGUGUCUGAGCAUCGCGUCAGGU | 150 | [symbol] | 304 | P- | CUGACGCGAUGCUCAGACACA | 458 | [symbol] | 612 | 5129 |
| 000172 | 000157 | | | | | 000172 | | | | | |
| SRS- | G- | UUGUGUCUGAGCAUCGCGUCAGG | 151 | [symbol] | 305 | P- | UGACGCGAUGCUCAGACACAA | 459 | [symbol] | 613 | 5130 |
| 000173 | 000158 | | | | | 000173 | | | | | |
| SRS- | G- | UUCUGUGUCUGAGCAUCGCGUCA | 152 | [symbol] | 306 | P- | ACGCGAUGCUCAGACACAGAA | 460 | [symbol] | 614 | 5132 |
| 000174 | 000159 | | | | | 000174 | | | | | |
| SRS- | G- | UUACAAUGCUUCCAGGACAUUUC | 153 | [symbol] | 307 | P- | AAUGUCCUGGAAGCAUUGUAA | 461 | [symbol] | 615 | 5505 |
| 000175 | 000160 | | | | | 000175 | | | | | |
| SRS- | G- | UCAGUGAUGUAACAUUCAGUCCU | 154 | [symbol] | 308 | P- | GACUGAAUGUUACAUCACUGA | 462 | [symbol] | 616 | 5866 |
| 000176 | 000161 | | | | | 000176 | | | | | |

As used herein, “A” refers to adenosine-3′-phosphate; “a” refers to 2′-O-methyladenosine-3′-phosphate; “Af” refers to 2′-fluoroadenosine-3′-phosphate; “C” refers to cytidine-3′-phosphate; “c” refers to 2′-O-methylcytidine-3′-phosphate; “Cf” refers to 2′-fluorocytidine-3′-phosphate; “G” refers to guanosine-3′-phosphate; “g” refers to 2′-O-methylguanosine-3′-phosphate; “Gf” refers to 2′-fluoroguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “s” refers to 3′-phosphorothioate. The “position at human transcript” is defined as the 5′ position of a 21-mer target site in human transcript (NCBI Reference Sequence No: NM_005577.4).
Note that, in some instances, the sense strand sequence is coupled to a targeting moiety (e.g., GalNAc or galactose (e.g., L96)) at the 3′ end of the sense strand. [symbol]
indicates data missing or illegible when filed

### Conjugation

### Targeting Moiety

In certain aspects, the polynucleotide molecule described herein is coupled or conjugated with one or more targeting moieties to form a polynucleotide-targeting moiety conjugate molecule. In some instances, a targeting moiety is selected based on its ability to target the conjugate molecule described herein to a desired cell population, tissue, or an organ selectively or preferably. In some instances, the targeting moiety targets the cell, tissue, or an organ that expresses the corresponding binding partner (e.g., either the corresponding receptor or ligand) of the targeting moiety. For example, the polynucleotide molecule described herein could be targeted to hepatocytes expressing asialoglycoprotein (ASGP-R) by selecting a targeting moiety containing N-acetyl galactosamine (GalNAc) as the targeting moiety. Any suitable GalNAc molecules that are known in the art to be used as a targeting moiety are contemplated. Exemplary GalNAc molecule includes a triantennary GalNAc (e.g., L96). A further example of the targeting moiety is galactose. The targeting moiety can also be a lipid, peptide, or small molecule.

A targeting moiety (i.e., an intracellular targeting moiety) that targets a desired site within the cell (e.g., endoplasmic reticulum, Golgi apparatus, nucleus, or mitochondria) may be included in the hybridized polynucleotide constructs disclosed herein. Non-limiting examples of the intracellular targeting moieties are provided in WO 2015/069932 and in WO 2015/188197; the disclosure of the intracellular targeting moieties in WO 2015/069932 and in WO 2015/188197 is incorporated herein by reference.

The polynucleotide molecule described herein, thus, may include one or more targeting moieties selected from the group consisting of intracellular targeting moieties, extracellular targeting moieties, and combinations thereof. Thus, the inclusion of one or more targeting moieties (e.g., extracellular targeting moieties including targeting moieties independently selected from the group consisting of folate, mannose, N-acetyl galactosamine, and prostate specific membrane antigen) and one or more intracellular targeting moiety (e.g., a moiety targeting endoplasmic reticulum, Golgi apparatus, nucleus, or mitochondria) in the polynucleotide molecule described herein can facilitate the delivery of the polynucleotides to a specific site within the specific cell population. In some aspects, the targeting moiety contains one or more mannose carbohydrates. Mannose targets the mannose receptor, which is a 175 KDa membrane-associated receptor that is expressed on sinusoidal liver cells and antigen presenting cells (e.g., macrophages and dendritic cells). It is a highly effective endocytotic/recycling receptor that binds and internalizes mannosylated pathogens and proteins (Lennartz et. al. J. Biol. Chem. 262:9942-9944, 1987; Taylor et. al. J. Biol. Chem. 265:12156-62, 1990).

Some of the targeting moieties are described herein. In some aspects, the targeting moiety contains or specifically binds to a protein selected from the group including insulin, insulin-like growth factor receptor 1 (IGF1R), IGF2R, insulin-like growth factor (IGF; e.g., IGF 1 or 2), mesenchymal epithelial transition factor receptor (c-met; also known as hepatocyte growth factor receptor (HGFR)), hepatocyte growth factor (HGF), epidermal growth factor receptor (EGFR), epidermal growth factor (EGF), heregulin, fibroblast growth factor receptor (FGFR), platelet-derived growth factor receptor (PDGFR), platelet-derived growth factor (PDGF), vascular endothelial growth factor receptor (VEGFR), vascular endothelial growth factor (VEGF), tumor necrosis factor receptor (TNFR), tumor necrosis factor alpha (TNF-α), TNF-β, folate receptor (FOLR), folate, transferrin, transferrin receptor (TfR), mesothelin, Fc receptor, c-kit receptor, c-kit, an integrin (e.g., an α4 integrin or a β-1 integrin), P-selectin, sphingosine-1-phosphate receptor-1 (S1PR), hyaluronate receptor, leukocyte function antigen-1 (LFA-1), CD4, CD11, CD18, CD20, CD25, CD27, CD52, CD70, CD80, CD85, CD95 (Fas receptor), CD106 (vascular cell adhesion molecule 1 (VCAM1), CD166 (activated leukocyte cell adhesion molecule (ALCAM)), CD178 (Fas ligand), CD253 (TNF-related apoptosis-inducing ligand (TRAIL)), ICOS ligand, CCR2, CXCR3, CCR5, CXCL12 (stromal cell-derived factor 1 (SDF-1)), interleukin 1 (IL-1), IL-Ira, IL-2, IL-3, IL-4, IL-6, IL-7, IL-8, CTLA-4, MART-1, gp100, MAGE-1, ephrin (Eph) receptor, mucosal addressin cell adhesion molecule 1 (MAdCAM-1), carcinoembryonic antigen (CEA), LewisY, MUC-1, epithelial cell adhesion molecule (EpCAM), cancer antigen 125 (CA125), prostate specific membrane antigen (PSMA), TAG-72 antigen, and fragments thereof. In further aspects, the targeting moiety contains erythroblastic leukemia viral oncogene homolog (ErbB) receptor (e.g., ErbB1 receptor; ErbB2 receptor; ErbB3 receptor; and ErbB4 receptor). In some aspects, the targeting moiety contains one or more (e.g., from 1 to 6) N-acetyl galactosamines (GalNAc). In some aspects, the targeting moiety contains one or more (e.g., from 1 to 6) galactose. In certain aspects, the targeting moiety contains one or more (e.g., from 1 to 6) mannoses. In other aspects, the targeting moiety contains a folate ligand. The folate ligand has the structure:

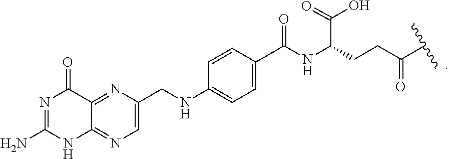

Certain targeting moieties may include bombesin, gastrin, gastrin-releasing peptide, tumor growth factors (TGF) (e.g., TGF-α or TGF-β), or vaccinia virus growth factor (VVGF). Non-peptidyl targeting moieties can also be used in the targeting moieties and may include, for example, steroids, carbohydrates, vitamins, and lectins. Some targeting moieties may include a polypeptide, such as somatostatin or somatostatin analog (e.g., octreotide or lanreotide), bombesin, or an antibody or antigen-binding fragment thereof. Antibodies may be of any recognized class or subclass, e.g., IgG, IgA, IgM, IgD, or IgE. Typical are those antibodies which fall within the IgG class. The antibodies can be derived from any species according to techniques known in the art. Typically, however, the antibody is of human, murine, or rabbit origin. In addition, the antibody may be polyclonal or monoclonal, but is typically monoclonal. Human or chimeric (e.g., humanized) antibodies may be used in targeting moieties. Targeting moieties may include an antigen-binding fragment of an antibody. Such antibody fragments may include, for example, the Fab′, F(ab′)2, Fv, or Fab fragments, single domain antibody, ScFv, or other antigen-binding fragments. Fc fragments may also be employed in targeting moieties. Such antibody fragments can be prepared, for example, by proteolytic enzyme digestion, for example, by pepsin or papain digestion, reductive alkylation, or recombinant techniques. The materials and methods for preparing antibody fragments are well-known to those skilled in the art. See, e.g., Parham, J. Immunology, 131:2895, 1983; Lamoyi et al., J. Immunological Methods, 56:235, 1983. Other peptides for use as a targeting auxiliary moiety in polynucleotide molecule described herein can be selected from KiSS peptides and analogs, urotensin II peptides and analogs, GnRH I and II peptides and analogs, depreotide, vapreotide, vasoactive intestinal peptide (VIP), cholecystokinin (CCK), RGD-containing peptides, melanocyte-stimulating hormone (MSH) peptide, neurotensin, calcitonin, glutathione, YIGSR (leukocyte-avid peptides, e.g., P483H, which contains the heparin-binding region of platelet factor-4 (PF-4) and a lysine-rich sequence), atrial natriuretic peptide (ANP), β-amyloid peptides, delta-opioid antagonists (such as ITIPP(psi)), annexin-V, endothelin, leukotriene B4 (LTB4), chemotactic peptides (e.g., N-formyl-methionyl-leucyl-phenylalanine-lysine (fMLFK), GP IIb/IIIa receptor antagonists (e.g., DMP444), human neutrophil elastase inhibitor (EPI-HNE-2 and EPI-HNE-4), plasmin inhibitor, antimicrobial peptides, apticide (P280 and P274), thrombospondin receptor (including analogs such as TP-1300), bitistatin, pituitary adenylyl cyclase type I receptor (PAC1), fibrin α-chain, peptides derived from phage display libraries, and conservative substitutions thereof.

One or more (e.g., from 1 to 6) targeting moieties can be linked to MOIETY or to X2 in formula (V′, V″, V′″, V″″, V′″″, V″″″) through -LinkA-.

In some aspects, the targeting moiety includes one or more (e.g., from 1 to 6 or from 1 to 3) asialoglycoprotein receptor ligands (e.g., GalNAc). In some aspects, an asialoglycoprotein receptor ligand (e.g., GalNAc) is attached to -LinkA- through an anomeric carbon (e.g., where the anomeric carbon is the carbon atom in an acetal or a hemiaminal). An asialoglycoprotein receptor ligand (e.g., GalNAc) attached to a linker through a hemiaminal may produce a hybridized polynucleotide construct having superior efficacy in gene silencing as compared to hybridized polynucleotide constructs having the asialoglycoprotein receptor ligand (e.g., GalNAc) attached to a linker through an acetal.

In some aspects, the linker and three asialoglycoprotein receptor targeting moieties, each of which comprises GalNAc, are as shown in Formula (V). In some instances, the conjugate described herein only comprises one asialoglycoprotein receptor targeting moiety, so the conjugate comprises a structure of Formula (V) with any two of the targeting moieties removed. In some instances, the conjugate described herein only comprises two asialoglycoprotein receptor targeting moieties, so the conjugate described herein comprises a structure of Formula (V) with any one of the targeting moieties removed.

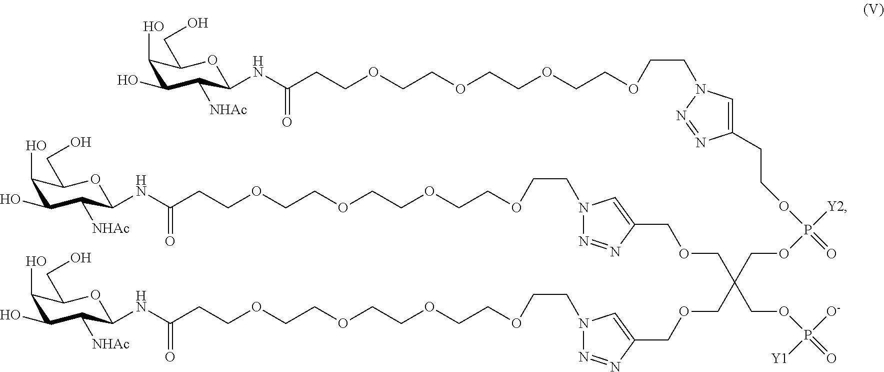

wherein one of Y1 and Y2 is nucleotide, or wherein both Y1 and Y2 are nucleotides and Y1 and Y2 are consecutive or neighboring nucleotides from the polynucleic acid molecule described herein.

In some aspects, the linker and the targeting moieties described herein are conjugated to 3′ end of the sense strand (e.g., as shown in Formula (V′, V″″, V′″″, V″″″)). In some aspects, the linker and the targeting moieties described herein are conjugated to 5′ end of the sense strand (e.g., as shown in Formula (V″) or (V′″)). In some aspects, the linker and the targeting moieties described herein are conjugated to 3′ end of the antisense strand(e.g., as shown in Formula (V′), (V″″), (V′″″), (V″″″)). In some aspects, the linker and the targeting moieties described herein are conjugated to 5′ end of the antisense strand (e.g., as shown in Formula (V″) or (V′″)).

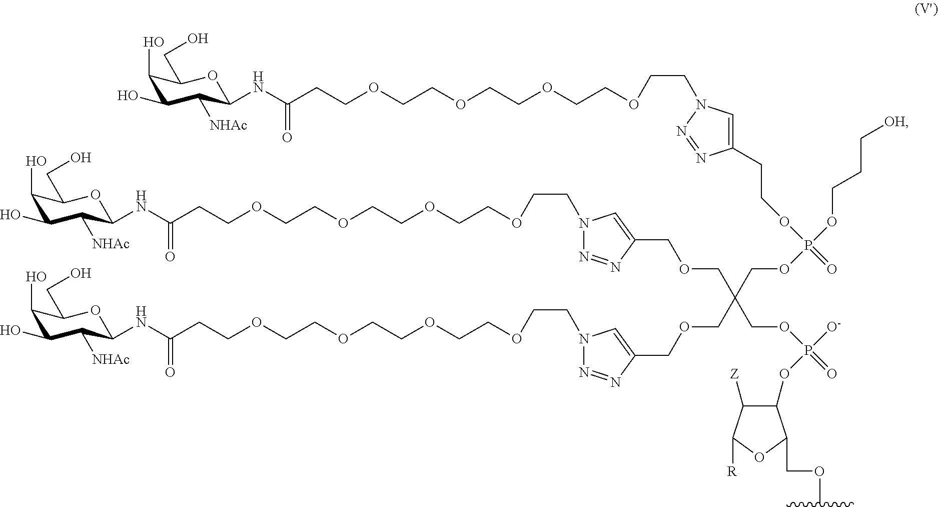

wherein Z in formula (V′) is a moiety that corresponds to one of the sugar modifications described herein (e.g., —H, —OH, —O-Methyl, —F, or —O-methoxyethyl) and R in formula (V′) is adenine, uracil, guanine, cytosine, thymine, abasic, or others;

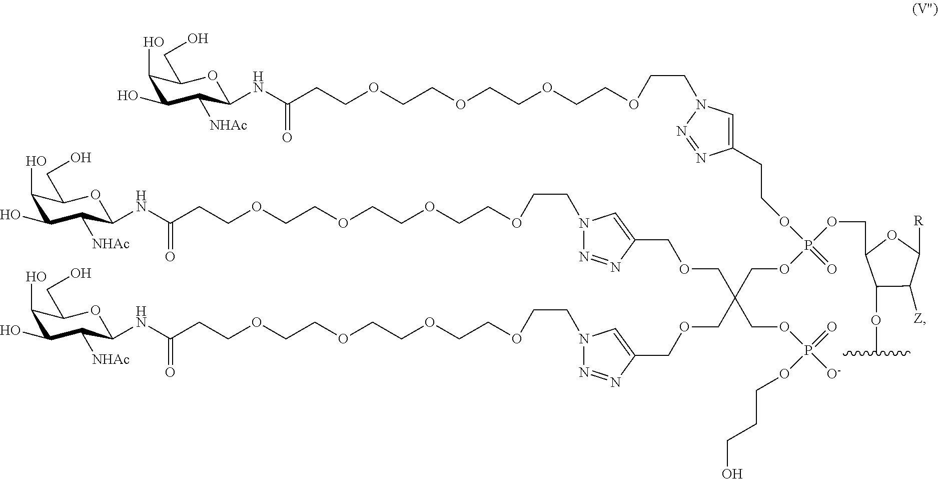

wherein Z in formula (V″) is a moiety that corresponds to one of the sugar modifications described herein (e.g., —H, —OH, —O-Methyl, —F, or —O-methoxyethyl) and R in formula (V″) is adenine, uracil, guanine, cytosine, thymine, abasic, or others.

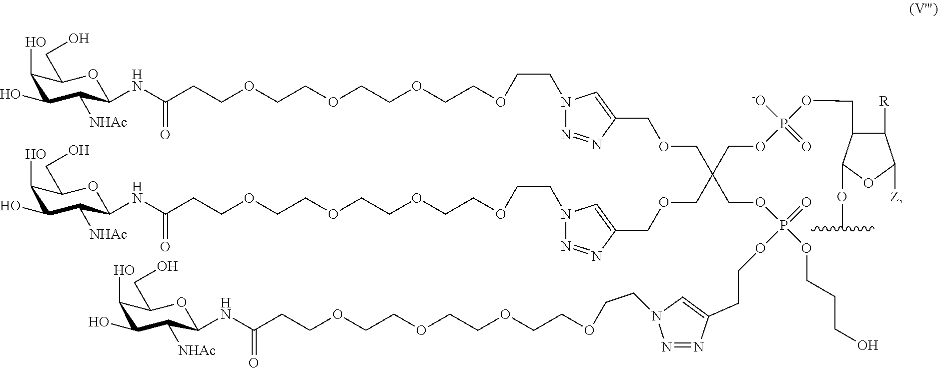

wherein Z in formula (V′″) is a moiety that corresponds to one of the sugar modifications described herein (e.g., —H, —OH, —O-Methyl, —F, or —O-methoxyethyl) and R in formula (V′″) is adenine, uracil, guanine, cytosine, thymine, abasic, or others.

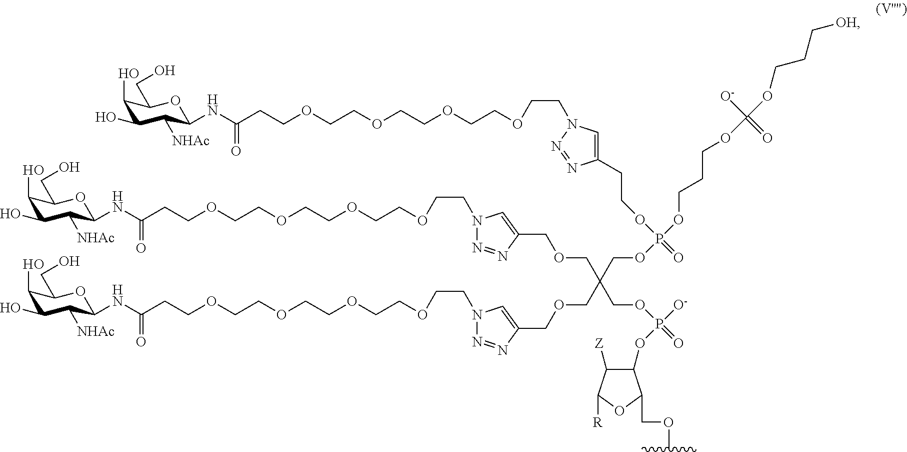

wherein Z in formula (V″″) is a moiety that corresponds to one of the sugar modifications described herein (e.g., —H, —OH, —O-Methyl, —F, or —O-methoxyethyl) and R in formula (V″″) is adenine, uracil, guanine, cytosine, thymine, abasic, or others.

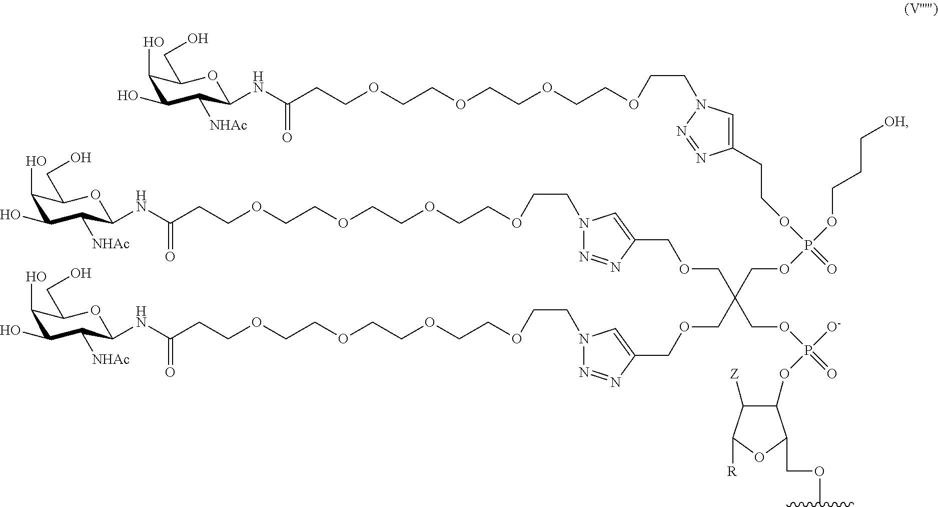

wherein Z in formula (V′″″) is a moiety that corresponds to one of the sugar modifications described herein (e.g., —H, —OH, —O-Methyl, —F, or —O-methoxyethyl) and R in formula (V′″″) is adenine, uracil, guanine, cytosine, thymine, abasic, or others.

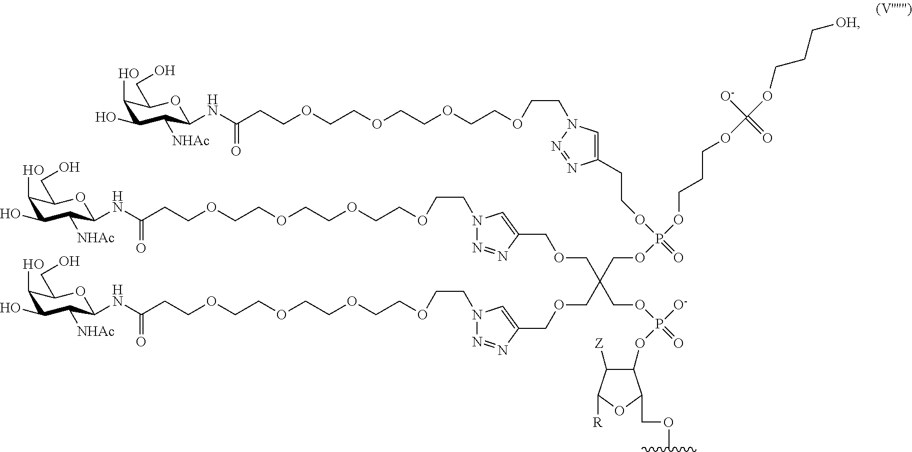

wherein Z in formula (V″″″) is a moiety that corresponds to one or the sugar modifications described herein (e.g., —H, —OH, —O-Methyl, —F, or —O-methoxyethyl) and R in formula (V″″″) is adenine, uracil, guanine, cytosine, thymine, abasic, or others.

In some instances, the 3′ end of passenger/sense strand from Table 1, Table 2, or Table 5 is conjugated with X2-GalNAc (see Formula (V), (V′), (V″″), (V′″″), (V″″″)). In some instances, the 5′ end of passenger/sense strand from Table 1, Table 2, or Table 5 is conjugated with X2-GalNAc (see Formula (V), (V″), or (V′″)). In some instances, a nucleic acid within passenger/sense strand (not at the 5′ or 3′ end) from Table 1, Table 2, or Table 5 is conjugated with X2-GalNAc (see Formula (V)). In some instances, the 3′ end of guide/antisense strand from Table 1, Table 2, or Table 5 is conjugated with X2-GalNAc (see Formula (V), (V′), (V″″), (V′″″), (V″″″)). In some instances, the 5′ end of guide/antisense strand from Table 1, Table 2, or Table 5 is conjugated with X2-GalNAc (see Formula (V), (V″), or (V′″)). In some instances, a nucleic acid within guide/antisense strand (not at the 5′ or 3′ end) from Table 1, Table 2, or Table 5 is conjugated with X2-GalNAc (see Formula (V)).

In some instances, one or more endosomal escape moieties (e.g., from 1 to 6 or from 1 to 3) can be attached to a polynucleotide construct or a hybridized polynucleotide construct disclosed herein as an auxiliary moiety. Exemplary endosomal escape moieties include chemotherapeutics (e.g., quinolones such as chloroquine); fusogenic lipids (e.g., dioleoylphosphatidyl-ethanolamine (DOPE)); and polymers such as polyethylenimine (PEI); poly(beta-amino ester)s; polypeptides, such as polyarginines (e.g., octaarginine) and polylysines (e.g., octalysine); proton sponges, viral capsids, and peptide transduction domains as described herein. For example, fusogenic peptides can be derived from the M2 protein of influenza A viruses; peptide analogs of the influenza virus hemagglutinin; the HEF protein of the influenza C virus; the transmembrane glycoprotein of filoviruses; the transmembrane glycoprotein of the rabies virus; the transmembrane glycoprotein (G) of the vesicular stomatitis virus; the fusion protein of the Sendai virus; the transmembrane glycoprotein of the Semliki forest virus; the fusion protein of the human respiratory syncytial virus (RSV); the fusion protein of the measles virus; the fusion protein of the Newcastle disease virus; the fusion protein of the visna virus, the fusion protein of murine leukemia virus; the fusion protein of the HTL virus; and the fusion protein of the simian immunodeficiency virus (SIV). Other moieties that can be employed to facilitate endosomal escape are described in Dominska et al., Journal of Cell Science, 123(8):1183-1189, 2010. Specific examples of endosomal escape moieties including moieties suitable for conjugation to the hybridized polynucleotide constructs disclosed herein are provided, e.g., in WO 2015/188197; the disclosure of these endosomal escape moieties is incorporated by reference herein.

One or more endosomal escape moieties (e.g., from 1 to 6 or from 1 to 3) can be attached to a MOIETY or X2 in formula (V′, V″, V′″, V″″, V′″″, or V″″″) through -LinkA-, as described herein.

One or more cell penetrating peptides (CPP) (e.g., from 1 to 6 or from 1 to 3) can be attached to a polynucleotide construct or a hybridized polynucleotide construct disclosed herein as an auxiliary moiety. The CPP can be linked to the hybridized polynucleotide bioreversibly through a disulfide linkage, as disclosed herein. Thus, upon delivery to a cell, the CPP can be cleaved intracellularly, e.g., by an intracellular enzyme(e.g., protein disulfide isomerase, thioredoxin, or a thioesterase) and thereby release the polynucleotide.

CPPs are known in the art (e.g., TAT or Arg8) (Snyder and Dowdy, 2005, Expert Opin. Drug Deliv. 2, 43-51). Specific examples of CPPs including moieties suitable for conjugation to the hybridized polynucleotide constructs disclosed herein are provided, e.g., in WO 2015/188197; the disclosure of these CPPs is incorporated by reference herein.

CPPs are positively charged peptides that are capable of facilitating the delivery of biological cargo to a cell. It is believed that the cationic charge of the CPPs is essential for their function. Moreover, the transduction of these proteins does not appear to be affected by cell type, and these proteins can efficiently transduce nearly all cells in culture with no apparent toxicity (Nagahara et al., Nat. Med. 4:1449-52, 1998). In addition to full-length proteins, CPPs have also been used successfully to induce the intracellular uptake of DNA (Abu-Amer, supra), antisense polynucleotides (Astriab-Fisher et al., Pharm. Res, 19:744-54, 2002), small molecules (Polyakov et al., Bioconjug. Chem. 11.762-71, 2000) and even inorganic 40 nm iron particles (Dodd et al., J. Immunol. Methods 256:89-105, 2001; Wunderbaldinger et al., Bioconjug. Chem. 13:264-8, 2002; Lewin et al., Nat. Biotechnol. 18:410-4, 2000; Josephson et al., Bioconjug. Chem. 10:186-91, 1999) suggesting that there is considerable flexibility in particle size in this process.

In one case, a CPP useful in the methods and compositions as described herein includes a peptide featuring substantial alpha-helicity. It has been discovered that transfection is optimized when the CPP exhibits significant alpha-helicity. In another case, the CPP includes a sequence containing basic amino acid residues that are substantially aligned along at least one face of the peptide. A CPP described herein may be a naturally occurring peptide or a synthetic peptide.

One or more cell penetrating peptides (e.g., from 1 to 6 or from 1 to 3) can be attached to a MOIETY or X2 in formula (I) through -LinkA-, as described herein.

The polynucleotide constructs and the hybridized polynucleotide constructs disclosed herein can also include covalently attached neutral polymer-based auxiliary moieties. Neutral polymers include poly(C1-6 alkylene oxide), e.g., poly(ethylene glycol) and poly(propylene glycol) and copolymers thereof, e.g., di- and triblock copolymers. Other examples of polymers include esterified poly(acrylic acid), esterified poly(glutamic acid), esterified poly(aspartic acid), poly(vinyl alcohol), poly(ethylene-co-vinyl alcohol), poly(N-vinyl pyrrolidone), poly(ethyloxazoline), poly(alkylacrylates), poly(acrylamide), poly(N-alkylacrylamides), poly(N-acryloylmorpholine), poly(lactic acid), poly(glycolic acid), poly(dioxanone), poly(caprolactone), styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyurethane, N-isopropylacrylamide polymers, and poly(N,N-dialkylacrylamides). Exemplary polymer auxiliary moieties may have molecular weights of less than 100, 300, 500, 1000, or 5000 Da (e.g., greater than 100 Da). Other polymers are known in the art.

One or more polymers (e.g., from 1 to 6 or from 1 to 3) can be attached to a MOIETY or X2 in formula (V′, V″, V′″, V″″, V′″″, V″″″) through -LinkA-, as described herein.

### Conjugation Linkers

In some aspects, the polynucleic acid molecules described herein comprises a sense or antisense strand bonded to at least one group of formula (I)

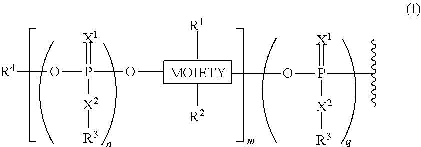

or a salt thereof, or a stereoisomer thereof,
where
each X¹ is independently O or S;
each X² is independently O, S, NH, or a bond;
MOIETY is optionally substituted C₂₋₁₀ alkane-tetrayl or a group -M¹-M²-M³-, wherein each M¹ and each M³ is independently absent or optionally substituted C₁₋₆ alkylene, and M² is optionally substituted C₃₋₉ heterocycle-tetrayl, optionally substituted C₆₋₁₀ arene-tetrayl, or optionally substituted C₃₋₈ cycloalkane-tetrayl;
each R¹ and each R² is independently H, optionally substituted C₁₋₆ alkyl, optionally substituted C₂₋₁₆ heteroalkyl, a conjugation moiety, or -LinkA(-T)ₚ, provided that at least one R or at least one R² is a conjugation moiety or -LinkA(-T)ₚ;
each R³ is independently H, optionally substituted C₁₋₁₆ alkyl, optionally substituted C₂₋₁₆ heteroalkyl, optionally substituted C₂₋₁₆ alkenyl, optionally substituted C₂₋₁₆ alkynyl, optionally substituted (C₁₋₉ heterocyclyl)-C₁₋₆-alkyl, optionally substituted (C₆₋₁₀ aryl)-C₁₋₆-alkyl, optionally substituted (C₃₋₈ cycloalkyl)-C₁₋₆-alkyl, a conjugation moiety, or -LinkA(-T)ₚ;
R⁴ is H, optionally substituted C₁₋₆ alkyl, -LinkA(-T)ₚ, or -Sol;
each LinkA is independently a multivalent linker (e.g., including —C(O)—N(H)— (e.g., at least one multivalent linker including —C(O)—N(H)— bonded to T));
each T is independently an auxiliary moiety;
Sol is solid support;
m is an integer from 1 to 6;
each n is independently 0 or 1;
each p is independently an integer from 1 to 6; and
q is an integer from 0 to 3.

The at least one group of formula (I) may be bonded to a 5′-terminus, 3′-terminus, internucleoside phosphate, internucleoside phosphorothioate, or internucleoside phosphorodithioate of the polynucleotide. When the at least one group of formula (I) is bonded to the internucleoside phosphate, internucleoside phosphorothioate, or internucleoside phosphorodithioate, q is 0. The polynucleotide construct contains no more than one Sol.

Group -LinkA- can include from 0 to 3 multivalent monomers (e.g., optionally substituted C1-6 alkane-triyl, optionally substituted C1-6 alkane-tetrayl, or trivalent nitrogen atom) and one or more divalent monomers (e.g., from 1 to 40), where each divalent monomer is independently optionally substituted C1-6 alkylene; optionally substituted C2-6 alkenylene; optionally substituted C2-6 alkynylene; optionally substituted C3-8 cycloalkylene; optionally substituted C3-8 cycloalkenylene; optionally substituted C6-14 arylene; optionally substituted C1-9 heteroarylene having 1 to 4 heteroatoms selected from N, O, and S; optionally substituted C1-9 heterocyclylene having 1 to 4 heteroatoms selected from N, O, and S; imino; optionally substituted N; O; or S(O)m, wherein m is 0, 1, or 2. In some aspects, each monomer is independently optionally substituted C1-6 alkylene; optionally substituted C3-8 cycloalkylene; optionally substituted C3-8 cycloalkenylene; optionally substituted C6-14 arylene; optionally substituted C1-9 heteroarylene having 1 to 4 heteroatoms selected from N, O, and S; optionally substituted C1-9 heterocyclylene having 1 to 4 heteroatoms selected from N, O, and S; imino; optionally substituted N; O; or S(O)m, where m is 0, 1, or 2 (e.g., m is 2). In certain aspects, each monomer is independently optionally substituted C1-6 alkylene; optionally substituted C3-8 cycloalkylene; optionally substituted C3-8 cycloalkenylene; optionally substituted C6-14 arylene; optionally substituted C1-9 heteroarylene having 1 to 4 heteroatoms selected from N, O, and S; optionally substituted C1-9 heterocyclylene having 1 to 4 heteroatoms selected from N, O, and S; optionally substituted N; O; or S(O)m, where m is 0, 1, or 2 (e.g., m is 2). The non-bioreversible linker connecting the auxiliary moiety to the conjugating moiety or to the reaction product thereof can include from 2 to 500 (e.g., from 2 to 300 or from 2 to 200) of such monomers. Group -LinkA- may include a poly(alkylene oxide) (e.g., polyethylene oxide, polypropylene oxide, poly(trimethylene oxide), polybutylene oxide, poly(tetramethylene oxide), and diblock or triblock co-polymers thereof). In some aspects, the non-bioreversible linker includes polyethylene oxide (e.g., poly(ethylene oxide) having a molecular weight of less than 1 kDa).

Group -LinkA(-T)p in formula (I) may be prepared by a process described in the sections below. In some instances, -LinkA(-T)p is of formula (II):

-Q¹-Q²([-Q³-Q⁴-Q⁵]ₛ-Q⁶-T)ₚ, (II)
where
each s is independently an integer from 0 to 20 (e.g., from 0 to 10), where the repeating units are the same or different;
Q¹ is a conjugation linker (e.g., [-Q³-Q⁴-Q⁵]ₛ-Q^{C}-, where Q^{C} is optionally substituted C₂₋₁₂ heteroalkylene (e.g., a heteroalkylene containing —C(O)—N(H)—, —N(H)—C(O)—, —S(O)₂—N(H)—, or —N(H)—S(O)₂—), optionally substituted C₁₋₁₂ thioheterocyclylene

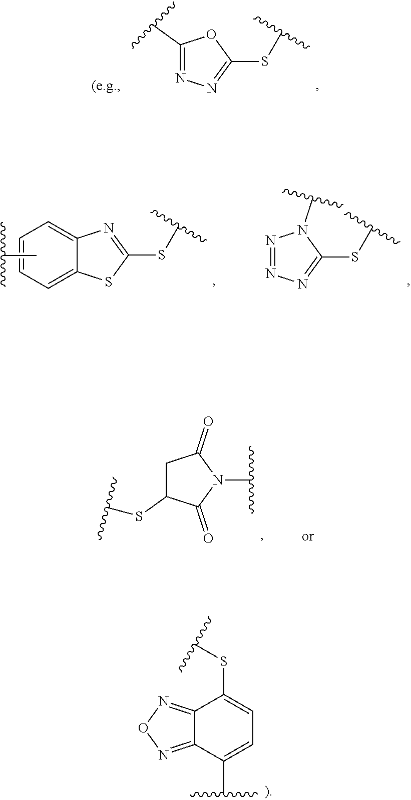

optionally substituted C₁₋₁₂ heterocyclylene (e.g., 1,2,3-triazole-1,4-diyl or

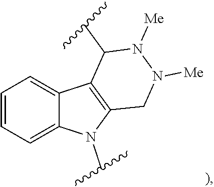 [SMILES: *C1c2c(n(*)c3ccccc23)CN(C)N1C]

cyclobut-3-ene-1,2-dione-3,4-diyl, or pyrid-2-yl hydrazone);
Q² is a linear group (e.g., [-Q³-Q⁴-Q⁵-]ₛ—), if p is 1, or a branched group (e.g., [-Q³-Q⁴-Q⁵]ₛ-Q⁷([-Q³-Q⁴-Q⁵]ₛ-(Q⁷)ₚ₁)ₚ₂, where p1 is 0 or 1, p2 is 0, 1, 2, or 3), if p is an integer from 2 to 6;
each Q³ and each Q⁶ is independently absent, —CO—, —NH—, —O—, —S—, —SO₂—, —OC(O)—, —COO—, —NHC(O)—, —C(O)NH—, —CH₂—, —CH₂NH—, —NHCH₂—, —CH₂O—, or —OCH₂—;
each Q⁴ is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, optionally substituted C₆₋₁₀ arylene, optionally substituted C₁₋₉ heteroarylene, or optionally substituted C₁₋₉ heterocyclylene;
each Q⁵ is independently absent, —CO—, —NH—, —O—, —S—, —SO₂—, —CH₂—, —C(O)O—, —OC(O)—, —C(O)NH—, —NH—C(O)—, —NH—CH(R^{a})—C(O)—, or —C(O)—CH(R^{a})—NH—;
each Q⁷ is independently optionally substituted C₁₋₆, alkane-triyl, optionally substituted C₁₋₆ alkane-tetrayl, optionally substituted C₂₋₆ heteroalkane-triyl, or optionally substituted C₂₋₆ heteroalkane-tetrayl; and
each R^{a} is independently H or an amino acid side chain;
provided that at least one of Q³, Q⁴, and Q⁵ is present.

In some aspects, each Q⁴ is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, or optionally substituted C₁₋₉ heterocyclylene. In certain aspects, s is 1, 2, 3, 4, 5, 6, 7, 8, 9, 10, 11, 12, 13, 14, 15, 16, 17, 18, 19, or 20.

Thus, in formula (II), LinkA may include a single branching point, if each p1 is 0, or multiple branching points, if at least one p1 is 1.

In formula (II), Q¹ may be —O-Q^{L}-Q^{C}-, where Q^{L} is optionally substituted C₂₋₁₂ heteroalkylene, optionally substituted C₁₋₁₂ alkylene, or -(optionally substituted C₁₋₆ alkylene)-(optionally substituted C₆₋₁₀ arylene)-. In some aspects, Q^{L} is optionally substituted C₂₋₁₂ heteroalkylene or optionally substituted C₁₋₁₂ alkylene. In formula (II), Q^{C} may be:

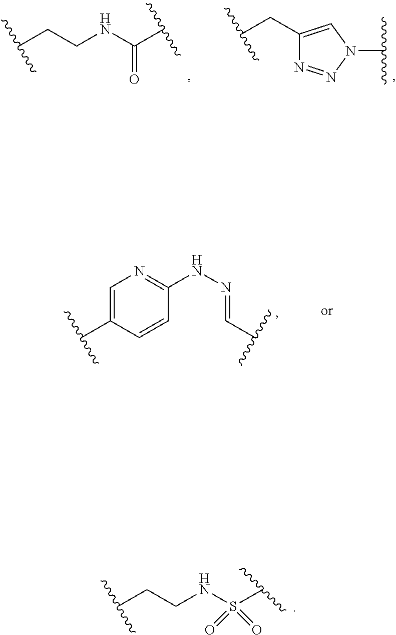 [SMILES: *CCNC(*)=O ; *Cc1cn(*)nn1 ; */C=N/Nc1ccc(*)cn1 ; *CCNS(*)(=O)=O]

In formula (II), Q² may be a linear group of formula [-Q³-Q⁴-Q⁵]-, where Q³, Q⁴, and Q⁵ are as defined for formula (II). Alternatively, Q² may be a branched group [-Q³-Q⁴-Q⁵]ₛ-Q⁷([-Q³-Q⁴-Q⁵]ₛ-(Q⁷)ₚ₁)ₚ₂, where each Q⁷ is independently optionally substituted C₁₋₆ alkane-triyl, optionally substituted C₁₋₆ alkane-tetrayl, optionally substituted C₂₋₆ heteroalkane-triyl, or optionally substituted C₂₋₆ heteroalkane-tetrayl;
where
p1 is 0 or 1;
p2 is 0, 1, 2, or 3;
where,
   when p1 is 0, LinkA is a trivalent or tetravalent linker, and,
   when p1 is 1, LinkA is a tetravalent, pentavalent, or hexavalent linker.

In certain aspects, p1 is 0.

In some aspects, Q⁷ is:

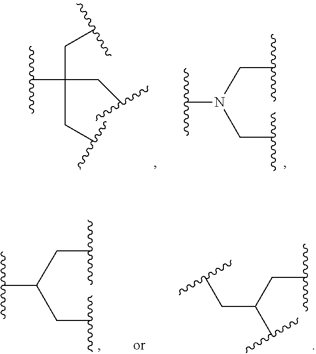 [SMILES: *CC(*)(C*)C* ; *CN(*)C* ; *CC(*)C* ; *CC(*)C*]

Compounds that may be used in the preparation of group -LinkA(-T)p in formula (I) are described herein as well as in WO 2015/188197. Non-limiting examples of -LinkA include:

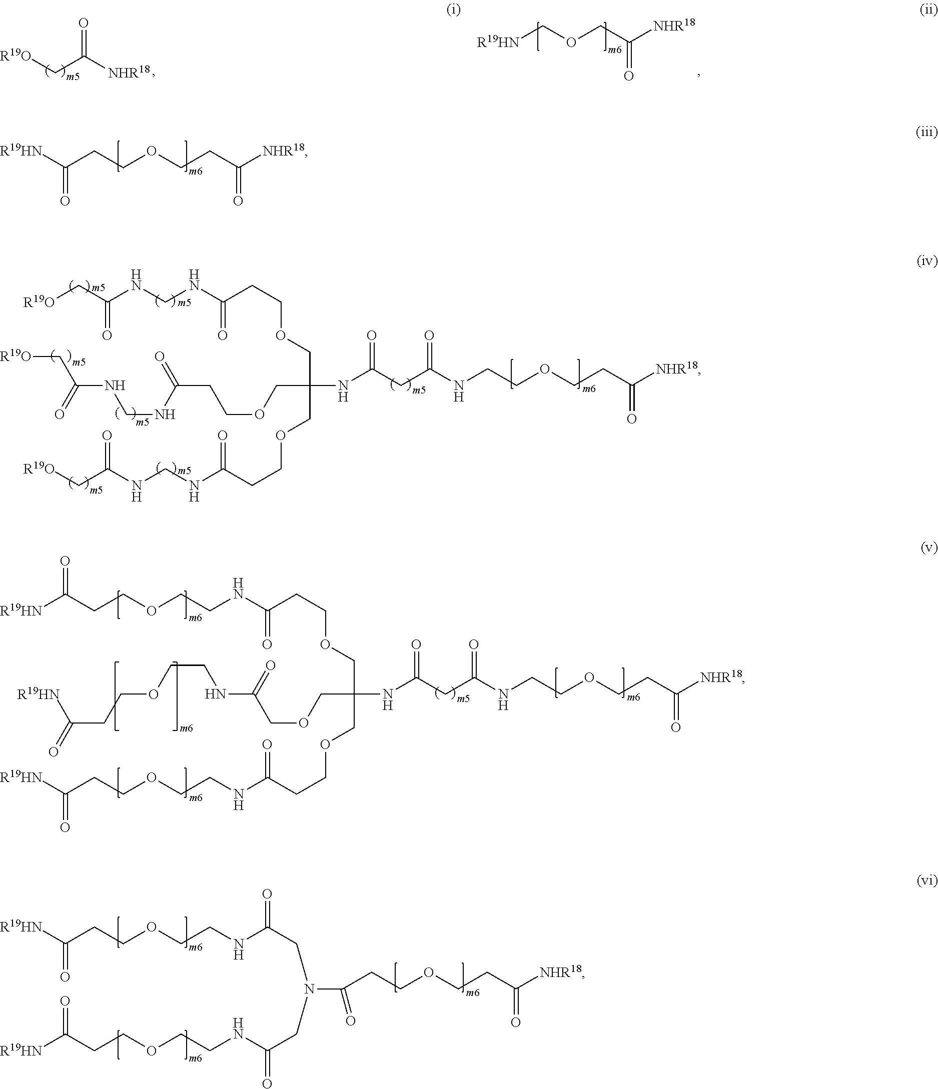

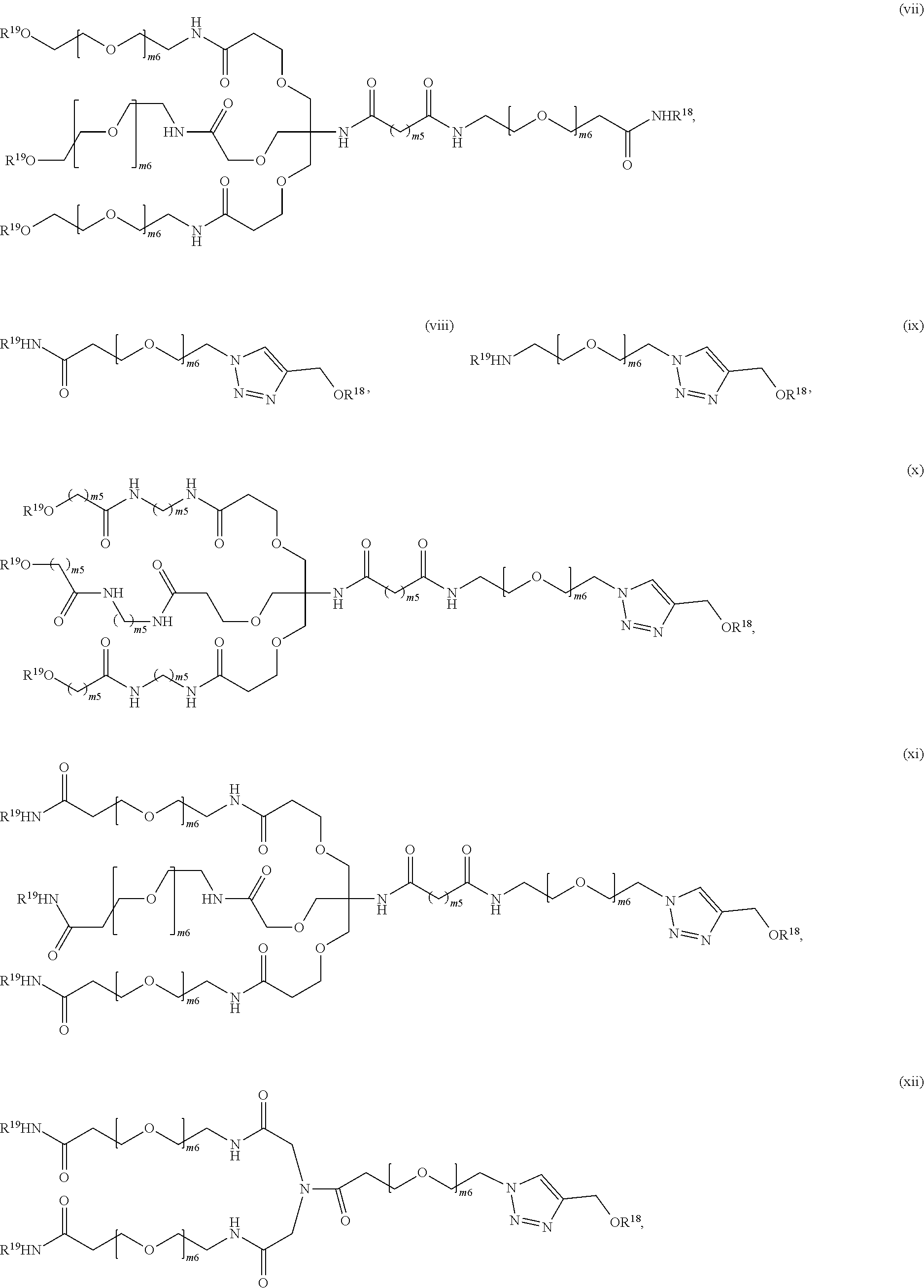

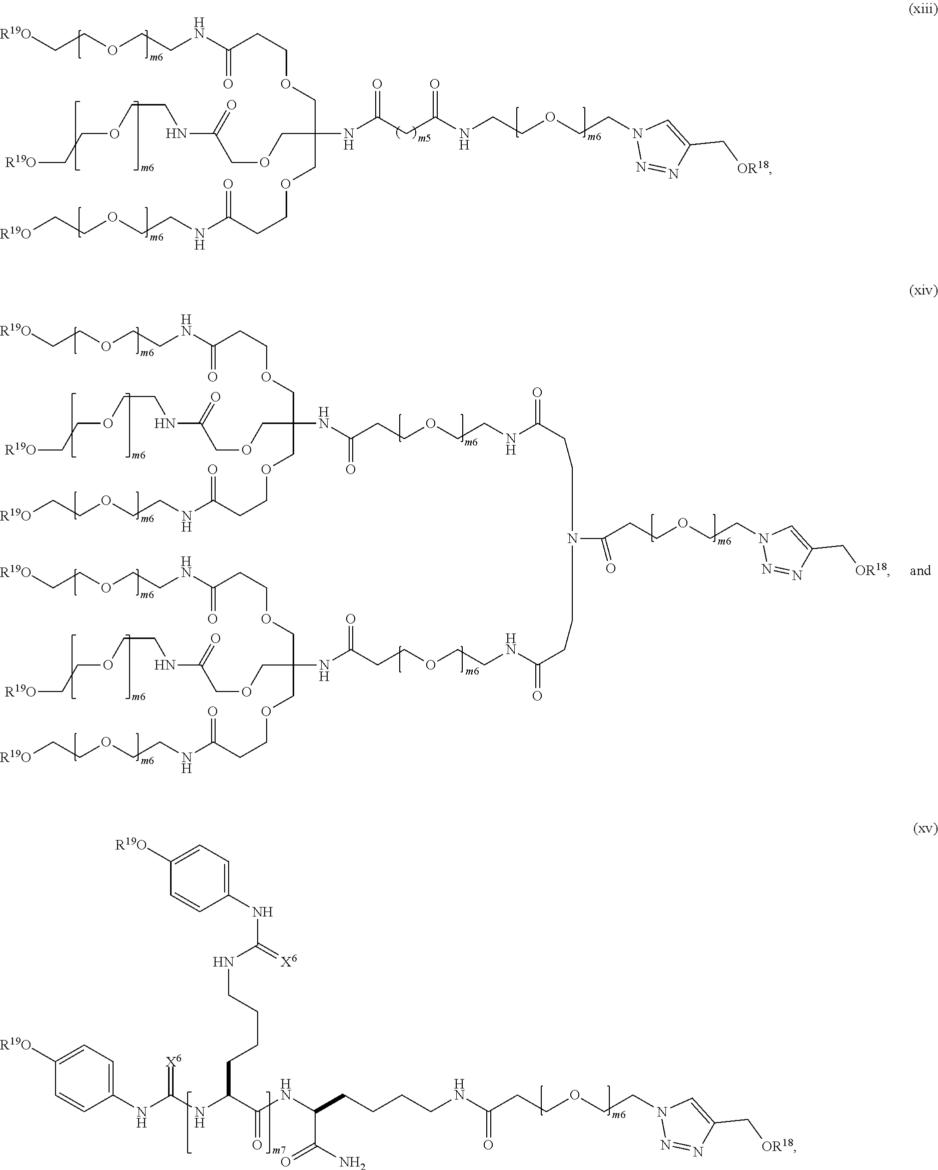

where
R¹⁸ is a bond to MOIETY,
each R¹⁹ is independently a bond to auxiliary moiety,
each m5 is independently an integer from 1 to 20,
each m6 is independently an integer from 1 to 10,
m7 is an integer from 1 to 6, and
each X⁶ is independently O or S.

In formula (II), when the conjugation linker is of formula [-Q³-Q⁴-Q⁵]ₛ-Q^{C}-, -Q²([-Q³-Q⁴-Q⁵]ₛ-Q⁶-T)ₚ may be:

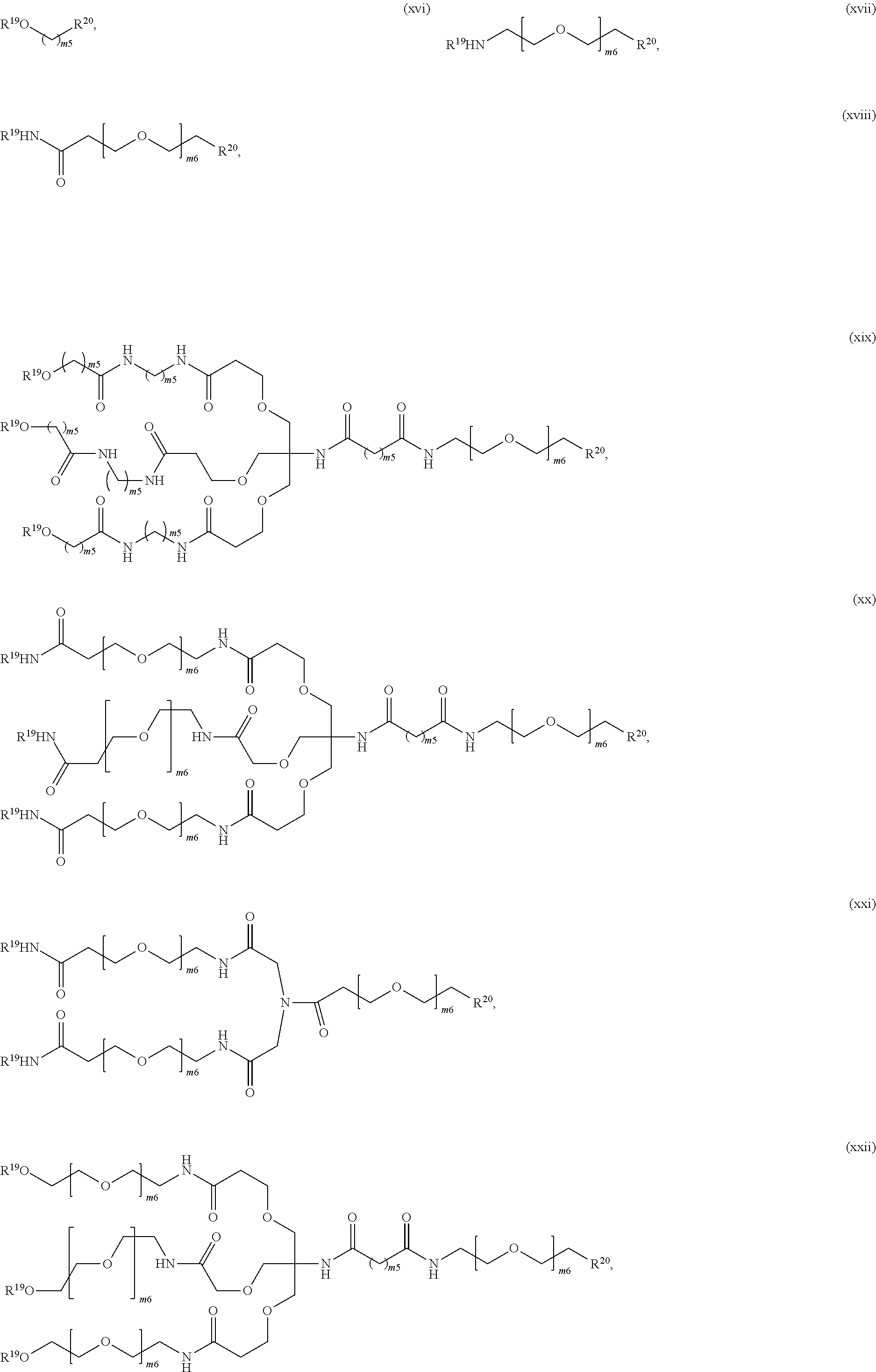

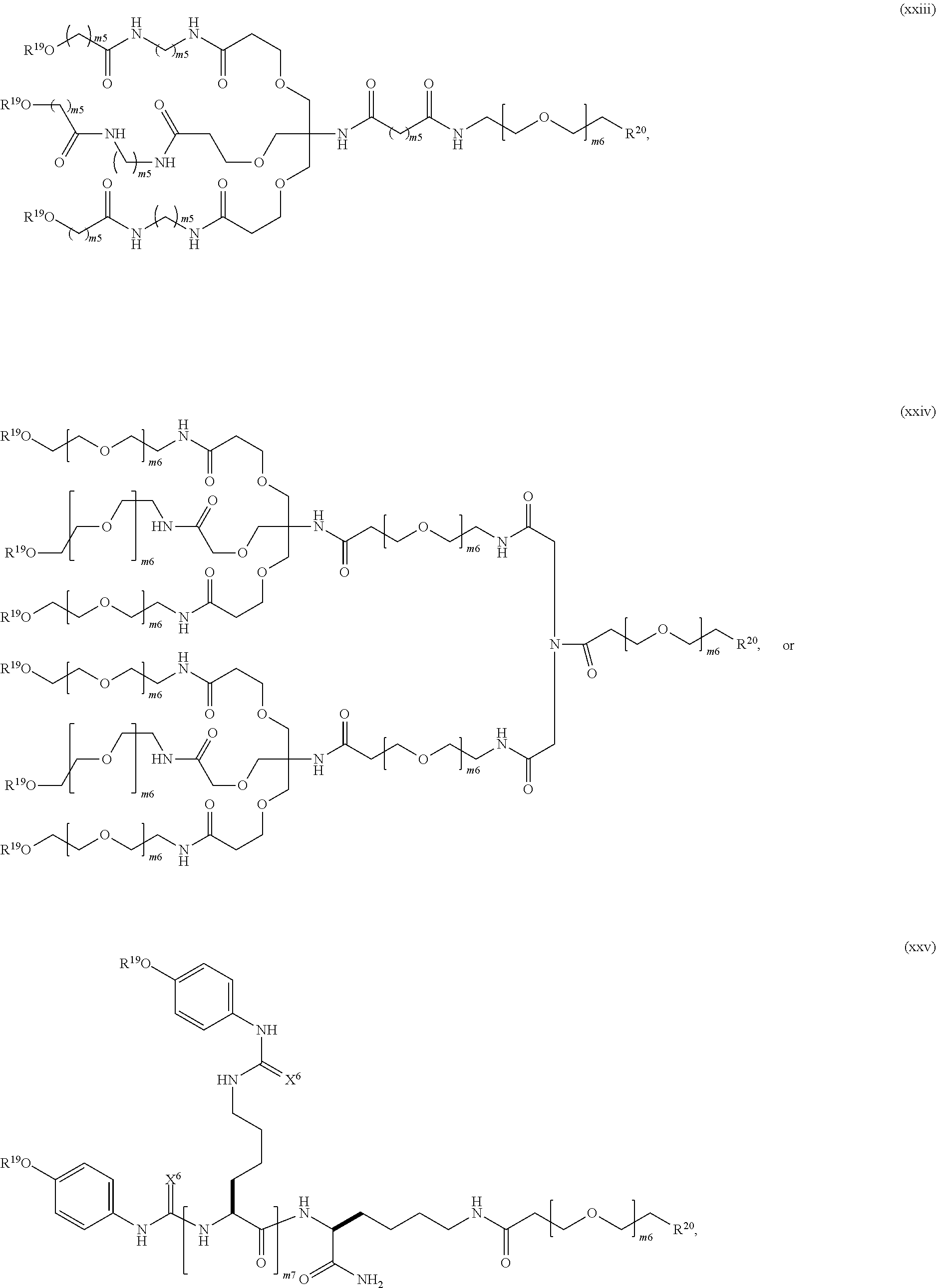

where
R²⁰ is a bond to Q^{C} in Q¹,
each R¹⁹ is independently a bond to an auxiliary moiety,
each m5 is independently an integer from 1 to 20,
each m6 is independently an integer from 1 to 10,
m7 is an integer from 1 to 6, and
each X⁶ is independently O or S.

In some aspects, the linker described herein is cleavable. In some aspects, the linker described herein is non-cleavable.

In some aspects, the polynucleic acid molecule described herein comprises a sense or antisense strand bonded to at least one group of formula (IV),

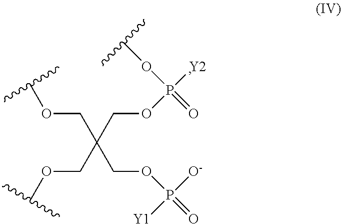

wherein at least one of Y1 and Y2 is a nucleotide from the polynucleic acid molecule.

In some instances, the Y1 is the last nucleotide on the 3′-terminus or the first nucleotide on the 5′ terminus of one of the strands of the polynucleic acid molecule. In some instances, the Y1 is the last nucleotide on the 3′-terminus or the first nucleotide on the 5′ terminus of the sense strand of the polynucleic acid molecule. In some instances, the Y1 is the last nucleotide on the 3′-terminus or the first nucleotide on the 5′ terminus of the sense strand of the polynucleic acid molecule, and the Y2 is a 3-hydroxy-propoxy group. In some instances, the Y2 is the first nucleotide on the 5′-terminus or the last nucleotide on the 3′-terminus of one of the strands of the polynucleic acid molecule. In some instances, the Y2 is the first nucleotide on the 5′-terminus or the last nucleotide on the 3′-terminus of the sense strand of the polynucleic acid molecule. In some instances, the Y2 is the first nucleotide or the last nucleotide on the 3′-terminus on the 5′-terminus of the sense strand of the polynucleic acid molecule, and the Y1 is a 3-hydroxy-propoxy group. In other instances, the Y1 and Y2 are two consecutive nucleotides in one of the strands of the polynucleic acid molecule.

In some aspects, the targeting moiety described herein is conjugated to 3′ end of the sense strand (e.g., formula (IV′) or (IV″″)). In some aspects, the targeting moiety described herein is conjugated to 5′ end of the sense strand (e.g., formula (IV″) or (IV′″)). In some aspects, the targeting moiety described herein is conjugated to 3′ end of the antisense strand (e.g., formula (IV′) or(IV″″)). In some aspects, the targeting moiety described herein is conjugated to 5′ end of the antisense strand (e.g., formula (IV″) or (IV′″)).

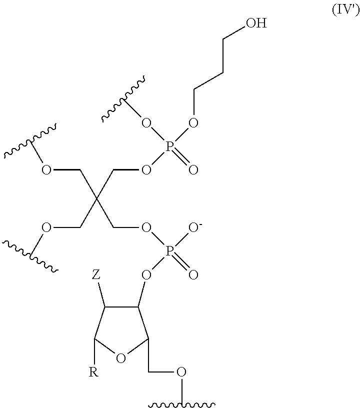

wherein Z in formula (IV′) is a moiety that corresponds to one of the sugar modifications described herein (e.g., —H, —OH, —O-Methyl, —F, or —O-methoxyethyl) and R in formula (IV′) is adenine, uracil, guanine, cytosine, thymine, abasic, or others.

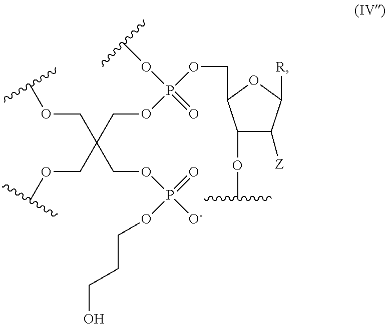

wherein Z in formula (IV″) is a moiety that corresponds to one of the sugar modifications described herein (e.g., —H, —OH, —O-Methyl, —F, or —O-methoxyethyl) and R in formula (IV″) is adenine, uracil, guanine, cytosine, thymine, abasic, or others.

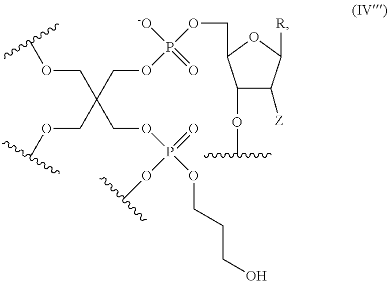

wherein Z in formula (IV′″) is a moiety that corresponds to one of the sugar modifications described herein (e.g., —H, —OH, —O-Methyl, —F, or —O-methoxyethyl) and R in formula (IV′″) is adenine, uracil, guanine, cytosine, thymine, abasic, or others.

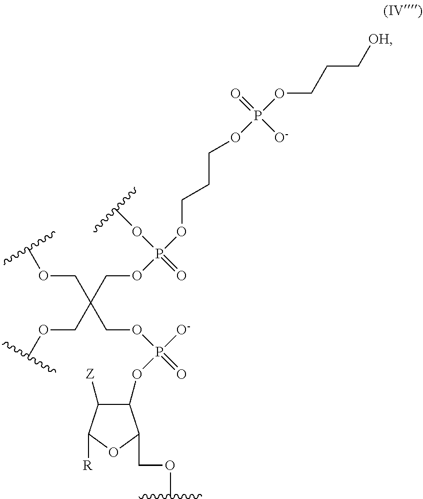

wherein Z in formula (IV″″) is a moiety that corresponds to one of the sugar modifications described herein (e.g., —H, —OH, —O-Methyl, —F, or —O-methoxyethyl) and R in formula (IV″″) is adenine, uracil, guanine, cytosine, thymine, abasic, or others.

### Pharmaceutical Compositions

Delivery of the polynucleotide molecules described herein can be achieved by contacting a cell with the polynucleotide molecules using a variety of methods. In particular aspects, the polynucleotide molecule described herein is formulated with various excipients, vehicles, and carriers, as described more fully elsewhere herein.

A pharmaceutical composition described herein can be prepared to include a hybridized polynucleotide construct disclosed herein, into a form suitable for administration to a subject using carriers, excipients, and vehicles. Frequently used excipients include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, and inert gases. Other pharmaceutically acceptable vehicles include aqueous solutions, non-toxic excipients, including salts, preservatives, buffers and the like, as described, for instance, in Remington: The Science and Practice of Pharmacy, 21st Ed., Gennaro, Ed., Lippencott Williams & Wilkins (2005), and The United States Pharmacopeia: The National Formulary (USP 36 NF31), published in 2013. The pH and exact concentration of the various components of the pharmaceutical composition are adjusted according to routine skills in the art. See Goodman and Gilman's, The Pharmacological Basis for Therapeutics.

The pharmaceutical compositions described herein may be administered locally or systemically. The therapeutically effective amounts will vary according to factors, such as the degree of infection in a subject, the age, sex, and weight of the individual. Dosage regimes can be adjusted to provide the optimum therapeutic response. For example, several divided doses can be administered daily or the dose can be proportionally reduced as indicated by the exigencies of the therapeutic situation.

The pharmaceutical composition can be administered in a convenient manner, such as by injection (e.g., subcutaneous, intravenous, intraorbital, and the like), oral administration, ophthalmic application, inhalation, topical application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. The composition will typically be sterile and fluid to the extent that easy syringability exists. Typically the composition will be stable under the conditions of manufacture and storage and preserved against the contaminating action of microorganisms, such as bacteria and fungi. The vehicle can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

It is especially advantageous to formulate parenteral compositions in dosage unit form for ease of administration and uniformity of dosage. Dosage unit form as used herein, refers to physically discrete units suited as unitary dosages for the subject to be treated; each unit containing a predetermined quantity of pharmaceutical composition is calculated to produce the desired therapeutic effect in association with the required pharmaceutical vehicle. The specification for the dosage unit forms is related to the characteristics of the pharmaceutical composition and the particular therapeutic effect to be achieve. The principal pharmaceutical composition is compounded for convenient and effective administration in effective amounts with a suitable pharmaceutically acceptable vehicle in an acceptable dosage unit. In the case of compositions containing supplementary active ingredients, the dosages are determined by reference to the usual dose and manner of administration of the ingredients.

The pharmaceutical composition can be orally administered, for example, in a carrier, e.g., in an enteric-coated unit dosage form. The pharmaceutical composition and other ingredients can also be enclosed in a hard or soft-shell gelatin capsule or compressed into tablets. For oral therapeutic administration, the pharmaceutical composition can be incorporated with excipients and used in the form of ingestible tablets, troches, capsules, pills, wafers, and the like. Such compositions and preparations should contain at least 1% by weight of active compound. The percentage of the compositions and preparations can, of course, be varied and can conveniently be between about 5% to about 80% of the weight of the unit. The tablets, troches, pills, capsules, and the like can also contain the following: a binder, such as gum tragacanth, acacia, corn starch, or gelatin; excipients such as dicalcium phosphate: a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Any material used in preparing any dosage unit form should be of pharmaceutically acceptable purity and substantially non-toxic in the amounts employed. In addition, the pharmaceutical composition can be incorporated into sustained-release preparations and formulations.

The pharmaceutical composition described herein may comprise one or more permeation enhancer that facilitates bioavailability of the polynucleotide molecule described herein. WO 2000/67798, Muranishi, 1990, Crit. Rev. Ther. Drug Carrier Systems, 7, 1, Lee et al., 1991, Crit. Rev. Ther. Drug Carrier Systems, 8, 91 are herein incorporated by reference in its entirety. In some aspects, the permeation enhancer is intestinal. In some aspects, the permeation enhancer is transdermal. In some aspects, the permeation enhancer is to facilitate crossing the brain-blood barrier. In some aspects, the permeation enhancer improves the permeability in the oral, nasal, buccal, pulmonary, vaginal, or corneal delivery model. In some aspects, the permeation enhancer is a fatty acid or a derivative thereof. In some aspects, the permeation enhancer is a surfactant or a derivative thereof. In some aspects, the permeation enhancer is a bile salt or a derivative thereof. In some aspects, the permeation enhancer is a chelating agent or a derivative thereof. In some aspects, the permeation enhancer is a non-chelating non-surfactant or a derivative thereof. In some aspects, the permeation enhancer is an ester or a derivative thereof. In some aspects, the permeation enhancer is an ether or a derivative thereof. In some aspects, the permeation enhancer is arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof. In one specific aspect, the permeation enhancer is sodium caprate (C10). In some aspects, the permeation enhancer is chenodeoxycholic acid (CDCA), ursodeoxychenodeoxycholic acid (UDCA), cholic acid, dehydrocholic acid, deoxycholic acid, glucholic acid, glycholic acid, glycodeoxycholic acid, taurocholic acid taurodeoxycholic acid, sodium tauro-24,25-dihydro-fusidate or sodium glycodihydrofusidate. In some aspects, the permeation enhancer is polyoxyethylene-9-lauryl ether, or polyoxyethylene-20-cetyl ether.

For the polynucleotide molecule described herein, suitable pharmaceutically acceptable salts include (i) salts formed with cations such as sodium, potassium, ammonium, magnesium, calcium, polyamines such as spermine and spermidine, etc.; (ii) acid addition salts formed with inorganic acids, for example hydrochloric acid, hydrobromic acid, sulfuric acid, phosphoric acid, and the like; and (iii) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like.

While the hybridized polynucleotide constructs described herein may not require the use of excipients for delivery to the target cell, the use of excipients may be advantageous in some aspects. Thus, for delivery to the target cell, the hybridized polynucleotide molecule described herein can non-covalently bind an excipient to form a complex. The excipient can be used to alter biodistribution after delivery, to enhance uptake, to increase half-life or stability of the strands in the hybridized polynucleotide constructs (e.g., improve nuclease resistance), and/or to increase targeting to a particular cell or tissue type.

Exemplary excipients include a condensing agent (e.g., an agent capable of attracting or binding a nucleic acid through ionic or electrostatic interactions); a fusogenic agent (e.g., an agent capable of fusing and/or being transported through a cell membrane); a protein to target a particular cell or tissue type (e.g., thyrotropin, melanotropin, lectin, glycoprotein, surfactant protein A, or any other protein); a lipid; a lipopolysaccharide; a lipid micelle or a liposome (e.g., formed from phospholipids, such as phosphotidylcholine, fatty acids, glycolipids, ceramides, glycerides, cholesterols, or any combination thereof); a nanoparticle (e.g., silica, lipid, carbohydrate, or other pharmaceutically-acceptable polymer nanoparticle); a polyplex formed from cationic polymers and an anionic agent (e.g., a CRO), where exemplary cationic polymers include polyamines (e.g., polylysine, polyarginine, polyamidoamine, and polyethylene imine); cholesterol; a dendrimer (e.g., a polyamidoamine (PAMAM) dendrimer); a serum protein (e.g., human serum albumin (HSA) or low-density lipoprotein (LDL)); a carbohydrate (e.g., dextran, pullulan, chitin, chitosan, inulin, cyclodextrin, or hyaluronic acid); a lipid; a synthetic polymer, (e.g., polylysine (PLL), polyethylenimine, poly-L-aspartic acid, poly-L-glutamic acid, styrene-maleic acid anhydride copolymer, poly(L-lactide-co-glycolic) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacrylic acid), N-isopropylacrylamide polymer, pseudopeptide-polyamine, peptidomimetic polyamine, or polyamine); a cationic moiety (e.g., cationic lipid, cationic porphyrin, quaternary salt of a polyamine, or alpha helical peptide); a multivalent sugar (e.g., multivalent lactose, multivalent galactose, N-acetyl-galactosamine, N-acetyl-glucosamine, multivalent mannose, or multivalent fucose); a vitamin (e.g., vitamin A, vitamin E, vitamin K, vitamin B, folic acid, vitamin B12, riboflavin, biotin, or pyridoxal); a cofactor; or a drug to disrupt cellular cytoskeleton to increase uptake (e.g., taxol, vincristine, vinblastine, cytochalasin, nocodazole, japlakinolide, latrunculin A, phalloidin, swinholide A, indanocine, or myoservin).

Other therapeutic agents as described herein may be included in a pharmaceutical composition described herein in combination with a polynucleotide molecule described herein.

### Methods of Treatment

In some aspects, described herein is a method of modulating expression of Lp(a) gene in a subject, comprising: administering to the subject a polynucleic acid molecule described herein, a polynucleic acid molecule conjugate described herein, or a pharmaceutical composition described herein, thereby modulating the expression of Lp(a) gene in the subject.

In some aspects, the method described herein reduces expression of Lp(a) gene (e.g., expression of Lp(a) mRNA) in a subject by about or at least 10% compared to a control. The term “control” as used herein refers to a subject or a cell receiving no treatment or placebo. In some aspects, the method described herein reduces expression of Lp(a) gene in a subject by about or at least 20% compared to a control. In some aspects, the method described herein reduces expression of Lp(a) gene in a subject by about or at least 30% compared to a control. In some aspects, the method described herein reduces expression of Lp(a) gene in a subject by about or at least 40% compared to a control. In some aspects, the method described herein reduces expression of Lp(a) gene in a subject by about or at least 50% compared to a control. In some aspects, the method described herein reduces expression of Lp(a) gene in a subject by about or at least 60% compared to a control. In some aspects, the method described herein reduces expression of Lp(a) gene in a subject by about or at least 70% compared to a control. In some aspects, the method described herein reduces expression of Lp(a) gene in a subject by about or at least 80% compared to a control. In some aspects, the method described herein reduces expression of Lp(a) gene in a subject by about or at least 90% compared to a control. In some aspects, the method described herein reduces expression of Lp(a) gene in a subject by about 100% compared to a control.

In some aspects, the method described herein achieves an IC50 value of about 5 nM. In some aspects, the method described herein achieves an IC50 value of about 10 nM. In some aspects, the method described herein achieves an IC50 value of about 15 nM. In some aspects, the method described herein achieves an IC50 value of about 20 nM. In some aspects, the method described herein achieves an IC50 value of about 25 nM. In some aspects, the method described herein achieves an IC50 value of about 30 nM. In some aspects, the method described herein achieves an IC50 value of about 35 nM. In some aspects, the method described herein achieves an IC50 value of about 40 nM. In some aspects, the method described herein achieves an IC50 value of about 45 nM. In some aspects, the method described herein achieves an IC50 value of about 50 nM. In some aspects, the method described herein achieves an IC50 value of about 55 nM. In some aspects, the method described herein achieves an IC50 value of about 60 nM. In some aspects, the method described herein achieves an IC50 value of about 65 nM. In some aspects, the method described herein achieves an IC50 value of about 70 nM. In some aspects, the method described herein achieves an IC50 value of about 75 nM. In some aspects, the method described herein achieves an IC50 value of about 80 nM. In some aspects, the method described herein achieves an IC50 value of about 85 nM. In some aspects, the method described herein achieves an IC50 value of about 90 nM. In some aspects, the method described herein achieves an IC50 value of about 95 nM. In some aspects, the method described herein achieves an IC50 value of about 100 nM.

In some aspects, the method described herein achieves an IC50 value of about 1 μM. In some aspects, the method described herein achieves an IC50 value of about 1.1 μM. In some aspects, the method described herein achieves an IC50 value of about 1.2 μM. In some aspects, the method described herein achieves an IC50 value of about 1.3 μM. In some aspects, the method described herein achieves an IC50 value of about 1.4 μM. In some aspects, the method described herein achieves an IC50 value of about 1.5 μM. In some aspects, the method described herein achieves an IC50 value of about 2 μM. In some aspects, the method described herein achieves an IC50 value of about 4 μM. In some aspects, the method described herein achieves an IC50 value of about 6 μM. In some aspects, the method described herein achieves an IC50 value of about 8 μM. In some aspects, the method described herein achieves an IC50 value of about 10 μM. In some aspects, the method described herein achieves an IC50 value of about 12 μM. In some aspects, the method described herein achieves an IC50 value of about 13 μM. In some aspects, the method described herein achieves an IC50 value of about 14 μM. In some aspects, the method described herein achieves an IC50 value of about 15 μM. In some aspects, the method described herein achieves an IC50 value of about 30 μM. In some aspects, the method described herein achieves an IC50 value of about 35 μM. In some aspects, the method described herein achieves an IC50 value of about 40 μM. In some aspects, the method described herein achieves an IC50 value of about 50 μM. In some aspects, the method described herein achieves an IC50 value of about 60 μM. In some aspects, the method described herein achieves an IC50 value of about 80 PM. In some aspects, the method described herein achieves an IC50 value of about 100 μM. In some aspects, the method described herein achieves an IC50 value of about 120 μM. In some aspects, the method described herein achieves an IC50 value of about 160 μM.

In some aspects, described herein is a method of modulating plasma Lp(a) level in a subject in need thereof, comprising administering to the subject a polynucleic acid molecule described herein, a polynucleic acid molecule conjugate described herein, or a pharmaceutical composition described herein, wherein the polynucleic acid molecule described herein, the polynucleic acid molecule conjugate described herein, or the pharmaceutical composition described herein reduces the plasma Lp(a) level in the subject.

In some aspects, the method described herein reduces plasma Lp(a) level in a subject by about or at least 10% compared to a control (e.g., untreated subject or a subject before the treatment). In some aspects, the method described herein reduces plasma Lp(a) level in a subject by about or at least 20% compared to a control. In some aspects, the method described herein reduces plasma Lp(a) level in a subject by about or at least 30% compared to a control. In some aspects, the method described herein reduces plasma Lp(a) level in a subject by about or at least 40% compared to a control. In some aspects, the method described herein reduces plasma Lp(a) level in a subject by about or at least 50% compared to a control. In some aspects, the method described herein reduces plasma Lp(a) level in a subject by about or at least 60% compared to a control. In some aspects, the method described herein reduces plasma Lp(a) level in a subject by about or at least 70% compared to a control. In some aspects, the method described herein reduces plasma Lp(a) level in a subject by about or at least 80% compared to a control. In some aspects, the method described herein reduces plasma Lp(a) level in a subject by about or at least 90% compared to a control. In some aspects, the method described herein reduces plasma Lp(a) level in a subject by about 100% compared to a control.

In some aspects, described herein is a method for treating or preventing a Lp(a) related disorder or symptoms thereof, cardiovascular disease or a lipid disorder, comprising administering to the subject a polynucleic acid molecule as disclosed herein, a polynucleic acid molecule conjugate as disclosed herein, or a pharmaceutical composition as disclosed herein.

In some aspects, the Lp(a) related disorder or cardiovascular disease includes coronary artery disease, acute myocardial infarction, asymptomatic carotid atherosclerosis, stroke, atrial fibrillation, or peripheral artery occlusive disease. In some aspects, the cardiovascular disease is hypercholesterolemia. In some aspects, the cardiovascular disease is myocardial infarction. In some aspects, the cardiovascular disease is the stroke. In some aspects, the cardiovascular disease is calcific aortic valve stenosis. In some aspects, the cardiovascular disease is cardiac arrest. In some aspects, the cardiovascular disease is peripheral arterial disease. In some aspects, the lipid disorder is hyperlipidemia or hypercholesterolemia.

### Combination Therapy

In some instances, the methods as disclosed herein comprises administering a polynucleic acid molecule as disclosed herein, a polynucleic acid molecule conjugate as disclosed herein, or a pharmaceutical composition as disclosed herein to a subject (e.g., a human patient) who is on a therapeutic regime for the treatment of Lp(a) related disorder or CVD at the time of, or just prior to, administration of the polynucleic acid molecule as disclosed herein, a polynucleic acid molecule conjugate as disclosed herein, or a pharmaceutical composition as disclosed herein. For example, a patient who has previously been diagnosed with atherosclerosis may have been prescribed and is taking a stable therapeutic regimen of another drug prior to and/or concurrent with administration of a polynucleic acid molecule as disclosed herein, a polynucleic acid molecule conjugate as disclosed herein, or a pharmaceutical composition as disclosed herein. The prior or concurrent therapeutic regimen can comprise, for example, (1) an agent which induces a cellular depletion of cholesterol synthesis by inhibiting 3-hydroxy-3-methylglutaryl (HMG)-coenzyme A (CoA) reductase, such as a statin (e.g., cerivastatin, atorvastatin, simvastatin, pitavastatin, rosuvastatin, fluvastatin, lovastatin, pravastatin, etc.); (2) an agent which inhibits cholesterol uptake and or bile acid re-absorption; (3) an agent which increase lipoprotein catabolism (such as niacin); and/or (4) activators of the LXR transcription factor that plays a role in cholesterol elimination such as 22-hydroxycholesterol.

### Patient Population

The methods disclosed herein are useful for reducing plasma Lp(a) levels in subjects that exhibit an elevated level of plasma Lp(a). The subject can be human or non-human primates. In some instances, the subject is otherwise healthy except for exhibiting elevated serum Lp(a). For example, the subject may not exhibit any other risk factor of cardiovascular, thrombotic or other diseases or disorders at the time of treatment. In other instances, however, the subject is selected on the basis of being diagnosed with, or at risk of developing, a disease or disorder that is caused by or correlated with elevated plasma Lp(a). For instance, at the time of, or prior to administration of the polynucleic acid molecule as disclosed herein, the polynucleic acid molecule conjugate as disclosed herein, or the pharmaceutical composition as disclosed herein, the subject may be diagnosed with or identified as being at risk of developing a cardiovascular disease or disorder, such as, e.g., coronary artery disease, acute myocardial infarction, asymptomatic carotid atherosclerosis, stroke, atrial fibrillation, peripheral artery occlusive disease, etc. The cardiovascular disease or disorder, in some instances, is hypercholesterolemia. For example, a subject may be selected for treatment with the methods as disclosed herein if the subject is diagnosed with or identified as being at risk of developing a hypercholesterolemia condition such as, e.g., heterozygous Familial Hypercholesterolemia (heFH), homozygous Familial Hypercholesterolemia (hoFH), as well as incidences of hypercholesterolemia that are distinct from Familial Hypercholesterolemia (nonFH). The subject may be diagnosed with or identified as being at risk of developing a lipid disease or disorder, such as, e.g., hyperlipidemia and hypercholesterolemia.

In some instances, at the time of, or prior to administration of the polynucleic acid molecule as disclosed herein, the polynucleic acid molecule conjugate as disclosed herein, or the pharmaceutical composition as disclosed herein, the subject may be diagnosed with or identified as being at risk of developing a thrombotic occlusive disease or disorder, such as, e.g., pulmonary embolism, central retinal vein occlusion, etc. The patient can be selected on the basis of being diagnosed with or at risk of developing a combination of two or more of the abovementioned diseases or disorders. For example, at the time of, or prior to administration of the pharmaceutical composition of the present invention, the subject may be diagnosed with or identified as being at risk of developing coronary artery disease and pulmonary embolism. Other diagnostic combinations (e.g., atherosclerosis and central retinal vein occlusion, heFH and stroke, etc.) are also included in the definition of the patient populations that are treatable by the methods as disclosed herein.

In some instances, the subject to be treated with the methods as disclosed herein is selected on the basis of one or more factors selected from the group consisting of age (e.g., older than 40, 45, 50, 55, 60, 65, 70, 75, or 80 years), race, gender (male or female), exercise habits (e.g., regular exerciser, non-exerciser), other preexisting medical conditions (e.g., type-II diabetes, high blood pressure, etc.), and current medication status (e.g., currently taking statins (e.g., cerivastatin, atorvastatin, simvastatin, pitavastatin, rosuvastatin, fluvastatin, lovastatin, pravastatin, etc.), beta blockers, niacin, etc.). The present disclosure also provides methods for reducing plasma Lp(a) levels in patients who are intolerant of, non-responsive to, or inadequately responsive to conventional statin therapy. Potential patients can be selected/screened on the basis of one or more of these factors (e.g., by questionnaire, diagnostic evaluation, etc.) before being treated with the methods as disclosed herein.

### Dosage

The amount of the polynucleic acid molecule as disclosed herein, the polynucleic acid molecule conjugate as disclosed herein, or the pharmaceutical composition as disclosed herein administered to a subject according to the methods as disclosed herein is, generally, a therapeutically effective amount. As used herein, the term “therapeutically effective amount” refers to a dose of the polynucleic acid molecule as disclosed herein, the polynucleic acid molecule conjugate as disclosed herein, or the pharmaceutical composition as disclosed herein that results in a detectable reduction in plasma Lp(a). For example, a therapeutically effective amount of the polynucleic acid molecule as disclosed herein, the polynucleic acid molecule conjugate as disclosed herein, or the pharmaceutical composition as disclosed herein includes, for example, an amount of the polynucleic acid molecule as disclosed herein, the polynucleic acid molecule conjugate as disclosed herein, or the pharmaceutical composition as disclosed herein that causes a reduction of at least 1%, 2%, 5%, 10%, 15%, 20%, 25%, 30%, 40%, 50%, or more in plasma Lp(a) levels when administered to a subject. In some instances, a therapeutically effective amount of the polynucleic acid molecule as disclosed herein can a dose that can cause a reduction of at least 1%, 2%, 5%, 10%, 15%, 20%, 25%, 30%, 40%, 50%, or more in Lp(a) mRNA levels when administered to a subject.

### Regimens

The polynucleic acid molecule as disclosed herein, the polynucleic acid molecule conjugate as disclosed herein, or the pharmaceutical composition as disclosed herein can be administered to a subject over a defined time course. The methods disclosed herein can comprise sequentially administering to a subject multiple doses of the polynucleic acid molecule as disclosed herein, the polynucleic acid molecule conjugate as disclosed herein, or the pharmaceutical composition as disclosed herein. As used herein, “sequentially administering” means that each dose of the polynucleic acid molecule, the polynucleic acid molecule conjugate, or the pharmaceutical composition is administered to the subject at a different point in time, e.g., on different days separated by a predetermined interval (e.g., hours, days, weeks or months). The present disclosure provides methods which comprise sequentially administering to the patient a single initial dose of the polynucleic acid molecule, the polynucleic acid molecule conjugate, or the pharmaceutical composition, followed by one or more secondary doses of the polynucleic acid molecule, the polynucleic acid molecule conjugate, or the pharmaceutical composition, and optionally followed by one or more tertiary doses of the polynucleic acid molecule, the polynucleic acid molecule conjugate, or the pharmaceutical composition.

The terms “initial dose,” “secondary doses,” and “tertiary doses,” refer to the temporal sequence of administration of the polynucleic acid molecule, the polynucleic acid molecule conjugate, or the pharmaceutical composition. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses can all contain the same amount of the polynucleic acid molecule, the polynucleic acid molecule conjugate, or the pharmaceutical composition, but will generally differ from one another in terms of frequency of administration. In some instances, the amount of the polynucleic acid molecule, the polynucleic acid molecule conjugate, or the pharmaceutical composition contained in the initial, secondary and/or tertiary doses will vary from one another (e.g., adjusted up or down as appropriate) during the course of treatment.

In some instances, each secondary and/or tertiary dose is administered 1 to 30 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more) days after the immediately preceding dose. The phrase “the immediately preceding dose,” as used herein, means, in a sequence of multiple administrations, the dose of the polynucleic acid molecule, the polynucleic acid molecule conjugate, or the pharmaceutical composition which is administered to a patient prior to the administration of the very next dose in the sequence with no intervening doses.

The methods as disclosed herein can comprise administering to a patient any number of secondary and/or tertiary doses of the polynucleic acid molecule, the polynucleic acid molecule conjugate, or the pharmaceutical composition. For example, in some instances, only a single secondary dose is administered to the patient. In other instances, two or more (e.g., 2, 3, 4, 5, 6, 7, 8, or more) secondary doses are administered to the patient. In some instances, only a single tertiary dose is administered to the patient. In other instances, two or more (e.g., 2, 3, 4, 5, 6, 7, 8, or more) tertiary doses are administered to the patient.

Where multiple secondary doses are administered, each secondary dose can be administered at the same frequency as the other secondary doses. For example, each secondary dose may be administered to the patient 1 to 29 days after the immediately preceding dose. Similarly, where multiple tertiary doses are administered, each tertiary dose may be administered at the same frequency as the other tertiary doses. For example, each tertiary dose may be administered to the patient 1 to 60 days after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

### Exemplary Embodiments

Embodiment 1. A polynucleic acid molecule for modulating expression of lipoprotein(a) (Lp(a)) gene, comprising a nucleic acid sequence that is at least 80%, at least 85%, at least 90% identical to a nucleic acid sequence selected from Table 1, Table 2, or Table 5.

Embodiment 2. The polynucleic acid molecule of embodiment 1, wherein the polynucleic acid molecule comprises a nucleic acid sequence selected from Table 1, Table 2, or Table 5.

Embodiment 3. The polynucleic acid molecule of any one of embodiments 1-2, wherein the polynucleic acid molecule is a single-stranded nucleic acid molecule.

Embodiment 4. The polynucleic acid molecule of embodiment 3, wherein the single-stranded nucleic acid molecule comprises at least 14, 15, 16, 17, 18 consecutive nucleotides that am complementary to a nucleic acid sequence selected from SEQ ID NOs: 309-462, with no more than 1, 2, 3, 4 mismatches.

Embodiment 5. The polynucleic acid molecule of embodiment 3, wherein the single-stranded nucleic acid molecule comprises at least 80%, at least 85%, at least 90%, at least 95%, at least 96%, at least 97%, at least 98%, at least 99% identical to a nucleic acid sequence selected from SEQ ID NOs: 1-154.

Embodiment 6. The polynucleic acid molecule of embodiment 1 or 2, wherein the polynucleic acid molecule is a double-stranded nucleic acid molecule comprising a sense strand and an antisense strand.

Embodiment 7. The polynucleic acid molecule of embodiment 5, wherein the sense strand comprises a nucleic acid sequence that is at least 80%, at least 85%, at least 90%, at least 95%, at least 96%, at least 97%, at least 98%, at least 99% identical to a nucleic acid sequence selected from SEQ ID NOs: 309-462.

Embodiment 8. The polynucleic acid molecule of embodiment 5 or embodiment 6, wherein the antisense strand comprises comprises a nucleic acid sequence that is at least 80%, at least 85%, at least 90%, at least 95%, at least 96%, at least 97%, at least 98%, at least 99% identical to a nucleic acid sequence selected from SEQ ID NOs: 1-154.

Embodiment 9. The polynucleic acid molecule of embodiment 5, wherein the sense strand comprises a nucleic acid sequence comprising at least 14, 15, 16, 17, 18, 19, or 20 consecutive sequences from a nucleic acid sequence selected from SEQ ID NOs: 309-462, with no more than 1, 2, 3, or 4 mismatches.

Embodiment 10. The polynucleic acid molecule of embodiment 6, wherein the antisense strand comprises a nucleic acid sequence comprising at least 14, 15, 16, 17, 18, 19, 20, 21, or 22 consecutive sequences of a nucleic acid sequence selected from SEQ ID NOs: 1-154, with no more than 1, 2, 3, or 4 mismatches.

Embodiment 11. The polynucleic acid molecule of any one of embodiments 6-10, wherein the sense strand comprises a nucleic acid sequence selected from SEQ ID NOs: 309-462 and the antisense strand comprises a nucleic acid sequence selected from SEQ ID NOs: 1-154.

Embodiment 12. The polynucleic acid molecule of any one of embodiments 1-11, wherein the polynucleic acid molecule comprises (1) a 2′-fluoro modified nucleotide; (2) a 2′-O-methyl modified nucleotide; or (3) a modified internucleotide linkage.

Embodiment 13. The polynucleic acid molecule of any one of embodiments 6-12, wherein the sense strand comprises at least two consecutive modified internucleotide linkages at the 5′ end.

Embodiment 14. The polynucleic acid molecule of any one of embodiments 6-13, wherein the antisense strand comprises at least two consecutive modified internucleotide linkages at the 5′ end and/or 3′ end.

Embodiment 15. The polynucleic acid molecule of any one of embodiments 6-14, wherein the sense strand comprises 5′-nnnnnnNfnNfnNfnnnnnnnnnn-3′, wherein the antisense strand comprises 5′-nNfnnnnNfnnnnNfnNfnNfnnnnnnn-3′, wherein n stands for a 2′-O-methyl modified nucleotide, and wherein Nf stands for a 2′-fluoro modified nucleotide.

Embodiment 16. The polynucleic acid molecule of any one of embodiments 12-15, wherein the modified internucleotide linkage is a phosphorothioate linkage.

Embodiment 17. The polynucleic acid molecule of embodiment 16, wherein the modified internucleotide linkage comprises a stereochemically enriched phosphorothioate internucleotide linkage.

Embodiment 18. The polynucleic acid molecule of any one of embodiments 12-17, wherein the modified internucleotide linkage is an SP chiral internucleotide phosphorothioate linkage.

Embodiment 19. The polynucleic acid molecule of embodiment 18, wherein the polynucleic acid comprises a plurality of modified internucleotide linkages, and at least 1, 2, 3, or 4 of the plurality of modified internucleotide linkages are stereochemically enriched phosphorothioate internucleotide linkages.

Embodiment 20. The polynucleic acid molecule of embodiment 19, wherein the stereochemically enriched phosphorothioate internucleotide linkages comprise both R- and S-isomers.

Embodiment 21. The polynucleic acid molecule of embodiment 19 or 20, wherein the at least one stereochemically enriched phosphorothioate is disposed between two consecutive nucleosides that are two of six 5′ or 3′-terminal nucleosides of the sense strand or the antisense strand.

Embodiment 22. The polynucleic acid molecule of any one of embodiments 6-21, wherein the sense strand or antisense strand is 19-25, or 21-23 nucleotides in length.

Embodiment 23. The polynucleic acid molecule of embodiments 1-5, wherein the polynucleic acid molecule is 19-25 or 21-23 nucleotides in length.

Embodiment 24. The polynucleic acid molecule of any one of embodiments 1-23, wherein the sense strand comprises a nucleic acid sequence that is at least 80%, at least 85%, at least 90%, at least 95% identical to a nucleic acid sequence selected from SEQ ID NOs: 463-616.

Embodiment 25. The polynucleic acid molecule of any one of embodiments 1-24, wherein the antisense strand comprises a nucleic acid sequence that is at least 80%, at least 85%, at least 90%, at least 95% identical to a nucleic acid sequence selected from SEQ ID NOs: 155-308.

Embodiment 26. The polynucleic acid molecule of any one of embodiments 1-25, wherein the sense strand comprises a sequence selected from a nucleic acid sequence of SEQ ID NOs: 463-616, and the antisense strand comprises a sequence selected from a nucleic acid sequence of SEQ ID NOs: 155-308.

Embodiment 27. A polynucleic acid molecule for modulating expression of Lipoprotein(a) (Lp(a)) gene, wherein polynucleic acid molecule comprises:
(a) an antisense strand comprising the nucleotide sequence of UAGAUGACCAAGCUUGGCAGGUC (SEQ ID NO: 4) and a sense strand comprising the nucleotide sequence of CCUGCCAAGCUUGGUCAUCUA (SEQ ID NO: 312);
(b) an antisense strand comprising the nucleotide sequence of UAUAGAUGACCAAGCUUGGCAGG (SEQ ID NO: 5) and a sense strand comprising the nucleotide sequence of UGCCAAGCUUGGUCAUCUAUA (SEQ ID NO:313);
(c) an antisense strand comprising the nucleotide sequence of UCAUAGAUGACCAAGCUUGGCAG (SEQ ID NO: 6) and a sense strand comprising the nucleotide sequence of GCCAAGCUUGGUCAUCUAUGA (SEQ ID NO: 314);
(d) an antisense strand comprising the nucleotide sequence of UCGACGGCAGUCCCUUCUGCGUC (SEQ ID NO: 11) and a sense strand comprising the nucleotide sequence of CGCAGAAGGGACUGCCGUCGA (SEQ ID NO: 319);
(e) an antisense strand comprising the nucleotide sequence of UUCUAGGCUUGGAACCGGGGUAA (SEQ ID NO: 18) and a sense strand comprising the nucleotide sequence of ACCCCGGUUCCAAGCCUAGAA (SEQ ID NO: 326);
(f) an antisense strand comprising the nucleotide sequence of UAGCCUCUAGGCUUGGAACCGGG (SEQ ID NO: 21) and a sense strand comprising the nucleotide sequence of CGGUUCCAAGCCUAGAGGCUA (SEQ ID NO: 329);
(g) an antisense strand comprising the nucleotide sequence of UUUACCGUGGUAGCACUCCUGCA (SEQ ID NO: 44) and a sense strand comprising the nucleotide sequence of CAGGAGUGCUACCACGGUAAA (SEQ ID NO: 352);
(h) an antisense strand comprising the nucleotide sequence of UUGUCCAUUACCGUGGUAGCACU (SEQ ID NO: 47) and a sense strand comprising the nucleotide sequence of UGCUACCACGGUAAUGGACAA (SEQ ID NO: 355);
(i) an antisense strand comprising the nucleotide sequence of UCUCUGUCCAUUACCGUGGUAGC (SEQ ID NO: 49) and a sense strand comprising the nucleotide sequence of UACCACGGUAAUGGACAGAGA (SEQ ID NO: 357);
(j) an antisense strand comprising the nucleotide sequence of UAUUGUGUCAGGUUGCAGUACUC (SEQ ID NO: 60) and a sense strand comprising the nucleotide sequence of GUACUGCAACCUGACACAAUA (SEQ ID NO: 368);
(k) an antisense strand comprising the nucleotide sequence of UUGCGUCUGAGCAUUGUGUCAGG (SEQ ID NO: 64) and a sense strand comprising the nucleotide sequence of UGACACAAUGCUCAGACGCAA (SEQ ID NO: 372);
(l) an antisense strand comprising the nucleotide sequence of UUAACUCUGUCCAUAAUGGUAGU (SEQ ID NO: 88) and a sense strand comprising the nucleotide sequence of UACCAUUAUGGACAGAGUUAA (SEQ ID NO: 396);
(m) an antisense strand comprising the nucleotide sequence of UCCAAGCUUGGCAAGUUCUUCCU (SEQ ID NO: 89) and a sense strand comprising the nucleotide sequence of GAAGAACUUGCCAAGCUUGGA (SEQ ID NO: 397);
(n) an antisense strand comprising the nucleotide sequence of UAGAUGACCAAGCUUGGCAAGUU (SEQ ID NO: 90) and a sense strand comprising the nucleotide sequence of CUUGCCAAGCUUGGUCAUCUA (SEQ ID NO: 398); or
(o) an antisense strand comprising the nucleotide sequence of UGGUCCGACUAUGCUGGUGUGGU (SEQ ID NO: 98) and a sense strand comprising the nucleotide sequence of CACACCAGCAUAGUCGGACCA (SEQ ID NO: 406).

Embodiment 28. A polynucleic acid molecule for modulating expression of Lipoprotein(a) (Lp(a)) gene, wherein polynucleic acid molecule comprises:
(a) an antisense strand comprising the nucleotide sequence of usAfsgaugAfccaaGfcUfuGfgcaggsusc (SEQ ID NO: 158) and a sense strand comprising the nucleotide sequence of cscsugccAfaGfcUfuggucaucua (SEQ ID NO: 466);
(b) an antisense strand comprising the nucleotide sequence of usAfsuagaUfgaccAfaGfcUfuggcasgsg (SEQ ID NO: 159) and a sense strand comprising the nucleotide sequence of usgsccaaGfcUfuGfgucaucuaua (SEQ ID NO: 467);
(c) an antisense strand comprising the nucleotide sequence of usCfsauagAfugacCfaAfgCfuuggcsasg (SEQ ID NO: 160) and a sense strand comprising the nucleotide sequence of gscscaagCfuUfgGfucaucuauga (SEQ ID NO: 468);
(d) an antisense strand comprising the nucleotide sequence of usCfsgacgGfcaguCfcCfuUfcugcgsusc (SEQ ID NO: 165) and a sense strand comprising the nucleotide sequence of csgscagaAfgGfgAfcugccgucga (SEQ ID NO: 473);
(e) an antisense strand comprising the nucleotide sequence of usUfscuagGfcuugGfaAfcCfggggusasa (SEQ ID NO: 172) and a sense strand comprising the nucleotide sequence of ascscccgGfuUfcCfaagccuagaa (SEQ ID NO: 480);
(f) an antisense strand comprising the nucleotide sequence of usAfsgccuCfuaggCfuUfgGfaaccgsgsg (SEQ ID NO: 175) and a sense strand comprising the nucleotide sequence of csgsguucCfaAfgCfcuagaggcua (SEQ ID NO: 483);
(g) an antisense strand comprising the nucleotide sequence of usUfsuaccGfugguAfgCfaCfuccugscsa (SEQ ID NO: 198) and a sense strand comprising the nucleotide sequence of csasggagUfgCfuAfccacgguaaa (SEQ ID NO: 506);
(h) an antisense strand comprising the nucleotide sequence of usUfsguccAfuuacCfgUfgGfuagcascsu (SEQ ID NO: 201) and a sense strand comprising the nucleotide sequence of usgscuacCfaCfgGfuaauggacaa (SEQ ID NO: 509);
(i) an antisense strand comprising the nucleotide sequence of usCfsucugUfccauUfaCfcGfugguasgsc (SEQ ID NO: 203) and a sense strand comprising the nucleotide sequence of usasccacGfgUfaAfuggacagaga (SEQ ID NO: 511);
(j) an antisense strand comprising the nucleotide sequence of usAfsuuguGfucagGfuUfgCfaguacsusc (SEQ ID NO: 214) and a sense strand comprising the nucleotide sequence of gsusacugCfaAfcCfugacacaaua (SEQ ID NO: 522);
(k) an antisense strand comprising the nucleotide sequence of usUfsgcguCfugagCfaUfuGfugucasgsg (SEQ ID NO: 218) and a sense strand comprising the nucleotide sequence of usgsacacAfaUfgCfucagacgcaa (SEQ ID NO: 526);
(l) an antisense strand comprising the nucleotide sequence of usUfsaacuCfugucCfaUfaAfugguasgsu (SEQ ID NO: 242) and a sense strand comprising the nucleotide sequence of usasccauUfaUfgGfacagaguuaa (SEQ ID NO: 550);
(m) an antisense strand comprising the nucleotide sequence of usCfscaagCfuuggCfaAfgUfucuucscsu (SEQ ID NO: 243) and a sense strand comprising the nucleotide sequence of gsasagaaCfuUfgCfcaagcuugga (SEQ ID NO: 551);
(n) an antisense strand comprising the nucleotide sequence of usAfsgaugAfccaaGfcUfuGfgcaagsusu (SEQ ID NO: 244) and a sense strand comprising the nucleotide sequence of csusugccAfaGfcUfuggucaucua (SEQ ID NO: 552); or
(o) an antisense strand comprising the nucleotide sequence of usGfsguccGfacuaUfgCfuGfgugugsgsu (SEQ ID NO: 252) and a sense strand comprising the nucleotide sequence of csascaccAfgCfaUfagucggacca (SEQ ID NO: 560),
wherein “A” refers to adenosine-3′-phosphate; “a” refers to 2′-O-methyladenosine-3′-phosphate; “Af” refers to 2′-fluoroadenosine-3′-phosphate; “C” refers to cytidine-3′-phosphate; “c” refers to 2′-O-methylcytidine-3′-phosphate; “Cf” refers to 2′-fluorocytidine-3′-phosphate; “G” refers to guanosine-3′-phosphate; “g” refers to 2′-O-methylguanosine-3′-phosphate; “Gf” refers to 2′-fluoroguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “s” refers to 3′-phosphorothioate.

Embodiment 29. A polynucleic acid molecule conjugate for modulating expression of lipoprotein(a) gene (Lp(a)), wherein the polynucleic acid molecule conjugate comprises a polynucleic acid molecule of any one of embodiments 1-23 and an asialoglycoprotein receptor targeting moiety.

Embodiment 30. The polynucleic acid molecule conjugate of embodiment 29, wherein the asialoglycoprotein receptor targeting moiety comprises N-Acetylgalactosamine (GalNAc) or galactose.

Embodiment 31. The polynucleic acid molecule conjugate of any one of embodiments 29-30, wherein the polynucleic acid molecule and the asialoglycoprotein receptor targeting moiety is coupled via a linker.

Embodiment 32. The polynucleic acid molecule conjugate of embodiment 31, wherein the linker is a cleavable linker.

Embodiment 33. The polynucleic acid molecule conjugate of any one of embodiments 30-31, wherein the GalNAc comprises an anomeric carbon bonded to trivalent, tetravalent linker, pentavalent, or hexavalent linker, wherein the anomeric carbon is part of a hemiaminal group.

Embodiment 34. The polynucleic acid molecule conjugate of any one of embodiments 31-33, wherein the linker comprises formula (IV) below,

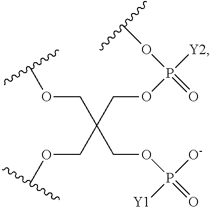

wherein at least one of Y1 and Y2 is a nucleotide in the polynucleic acid molecule.

Embodiment 35. The polynucleic acid molecule conjugate of embodiment 34, wherein the Y1 is the last nucleotide on the 3′-terminus of the sense strand of the polynucleic acid molecule.

Embodiment 36. The polynucleic acid molecule conjugate of any one of embodiments 31-35, wherein the linker and the asialoglycoprotein receptor targeting moiety with the last nucleotide on the 3′-terminus of the sense strand of the polynucleic acid molecule are shown in Formula (V′), Formula (V″″), Formula (V′″″), or Formula (V″″″):

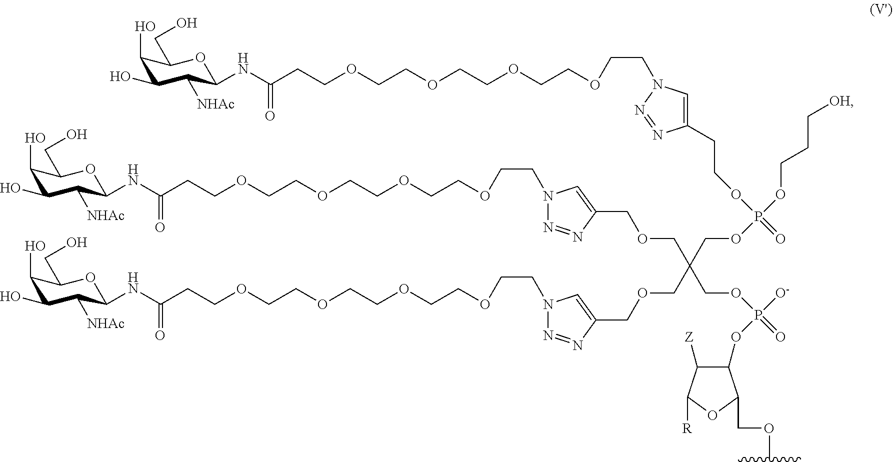

wherein Z in formula (V′) is —H, —OH, —O-Methyl, —F, or —O-methoxyethyl and R in formula (V′) is adenine, uracil, guanine, cytosine, thymine, abasic, or others;

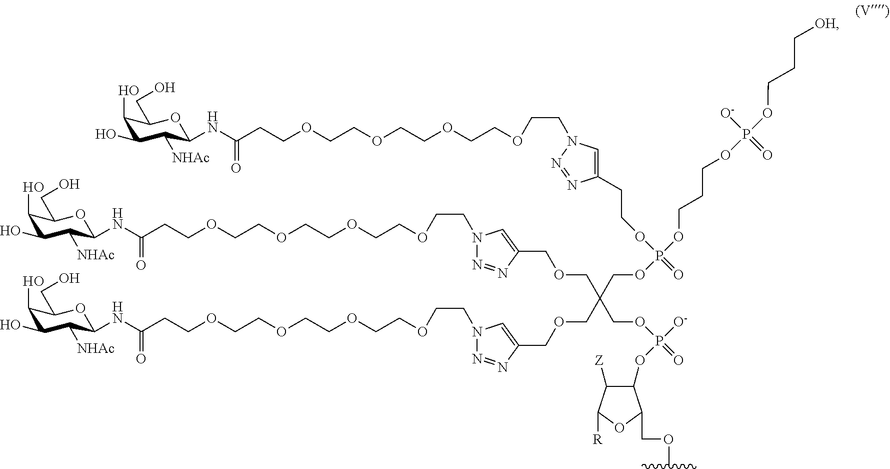

wherein Z in formula (V″″) is a moiety that corresponds to one of the sugar modifications described herein (e.g., —H, —OH, —O-Methyl, —F, or —O-methoxyethyl) and R in formula (V″″) is adenine, uracil, guanine, cytosine, thymine, abasic, or others.

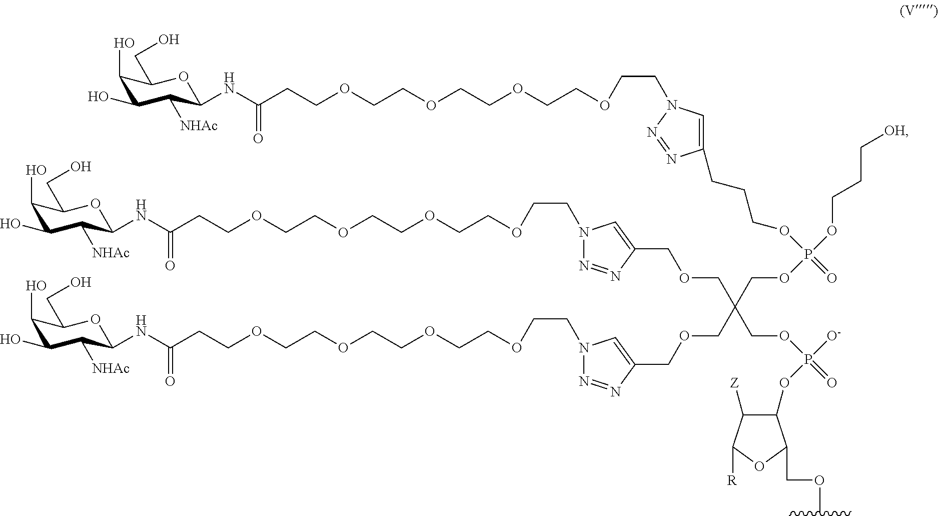

wherein Z in formula (V′″″) is a moiety that corresponds to one of the sugar modifications described herein (e.g., —H, —OH, —O-Methyl, —F, or —O-methoxyethyl) and R in formula (V′″″) is adenine, uracil, guanine, cytosine, thymine, abasic, or others; or

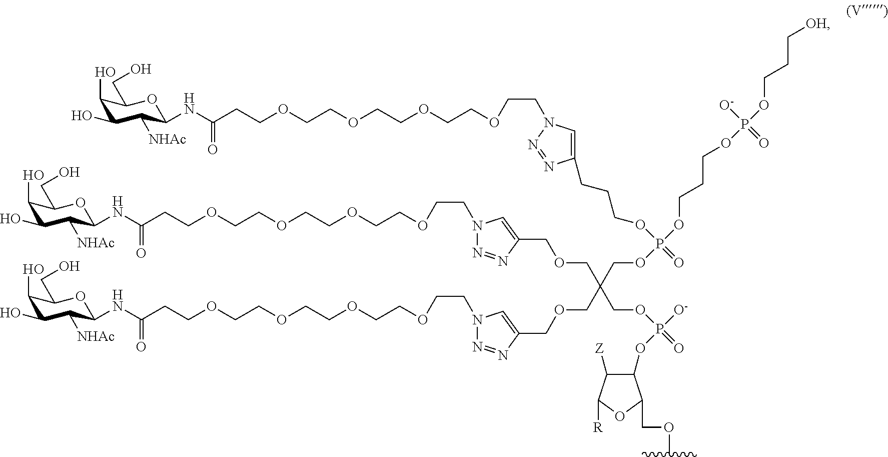

wherein Z in formula (V″″″) is a moiety that corresponds to one of the sugar modifications described herein (e.g., —H, —OH, —O-Methyl, —F, or —O-methoxyethyl) and R in formula (V″″″) is adenine, uracil, guanine, cytosine, thymine, abasic, or others.

Embodiment 37. The polynucleic acid molecule conjugate of embodiment 36, wherein the linker and the asialoglycoprotein receptor targeting moiety with the last nucleotide on the 3′-terminus of the sense strand of the polynucleic acid molecule are shown in Formula (V′):

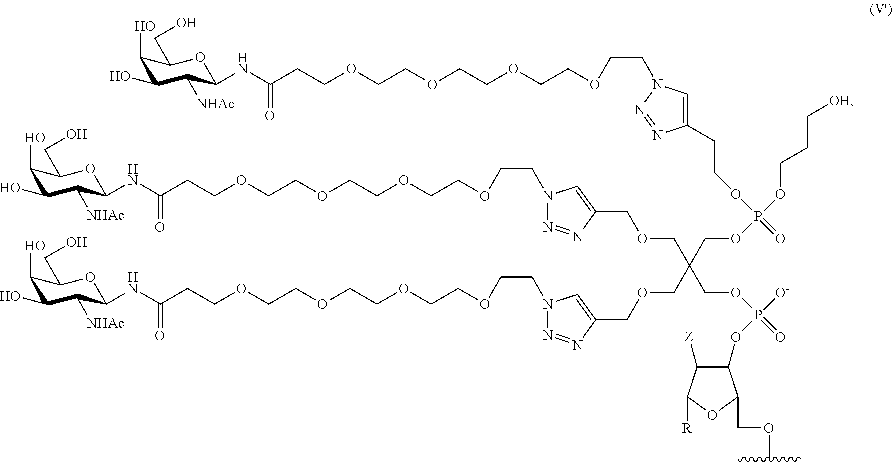

wherein Z in formula (V′) is —H, —OH, —O-Methyl, —F, or —O-methoxyethyl and R in formula (V′) is adenine, uracil, guanine, cytosine, thymine, abasic, or others.

Embodiment 38. A pharmaceutical composition comprising a polynucleic acid molecule of any one of embodiments 1-28 or a polynucleic acid molecule conjugate of any one of embodiments 29-37, and a pharmaceutically acceptable excipient.

Embodiment 39. The pharmaceutical composition of embodiment 38, wherein the pharmaceutical composition is formulated as a nanoparticle formulation.

Embodiment 40. The pharmaceutical composition of embodiment 38 or embodiment 39, wherein the pharmaceutical composition is formulated for parenteral, oral, intranasal, buccal, rectal, transdermal, intravenous, subcutaneous, or intrathecal administration.

Embodiment 41. A method of modulating expression of lipoprotein(a) (Lp(a)) gene in a subject in need thereof, comprising:
administering to the subject a polynucleic acid molecule of any one of embodiments 1-28, a polynucleic acid molecule conjugate of any one of embodiments 29-37, or a pharmaceutical composition of any one of embodiments 38-40, thereby modulating the expression of Lp(a) gene in the subject in need thereof.

Embodiment 42. A method for treating or preventing a cardiovascular disease or a lipid disorder, comprising
administering to the subject a polynucleic acid molecule of any one of embodiments 1-28, a polynucleic acid molecule conjugate of any one of embodiments 29-37, or a pharmaceutical composition of any one of embodiments 38-40, thereby modulating the expression of Lp(a) gene in the subject in need thereof.

Embodiment 43. The method of embodiment 41 or 42, wherein the polynucleic acid molecule is administered at a dose sufficient to decrease the expression of Lp(a) gene in a cell of said subject by at least about 10%, 20%, 30%, 40%, 50%, 60%, 70%, 80%, or 90% compared to a control.

Embodiment 44. The method of embodiment 41 or 42, wherein the polynucleic acid molecule is administered at a dose sufficient to decrease plasma Lp(a) level of said subject by at least about 10%, 20%, 30%, 40%, 50%, 60%, 70%, 80%, or 90% compared to a control.

Embodiment 45. The method of any one of embodiments 41-44, wherein the polynucleic acid molecule is administered in at least two doses comprising an initial dose and a secondary dose.

Embodiment 46. The method of embodiment 45, wherein the secondary dose is administered 1 to 30 days after the initial dose is administered.

Embodiment 47. The method of any one of embodiments 42-46, wherein the cardiovascular disease is coronary artery disease, acute myocardial infarction, asymptomatic carotid atherosclerosis, stroke, atrial fibrillation, or peripheral artery occlusive disease.

Embodiment 48. The method of any one of embodiments 42-46, wherein the lipid disorder is hyperlipidemia or hypercholesterolemia.

### EXAMPLES

These examples are provided for illustrative purposes only and not to limit the scope of the claims provided herein.

### Example 1—In Vitro Efficacy of siRNAs Targeting Lp(a) in Primary Human Hepatocytes (PHH)

Primary human hepatocytes were thawed and plated on collagen-coated 96-well plates at a density of 9×10⁴ cells per well. Hepatocytes were treated with conjugated siRNAs in the absence of transfection reagents (free uptake). Cells were treated with siRNAs with a concentrations at 10 μM or 0.5 μM. Untreated PHHs were used as negative control. An siRNA targeting an unrelated gene (Ahsa1) was also used as a negative control. Cells were incubated at 37° C., 5% CO₂ for 48 h. At the end of the incubation period, the cells were lysed, and the relative expression of the target gene was measured by branched DNA (bDNA) assay and normalized to a house-keeping gene using standard protocols.

The modifications of the sense and antisense strands of the siRNAs tested are listed in Table 1. Note that each of the sense strand sequences was coupled to a GalNac-L96 at its 3′ end. The in vitro potency data of the siRNAs is provided in Table 3.

Forty-eight (48) Lp(a) siRNAs were selected for dose response relationship according to standard dose response curve protocol. The doses tested include 10.0, 3.33, 1.11, 0.37, 0.12, 0.04, 0.01, 0.005, 0.002, and 0.001 μM of the siRNA. The siRNAs were purified to at least approximately 85%.

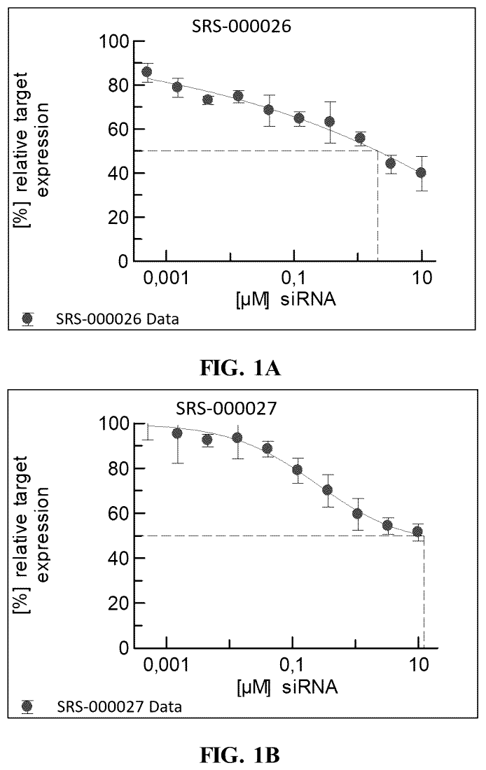

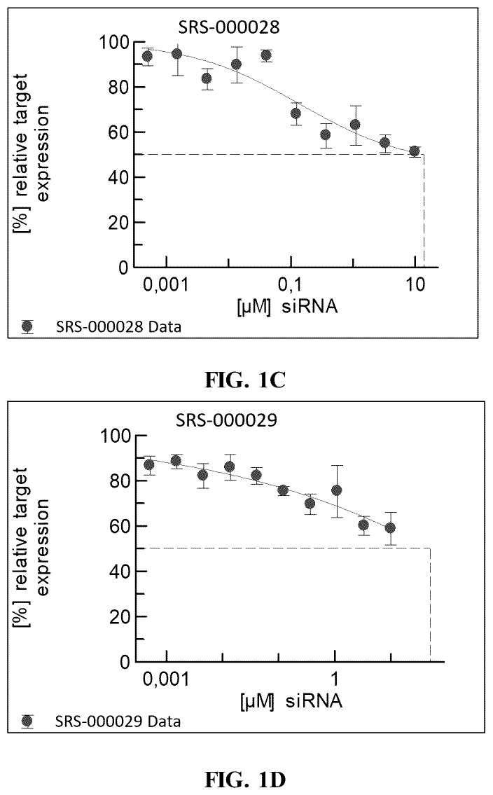

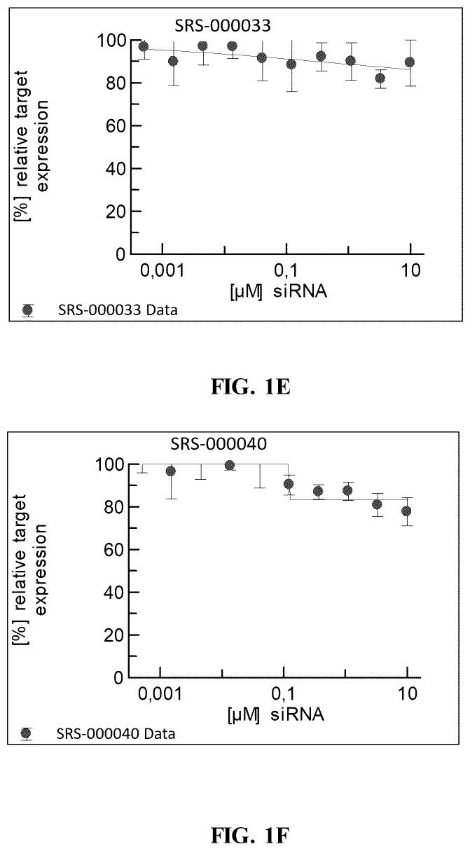

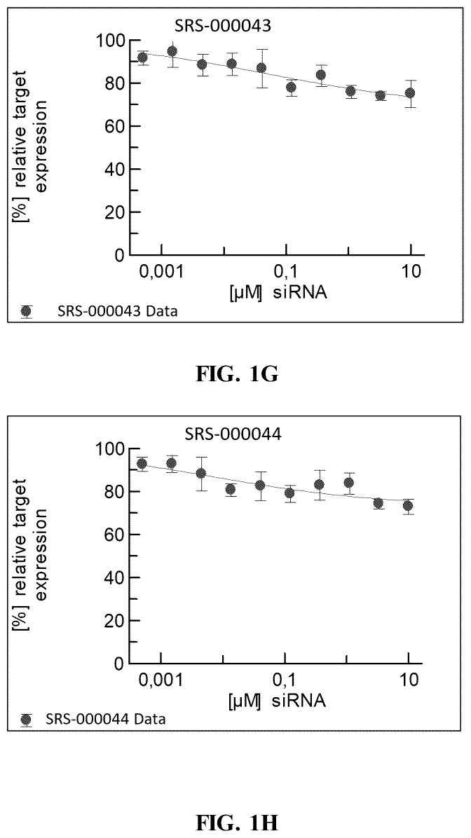

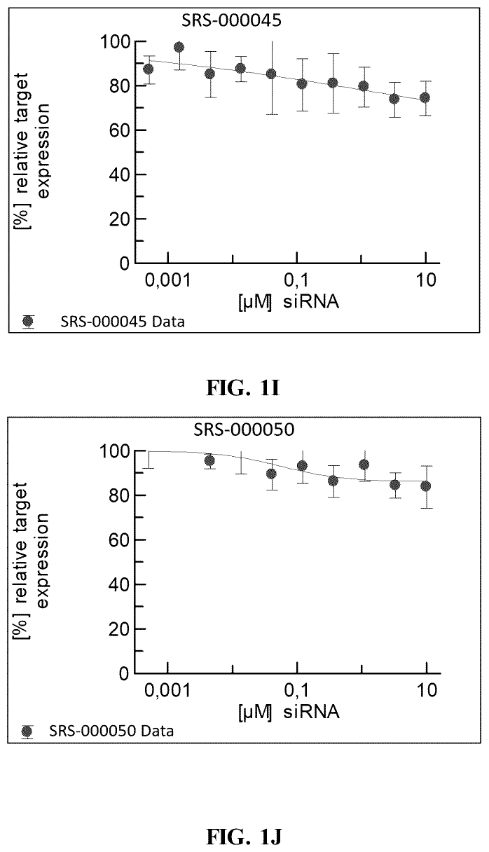

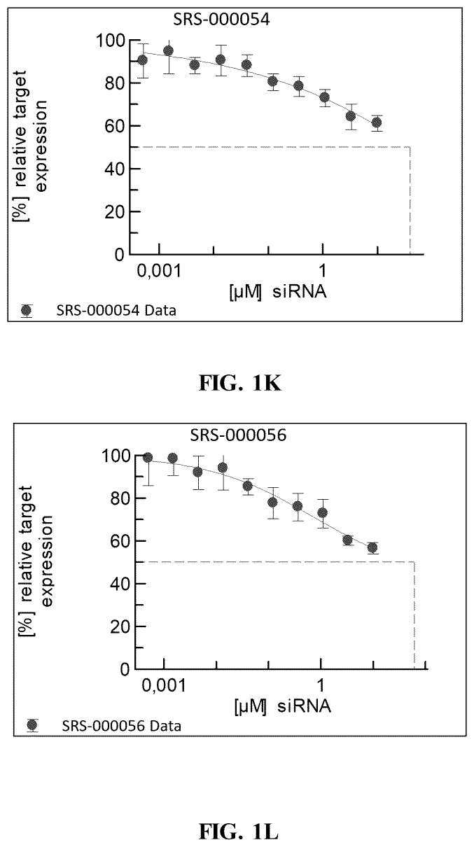

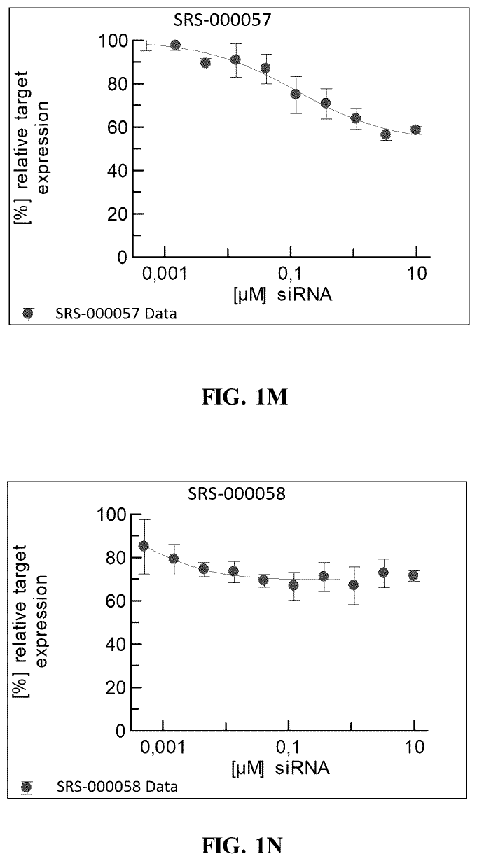

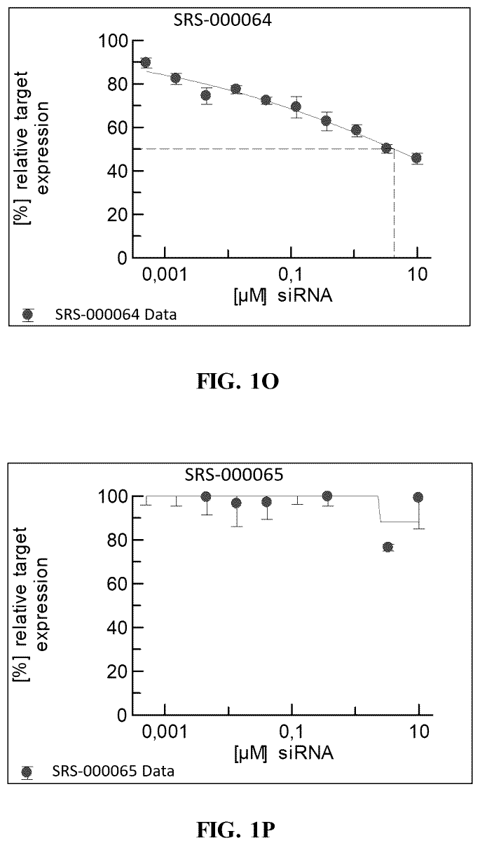

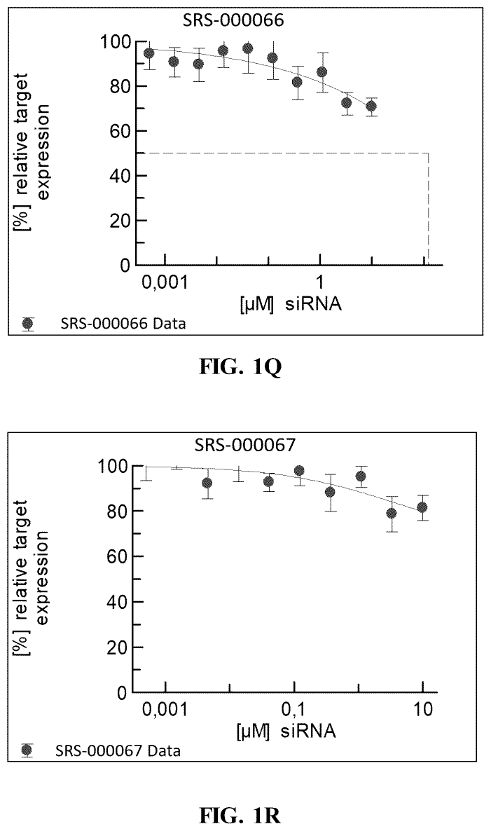

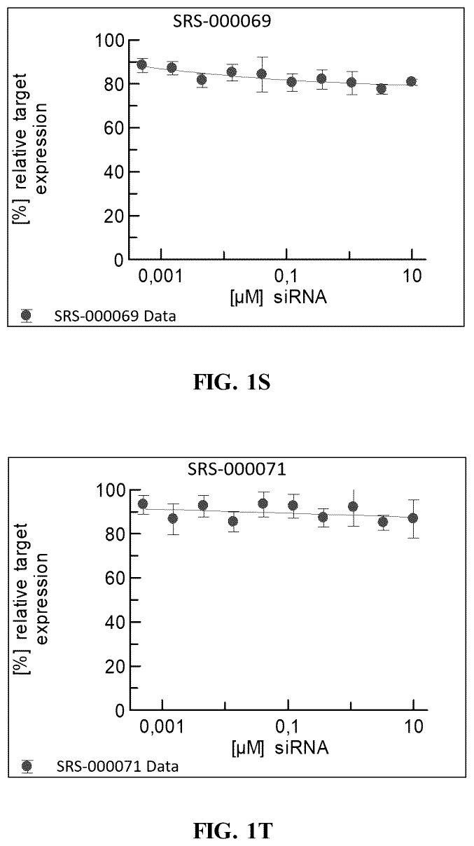

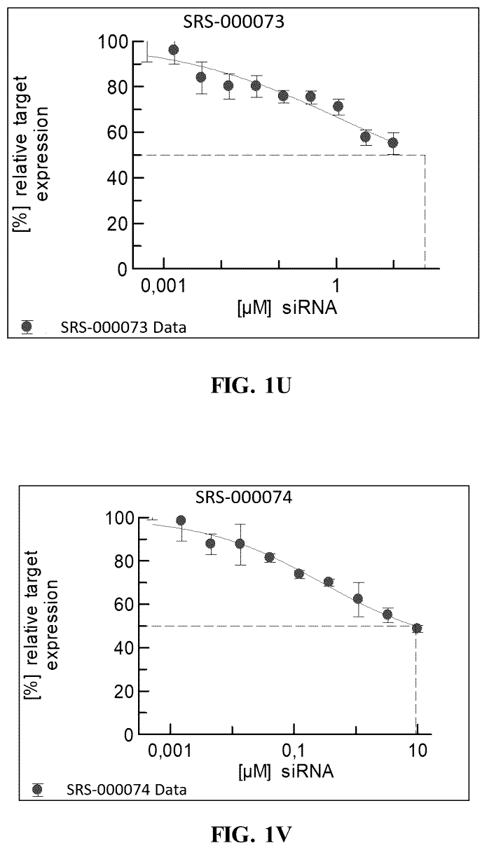

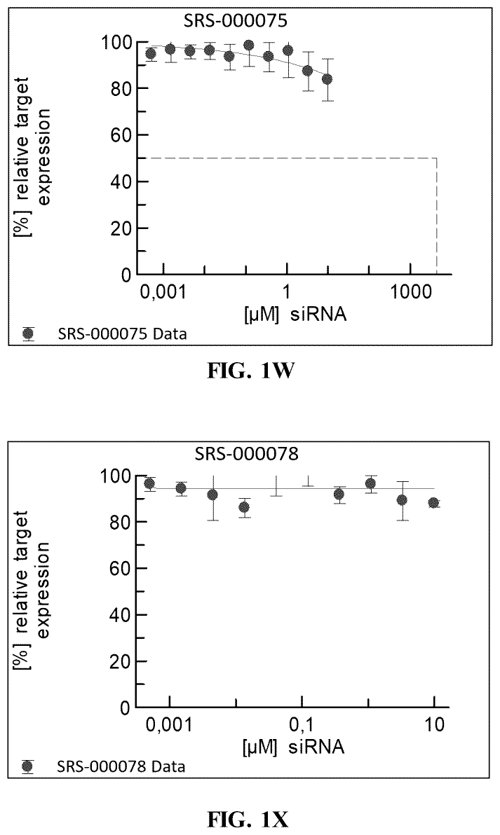

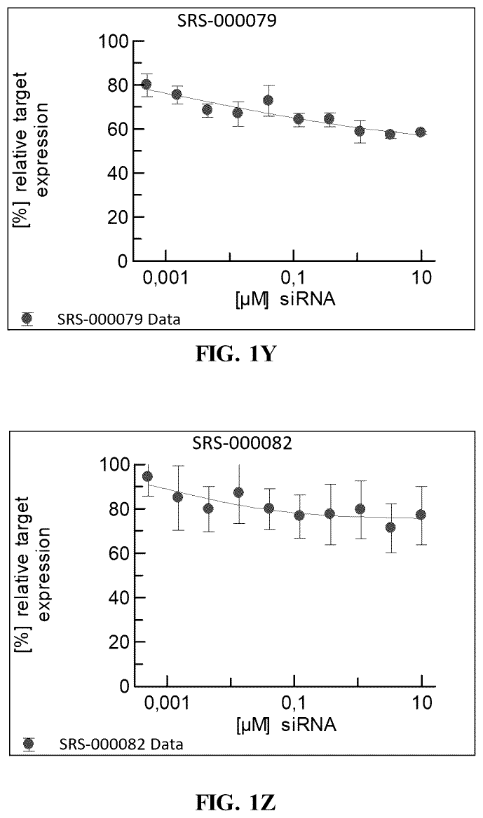

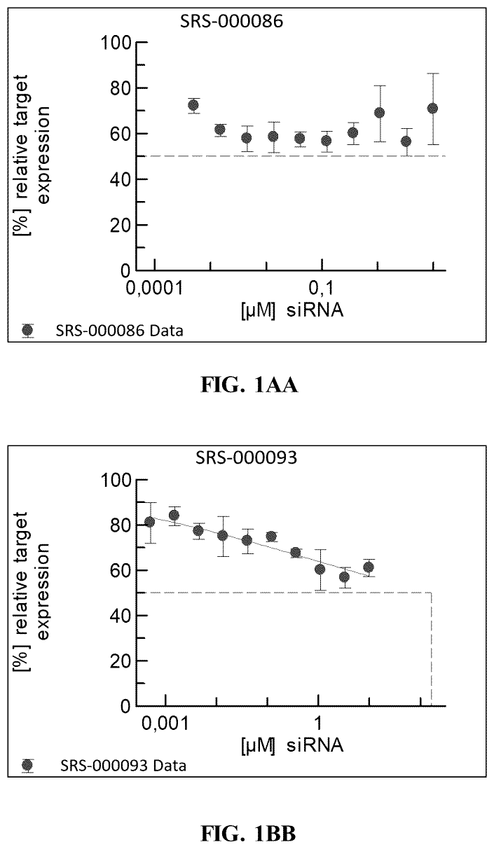

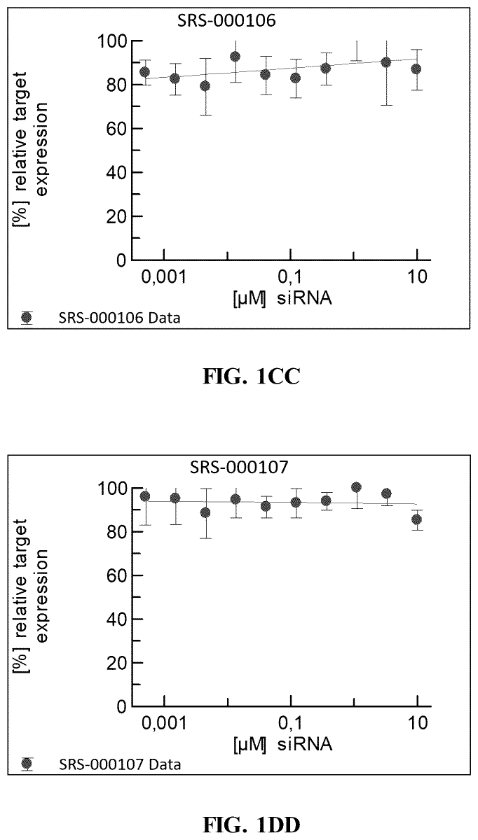

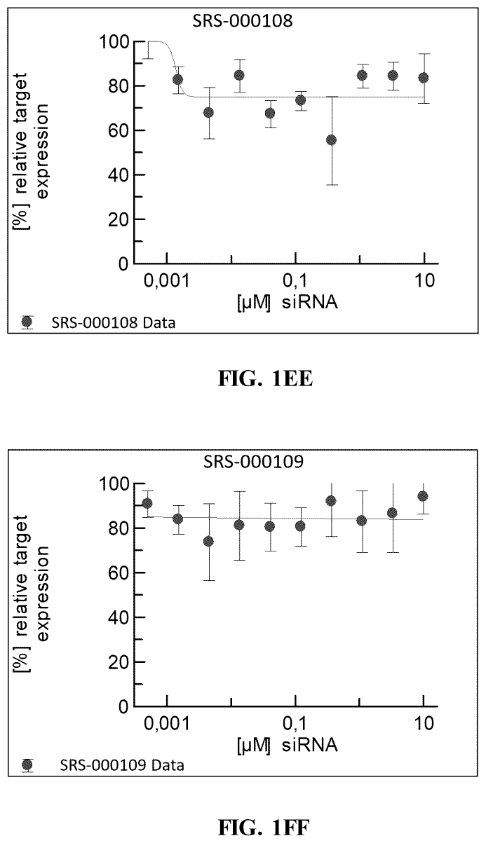

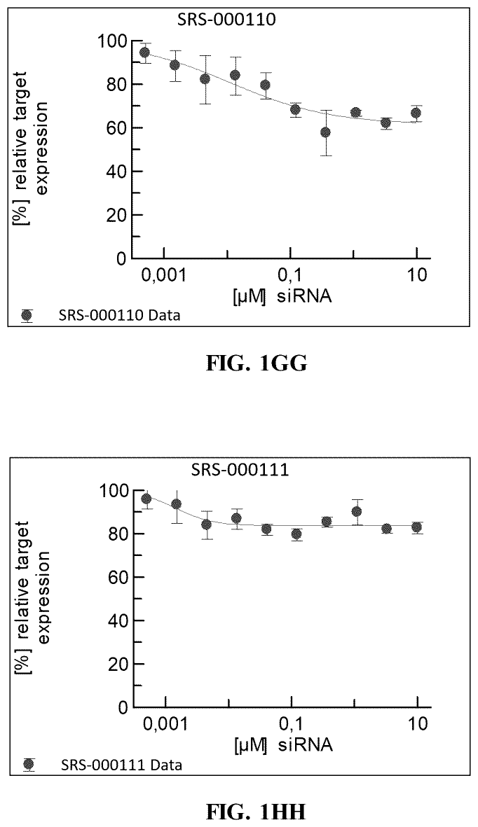

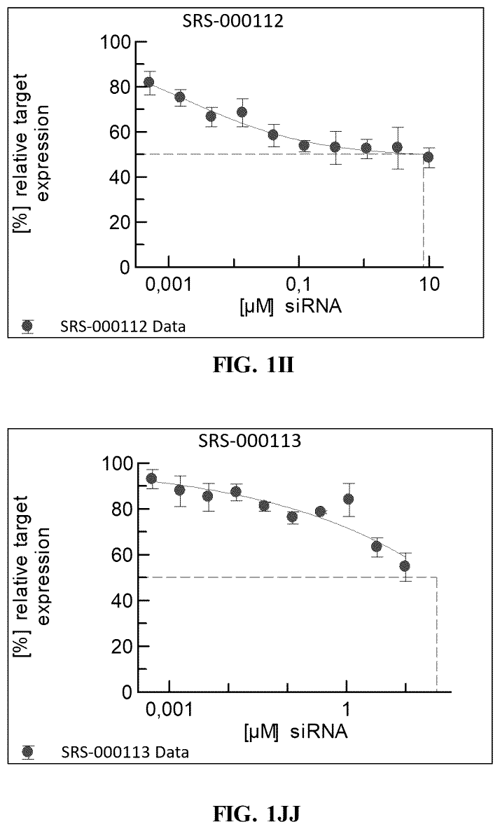

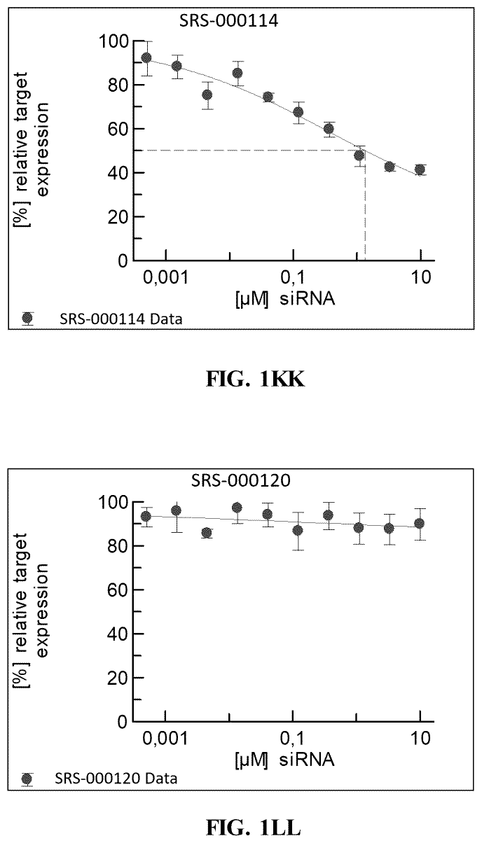

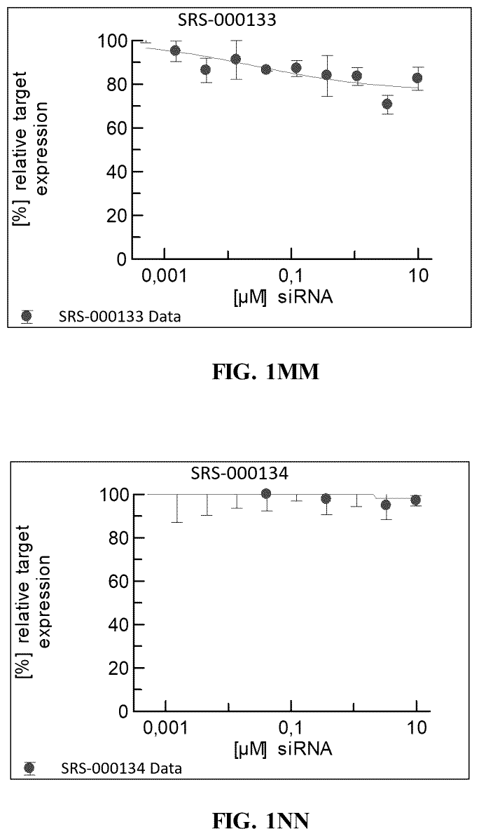

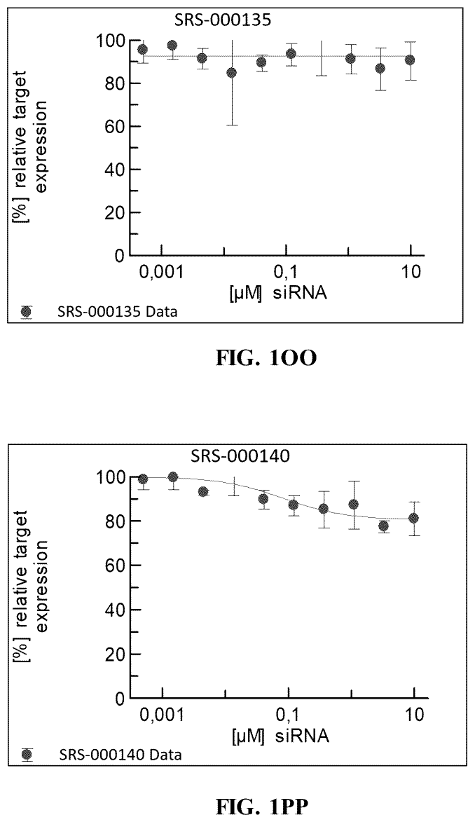

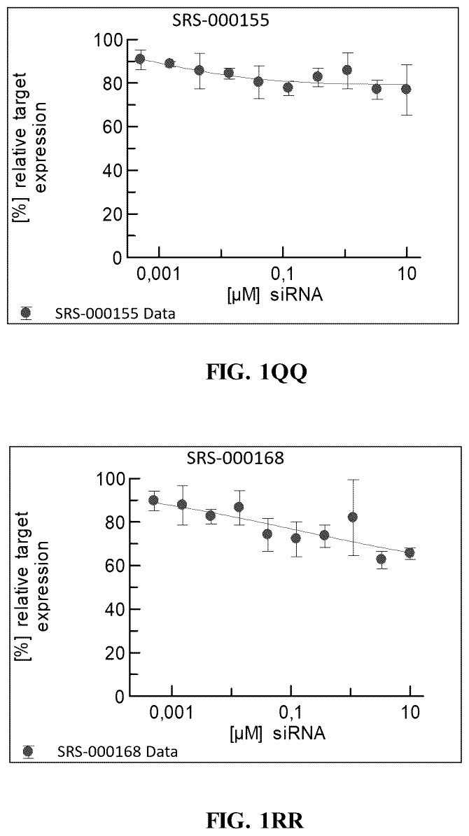

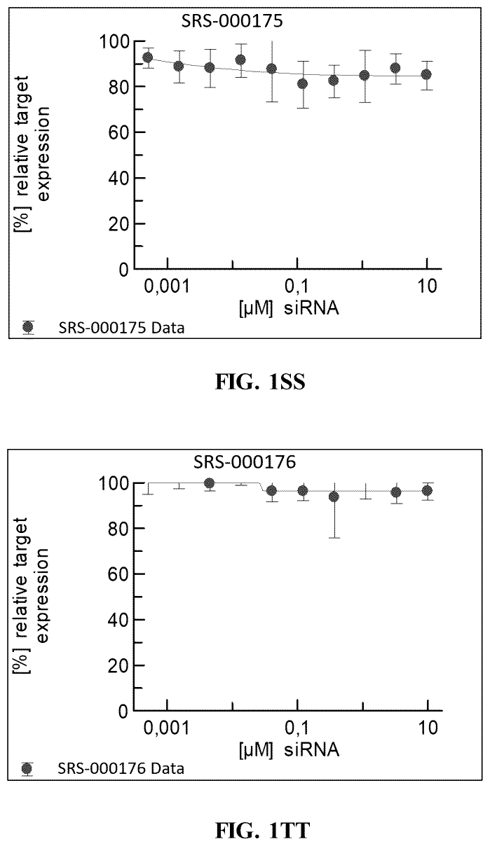

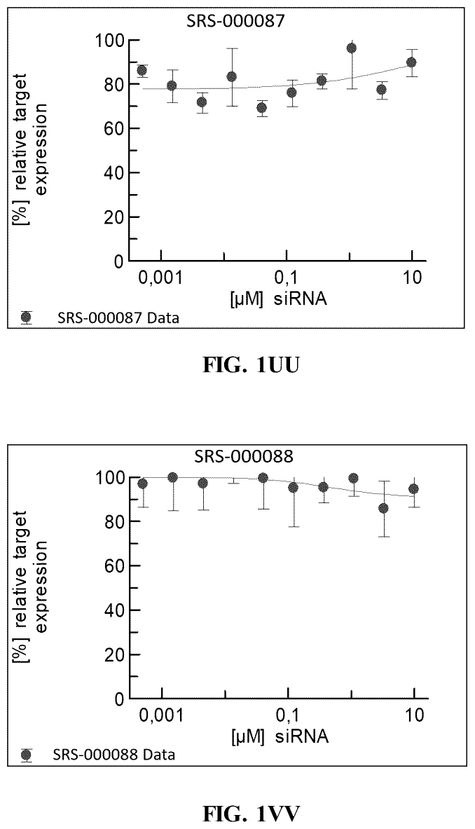

A summary of the dose response curve data is provided in Table 4. The dose response curves are provided in FIGS. 1A-1VV.

### Example 2—Testing Lp(a) siRNAs in Non-Human Primates

Fifteen (15) Lp(a) siRNAs (denoted as “SRS-000008” to “SRS-000022” from Table 2) were tested in a non-human primate study. For each siRNA, the 3′ end of the passenger/sense strand was conjugated with a GalNAc via X2 (see Formula (V′)). The Lp(a) siRNAs had sequences which were cross reactive with the cynomolgus monkey LPA gene. Male cynomolgus monkeys (n=4 per treatment group/siRNA) were administered a single 3 mg/kg subcutaneous injection of Lp(a) siRNA constructs SRS-000008 through SRS-000022. Animals were fasted overnight prior to collection of blood samples on day-1 (pre-dose) and on, days 3, 7, 10, 14, 21, 28, 35, 42, 49, 56, 63, 70, 77, and 84. Lp(a) circulating protein levels in all serum samples were analyzed using an Lp(a) ELISA assay (Abcam, catalog #ab212165).

### Formulation Preparation

Preparation of siRNAs was conducted on the dosing day under aseptic conditions at room temperature. The siRNAs were dissolved with sodium chloride and swirled or stirred until fully dissolved. The solution was filtered with a 0.22 μM sterile filter and the solution was collected in a sterile container. The formulation was stored at 2-8° C. The siRNAs were dissolved by the appropriate amount of vehicle to get the concentration of 10 mg/mL as the dosing formulations, respectively. On the day of dosing, siRNA formulations were stored at 2-8° C. for dosing. The dosing procedure was finished within 6 hours.

### Animals

Cynomolgus monkeys were used in the study. Animals were housed in a group of 5 or less of the same dose level in stainless steel cages, and in an environmentally monitored, well-ventilated room (Conventional grade) maintained at a temperature of 18° C.-26° C. and a relative humidity of 40%-70%. Fluorescent lighting provided illumination approximately 12 hours per day.

### Experimental Design

To obtain groups that were comparable by body weight in the same sex, all monkeys were randomly assigned to respective treatment groups using a computer-generated randomization procedure. The body weights required for randomization were obtained on Day −5 (5 days prior to the first dosing). After randomization, monkeys were assigned to one of the treatment groups.

Each monkey in each group was given an siRNA formulation via subcutaneous injection with single or multipoint on skin of on the hindlimb and/or neck and back using a suitable disposable syringe and supporting needle. The hair around the injection site was shaved prior to dosing, and each injection point received no more than 2 mL. Doses were calculated based upon the most currently scheduled body weight.

Blood samples for lipoprotein A (Lp(a)) serum concentration detection were collected via forelimb or hindlimb subcutaneous vein. Monkeys were fasted overnight prior to blood collection for Lp(a) serum concentration evaluation.

Samples of whole blood were added into centrifuge tubes coated with separation gel and coagulator, and stored temporarily and transferred at room temperature. The blood samples were handled within 2 hours and centrifuged at 1500 g for 10 minutes at room temperature. After the centrifugation, the serum was aliquoted into 2 new tubes and transferred into newly labeled centrifuge tubes. All samples were stored equal to or below −20° C. and avoided repeated freeze-thaws. Watson LIMS 7.5 system was used to manage the samples. ELISA kits (Catalog number: ab212165; Manufacturer: Abcam) were used for Lp(a) serum concentration analysis.

### Data Analysis

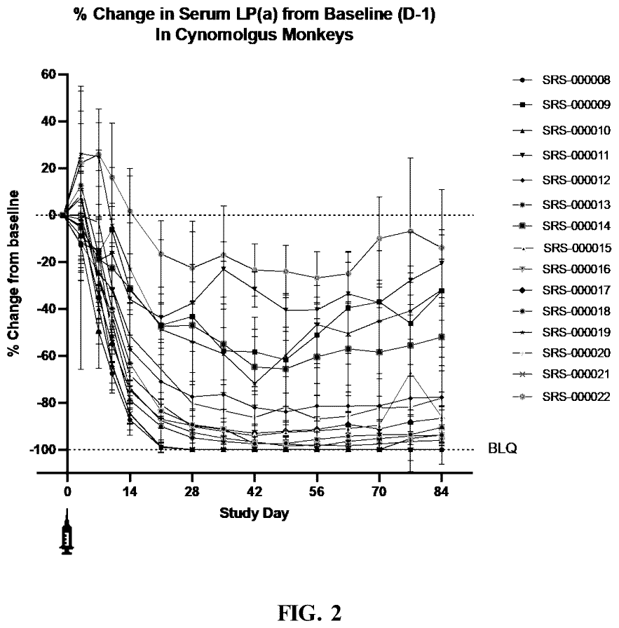

Lp(a) serum concentrations were determined from serum samples collected on day −1 (pre-dose) and days 3, 7, 10, 14, 21, 28, 35, 42, 49, 56, 63, 70, 77, and 84 using an ELISA assay (Abcam, catalog #ab212165). Results for each individual animal were expressed as the % change of circulating Lp(a) protein relative to the pre-dose (day-1) timepoint. The mean % change for each treatment group along with standard deviation was presented in Table 6 and plotted in FIG. 2.

### Example 3—Testing Lp(a) siRNAs in Non-Human Primates

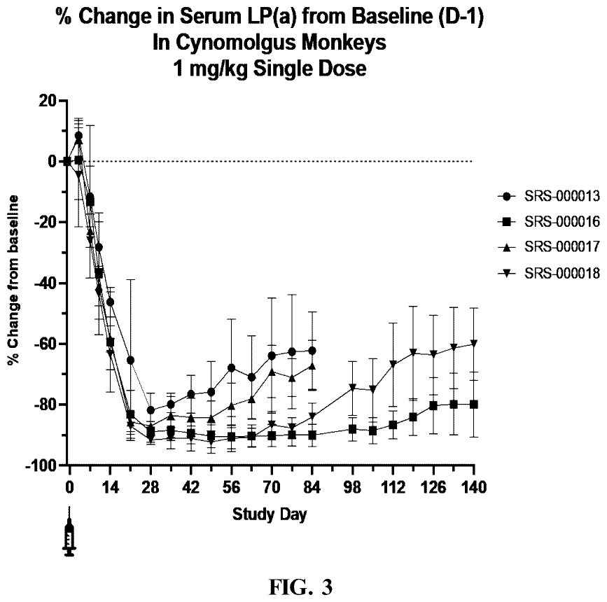

In a similar study described in example 2, four Lp(a) siRNAs, SRS-000013, SRS-000016, SRS-000017, and SRS-000018 were tested in cynomolgus monkeys. For each siRNA, the 3′ end of the passenger/sense strand was conjugated with a GalNAc via X2 (see Formula (V′)). The Lp(a) siRNAs had sequences which were cross reactive with the cynomolgus monkey LPA gene. Male cynomolgus monkeys (n=4 per treatment group/siRNA) were administered a single 1 mg/kg subcutaneous injection of each Lp(a) siRNA. Animals were fasted overnight prior to collection of blood samples on day −1 (pre-dose) and on days 3, 7, 10, 14, 21, 28, 35, 42, 49, 56, 63, 70, 77, and 85. Additional blood samples were collected on days 98, 105, 112, 119, 126, 133, and 140 for SRS-000016 and SRS-000018 treated groups. Lp(a) circulating protein levels in all serum samples were analyzed using an Lp(a) ELISA assay (Abcam, catalog #ab212165). Results for each individual animal were expressed as the % change of circulating Lp(a) protein relative to the pre-dose (day −1) timepoint. The mean % change for each treatment group along with standard deviation was presented in Table 7 and plotted in FIG. 3.

While preferred aspects of the present disclosure have been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby

TABLE 2

| | | | | Modified | | | | | Modified | | Position |
|---|---|---|---|---|---|---|---|---|---|---|---|
| | | Unmodified | SEQ | Antisense | SEQ | | Unmodified | SEQ | Sense | SEQ | at |
| Compd | siRNA | Antisense Sequence | ID | Sequence | ID | siRNA | Sense Sequence | ID | Sequence | ID | Human |
| ID | ID | (5′→3′) | NO. | (5′→3′) | NO. | ID | (5′→3′) | NO. | (5′→3′) | NO. | Transcript |
| SRS- | G- | UAGAUGACCAAGCUUGGCAG | 4 | [symbol] | 158 | P- | CCUGCCAAGCUUGGUCAUC | 312 | [symbol] | 466 | 204 |
| 000008 | 000008 | GUC | | | | 000008 | UA | | | | |
| SRS- | G- | UAUAGAUGACCAAGCUUGGC | 5 | [symbol] | 159 | P- | UGCCAAGCUUGGUCAUCUA | 313 | [symbol] | 467 | 206 |
| 000009 | 000009 | AGG | | | | 000009 | UA | | | | |
| SRS- | G- | UCAUAGAUGACCAAGCUUGG | 6 | [symbol] | 160 | P- | GCCAAGCUUGGUCAUCUAU | 314 | [symbol] | 468 | 207 |
| 000010 | 000010 | CAG | | | | 000010 | GA | | | | |
| SRS- | G- | UCGACGGCAGUCCCUUCUGC | 11 | [symbol] | 165 | P- | CGCAGAAGGGACUGCCGUC | 319 | [symbol] | 473 | 382 |
| 000011 | 000011 | GUC | | | | 000011 | GA | | | | |
| SRS- | G- | UUCUAGGCUUGGAACCGGGG | 18 | [symbol] | 172 | P- | ACCCCGGUUCCAAGCCUAG | 326 | [symbol] | 480 | 416 |
| 000012 | 000012 | UAA | | | | 000012 | AA | | | | |
| SRS- | G- | UAGCCUCUAGGCUUGGAACC | 21 | [symbol] | 175 | P- | CGGUUCCAAGCCUAGAGGC | 329 | [symbol] | 483 | 420 |
| 000013 | 000013 | GGG | | | | 000013 | UA | | | | |
| SRS- | G- | UUUACCGUGGUAGCACUCCU | 44 | [symbol] | 198 | P- | CAGGAGUGCUACCACGGUA | 352 | [symbol] | 506 | 1163 |
| 000014 | 000014 | GCA | | | | 000014 | AA | | | | |
| SRS- | G- | UUGUCCAUUACCGUGGUAGC | 47 | [symbol] | 201 | P- | UGCUACCACGGUAAUGGAC | 355 | [symbol] | 509 | 1169 |
| 000015 | 000015 | ACU | | | | 000015 | AA | | | | |
| SRS- | G- | UCUCUGUCCAUUACCGUGGU | 49 | [symbol] | 203 | P- | UACCACGGUAAUGGACAGA | 357 | [symbol] | 511 | 1172 |
| 000016 | 000016 | AGC | | | | 000016 | GA | | | | |
| SRS- | G- | UAUUGUGUCAGGUUGCAGUA | 60 | [symbol] | 214 | P- | GUACUGCAACCUGACACAA | 368 | [symbol] | 522 | 2749 |
| 000017 | 000017 | CUC | | | | 000017 | UA | | | | |
| SRS- | G- | UUGCGUCUGAGCAUUGUGUC | 64 | [symbol] | 218 | P- | UGACACAAUGCUCAGACGC | 372 | [symbol] | 526 | 2760 |
| 000018 | 000018 | AGG | | | | 000018 | AA | | | | |
| SRS- | G- | UUAACUCUGUCCAUAAUGGU | 88 | [symbol] | 242 | P- | UACCAUUAUGGACAGAGUU | 396 | [symbol] | 550 | 3227 |
| 000019 | 000019 | AGU | | | | 000019 | AA | | | | |
| SRS- | G- | UCCAAGCUUGGCAAGUUCUU | 89 | [symbol] | 243 | P- | GAAGAACUUGCCAAGCUUG | 397 | [symbol] | 551 | 3276 |
| 000020 | 000020 | CCU | | | | 000020 | GA | | | | |
| SRS- | G- | UAGAUGACCAAGCUUGGCAA | 90 | [symbol] | 244 | P- | CUUGCCAAGCUUGGUCAUC | 398 | [symbol] | 552 | 3282 |
| 000021 | 000021 | GUU | | | | 000021 | UA | | | | |
| SRS- | G- | UGGUCCGACUAUGCUGGUGU | 98 | [symbol] | 252 | P- | CACACCAGCAUAGUCGGAC | 406 | [symbol] | 560 | 3309 |
| 000022 | 000022 | GGU | | | | 000022 | CA | | | | |

As used herein, “A” refers to adenosine-3′-phosphate; “a” refers to 2′-O-methyladenosine-3′-phosphate, “Af” refers to 2′-fluoroadenosine-3′-phosphate; “C” refers to cytidine-3′-phosphate; “c” refers to 2′-O-methylcytidine-3′-phosphate; “Cf” refers to 2′-fluorocytidine-3′-phosphate, “G” refers to guanosine-3′-phosphate; “g” refers to 2′-O-methylguanosine-3′-phosphate; “Gf” refers to 2′-fluoroguanosine-3′-phosphate; “U” refers to uridine-3′-phosphate, “u” refers to 2′-O-methyluridine-3′-phosphate; “Uf” refers to 2′-fluorouridine-3′-phosphate, “T” refers to 5-methyluridine-3′-phosphate; “t” refers to 2′-O-methyl-5-methylunidine-3′-phosphate; “Tf” refers to 2′-fluoro-5-methyluridine-3′-phosphate; “s” refers to 3′-phosphorothioate. The “position at human transcript” is defined as the 5′ position of a 21-mer target site in human transcript (NCBI Reference Sequence No: NM_005577.4). Each modified sense sequence is coupled to a targeting moiety (e.g., GalNAc or galactose) at its 3′ end via a linker (e.g., Formula V′, V′′′′, V′′′′′, or V′′′′′′) [symbol]
indicates data missing or illegible when filed

TABLE 3
siRNA efficacy in primary human hepatocytes (PHH)

| | 10 μM | | 0.5 μM | | |
|---|---|---|---|---|---|
| | MV | | MV | | |
| | quadruplicates | | quadruplicates | | Position of |
| | [% residual | SD | [% residual | SD | 21mer Sense |
| Compd ID | target mRNA] | quadruplicates | target mRNA] | quadruplicates | Strand |
| Untreated | 118 | 8 | 94 | 10 | n/a |
| SRS-000023 | 98 | 10 | 87 | 3 | 198 |
| SRS-000024 | 85 | 11 | 79 | 4 | 199 |
| SRS-000025 | 100 | 2 | 83 | 6 | 202 |
| SRS-000026 | 73 | 6 | 63 | 5 | 204 |
| SRS-000027 | 82 | 14 | 85 | 1 | 206 |
| SRS-000028 | 66 | 11 | 68 | 4 | 207 |
| SRS-000029 | 78 | 6 | 70 | 3 | 260 |
| SRS-000030 | 97 | 9 | 83 | 6 | 374 |
| SRS-000031 | 107 | 10 | 91 | 6 | 379 |
| SRS-000032 | 100 | 9 | 80 | 10 | 381 |
| Untreated | 102 | 10 | 94 | 6 | n/a |
| Control | 96 | 15 | 86 | 6 | n/a |
| (Ahsa1) | | | | | |
| SRS-000033 | 82 | 13 | 79 | 6 | 382 |
| SRS-000034 | 96 | 17 | 79 | 3 | 383 |
| SRS-000035 | 94 | 12 | 76 | 3 | 406 |
| SRS-000036 | 92 | 6 | 92 | 11 | 407 |
| SRS-000037 | 94 | 5 | 81 | 7 | 408 |
| SRS-000038 | 93 | 2 | 82 | 6 | 411 |
| SRS-000039 | 87 | 3 | 74 | 4 | 412 |
| SRS-000040 | 73 | 11 | 75 | 1 | 416 |
| SRS-000041 | 91 | 9 | 86 | 5 | 417 |
| SRS-000042 | 93 | 8 | 85 | 2 | 419 |
| untreated | 96 | 3 | 90 | 5 | n/a |
| untreated | 100 | 9 | 102 | 2 | n/a |
| SRS-000043 | 69 | 5 | 80 | 3 | 420 |
| SRS-000044 | 81 | 6 | 89 | 3 | 421 |
| SRS-000045 | 82 | 11 | 87 | 6 | 425 |
| SRS-000046 | 86 | 9 | 97 | 7 | 427 |
| SRS-000047 | 83 | 11 | 91 | 10 | 430 |
| SRS-000048 | 95 | 14 | 99 | 6 | 435 |
| SRS-000049 | 90 | 3 | 80 | 27 | 436 |
| SRS-000050 | 70 | 31 | 97 | 4 | 438 |
| SRS-000051 | 88 | 9 | 94 | 10 | 439 |
| SRS-000052 | 85 | 5 | 94 | 8 | 442 |
| untreated | 96 | 8 | 96 | 6 | n/a |
| Control | 105 | 1 | 117 | 5 | n/a |
| (Ahsa1) | | | | | |
| SRS-000053 | 97 | 8 | 98 | 2 | 446 |
| SRS-000054 | 67 | 4 | 89 | 1 | 470 |
| SRS-000055 | 82 | 3 | 94 | 5 | 471 |
| SRS-000056 | 77 | 6 | 87 | 3 | 493 |
| SRS-000057 | 69 | 8 | 82 | 4 | 494 |
| SRS-000058 | 63 | 7 | 73 | 3 | 503 |
| SRS-000059 | 100 | 5 | 99 | 8 | 504 |
| SRS-000060 | 100 | 6 | 103 | 8 | 505 |
| SRS-000061 | 99 | 8 | 98 | 2 | 515 |
| SRS-000062 | 86 | 4 | 89 | 3 | 541 |
| untreated | 100 | 3 | 85 | 8 | n/a |
| untreated | 89 | 7 | 102 | 3 | n/a |
| SRS-000063 | 80 | 6 | 92 | 5 | 544 |
| SRS-000064 | 63 | 10 | 69 | 4 | 546 |
| SRS-000065 | 71 | 4 | 89 | 9 | 1162 |
| SRS-000066 | 66 | 4 | 83 | 3 | 1163 |
| SRS-000067 | 65 | 4 | 96 | 31 | 1165 |
| SRS-000068 | 87 | 5 | 93 | 5 | 1168 |
| SRS-000069 | 67 | 3 | 82 | 11 | 1169 |
| SRS-000070 | 92 | 6 | 91 | 5 | 1171 |
| SRS-000071 | 69 | 3 | 79 | 6 | 1172 |
| SRS-000072 | 86 | 3 | 89 | 5 | 1174 |
| untreated | 93 | 11 | 100 | 7 | n/a |
| Control | 94 | 7 | 118 | 14 | n/a |
| (Ahsa1) | | | | | |
| SRS-000073 | 66 | 1 | 76 | 5 | 1177 |
| SRS-000074 | 62 | 2 | 77 | 5 | 1178 |
| SRS-000075 | 76 | 2 | 89 | 6 | 2684 |
| SRS-000076 | 83 | 2 | 98 | 4 | 2698 |
| SRS-000077 | 87 | 5 | 102 | 5 | 2701 |
| SRS-000078 | 78 | 9 | 89 | 6 | 2705 |
| SRS-000079 | 81 | 2 | 92 | 34 | 2706 |
| SRS-000080 | 90 | 8 | 99 | 11 | 2707 |
| SRS-000081 | 90 | 3 | 91 | 9 | 2747 |
| SRS-000082 | 70 | 4 | 82 | 8 | 2749 |
| untreated | 95 | 2 | 91 | 7 | n/a |
| untreated | 101 | 7 | 109 | 11 | n/a |
| SRS-000083 | 89 | 4 | 95 | 10 | 2751 |
| SRS-000084 | 84 | 19 | 98 | 9 | 2753 |
| SRS-000085 | 85 | 6 | 102 | 8 | 2754 |
| SRS-000086 | 78 | 2 | 78 | 7 | 2760 |
| SRS-000089 | 81 | 7 | 90 | 7 | 2815 |
| SRS-000090 | 85 | 6 | 92 | 5 | 2823 |
| SRS-000091 | 83 | 6 | 88 | 5 | 2841 |
| SRS-000092 | 96 | 3 | 101 | 3 | 2843 |
| SRS-000093 | 80 | 7 | 76 | 4 | 2894 |
| SRS-000094 | 91 | 6 | 96 | 1 | 3023 |
| untreated | 101 | 7 | 96 | 4 | n/a |
| Control | 108 | 5 | 94 | 7 | n/a |
| (Ahsa1) | | | | | |
| SRS-000095 | 86 | 4 | 78 | 3 | 3044 |
| SRS-000096 | 92 | 8 | 86 | 4 | 3047 |
| SRS-000097 | 87 | 7 | 91 | 9 | 3049 |
| SRS-000098 | 92 | 7 | 86 | 5 | 3050 |
| SRS-000099 | 90 | 3 | 92 | 7 | 3090 |
| SRS-000100 | 92 | 4 | 93 | 5 | 3094 |
| SRS-000101 | 97 | 8 | 93 | 7 | 3095 |
| SRS-000102 | 92 | 5 | 93 | 4 | 3096 |
| SRS-000103 | 99 | 6 | 100 | 7 | 3098 |
| SRS-000104 | 97 | 7 | 106 | 3 | 3158 |
| untreated | 90 | 20 | 101 | 9 | n/a |
| untreated | 97 | 10 | 144 | 64 | n/a |
| SRS-000105 | 83 | 5 | 88 | 7 | 3218 |
| SRS-000106 | 79 | 5 | 84 | 15 | 3219 |
| SRS-000107 | 82 | 7 | 77 | 5 | 3220 |
| SRS-000108 | 82 | 8 | 78 | 6 | 3223 |
| SRS-000109 | 83 | 7 | 79 | 10 | 3226 |
| SRS-000110 | 66 | 7 | 73 | 18 | 3227 |
| SRS-000111 | 70 | 5 | 71 | 8 | 3276 |
| SRS-000112 | 56 | 7 | 56 | 13 | 3282 |
| SRS-000113 | 56 | 1 | 60 | 2 | 3284 |
| SRS-000114 | 48 | 3 | 57 | 3 | 3285 |
| untreated | 91 | 9 | 77 | 2 | n/a |
| Control | 108 | 5 | 95 | 1 | n/a |
| (Ahsa1) | | | | | |
| SRS-000115 | 97 | 6 | 82 | 6 | 3303 |
| SRS-000116 | 93 | 9 | 89 | 10 | 3304 |
| SRS-000117 | 89 | 7 | 83 | 9 | 3305 |
| SRS-000118 | 91 | 5 | 77 | 7 | 3307 |
| SRS-000119 | 89 | 4 | 75 | 4 | 3308 |
| SRS-000120 | 79 | 4 | 77 | 4 | 3309 |
| SRS-000121 | 95 | 5 | 79 | 6 | 3316 |
| SRS-000122 | 94 | 9 | 84 | 5 | 3317 |
| SRS-000123 | 91 | 10 | 78 | 2 | 3320 |
| SRS-000124 | 97 | 13 | 82 | 10 | 3396 |
| untreated | 104 | 13 | 83 | 6 | n/a |
| untreated | 99 | 6 | 99 | 3 | n/a |
| SRS-000125 | 96 | 7 | 91 | 5 | 3397 |
| SRS-000126 | 93 | 11 | 90 | 7 | 3398 |
| SRS-000127 | 91 | 12 | 87 | 4 | 3399 |
| SRS-000128 | 93 | 10 | 86 | 5 | 3406 |
| SRS-000129 | 91 | 7 | 90 | 2 | 3407 |
| SRS-000130 | 91 | 9 | 88 | 7 | 3464 |
| SRS-000131 | 88 | 12 | 92 | 6 | 3735 |
| SRS-000132 | 91 | 13 | 85 | 5 | 3737 |
| SRS-000133 | 77 | 2 | 84 | 5 | 3738 |
| SRS-000134 | 85 | 10 | 81 | 3 | 3741 |
| untreated | 97 | 7 | 89 | 5 | n/a |
| Control | 108 | 4 | 105 | 5 | n/a |
| (Ahsa1) | | | | | |
| SRS-000135 | 94 | 4 | 89 | 11 | 3778 |
| SRS-000136 | 90 | 2 | 94 | 9 | 3779 |
| SRS-000137 | 89 | 3 | 90 | 5 | 3780 |
| SRS-000138 | 88 | 5 | 86 | 5 | 3783 |
| SRS-000139 | 94 | 9 | 86 | 4 | 3790 |
| SRS-000140 | 80 | 6 | 82 | 6 | 3991 |
| SRS-000141 | 90 | 4 | 91 | 5 | 4049 |
| SRS-000142 | 92 | 2 | 83 | 9 | 4053 |
| SRS-000143 | 96 | 2 | 75 | 17 | 4055 |
| SRS-000144 | 94 | 11 | 85 | 12 | 4056 |
| untreated | 96 | 9 | 95 | 10 | n/a |
| untreated | 101 | 3 | 94 | 3 | n/a |
| SRS-000145 | 99 | 9 | 90 | 9 | 4066 |
| SRS-000146 | 91 | 12 | 78 | 5 | 4067 |
| SRS-000147 | 93 | 7 | 75 | 4 | 4138 |
| SRS-000148 | 91 | 14 | 80 | 3 | 4139 |
| SRS-000149 | 95 | 12 | 80 | 4 | 4150 |
| SRS-000150 | 93 | 14 | 70 | 5 | 4450 |
| SRS-000151 | 96 | 12 | 74 | 3 | 4645 |
| SRS-000152 | 97 | 9 | 73 | 4 | 4646 |
| SRS-000153 | 100 | 12 | 73 | 2 | 4733 |
| SRS-000154 | 104 | 10 | 70 | 2 | 4802 |
| untreated | 103 | 5 | 77 | 3 | n/a |
| Control | 97 | 6 | 110 | 17 | n/a |
| (Ahsa1) | | | | | |
| SRS-000155 | 81 | 15 | 83 | 8 | 4803 |
| SRS-000156 | 83 | 2 | 110 | 42 | 4805 |
| SRS-000157 | 84 | 5 | 82 | 10 | 4806 |
| SRS-000158 | 87 | 2 | 76 | 5 | 4808 |
| SRS-000159 | 83 | 3 | 75 | 6 | 4812 |
| SRS-000160 | 84 | 7 | 79 | 7 | 4823 |
| SRS-000161 | 89 | 3 | 75 | 10 | 4926 |
| SRS-000162 | 89 | 2 | 80 | 10 | 4969 |
| SRS-000163 | 90 | 4 | 84 | 14 | 4970 |
| SRS-000164 | 97 | 14 | 86 | 14 | 5073 |
| untreated | 99 | 8 | 96 | 12 | n/a |
| untreated | 121 | 18 | 102 | 7 | n/a |
| SRS-000165 | 106 | 5 | 98 | 2 | 5121 |
| SRS-000166 | 101 | 9 | 92 | 4 | 5122 |
| SRS-000167 | 96 | 9 | 92 | 6 | 5123 |
| SRS-000168 | 82 | 1 | 78 | 10 | 5124 |
| SRS-000169 | 104 | 7 | 91 | 7 | 5126 |
| SRS-000170 | 95 | 10 | 81 | 13 | 5127 |
| SRS-000171 | 100 | 10 | 92 | 8 | 5128 |
| SRS-000172 | 96 | 6 | 86 | 11 | 5129 |
| SRS-000173 | 92 | 8 | 79 | 5 | 5130 |
| SRS-000174 | 100 | 5 | 87 | 8 | 5132 |
| untreated | 101 | 5 | 84 | 10 | n/a |
| Control | 92 | 14 | 117 | 11 | n/a |
| (Ahsa1) | | | | | |
| SRS-000175 | 75 | 8 | 81 | 10 | 5505 |
| SRS-000176 | 76 | 6 | 92 | 7 | 5866 |
| SRS-000087 | 85 | 5 | 88 | 3 | 2762 |
| SRS-000088 | 81 | 3 | 90 | 3 | 2764 |
| untreated | 86 | 8 | 97 | 4 | n/a |

MV: meanvalue.
SD: standard deviation.
The “position of 21mer sense strand” is defined as the 5′ position of a 21-mer sense strand target site in human transcript (NCBI Reference Sequence No: NM_005577.4)

TABLE 4
Dose Response Curve in Primary Human Hepatocyte

| | Transcript Start | | | |
|---|---|---|---|---|
| | Site (5′ pos of | | MV quadruplicates | |
| | 21mer Sense | dose | [% residual | SD |
| Compd ID | Strand) | [μM] | target mRNA] | quadruplicates |
| SRS-000026 | 204 | Mock | 99 | 8 |
| | | 10.0 | 40 | 8 |
| | | 3.33 | 44 | 4 |
| | | 1.11 | 56 | 3 |
| | | 0.37 | 63 | 9 |
| | | 0.12 | 65 | 3 |
| | | 0.04 | 68 | 7 |
| | | 0.01 | 75 | 3 |
| | | 0.005 | 73 | 2 |
| | | 0.002 | 79 | 4 |
| | | 0.001 | 86 | 4 |
| Control (Ahsa1) | | 1.11 μM | 95 | 2 |
| | | Mock | 101 | 9 |
| SRS-000027 | 206 | 10.0 | 51 | 4 |
| | | 3.33 | 54 | 4 |
| | | 1.11 | 59 | 7 |
| | | 0.37 | 70 | 7 |
| | | 0.12 | 79 | 6 |
| | | 0.04 | 88 | 4 |
| | | 0.01 | 93 | 9 |
| | | 0.005 | 92 | 3 |
| | | 0.002 | 95 | 13 |
| | | 0.001 | 102 | 10 |
| | | Mock | 105 | 15 |
| | | Mock | 97 | 5 |
| SRS-000028 | 207 | 10.0 | 51 | 2 |
| | | 3.33 | 55 | 4 |
| | | 1.11 | 63 | 9 |
| | | 0.37 | 58 | 5 |
| | | 0.12 | 68 | 5 |
| | | 0.04 | 94 | 3 |
| | | 0.01 | 90 | 8 |
| | | 0.005 | 83 | 5 |
| | | 0.002 | 94 | 9 |
| | | 0.001 | 93 | 4 |
| Control (Ahsa1) | | 1.11 μM | 99 | 6 |
| | | Mock | 105 | 2 |
| SRS-000029 | 260 | 10.0 | 59 | 7 |
| | | 3.33 | 60 | 4 |
| | | 1.11 | 75 | 11 |
| | | 0.37 | 70 | 4 |
| | | 0.12 | 75 | 2 |
| | | 0.04 | 82 | 4 |
| | | 0.01 | 86 | 6 |
| | | 0.005 | 82 | 5 |
| | | 0.002 | 88 | 3 |
| | | 0.001 | 87 | 4 |
| | | Mock | 100 | 10 |
| | | Mock | 98 | 11 |
| SRS-000033 | 382 | 10.0 | 89 | 11 |
| | | 3.33 | 82 | 4 |
| | | 1.11 | 90 | 9 |
| | | 0.37 | 92 | 7 |
| | | 0.12 | 88 | 12 |
| | | 0.04 | 91 | 10 |
| | | 0.01 | 97 | 6 |
| | | 0.005 | 97 | 9 |
| | | 0.002 | 90 | 11 |
| | | 0.001 | 97 | 5 |
| Control (Ahsa1) | | 1.11 μM | 99 | 9 |
| | | Mock | 102 | 3 |
| SRS-000040 | 416 | 10.0 | 78 | 7 |
| | | 3.33 | 81 | 6 |
| | | 1.11 | 87 | 4 |
| | | 0.37 | 87 | 3 |
| | | 0.12 | 90 | 5 |
| | | 0.04 | 111 | 23 |
| | | 0.01 | 99 | 2 |
| | | 0.005 | 100 | 7 |
| | | 0.002 | 96 | 13 |
| | | 0.001 | 102 | 6 |
| | | Mock | 101 | 6 |
| | | Mock | 100 | 4 |
| SRS-000043 | 420 | 10.0 | 75 | 6 |
| | | 3.33 | 74 | 2 |
| | | 1.11 | 76 | 3 |
| | | 0.37 | 83 | 5 |
| | | 0.12 | 78 | 4 |
| | | 0.04 | 87 | 9 |
| | | 0.01 | 89 | 5 |
| | | 0.005 | 88 | 5 |
| | | 0.002 | 94 | 7 |
| | | 0.001 | 92 | 3 |
| Control (Ahsa1) | | 1.11 μM | 102 | 5 |
| | | Mock | 100 | 5 |
| SRS-000044 | 421 | 10.0 | 73 | 4 |
| | | 3.33 | 74 | 2 |
| | | 1.11 | 84 | 5 |
| | | 0.37 | 83 | 7 |
| | | 0.12 | 79 | 4 |
| | | 0.04 | 82 | 7 |
| | | 0.01 | 81 | 3 |
| | | 0.005 | 88 | 8 |
| | | 0.002 | 93 | 4 |
| | | 0.001 | 93 | 3 |
| | | Mock | 99 | 6 |
| | | Mock | 89 | 8 |
| SRS-000045 | 425 | 10.0 | 74 | 8 |
| | | 3.33 | 74 | 8 |
| | | 1.11 | 79 | 9 |
| | | 0.37 | 81 | 13 |
| | | 0.12 | 80 | 12 |
| | | 0.04 | 85 | 18 |
| | | 0.01 | 87 | 6 |
| | | 0.005 | 85 | 10 |
| | | 0.002 | 97 | 10 |
| | | 0.001 | 87 | 6 |
| Control (Ahsa1) | | 1.11 μM | 99 | 6 |
| | | Mock | 106 | 19 |
| SRS-000050 | 438 | 10.0 | 84 | 10 |
| | | 3.33 | 84 | 6 |
| | | 1.11 | 93 | 7 |
| | | 0.37 | 86 | 7 |
| | | 0.12 | 93 | 8 |
| | | 0.04 | 89 | 7 |
| | | 0.01 | 101 | 11 |
| | | 0.005 | 95 | 3 |
| | | 0.002 | 105 | 2 |
| | | 0.001 | 101 | 9 |
| | | Mock | 107 | 8 |
| | | Mock | 101 | 5 |
| SRS-000054 | 470 | 10.0 | 61 | 4 |
| | | 3.33 | 64 | 6 |
| | | 1.11 | 73 | 4 |
| | | 0.37 | 78 | 5 |
| | | 0.12 | 80 | 4 |
| | | 0.04 | 88 | 5 |
| | | 0.01 | 90 | 7 |
| | | 0.005 | 88 | 4 |
| | | 0.002 | 95 | 10 |
| | | 0.001 | 90 | 8 |
| Control (Ahsa1) | | 1.11 μM | 102 | 11 |
| | | Mock | 95 | 7 |
| SRS-000056 | 493 | 10.0 | 57 | 3 |
| | | 3.33 | 60 | 2 |
| | | 1.11 | 73 | 7 |
| | | 0.37 | 76 | 6 |
| | | 0.12 | 78 | 7 |
| | | 0.04 | 85 | 4 |
| | | 0.01 | 94 | 10 |
| | | 0.005 | 92 | 8 |
| | | 0.002 | 98 | 8 |
| | | 0.001 | 99 | 13 |
| | | Mock | 102 | 4 |
| | | Mock | 102 | 7 |
| SRS-000057 | 494 | 10.0 | 58 | 2 |
| | | 3.33 | 56 | 2 |
| | | 1.11 | 64 | 5 |
| | | 0.37 | 71 | 7 |
| | | 0.12 | 75 | 8 |
| | | 0.04 | 87 | 7 |
| | | 0.01 | 91 | 8 |
| | | 0.005 | 89 | 2 |
| | | 0.002 | 98 | 2 |
| | | 0.001 | 101 | 5 |
| Control (Ahsa1) | | 1.11 μM | 101 | 3 |
| | | Mock | 98 | 4 |
| SRS-000058 | 503 | 10.0 | 71 | 2 |
| | | 3.33 | 73 | 6 |
| | | 1.11 | 67 | 9 |
| | | 0.37 | 71 | 7 |
| | | 0.12 | 67 | 6 |
| | | 0.04 | 69 | 3 |
| | | 0.01 | 73 | 5 |
| | | 0.005 | 74 | 3 |
| | | 0.002 | 79 | 7 |
| | | 0.001 | 85 | 13 |
| | | Mock | 98 | 10 |
| | | Mock | 107 | 6 |
| SRS-000064 | 546 | 10.0 | 46 | 2 |
| | | 3.33 | 50 | 2 |
| | | 1.11 | 58 | 3 |
| | | 0.37 | 63 | 4 |
| | | 0.12 | 69 | 5 |
| | | 0.04 | 72 | 2 |
| | | 0.01 | 77 | 2 |
| | | 0.005 | 74 | 4 |
| | | 0.002 | 82 | 2 |
| | | 0.001 | 90 | 2 |
| Control (Ahsa1) | | 1.11 μM | 92 | 6 |
| | | Mock | 108 | 4 |
| SRS-000065 | 1162 | 10.0 | 99 | 14 |
| | | 3.33 | 76 | 2 |
| | | 1.11 | 104 | 4 |
| | | 0.37 | 100 | 4 |
| | | 0.12 | 104 | 8 |
| | | 0.04 | 97 | 8 |
| | | 0.01 | 96 | 10 |
| | | 0.005 | 99 | 8 |
| | | 0.002 | 103 | 8 |
| | | 0.001 | 102 | 6 |
| | | Mock | 93 | 6 |
| | | Mock | 101 | 10 |
| SRS-000066 | 1163 | 10.0 | 71 | 4 |
| | | 3.33 | 72 | 5 |
| | | 1.11 | 86 | 9 |
| | | 0.37 | 81 | 8 |
| | | 0.12 | 92 | 9 |
| | | 0.04 | 96 | 11 |
| | | 0.01 | 95 | 7 |
| | | 0.005 | 89 | 8 |
| | | 0.002 | 91 | 6 |
| | | 0.001 | 94 | 7 |
| Control (Ahsa1) | | 1.11 μM | 97 | 3 |
| SRS-000067 | 1165 | 10.0 | 81 | 5 |
| | | 3.33 | 79 | 8 |
| | | 1.11 | 95 | 5 |
| | | 0.37 | 88 | 8 |
| | | 0.12 | 97 | 6 |
| | | 0.04 | 93 | 4 |
| | | 0.01 | 102 | 9 |
| | | 0.005 | 92 | 7 |
| | | 0.002 | 103 | 5 |
| | | 0.001 | 101 | 7 |
| | | Mock | 98 | 4 |
| | | Mock | 106 | 1 |
| SRS-000069 | 1169 | 10.0 | 81 | 1 |
| | | 3.33 | 77 | 2 |
| | | 1.11 | 80 | 5 |
| | | 0.37 | 82 | 4 |
| | | 0.12 | 81 | 4 |
| | | 0.04 | 84 | 8 |
| | | 0.01 | 85 | 4 |
| | | 0.005 | 81 | 3 |
| | | 0.002 | 87 | 3 |
| | | 0.001 | 88 | 3 |
| Control (Ahsa1) | | 1.11 μM | 93 | 5 |
| | | Mock | 102 | 6 |
| SRS-000071 | 1172 | 10.0 | 87 | 9 |
| | | 3.33 | 85 | 3 |
| | | 1.11 | 92 | 9 |
| | | 0.37 | 87 | 4 |
| | | 0.12 | 92 | 5 |
| | | 0.04 | 93 | 6 |
| | | 0.01 | 85 | 4 |
| | | 0.005 | 93 | 5 |
| | | 0.002 | 87 | 7 |
| | | 0.001 | 93 | 4 |
| | | Mock | 98 | 5 |
| | | Mock | 99 | 12 |
| SRS-000073 | 1177 | 10.0 | 55 | 5 |
| | | 3.33 | 58 | 3 |
| | | 1.11 | 71 | 4 |
| | | 0.37 | 75 | 3 |
| | | 0.12 | 76 | 3 |
| | | 0.04 | 80 | 5 |
| | | 0.01 | 80 | 6 |
| | | 0.005 | 84 | 7 |
| | | 0.002 | 96 | 6 |
| | | 0.001 | 100 | 9 |
| Control (Ahsa1) | | 1.11 μM | 100 | 8 |
| | | Mock | 104 | 7 |
| SRS-000074 | 1178 | 10.0 | 49 | 2 |
| | | 3.33 | 55 | 3 |
| | | 1.11 | 62 | 8 |
| | | 0.37 | 70 | 2 |
| | | 0.12 | 74 | 2 |
| | | 0.04 | 81 | 2 |
| | | 0.01 | 88 | 9 |
| | | 0.005 | 88 | 5 |
| | | 0.002 | 98 | 9 |
| | | 0.001 | 102 | 3 |
| | | Mock | 97 | 3 |
| | | Mock | 96 | 8 |
| SRS-000075 | 2684 | 10.0 | 84 | 9 |
| | | 3.33 | 87 | 8 |
| | | 1.11 | 96 | 11 |
| | | 0.37 | 93 | 6 |
| | | 0.12 | 98 | 9 |
| | | 0.04 | 93 | 5 |
| | | 0.01 | 96 | 4 |
| | | 0.005 | 96 | 3 |
| | | 0.002 | 96 | 5 |
| | | 0.001 | 95 | 3 |
| Control (Ahsa1) | | 1.11 μM | 98 | 3 |
| | | Mock | 111 | 2 |
| SRS-000078 | 2705 | 10.0 | 88 | 1 |
| | | 3.33 | 89 | 8 |
| | | 1.11 | 96 | 4 |
| | | 0.37 | 92 | 4 |
| | | 0.12 | 107 | 11 |
| | | 0.04 | 107 | 16 |
| | | 0.01 | 86 | 4 |
| | | 0.005 | 91 | 11 |
| | | 0.002 | 94 | 3 |
| | | 0.001 | 96 | 3 |
| | | Mock | 95 | 8 |
| | | Mock | 88 | 6 |
| SRS-000079 | 2706 | 10.0 | 58 | 1 |
| | | 3.33 | 57 | 2 |
| | | 1.11 | 59 | 5 |
| | | 0.37 | 64 | 3 |
| | | 0.12 | 64 | 3 |
| | | 0.04 | 73 | 7 |
| | | 0.01 | 67 | 6 |
| | | 0.005 | 68 | 3 |
| | | 0.002 | 75 | 4 |
| | | 0.001 | 80 | 5 |
| Control (Ahsa1) | | 1.11 μM | 88 | 13 |
| | | Mock | 125 | 12 |
| SRS-000082 | 2749 | 10.0 | 77 | 13 |
| | | 3.33 | 71 | 11 |
| | | 1.11 | 80 | 13 |
| | | 0.37 | 77 | 14 |
| | | 0.12 | 77 | 10 |
| | | 0.04 | 80 | 9 |
| | | 0.01 | 87 | 14 |
| | | 0.005 | 80 | 10 |
| | | 0.002 | 85 | 14 |
| | | 0.001 | 94 | 8 |
| | | Mock | 100 | 14 |
| | | Mock | 90 | 21 |
| SRS-000086 | 2760 | 10.0 | 71 | 16 |
| | | 3.33 | 56 | 6 |
| | | 1.11 | 69 | 12 |
| | | 0.37 | 60 | 5 |
| | | 0.12 | 56 | 5 |
| | | 0.04 | 57 | 3 |
| | | 0.01 | 58 | 7 |
| | | 0.005 | 58 | 6 |
| | | 0.002 | 61 | 3 |
| | | 0.001 | 72 | 3 |
| Control (Ahsa1) | | 1.11 μM | 109 | 27 |
| | | Mock | 99 | 4 |
| SRS-000093 | 2894 | 10.0 | 61 | 4 |
| | | 3.33 | 57 | 5 |
| | | 1.11 | 60 | 9 |
| | | 0.37 | 67 | 2 |
| | | 0.12 | 75 | 2 |
| | | 0.04 | 73 | 5 |
| | | 0.01 | 75 | 9 |
| | | 0.005 | 77 | 3 |
| | | 0.002 | 84 | 4 |
| | | 0.001 | 81 | 9 |
| | | Mock | 103 | 10 |
| | | Mock | 98 | 4 |
| SRS-000106 | 3219 | 10.0 | 87 | 9 |
| | | 3.33 | 90 | 19 |
| | | 1.11 | 102 | 11 |
| | | 0.37 | 87 | 7 |
| | | 0.12 | 83 | 9 |
| | | 0.04 | 84 | 9 |
| | | 0.01 | 92 | 12 |
| | | 0.005 | 79 | 13 |
| | | 0.002 | 82 | 7 |
| | | 0.001 | 85 | 6 |
| Control (Ahsa1) | | 1.11 μM | 100 | 9 |
| | | Mock | 99 | 19 |
| SRS-000107 | 3220 | 10.0 | 85 | 5 |
| | | 3.33 | 97 | 5 |
| | | 1.11 | 100 | 9 |
| | | 0.37 | 94 | 4 |
| | | 0.12 | 93 | 7 |
| | | 0.04 | 91 | 5 |
| | | 0.01 | 94 | 8 |
| | | 0.005 | 88 | 11 |
| | | 0.002 | 95 | 12 |
| | | 0.001 | 96 | 13 |
| | | Mock | 103 | 6 |
| | | Mock | 88 | 15 |
| SRS-000108 | 3223 | 10.0 | 83 | 11 |
| | | 3.33 | 84 | 6 |
| | | 1.11 | 84 | 5 |
| | | 0.37 | 55 | 20 |
| | | 0.12 | 73 | 4 |
| | | 0.04 | 67 | 6 |
| | | 0.01 | 84 | 8 |
| | | 0.005 | 68 | 11 |
| | | 0.002 | 82 | 6 |
| | | 0.001 | 100 | 8 |
| Control (Ahsa1) | | 1.11 μM | 95 | 8 |
| | | Mock | 109 | 2 |
| SRS-000109 | 3226 | 10.0 | 94 | 7 |
| | | 3.33 | 86 | 17 |
| | | 1.11 | 83 | 14 |
| | | 0.37 | 92 | 16 |
| | | 0.12 | 80 | 9 |
| | | 0.04 | 80 | 11 |
| | | 0.01 | 81 | 15 |
| | | 0.005 | 74 | 17 |
| | | 0.002 | 84 | 6 |
| | | 0.001 | 91 | 6 |
| | | Mock | 108 | 9 |
| | | Mock | 92 | 8 |
| SRS-000110 | 3227 | 10.0 | 66 | 4 |
| | | 3.33 | 62 | 3 |
| | | 1.11 | 67 | 1 |
| | | 0.37 | 58 | 11 |
| | | 0.12 | 68 | 3 |
| | | 0.04 | 79 | 6 |
| | | 0.01 | 84 | 9 |
| | | 0.005 | 82 | 11 |
| | | 0.002 | 88 | 7 |
| | | 0.001 | 94 | 4 |
| Control (Ahsa1) | | 1.11 μM | 101 | 4 |
| | | Mock | 107 | 9 |
| SRS-000111 | 3276 | 10.0 | 83 | 3 |
| | | 3.33 | 82 | 2 |
| | | 1.11 | 90 | 6 |
| | | 0.37 | 85 | 2 |
| | | 0.12 | 79 | 3 |
| | | 0.04 | 82 | 3 |
| | | 0.01 | 87 | 5 |
| | | 0.005 | 84 | 7 |
| | | 0.002 | 93 | 8 |
| | | 0.001 | 96 | 4 |
| | | Mock | 100 | 8 |
| | | Mock | 92 | 4 |
| SRS-000112 | 3282 | 10.0 | 48 | 5 |
| | | 3.33 | 53 | 9 |
| | | 1.11 | 52 | 4 |
| | | 0.37 | 53 | 7 |
| | | 0.12 | 54 | 3 |
| | | 0.04 | 58 | 5 |
| | | 0.01 | 68 | 6 |
| | | 0.005 | 66 | 4 |
| | | 0.002 | 75 | 4 |
| | | 0.001 | 82 | 5 |
| Control (Ahsa1) | | 1.11 μM | 100 | 3 |
| | | Mock | 91 | 5 |
| SRS-000113 | 3284 | 10.0 | 55 | 6 |
| | | 3.33 | 63 | 4 |
| | | 1.11 | 84 | 7 |
| | | 0.37 | 79 | 1 |
| | | 0.12 | 76 | 3 |
| | | 0.04 | 81 | 2 |
| | | 0.01 | 87 | 4 |
| | | 0.005 | 85 | 6 |
| | | 0.002 | 88 | 7 |
| | | 0.001 | 93 | 4 |
| | | Mock | 95 | 5 |
| | | Mock | 93 | 6 |
| SRS-000114 | 3285 | 10.0 | 41 | 2 |
| | | 3.33 | 42 | 2 |
| | | 1.11 | 47 | 5 |
| | | 0.37 | 60 | 3 |
| | | 0.12 | 67 | 5 |
| | | 0.04 | 74 | 2 |
| | | 0.01 | 85 | 6 |
| | | 0.005 | 75 | 6 |
| | | 0.002 | 88 | 5 |
| | | 0.001 | 92 | 8 |
| Control (Ahsa1) | | 1.11 μM | 99 | 6 |
| | | Mock | 107 | 10 |
| SRS-000120 | 3309 | 10.0 | 90 | 7 |
| | | 3.33 | 87 | 7 |
| | | 1.11 | 88 | 7 |
| | | 0.37 | 93 | 6 |
| | | 0.12 | 87 | 9 |
| | | 0.04 | 94 | 5 |
| | | 0.01 | 97 | 7 |
| | | 0.005 | 86 | 2 |
| | | 0.002 | 96 | 10 |
| | | 0.001 | 93 | 5 |
| | | Mock | 77 | 52 |
| | | Mock | 94 | 8 |
| SRS-000133 | 3738 | 10.0 | 82 | 5 |
| | | 3.33 | 71 | 4 |
| | | 1.11 | 83 | 4 |
| | | 0.37 | 84 | 9 |
| | | 0.12 | 87 | 4 |
| | | 0.04 | 86 | 0 |
| | | 0.01 | 91 | 9 |
| | | 0.005 | 86 | 6 |
| | | 0.002 | 95 | 5 |
| | | 0.001 | 102 | 3 |
| Control (Ahsa1) | | 1.11 μM | 106 | 10 |
| | | Mock | 103 | 5 |
| SRS-000134 | 3741 | 10.0 | 97 | 2 |
| | | 3.33 | 95 | 6 |
| | | 1.11 | 103 | 8 |
| | | 0.37 | 98 | 7 |
| | | 0.12 | 102 | 5 |
| | | 0.04 | 100 | 8 |
| | | 0.01 | 105 | 11 |
| | | 0.005 | 103 | 12 |
| | | 0.002 | 104 | 17 |
| | | 0.001 | 111 | 6 |
| | | Mock | 97 | 14 |
| | | Mock | 103 | 4 |
| SRS-000135 | 3778 | 10.0 | 90 | 9 |
| | | 3.33 | 87 | 10 |
| | | 1.11 | 91 | 7 |
| | | 0.37 | 106 | 23 |
| | | 0.12 | 93 | 5 |
| | | 0.04 | 89 | 4 |
| | | 0.01 | 85 | 24 |
| | | 0.005 | 91 | 5 |
| | | 0.002 | 97 | 6 |
| | | 0.001 | 95 | 6 |
| Control (Ahsa1) | | 1.11 μM | 94 | 4 |
| | | Mock | 105 | 12 |
| SRS-000140 | 3991 | 10.0 | 81 | 8 |
| | | 3.33 | 77 | 3 |
| | | 1.11 | 87 | 11 |
| | | 0.37 | 85 | 8 |
| | | 0.12 | 87 | 5 |
| | | 0.04 | 90 | 4 |
| | | 0.01 | 105 | 14 |
| | | 0.005 | 93 | 1 |
| | | 0.002 | 100 | 5 |
| | | 0.001 | 99 | 4 |
| | | Mock | 98 | 3 |
| | | Mock | 114 | 17 |
| SRS-000155 | 4803 | 10.0 | 77 | 12 |
| | | 3.33 | 77 | 4 |
| | | 1.11 | 86 | 8 |
| | | 0.37 | 83 | 4 |
| | | 0.12 | 78 | 3 |
| | | 0.04 | 80 | 8 |
| | | 0.01 | 84 | 2 |
| | | 0.005 | 85 | 8 |
| | | 0.002 | 89 | 3 |
| | | 0.001 | 91 | 4 |
| Control (Ahsa1) | | 1.11 μM | 90 | 2 |
| | | Mock | 104 | 6 |
| SRS-000168 | 5124 | 10.0 | 65 | 3 |
| | | 3.33 | 63 | 4 |
| | | 1.11 | 82 | 17 |
| | | 0.37 | 74 | 5 |
| | | 0.12 | 72 | 8 |
| | | 0.04 | 74 | 8 |
| | | 0.01 | 86 | 8 |
| | | 0.005 | 83 | 3 |
| | | 0.002 | 88 | 9 |
| | | 0.001 | 90 | 4 |
| | | Mock | 92 | 8 |
| | | Mock | 97 | 4 |
| SRS-000175 | 5505 | 10.0 | 85 | 6 |
| | | 3.33 | 88 | 7 |
| | | 1.11 | 85 | 11 |
| | | 0.37 | 82 | 7 |
| | | 0.12 | 81 | 10 |
| | | 0.04 | 87 | 14 |
| | | 0.01 | 91 | 7 |
| | | 0.005 | 88 | 8 |
| | | 0.002 | 89 | 7 |
| | | 0.001 | 92 | 4 |
| Control (Ahsa1) | | 1.11 μM | 102 | 4 |
| | | Mock | 96 | 14 |
| SRS-000176 | 5866 | 10.0 | 96 | 4 |
| | | 3.33 | 95 | 5 |
| | | 1.11 | 101 | 8 |
| | | 0.37 | 93 | 18 |
| | | 0.12 | 96 | 4 |
| | | 0.04 | 96 | 5 |
| | | 0.01 | 103 | 4 |
| | | 0.005 | 99 | 3 |
| | | 0.002 | 100 | 3 |
| | | 0.001 | 101 | 6 |
| | | Mock | 106 | 9 |
| | | Mock | 92 | 7 |
| SRS-000087 | 2762 | 10.0 | 89 | 6 |
| | | 3.33 | 77 | 4 |
| | | 1.11 | 96 | 18 |
| | | 0.37 | 81 | 3 |
| | | 0.12 | 76 | 6 |
| | | 0.04 | 69 | 4 |
| | | 0.01 | 83 | 13 |
| | | 0.005 | 72 | 5 |
| | | 0.002 | 79 | 7 |
| | | 0.001 | 86 | 3 |
| Control (Ahsa1) | | 1.11 μM | 94 | 4 |
| | | Mock | 108 | 15 |
| SRS-000088 | 2764 | 10.0 | 94 | 8 |
| | | 3.33 | 86 | 13 |
| | | 1.11 | 99 | 8 |
| | | 0.37 | 95 | 7 |
| | | 0.12 | 95 | 17 |
| | | 0.04 | 99 | 14 |
| | | 0.01 | 105 | 7 |
| | | 0.005 | 97 | 12 |
| | | 0.002 | 99 | 15 |
| | | 0.001 | 97 | 10 |
| | | Control | 106 | 6 |

MV: mean value.
SD: standard deviation.
The “Transcript Start Site” is defined as the 5′ position of a 21-mer sense strand target site in human transcript (NCBI Reference Sequence No: NM_005577.4)

TABLE 5
Final dose responses in primary human hepatocyte

| | | | | | | | | | Transcript | | |
|---|---|---|---|---|---|---|---|---|---|---|---|
| | | | | | | | | | Start | MV max | |
| | | Modified | SEQ | | Modified | SEQ | | | Site | KD at | |
| | siRNA | Antisense Sequence | ID | siRNA | Sense Sequence | ID | IC50 | IC80 | in NM_ | highest | SD max |
| Compd ID | ID | (5′→3′) | NO. | ID | (5′→3′) | NO. | (μM) | (μM) | 005577.4 | dose | KD |
| SRS-000026 | G-000008 | [symbol] | 158 | P-000026 | [symbol] | 466 | 2.046722 | 127.9168 | 204 | 60.20508 | 7.820476 |
| SRS-000027 | G-000009 | [symbol] | 159 | P-000027 | [symbol] | 467 | 12.28532 | #N/A | 206 | 48.55853 | 3.722845 |
| SRS-000028 | G-000010 | [symbol] | 160 | P-000028 | [symbol] | 468 | 13.86981 | #N/A | 207 | 48.81948 | 2.262899 |
| SRS-000029 | G-000026 | [symbol] | 161 | P-000029 | [symbol] | 469 | 49.46417 | 4399.698 | 260 | 41.22481 | 7.184138 |
| SRS-000033 | G-000011 | [symbol] | 165 | P-000033 | [symbol] | 473 | #N/A | #N/A | 382 | 10.80257 | 10.65991 |
| SRS-000040 | G-000012 | [symbol] | 172 | P-000040 | [symbol] | 480 | #N/A | #N/A | 416 | 22.43361 | 6.593784 |
| SRS-000043 | G-000013 | [symbol] | 175 | P-000043 | [symbol] | 483 | #N/A | #N/A | 420 | 25.05174 | 6.339611 |
| SRS-000044 | G-000038 | [symbol] | 176 | P-000044 | [symbol] | 484 | #N/A | #N/A | 421 | 27.05507 | 3.55983 |
| SRS-000045 | G-000039 | [symbol] | 177 | P-000045 | [symbol] | 485 | #N/A | #N/A | 425 | 25.79898 | 7.67276 |
| SRS-000050 | G-000044 | [symbol] | 182 | P-000050 | [symbol] | 490 | #N/A | #N/A | 438 | 16.28757 | 9.523407 |
| SRS-000054 | G-000048 | [symbol] | 186 | P-000054 | [symbol] | 494 | 39.77935 | 1302.725 | 470 | 38.88559 | 3.700684 |
| SRS-000056 | G-000050 | [symbol] | 188 | P-000056 | [symbol] | 496 | 61.08738 | #N/A | 493 | 43.48794 | 2.714998 |
| SRS-000057 | G-000051 | [symbol] | 189 | P-000057 | [symbol] | 497 | #N/A | #N/A | 494 | 41.58673 | 1.838853 |
| SRS-000058 | G-000052 | [symbol] | 190 | P-000058 | [symbol] | 498 | #N/A | #N/A | 503 | 28.61084 | 2.377041 |
| SRS-000064 | G-000058 | [symbol] | 196 | P-000064 | [symbol] | 504 | 4.327232 | 961.917 | 546 | 54.44387 | 2.479078 |
| SRS-000065 | G-000059 | [symbol] | 197 | P-000065 | [symbol] | 505 | #N/A | #N/A | 1162 | 1.002411 | 13.93118 |
| SRS-000066 | G-000014 | [symbol] | 198 | P-000066 | [symbol] | 506 | 123.3882 | 1424.798 | 1163 | 29.36707 | 4.073732 |
| SRS-000067 | G-000060 | [symbol] | 199 | P-000067 | [symbol] | 507 | #N/A | #N/A | 1165 | 18.72208 | 5.49196 |
| SRS-000069 | G-000015 | [symbol] | 201 | P-000069 | [symbol] | 509 | #N/A | #N/A | 1169 | 19.26548 | 1.456436 |
| SRS-000071 | G-000016 | [symbol] | 203 | P-000071 | [symbol] | 511 | #N/A | #N/A | 1172 | 13.3161 | 8.604597 |
| SRS-000073 | G-000064 | [symbol] | 205 | P-000073 | [symbol] | 513 | 35.23821 | #N/A | 1177 | 45.02429 | 4.735237 |
| SRS-000074 | G-000065 | [symbol] | 206 | P-000074 | [symbol] | 514 | 9.343352 | #N/A | 1178 | 51.44863 | 1.590263 |
| SRS-000075 | G-000066 | [symbol] | 207 | P-000075 | [symbol] | 515 | 4491.018 | 56880.83 | 2684 | 16.44153 | 9.013653 |
| SRS-000078 | G-000069 | [symbol] | 210 | P-000078 | [symbol] | 518 | #N/A | #N/A | 2705 | 12.1747 | 1.347575 |
| SRS-000079 | G-000070 | [symbol] | 211 | P-000079 | [symbol] | 519 | #N/A | #N/A | 2706 | 41.82359 | 0.808165 |
| SRS-000082 | G-000017 | [symbol] | 214 | P-000082 | [symbol] | 522 | #N/A | #N/A | 2749 | 23.07176 | 13.12197 |
| SRS-000086 | G-000018 | [symbol] | 218 | P-000086 | [symbol] | 526 | #N/A | #N/A | 2760 | 29.35791 | 15.60843 |
| SRS-000087 | G-000076 | [symbol] | 219 | P-000087 | [symbol] | 527 | #N/A | #N/A | 2762 | 10.5034 | 6.168005 |
| SRS-000088 | G-000077 | [symbol] | 220 | P-000088 | [symbol] | 528 | #N/A | #N/A | 2764 | 5.712127 | 7.947043 |
| SRS-000093 | G-000082 | [symbol] | 225 | P-000093 | [symbol] | 533 | 163.9122 | #N/A | 2894 | 39.04343 | 3.861572 |
| SRS-000106 | G-000095 | [symbol] | 238 | P-000106 | [symbol] | 546 | #N/A | #N/A | 3219 | 13.31607 | 9.205419 |
| SRS-000107 | G-000096 | [symbol] | 239 | P-000107 | [symbol] | 547 | #N/A | #N/A | 3220 | 14.80472 | 4.612617 |
| SRS-000108 | G-000097 | [symbol] | 240 | P-000108 | [symbol] | 548 | #N/A | #N/A | 3223 | 16.80029 | 11.10403 |
| SRS-000109 | G-000098 | [symbol] | 241 | P-000109 | [symbol] | 549 | #N/A | #N/A | 3226 | 6.153194 | 7.498487 |
| SRS-000110 | G-000019 | [symbol] | 242 | P-000110 | [symbol] | 550 | #N/A | #N/A | 3227 | 33.61694 | 3.644445 |
| SRS-000111 | G-000020 | [symbol] | 243 | P-000111 | [symbol] | 551 | #N/A | #N/A | 3276 | 17.35972 | 2.580181 |
| SRS-000112 | G-000021 | [symbol] | 244 | P-000112 | [symbol] | 552 | 8.297705 | #N/A | 3282 | 51.59325 | 4.521415 |
| SRS-000113 | G-000099 | [symbol] | 245 | P-000113 | [symbol] | 553 | 33.84077 | 659.9335 | 3284 | 45.41711 | 6.175907 |
| SRS-000114 | G-000100 | [symbol] | 246 | P-000114 | [symbol] | 554 | 1.349305 | 11004.53 | 3285 | 58.87523 | 2.300955 |
| SRS-000120 | G-000022 | [symbol] | 252 | P-000120 | [symbol] | 560 | #N/A | #N/A | 3309 | 10.24371 | 7.209576 |
| SRS-000133 | G-000118 | [symbol] | 265 | P-000133 | [symbol] | 573 | #N/A | #N/A | 3738 | 17.57087 | 5.286225 |
| SRS-000134 | G-000119 | [symbol] | 266 | P-000134 | [symbol] | 574 | #N/A | #N/A | 3741 | 2.983854 | 2.420011 |
| SRS-000135 | G-000120 | [symbol] | 267 | P-000135 | [symbol] | 575 | #N/A | #N/A | 3778 | 9.608682 | 8.804995 |
| SRS-000140 | G-000125 | [symbol] | 272 | P-000140 | [symbol] | 580 | #N/A | #N/A | 3991 | 19.11657 | 7.678151 |
| SRS-000155 | G-000140 | [symbol] | 287 | P-000155 | [symbol] | 595 | #N/A | #N/A | 4803 | 23.19757 | 11.52321 |
| SRS-000168 | G-000153 | [symbol] | 300 | P-000168 | [symbol] | 608 | #N/A | #N/A | 5124 | 34.5264 | 2.589133 |
| SRS-000175 | G-000160 | [symbol] | 307 | P-000175 | [symbol] | 615 | #N/A | #N/A | 5505 | 15.05201 | 6.295908 |
| SRS-000176 | G-000161 | [symbol] | 308 | P-000176 | [symbol] | 616 | #N/A | #N/A | 5866 | 3.802795 | 3.789188 |

As used herein, “A” refers to adenosine-3′-phosphate; “a” refers to 2′-O-methyladenosine-3′-phosphate; “Af” refers to 2′-fluoroadenosine-3′-phosphate; “C” refers to cytidine-3′-phosphate; “c” refers to 2′-O-methylcytidine-3′-phosphate; ″Cf′ refers to 2′ -fluorocytidine-3′-phosphate, “G” refers to guanosine-3′-phosphate; “g” refers to 2′-O-methylguanosine-3′-phosphate; “Gf” refers to 2′- fluoroguanosine-3′-phosphate; “U” refers to uridine-3′-phosphate; “u” refers to 2′-O-methyluridine-3′-phosphate; “Uf” refers to 2′-fluorouridine-3′-phosphate; “T” refers to 5-methyluridine-3′-phosphate; “1” refers to 2′-O-methyl-5-methyluridine-3′-phosphate; “Tf” refers to 2′-fluoro-5-methyluridine-3′-phosphate; “s” refers to 3′-phosphorothicate. The “Transcript Start Site in NM_005577.4” is defined as the 5′ position of a 21-mer target site in human transcript (NCBI Reference Sequence No: NM_0055774).
Note that the sense strand sequenceis coupled to a targeting moiety (e.g., GalNAc or galactose) at the 3′ end of the sense strand. MV: mean value. SD: standard deviation. [symbol]
indicates data missing or illegible when filed

TABLE 6
% Change in Cynomolgus Monkey Circulating LP(a) Serum Concentration Following Single 3 mg/kg Dose

| | | | | SRS ID | | | | | | | |
|---|---|---|---|---|---|---|---|---|---|---|---|
| | | | | SRS- | SRS- | SRS- | SRS- | SRS- | SRS- | SRS- | SRS- |
| | | | | 000008 | 000009 | 000010 | 000011 | 000012 | 000013 | 000014 | 000015 |
| | | | | Position of 21mer Sense Strand | | | | | | | |
| | | | | 204 | 206 | 207 | 382 | 416 | 420 | 1163 | 1169 |
| % Change | Study | −1 | Avg | 0.0 | 0.0 | 0.0 | 0.0 | 0.0 | 0.0 | 0.0 | 0.0 |
| in Cyno | Day | | StDev | 0.0 | 0.0 | 0.0 | 0.0 | 0.0 | 0.0 | 0.0 | 0.0 |
| Serum | | 3 | Avg | −12.9 | −8.9 | −1.7 | −4.5 | −11.9 | −11.9 | −5.3 | 8.8 |
| Lipoprotein | | | StDev | 10.8 | 8.6 | 20.6 | 15.0 | 16.0 | 12.6 | 60.4 | 9.8 |
| A | | 7 | Avg | −49.7 | −15.7 | −40.0 | −19.2 | −24.8 | −15.2 | −18.7 | −26.6 |
| concentration | | | StDev | 5.3 | 7.9 | 10.3 | 18.0 | 10.7 | 14.7 | 46.4 | 12.5 |
| from | | 10 | Avg | −67.5 | −6.3 | −63.9 | −16.4 | −31.6 | −39.9 | −22.5 | −43.0 |
| Baseline | | | StDev | 8.4 | 10.1 | 10.7 | 14.6 | 8.5 | 8.8 | 43.0 | 8.3 |
| (Day −1) | | 14 | Avg | −87.3 | −31.8 | −84.6 | −35.8 | −57.2 | −63.2 | −31.2 | −68.1 |
| | | | StDev | 4.4 | 5.3 | 4.5 | 4.4 | 6.1 | 7.1 | 48.1 | 4.8 |
| | | 21 | Avg | −98.9 | −46.8 | −98.7 | −43.8 | −71.0 | −83.6 | −47.5 | −80.7 |
| | | | StDev | 2.2 | 9.9 | 2.7 | 10.3 | 7.3 | 3.1 | 37.1 | 5.1 |
| | | 28 | Avg | −100.0 | −43.2 | −100.0 | −37.5 | −77.4 | −89.4 | −47.0 | −90.2 |
| | | | StDev | 0.0 | 14.8 | 0.0 | 16.5 | 5.2 | 3.5 | 44.4 | 7.0 |
| | | 35 | Avg | −100.0 | −57.9 | −100.0 | −23.0 | −76.5 | −91.2 | −55.0 | −92.2 |
| | | | StDev | 0.0 | 17.9 | 0.0 | 11.8 | 6.4 | 3.1 | 35.3 | 5.6 |
| | | 42 | Avg | −100.0 | −58.4 | −100.0 | −31.6 | −82.1 | −97.5 | −64.8 | −94.1 |
| | | | StDey | 0.0 | 14.9 | 0.0 | 7.6 | 7.2 | 3.0 | 16.2 | 4.2 |
| | | 49 | Avg | −100.0 | −61.7 | −100.0 | −40.5 | −83.9 | −97.3 | −65.5 | −92.6 |
| | | | StDev | 0.0 | 27.7 | 0.0 | 7.2 | 8.0 | 3.2 | 15.9 | 5.1 |
| | | 56 | Avg | −100.0 | −51.1 | −100.0 | −40.4 | −81.4 | −95.7 | −60.4 | −91.9 |
| | | | StDev | 0.0 | 13.9 | 0.0 | 5.5 | 9.7 | 3.2 | 16.3 | 5.8 |
| | | 63 | Avg | −100.0 | −39.5 | −100.0 | −33.5 | −81.4 | −94.2 | −57.0 | −91.0 |
| | | | StDev | 0.0 | 19.4 | 0.0 | 18.1 | 8.9 | 4.3 | 20.0 | 6.7 |
| | | 70 | Avg | −100.0 | −37.0 | −100.0 | −37.3 | −81.3 | −93.7 | −58.4 | −89.6 |
| | | | StDev | 0.0 | 21.8 | 0.0 | 8.1 | 11.6 | 5.9 | 18.9 | 7.8 |
| | | 77 | Avg | −100.0 | −46.1 | −100.0 | −27.7 | −78.0 | −93.6 | −55.6 | −67.0 |
| | | | StDev | 0.0 | 38.8 | 0.0 | 6.2 | 14.3 | 4.5 | 13.0 | 42.5 |
| | | 84 | Avg | −100.0 | −32.3 | −100.0 | −20.7 | −77.6 | −90.5 | −52.0 | −86.1 |
| | | | StDev | 0.0 | 24.0 | 0.0 | 14.5 | 13.4 | 7.6 | 14.4 | 10.8 |

| | | | | SRS ID | | | | | | |
|---|---|---|---|---|---|---|---|---|---|---|
| | | | | SRS- | SRS- | SRS- | SRS- | SRS- | SRS- | SRS- |
| | | | | 000016 | 000017 | 000018 | 000019 | 000020 | 000021 | 000022 |
| | | | | Position of 21mer Sense Strand | | | | | | |
| | | | | 1172 | 2749 | 2760 | 3227 | 3276 | 3282 | 3309 |
| % Change | Study | −1 | Avg | 0.0 | 0.0 | 0.0 | 0.0 | 0.0 | 0.0 | 0.0 |
| in Cyno | Day | | StDev | 0.0 | 0.0 | 0.0 | 0.0 | 0.0 | 0.0 | 0.0 |
| Serum | | 3 | Avg | 0.2 | −4.9 | 12.8 | 26.2 | −0.1 | 7.3 | 22.3 |
| Lipoprotein | | | StDev | 20.8 | 22.8 | 31.6 | 26.6 | 11.6 | 17.1 | 16.8 |
| A | | 7 | Avg | −27.7 | −35.1 | −17.0 | 25.0 | −3.1 | −30.7 | 25.9 |
| concentration | | | StDev | 16.3 | 15.5 | 15.2 | 20.3 | 22.1 | 15.2 | 13.6 |
| from | | 10 | Avg | −45.9 | −54.9 | −51.9 | −4.1 | −30.8 | −63.6 | 16.1 |
| Baseline | | | StDev | 11.4 | 7.4 | 10.8 | 9.2 | 19.1 | 7.9 | 23.2 |
| (Day −1) | | 14 | Avg | −73.8 | −74.6 | −78.9 | −22.7 | −51.3 | −84.7 | 1.8 |
| | | | StDev | 3.1 | 7.6 | 15.0 | 19.5 | 13.7 | 4.1 | 18.2 |
| | | 21 | Avg | −87.6 | −87.1 | −90.1 | −48.7 | −65.7 | −99.0 | −16.6 |
| | | | StDev | 1.2 | 3.8 | 7.2 | 10.8 | 12.8 | 2.0 | 14.2 |
| | | 28 | Avg | −92.6 | −89.7 | −94.9 | −53.9 | −80.3 | −100.0 | −22.5 |
| | | | StDev | 2.4 | 3.9 | 4.3 | 17.0 | 13.3 | 0.0 | 15.4 |
| | | 35 | Avg | −95.2 | −91.4 | −96.5 | −59.1 | −83.4 | −100.0 | −17.0 |
| | | | StDev | 1.3 | 3.1 | 3.1 | 11.1 | 11.2 | 0.0 | 20.9 |
| | | 42 | Avg | −96.7 | −93.0 | −97.5 | −71.8 | −86.3 | −100.0 | −23.4 |
| | | | StDey | 2.2 | 3.9 | 3.1 | 20.0 | 9.7 | 0.0 | 11.2 |
| | | 49 | Avg | −98.5 | −92.0 | −97.4 | −59.7 | −81.8 | −100.0 | −24.0 |
| | | | StDev | 2.0 | 3.8 | 3.2 | 6.2 | 12.8 | 0.0 | 11.1 |
| | | 56 | Avg | −98.1 | −91.3 | −98.5 | −46.6 | −86.9 | −100.0 | −26.7 |
| | | | StDev | 2.3 | 3.7 | 1.9 | 16.7 | 9.9 | 0.0 | 11.1 |
| | | 63 | Avg | −98.3 | −89.2 | −96.5 | −50.5 | −85.7 | −100.0 | −25.0 |
| | | | StDev | 2.3 | 5.4 | 3.5 | 12.9 | 10.3 | 0.0 | 9.1 |
| | | 70 | Avg | −97.6 | −91.2 | −95.3 | −45.1 | −82.3 | −100.0 | −9.9 |
| | | | StDev | 1.7 | 4.0 | 4.2 | 14.4 | 14.8 | 0.0 | 17.8 |
| | | 77 | Avg | −96.5 | −88.2 | −94.2 | −40.8 | −81.9 | −95.3 | −6.8 |
| | | | StDev | 2.6 | 5.0 | 5.3 | 12.1 | 13.9 | 9.4 | 31.3 |
| | | 84 | Avg | −96.0 | −86.6 | −93.8 | −31.8 | −78.0 | −93.7 | −13.9 |
| | | | StDev | 1.2 | 5.0 | 4.6 | 13.1 | 17.6 | 12.5 | 24.9 |

TABLE 7
% Change in Cynomolgus Monkey Circulating LP(a)
Serum Concentration Following Single 1 mg/kg Dose
% Change in Cyno Serum Lipoprotein A Concentration
from Baseline (Day −1)

| | SRS ID | | | | | | | |
|---|---|---|---|---|---|---|---|---|
| | SRS- | | SRS- | | SRS- | | SRS- | |
| | 000013 | | 000016 | | 000017 | | 000018 | |
| | Position of 21mer Sense Strand | | | | | | | |
| | 420 | | 1172 | | 2749 | | 2760 | |
| Study Day | Avg | StDev | Avg | StDev | Avg | StDev | Avg | StDev |
| −1 | 0.0 | 0.0 | 0.0 | 0.0 | 0.0 | 0.0 | 0.0 | 0.0 |
| 3 | 8.5 | 2.7 | 0.5 | 13.0 | 7.6 | 6.7 | −4.5 | 16.9 |
| 7 | −11.5 | 9.9 | −13.2 | 25.0 | −22.8 | 5.4 | −26.1 | 12.2 |
| 10 | −28.2 | 8.3 | −37.0 | 20.1 | −41.0 | 6.4 | −43.5 | 8.4 |
| 14 | −46.2 | 4.9 | −59.4 | 16.4 | −58.3 | 4.3 | −63.4 | 5.2 |
| 21 | −65.3 | 26.5 | −83.1 | 7.9 | −85.6 | 3.2 | −87.0 | 2.8 |
| 28 | −81.8 | 5.7 | −88.9 | 3.4 | −87.0 | 5.2 | −91.6 | 1.6 |
| 35 | −79.9 | 2.6 | −88.4 | 3.5 | −83.5 | 7.3 | −91.1 | 3.5 |
| 42 | −76.6 | 6.3 | −89.3 | 3.6 | −84.1 | 6.3 | −91.1 | 4.1 |
| 49 | −75.8 | 10.0 | −90.3 | 3.7 | −84.3 | 5.5 | −92.3 | 3.6 |
| 56 | −67.9 | 15.9 | −90.6 | 3.9 | −80.2 | 7.4 | −91.0 | 4.4 |
| 63 | −70.9 | 13.6 | −90.3 | 3.7 | −78.0 | 6.8 | −90.7 | 3.1 |
| 70 | −63.9 | 18.9 | −90.2 | 3.7 | −69.1 | 8.6 | −86.6 | 4.5 |
| 77 | −62.6 | 18.8 | −89.9 | 3.6 | −71.1 | 6.2 | −87.7 | 3.3 |
| 85 | −62.2 | 12.7 | −90.0 | 3.6 | −67.1 | 8.2 | −84.0 | 4.6 |
| 98 | NA | NA | −88.0 | 3.9 | NA | NA | −74.6 | 8.9 |
| 105 | NA | NA | −88.5 | 4.4 | NA | NA | −75.2 | 10.5 |
| 112 | NA | NA | −86.6 | 4.7 | NA | NA | −66.9 | 13.8 |
| 119 | NA | NA | −84.1 | 6.1 | NA | NA | −63.1 | 15.5 |
| 126 | NA | NA | −80.3 | 9.2 | NA | NA | −63.6 | 13.1 |
| 133 | NA | NA | −79.8 | 10.2 | NA | NA | −61.3 | 13.4 |
| 140 | NA | NA | −79.9 | 10.7 | NA | NA | −60.1 | 11.8 |

What is claimed is:

1. A polynucleic acid molecule for modulating expression of lipoprotein(a) (Lp(a)) gene, comprising a sense strand and an antisense strand, wherein the antisense strand comprises a nucleic acid sequence that is at least 80%, at least 85%, at least 90%, at least 95%, at least 96%, at least 97%, at least 98%, at least 99% identical to a nucleic acid sequence selected from SEQ ID NOs: 1-154.

2. The polynucleic acid of claim 1, wherein the sense strand comprises comprises a nucleic acid sequence that is at least 80%, at least 85%, at least 90%, at least 95%, at least 96%, at least 97%, at least 98%, at least 99% identical to a nucleic acid sequence selected from SEQ ID NOs: 309-462.

3. The polynucleic acid molecule of any one of claims 1-2, wherein the antisense strand comprises a nucleic acid sequence comprising at least 14, 15, 16, 17, 18, 19, 20, 21, or 22 consecutive sequences of a nucleic acid sequence selected from SEQ ID NOs: 1-154, with no more than 1, 2, 3, or 4 mismatches.

4. The polynucleic acid molecule of any one of claims 1-3, wherein the sense strand comprises a nucleic acid sequence comprising at least 14, 15, 16, 17, 18, 19, or 20 consecutive sequences from a nucleic acid sequence selected from SEQ ID NOs: 309-462, with no more than 1, 2, 3, or 4 mismatches.

5. The polynucleic acid molecule of any one of claims 1-4, wherein the sense strand comprises one of SEQ ID NOs: 309-462 and the antisense strand comprises a nucleic acid sequence that is selected from SEQ ID NOs: 1-154.

6. The polynucleic acid molecule of any one of claims 1-5, wherein the sense strand comprises at least two consecutive modified internucleotide linkages at the 5′ end.

7. The polynucleic acid molecule of any one of claims 1-6, wherein the antisense strand comprises at least two consecutive modified internucleotide linkages at the 5′ end and/or 3′ end.

8. The polynucleic acid molecule of any one of claims 6-7, wherein the modified internucleotide linkage is a phosphorothioate linkage.

9. The polynucleic acid molecule of claim 8, wherein the modified internucleotide linkage comprises a stereochemically enriched phosphorothioate internucleotide linkage.

10. The polynucleic acid molecule of any one of claims 8-9, wherein the modified internucleotide linkage is an S_{P} chiral internucleotide phosphorothioate linkage.

11. The polynucleic acid molecule of claim 10, wherein the polynucleic acid comprises a plurality of modified internucleotide linkages, and at least 1, 2, 3, or 4 of the plurality of modified internucleotide linkages are stereochemically enriched phosphorothioate internucleotide linkages.

12. The polynucleic acid molecule of any one of claims 9-11, wherein at least one stereochemically enriched phosphorothioate is disposed between two consecutive nucleosides that are two of six 5′ or 3′-terminal nucleosides of the sense strand or the antisense strand.

13. The polynucleic acid molecule of any one of claims 1-12, wherein the sense strand comprises 5′-nnnnnnNfnNfnNfnnnnnnnnnn-3′, wherein the antisense strand comprises 5′-nNfnnnnNfnnnnNfnNfnNfnnnnnnn-3′, wherein n stands for a 2′-O-methyl modified nucleotide, and wherein Nf stands for a 2′-fluoro modified nucleotide.

14. The polynucleic acid molecule of any one of claims 1-13, wherein the sense strand or antisense strand is about 19-25, or about 21-23 nucleotides in length.

15. The polynucleic acid molecule of any one of preceding claims, wherein the sense strand comprises a nucleic acid sequence that is at least 80%, at least 85%, at least 90%, at least 95% identical to a nucleic acid sequence selected from SEQ ID NOs: 463-616.

16. The polynucleic acid molecule of any one of preceding claims, wherein the antisense strand comprises a nucleic acid sequence that is at least 80%, at least 85%, at least 90%, at least 95% identical to a nucleic acid sequence selected from SEQ ID NOs: 155-308.

17. The polynucleic acid molecule of any one of preceding claims, wherein the sense strand comprises a sequence selected from a nucleic acid sequence of SEQ ID NOs: 463-616, and the antisense strand comprises a sequence selected from a nucleic acid sequence of SEQ ID NOs: 155-308.

18. A polynucleic acid molecule for modulating expression of Lipoprotein(a) (Lp(a)) gene, wherein polynucleic acid molecule comprises:
(a) an antisense strand comprising the nucleotide sequence of UAGAUGACCAAGCUUGGCAGGUC (SEQ ID NO: 4) and a sense strand comprising the nucleotide sequence of CCUGCCAAGCUUGGUCAUCUA (SEQ ID NO: 312);
(b) an antisense strand comprising the nucleotide sequence of UAUAGAUGACCAAGCUUGGCAGG (SEQ ID NO: 5) and a sense strand comprising the nucleotide sequence of UGCCAAGCUUGGUCAUCUAUA (SEQ ID NO:313);
(c) an antisense strand comprising the nucleotide sequence of UCAUAGAUGACCAAGCUUGGCAG (SEQ ID NO: 6) and a sense strand comprising the nucleotide sequence of GCCAAGCUUGGUCAUCUAUGA (SEQ ID NO: 314);
(d) an antisense strand comprising the nucleotide sequence of UCGACGGCAGUCCCUUCUGCGUC (SEQ ID NO: 11) and a sense strand comprising the nucleotide sequence of CGCAGAAGGGACUGCCGUCGA (SEQ ID NO: 319);
(e) an antisense strand comprising the nucleotide sequence of UUCUAGGCUUGGAACCGGGGUAA (SEQ ID NO: 18) and a sense strand comprising the nucleotide sequence of ACCCCGGUUCCAAGCCUAGAA (SEQ ID NO: 326);
(f) an antisense strand comprising the nucleotide sequence of UAGCCUCUAGGCUUGGAACCGGG (SEQ ID NO: 21) and a sense strand comprising the nucleotide sequence of CGGUUCCAAGCCUAGAGGCUA (SEQ ID NO: 329);
(g) an antisense strand comprising the nucleotide sequence of UUUACCGUGGUAGCACUCCUGCA (SEQ ID NO: 44) and a sense strand comprising the nucleotide sequence of CAGGAGUGCUACCACGGUAAA (SEQ ID NO: 352);
(h) an antisense strand comprising the nucleotide sequence of UUGUCCAUUACCGUGGUAGCACU (SEQ ID NO: 47) and a sense strand comprising the nucleotide sequence of UGCUACCACGGUAAUGGACAA (SEQ ID NO: 355);
(i) an antisense strand comprising the nucleotide sequence of UCUCUGUCCAUUACCGUGGUAGC (SEQ ID NO: 49) and a sense strand comprising the nucleotide sequence of UACCACGGUAAUGGACAGAGA (SEQ ID NO: 357);
(j) an antisense strand comprising the nucleotide sequence of UAUUGUGUCAGGUUGCAGUACUC (SEQ ID NO: 60) and a sense strand comprising the nucleotide sequence of GUACUGCAACCUGACACAAUA (SEQ ID NO: 368);
(k) an antisense strand comprising the nucleotide sequence of UUGCGUCUGAGCAUUGUGUCAGG (SEQ ID NO: 64) and a sense strand comprising the nucleotide sequence of UGACACAAUGCUCAGACGCAA (SEQ ID NO: 372);
(l) an antisense strand comprising the nucleotide sequence of UUAACUCUGUCCAUAAUGGUAGU (SEQ ID NO: 88) and a sense strand comprising the nucleotide sequence of UACCAUUAUGGACAGAGUUAA (SEQ ID NO: 396);
(m) an antisense strand comprising the nucleotide sequence of UCCAAGCUUGGCAAGUUCUUCCU (SEQ ID NO: 89) and a sense strand comprising the nucleotide sequence of GAAGAACUUGCCAAGCUUGGA (SEQ ID NO: 397);
(n) an antisense strand comprising the nucleotide sequence of UAGAUGACCAAGCUUGGCAAGUU (SEQ ID NO: 90) and a sense strand comprising the nucleotide sequence of CUUGCCAAGCUUGGUCAUCUA (SEQ ID NO: 398); or
(o) an antisense strand comprising the nucleotide sequence of UGGUCCGACUAUGCUGGUGUGGU (SEQ ID NO: 98) and a sense strand comprising the nucleotide sequence of CACACCAGCAUAGUCGGACCA (SEQ ID NO: 406).

19. A polynucleic acid molecule for modulating expression of Lipoprotein(a) (Lp(a)) gene, wherein polynucleic acid molecule comprises:
(a) an antisense strand comprising the nucleotide sequence of usAfsgaugAfccaaGfcUfuGfgcaggsusc (SEQ ID NO: 158) and a sense strand comprising the nucleotide sequence of cscsugccAfaGfcUfuggucaucua (SEQ ID NO: 466);
(b) an antisense strand comprising the nucleotide sequence of usAfsuagaUfgaccAfaGfcUfuggcasgsg (SEQ ID NO: 159) and a sense strand comprising the nucleotide sequence of usgsccaaGfcUfuGfgucaucuaua (SEQ ID NO: 467);
(c) an antisense strand comprising the nucleotide sequence of usCfsauagAfugacCfaAfgCfuuggcsasg (SEQ ID NO: 160) and a sense strand comprising the nucleotide sequence of gscscaagCfuUfgGfucaucuauga (SEQ ID NO: 468);
(d) an antisense strand comprising the nucleotide sequence of usCfsgacgGfcaguCfcCfuUfcugcgsusc (SEQ ID NO: 165) and a sense strand comprising the nucleotide sequence of csgscagaAfgGfgAfcugccgucga (SEQ ID NO: 473);
(e) an antisense strand comprising the nucleotide sequence of usUfscuagGfcuugGfaAfcCfggggusasa (SEQ ID NO: 172) and a sense strand comprising the nucleotide sequence of ascscccgGfuUfcCfaagccuagaa (SEQ ID NO: 480);
(f) an antisense strand comprising the nucleotide sequence of usAfsgccuCfuaggCfuUfgGfaaccgsgsg (SEQ ID NO: 175) and a sense strand comprising the nucleotide sequence of csgsguucCfaAfgCfcuagaggcua (SEQ ID NO: 483);
(g) an antisense strand comprising the nucleotide sequence of usUfsuaccGfugguAfgCfaCfuccugscsa (SEQ ID NO: 198) and a sense strand comprising the nucleotide sequence of csasggagUfgCfuAfccacgguaaa (SEQ ID NO: 506);
(h) an antisense strand comprising the nucleotide sequence of usUfsguccAfuuacCfgUfgGfuagcascsu (SEQ ID NO: 201) and a sense strand comprising the nucleotide sequence of usgscuacCfaCfgGfuaauggacaa (SEQ ID NO: 509);
(i) an antisense strand comprising the nucleotide sequence of usCfsucugUfccauUfaCfcGfugguasgsc (SEQ ID NO: 203) and a sense strand comprising the nucleotide sequence of usasccacGfgUfaAfuggacagaga (SEQ ID NO: 511);
(j) an antisense strand comprising the nucleotide sequence of usAfsuuguGfucagGfuUfgCfaguacsusc (SEQ ID NO: 214) and a sense strand comprising the nucleotide sequence of gsusacugCfaAfcCfugacacaaua (SEQ ID NO: 522);
(k) an antisense strand comprising the nucleotide sequence of usUfsgcguCfugagCfaUfuGfugucasgsg (SEQ ID NO: 218) and a sense strand comprising the nucleotide sequence of usgsacacAfaUfgCfucagacgcaa (SEQ ID NO: 526);
(l) an antisense strand comprising the nucleotide sequence of usUfsaacuCfugucCfaUfaAfugguasgsu (SEQ ID NO: 242) and a sense strand comprising the nucleotide sequence of usasccauUfaUfgGfacagaguuaa (SEQ ID NO: 550);
(m) an antisense strand comprising the nucleotide sequence of usCfscaagCfuuggCfaAfgUfucuucscsu (SEQ ID NO: 243) and a sense strand comprising the nucleotide sequence of gsasagaaCfuUfgCfcaagcuugga (SEQ ID NO: 551);
(n) an antisense strand comprising the nucleotide sequence of usAfsgaugAfccaaGfcUfuGfgcaagsusu (SEQ ID NO: 244) and a sense strand comprising the nucleotide sequence of csusugccAfaGfcUfuggucaucua (SEQ ID NO: 552); or
(o) an antisense strand comprising the nucleotide sequence of usGfsguccGfacuaUfgCfuGfgugugsgsu (SEQ ID NO: 252) and a sense strand comprising the nucleotide sequence of csascaccAfgCfaUfagucggacca (SEQ ID NO: 560),
wherein “A” refers to adenosine-3′-phosphate; “a” refers to 2′-O-methyladenosine-3′-phosphate; “Af” refers to 2′-fluoroadenosine-3′-phosphate; “C” refers to cytidine-3′-phosphate; “c” refers to 2′-O-methylcytidine-3′-phosphate; “Cf” refers to 2′-fluorocytidine-3′-phosphate; “G” refers to guanosine-3′-phosphate; “g” refers to 2′-O-methylguanosine-3′-phosphate; “Gf” refers to 2′-fluoroguanosine-3′-phosphate; “U” refers to uridine-3′-phosphate; “u” refers to 2′-O-methyluridine-3′-phosphate; “Uf” refers to 2′-fluorouridine-3′-phosphate: “T” refers to 5-methyluridine-3′-phosphate; “t” refers to 2′-O-methyl-5-methyluridine-3′-phosphate; “Tf” refers to 2′-fluoro-5-methyluridine-3′-phosphate; “s” refers to 3′-phosphorothioate.

20. A polynucleic acid molecule conjugate for modulating expression of lipoprotein(a) gene (Lp(a)), wherein the polynucleic acid molecule conjugate comprises a polynucleic acid molecule of any one of claims [0221]-19 and an asialoglycoprotein receptor targeting moiety.

21. The polynucleic acid molecule conjugate of claim 20, wherein the asialoglycoprotein receptor targeting moiety comprises N-Acetylgalactosamine (GalNAc) or galactose.

22. The polynucleic acid molecule conjugate of one of claims 20-21, wherein the polynucleic acid molecule and the asialoglycoprotein receptor targeting moiety is coupled via a linker.

23. The polynucleic acid molecule conjugate of claim 22, wherein the linker is a cleavable linker.

24. The polynucleic acid molecule conjugate of any one of claims 21-23, wherein the GalNAc comprises an anomeric carbon bonded to trivalent, tetravalent linker, pentavalent, or hexavalent linker, wherein the anomeric carbon is part of a hemiaminal group.

25. The polynucleic acid molecule conjugate of any one of claims 22-24, wherein the linker comprises formula (IV) below, 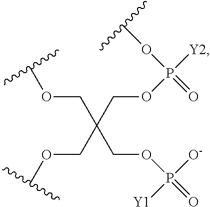
wherein at least one of Y1 and Y2 is a nucleotide in the polynucleic acid molecule.

26. The polynucleic acid molecule conjugate of claim 25, wherein the Y1 is the last nucleotide on the 3′-terminus of the sense strand of the polynucleic acid molecule.

27. The polynucleic acid molecule conjugate of any one of claims 22-26, wherein the linker and the asialoglycoprotein receptor targeting moiety with the last nucleotide on the 3′-terminus of the sense strand of the polynucleic acid molecule are shown in Formula (V′), Formula (V″″), Formula (V′∝″), or Formula (V″″″): 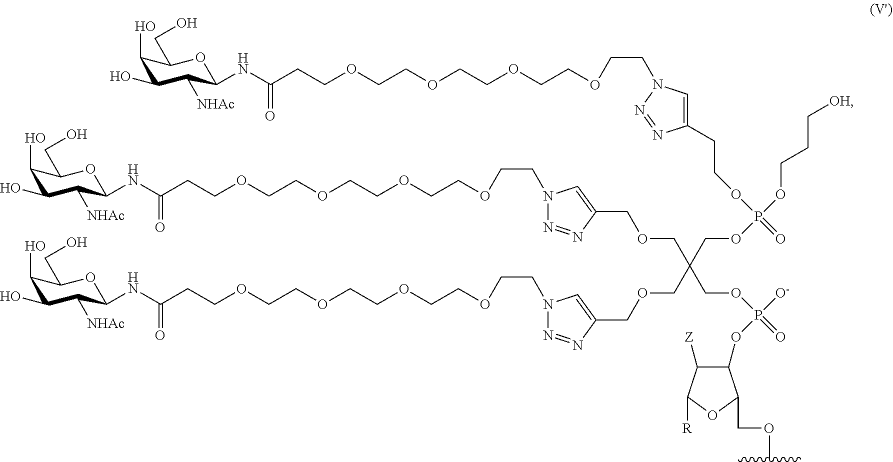
wherein Z in formula (V′) is —H, —OH, —O-Methyl, —F, or —O-methoxyethyl and R in formula (V′) is adenine, uracil, guanine, cytosine, thymine, abasic, or others; 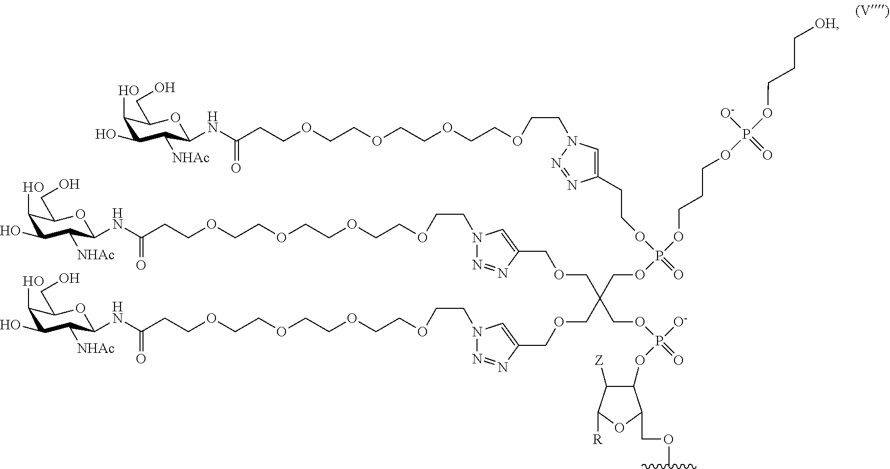
wherein Z in formula (V″″) is a moiety that corresponds to one of the sugar modifications described herein (e.g., —H, —OH, —O-Methyl, —F, or —O-methoxyethyl) and R in formula (V″″) is adenine, uracil, guanine, cytosine, thymine, abasic, or others. 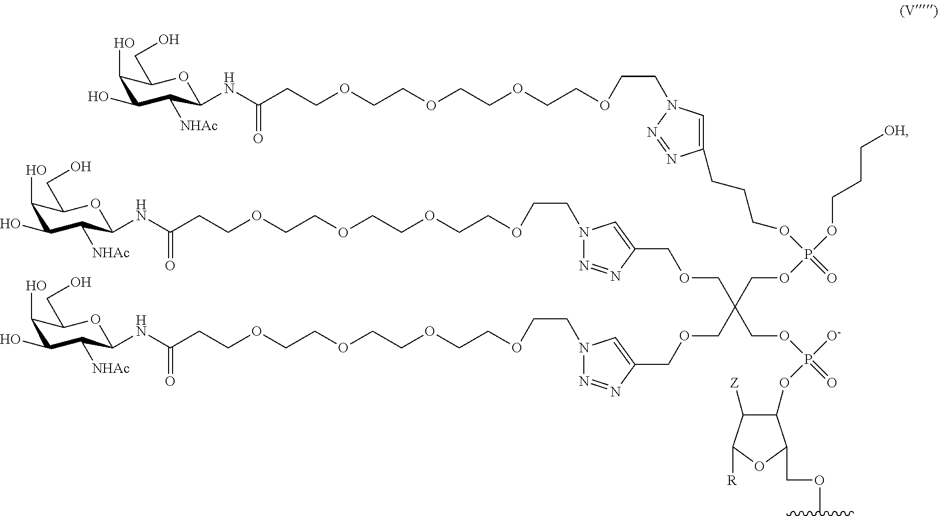
wherein Z in formula (V′″″) is a moiety that corresponds to one of the sugar modifications described herein (e.g., —H, —OH, —O-Methyl, —F, or —O-methoxyethyl) and R in formula (V′″″) is adenine, uracil, guanine, cytosine, thymine, abasic, or others; or 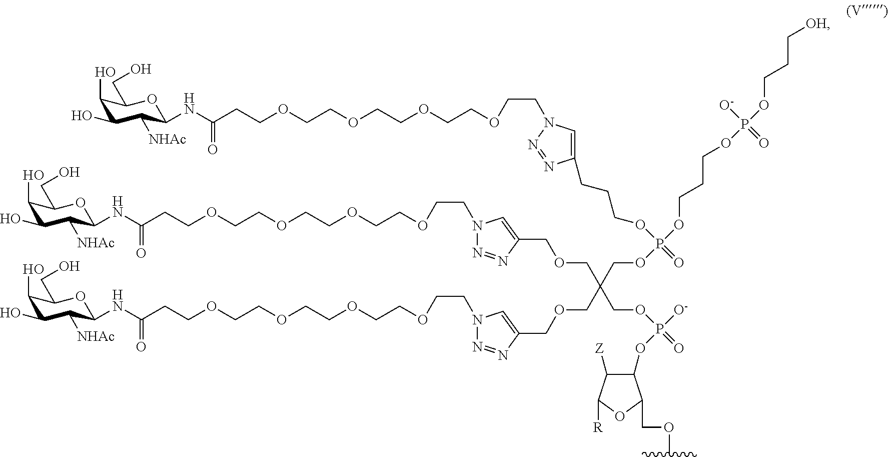
wherein Z in formula (V″″″) is a moiety that corresponds to one of the sugar modifications described herein (e.g., —H, —OH, —O-Methyl, —F, or —O-methoxyethyl) and R in formula (V″″″) is adenine, uracil, guanine, cytosine, thymine, abasic, or others.

28. The polynucleic acid molecule conjugate of claim 27, wherein the linker and the asialoglycoprotein receptor targeting moiety with the last nucleotide on the 3′-terminus of the sense strand of the polynucleic acid molecule are shown in Formula (V′): 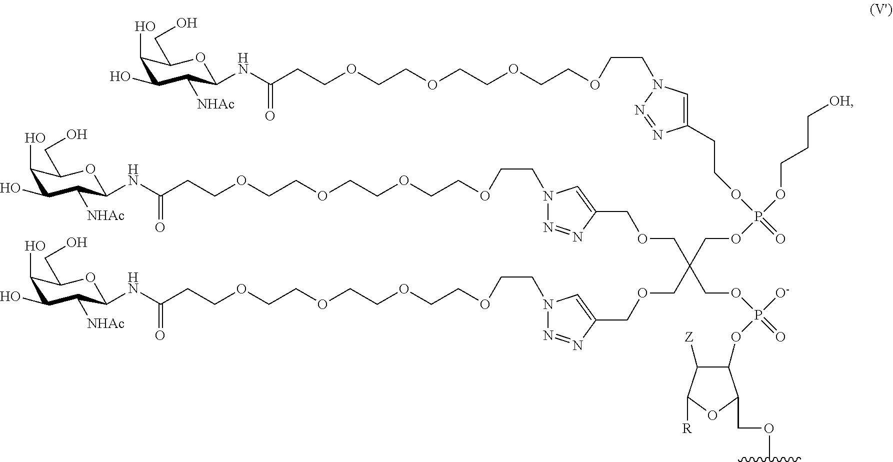
wherein Z in formula (V′) is —H, —OH, —O-Methyl, —F, or —O-methoxyethyl and R in formula (V′) is adenine, uracil, guanine, cytosine, thymine, abasic, or others.

29. A pharmaceutical composition comprising a polynucleic acid molecule of any one of claims [0221]-19 or a polynucleic acid molecule conjugate of any one of claims 20-28, and a pharmaceutically acceptable excipient.

30. The pharmaceutical composition of claim 29, wherein the pharmaceutical composition is formulated for parenteral, oral, intranasal, buccal, rectal, transdermal, intravenous, subcutaneous, or intrathecal administration.

31. A method of modulating expression of lipoprotein(a)(Lp(a)) gene in a subject in need thereof, comprising:
administering to the subject a polynucleic acid molecule of any one of claims 1-19, a polynucleic acid molecule conjugate of any one of claim s 20-28, or a pharmaceutical composition of any one of claims 29-30, thereby modulating the expression of Lp(a) gene in the subject in need thereof.

32. A method for treating or preventing a cardiovascular disease or a lipid disorder, comprising
administering to the subject a polynucleic acid molecule of any one of claims 1-19, a polynucleic acid molecule conjugate of any one of claims 20-28, or a pharmaceutical composition of any one of claims 29-30, thereby modulating the expression of Lp(a) gene in the subject in need thereof.

33. The method of any one of claims 31-32, wherein the polynucleic acid molecule is administered at a dose sufficient to decrease the expression of Lp(a) gene in a cell of said subject or to decrease plasma Lp(a) level of said subject by at least about 10%, 20%, 30%, 40%, 50%, 60%, 70%, 80%, or 90% compared to a control.

34. The method of any one of claims 32-33, wherein the cardiovascular disease is coronary artery disease, acute myocardial infarction, asymptomatic carotid atherosclerosis, stroke, atrial fibrillation, hypercholesterolemia, or peripheral artery occlusive disease.

35. The method of any one of claims 32-33, wherein the lipid disorder is hyperlipidemia or hypercholesterolemia.


SEQUENCE LISTING (618 sequences)

SEQ ID NO: 1 - RNA, 23 nt, synthetic construct
  source 1..23: mol_type = other RNA
tccaagcttggcaggtccttcct

SEQ ID NO: 2 - RNA, 23 nt, synthetic construct
  source 1..23: mol_type = other RNA
taccaagcttggcaggtccttcc

SEQ ID NO: 3 - RNA, 23 nt, synthetic construct
  source 1..23: mol_type = other RNA
tatgaccaagcttggcaggtcct

SEQ ID NO: 4 - RNA, 23 nt, synthetic construct
  source 1..23: mol_type = other RNA
tagatgaccaagcttggcaggtc

SEQ ID NO: 5 - RNA, 23 nt, synthetic construct
  source 1..23: mol_type = other RNA
tatagatgaccaagcttggcagg

SEQ ID NO: 6 - RNA, 23 nt, synthetic construct
  source 1..23: mol_type = other RNA
tcatagatgaccaagcttggcag

SEQ ID NO: 7 - RNA, 23 nt, synthetic construct
  source 1..23: mol_type = other RNA
tatcaagccagcatttgggtagt

SEQ ID NO: 8 - RNA, 23 nt, synthetic construct
  source 1..23: mol_type = other RNA
tgtcccttctgcgtctgagcatt

SEQ ID NO: 9 - RNA, 23 nt, synthetic construct
  source 1..23: mol_type = other RNA
tcggcagtcccttctgcgtctga

SEQ ID NO: 10 - RNA, 23 nt, synthetic construct
  source 1..23: mol_type = other RNA
tgacggcagtcccttctgcgtct

SEQ ID NO: 11 - RNA, 23 nt, synthetic construct
  source 1..23: mol_type = other RNA
tcgacggcagtcccttctgcgtc

SEQ ID NO: 12 - RNA, 23 nt, synthetic construct
  source 1..23: mol_type = other RNA
tgcgacggcagtcccttctgcgt

SEQ ID NO: 13 - RNA, 23 nt, synthetic construct
  source 1..23: mol_type = other RNA
tgaaccggggtaacagtcggagg

SEQ ID NO: 14 - RNA, 23 nt, synthetic construct
  source 1..23: mol_type = other RNA
tggaaccggggtaacagtcggag

SEQ ID NO: 15 - RNA, 23 nt, synthetic construct
  source 1..23: mol_type = other RNA
ttggaaccggggtaacagtcgga

SEQ ID NO: 16 - RNA, 23 nt, synthetic construct
  source 1..23: mol_type = other RNA
tgcttggaaccggggtaacagtc

SEQ ID NO: 17 - RNA, 23 nt, synthetic construct
  source 1..23: mol_type = other RNA
tggcttggaaccggggtaacagt

SEQ ID NO: 18 - RNA, 23 nt, synthetic construct
  source 1..23: mol_type = other RNA
ttctaggcttggaaccggggtaa

SEQ ID NO: 19 - RNA, 23 nt, synthetic construct
  source 1..23: mol_type = other RNA
tctctaggcttggaaccggggta

SEQ ID NO: 20 - RNA, 23 nt, synthetic construct
  source 1..23: mol_type = other RNA
tgcctctaggcttggaaccgggg

SEQ ID NO: 21 - RNA, 23 nt, synthetic construct
  source 1..23: mol_type = other RNA
tagcctctaggcttggaaccggg

SEQ ID NO: 22 - RNA, 23 nt, synthetic construct
  source 1..23: mol_type = other RNA
tgagcctctaggcttggaaccgg

SEQ ID NO: 23 - RNA, 23 nt, synthetic construct
  source 1..23: mol_type = other RNA
tgaaggagcctctaggcttggaa

SEQ ID NO: 24 - RNA, 23 nt, synthetic construct
  source 1..23: mol_type = other RNA
tcggaaggagcctctaggcttgg

SEQ ID NO: 25 - RNA, 23 nt, synthetic construct
  source 1..23: mol_type = other RNA
tgttcggaaggagcctctaggct

SEQ ID NO: 26 - RNA, 23 nt, synthetic construct
  source 1..23: mol_type = other RNA
ttgcttgttcggaaggagcctct

SEQ ID NO: 27 - RNA, 23 nt, synthetic construct
  source 1..23: mol_type = other RNA
tgtgcttgttcggaaggagcctc

SEQ ID NO: 28 - RNA, 23 nt, synthetic construct
  source 1..23: mol_type = other RNA
tcggtgcttgttcggaaggagcc

SEQ ID NO: 29 - RNA, 23 nt, synthetic construct
  source 1..23: mol_type = other RNA
ttcggtgcttgttcggaaggagc

SEQ ID NO: 30 - RNA, 23 nt, synthetic construct
  source 1..23: mol_type = other RNA
tcagtcggtgcttgttcggaagg

SEQ ID NO: 31 - RNA, 23 nt, synthetic construct
  source 1..23: mol_type = other RNA
ttgctcagtcggtgcttgttcgg

SEQ ID NO: 32 - RNA, 23 nt, synthetic construct
  source 1..23: mol_type = other RNA
ttagcactcctgcaccccaggcc

SEQ ID NO: 33 - RNA, 23 nt, synthetic construct
  source 1..23: mol_type = other RNA
tgtagcactcctgcaccccaggc

SEQ ID NO: 34 - RNA, 23 nt, synthetic construct
  source 1..23: mol_type = other RNA
tgataactctgtccattaccatg

SEQ ID NO: 35 - RNA, 23 nt, synthetic construct
  source 1..23: mol_type = other RNA
tcgataactctgtccattaccat

SEQ ID NO: 36 - RNA, 23 nt, synthetic construct
  source 1..23: mol_type = other RNA
ttatgtgcctcgataactctgtc

SEQ ID NO: 37 - RNA, 23 nt, synthetic construct
  source 1..23: mol_type = other RNA
tgtatgtgcctcgataactctgt

SEQ ID NO: 38 - RNA, 23 nt, synthetic construct
  source 1..23: mol_type = other RNA
tagtatgtgcctcgataactctg

SEQ ID NO: 39 - RNA, 23 nt, synthetic construct
  source 1..23: mol_type = other RNA
tacagtggtggagtatgtgcctc

SEQ ID NO: 40 - RNA, 23 nt, synthetic construct
  source 1..23: mol_type = other RNA
taccaagcttggcaggttcttcc

SEQ ID NO: 41 - RNA, 23 nt, synthetic construct
  source 1..23: mol_type = other RNA
tatgaccaagcttggcaggttct

SEQ ID NO: 42 - RNA, 23 nt, synthetic construct
  source 1..23: mol_type = other RNA
tagatgaccaagcttggcaggtt

SEQ ID NO: 43 - RNA, 23 nt, synthetic construct
  source 1..23: mol_type = other RNA
ttaccgtggtagcactcctgcac

SEQ ID NO: 44 - RNA, 23 nt, synthetic construct
  source 1..23: mol_type = other RNA
tttaccgtggtagcactcctgca

SEQ ID NO: 45 - RNA, 23 nt, synthetic construct
  source 1..23: mol_type = other RNA
tcattaccgtggtagcactcctg

SEQ ID NO: 46 - RNA, 23 nt, synthetic construct
  source 1..23: mol_type = other RNA
tgtccattaccgtggtagcactc

SEQ ID NO: 47 - RNA, 23 nt, synthetic construct
  source 1..23: mol_type = other RNA
ttgtccattaccgtggtagcact

SEQ ID NO: 48 - RNA, 23 nt, synthetic construct
  source 1..23: mol_type = other RNA
ttctgtccattaccgtggtagca

SEQ ID NO: 49 - RNA, 23 nt, synthetic construct
  source 1..23: mol_type = other RNA
tctctgtccattaccgtggtagc

SEQ ID NO: 50 - RNA, 23 nt, synthetic construct
  source 1..23: mol_type = other RNA
taactctgtccattaccgtggta

SEQ ID NO: 51 - RNA, 23 nt, synthetic construct
  source 1..23: mol_type = other RNA
tgataactctgtccattaccgtg

SEQ ID NO: 52 - RNA, 23 nt, synthetic construct
  source 1..23: mol_type = other RNA
tcgataactctgtccattaccgt

SEQ ID NO: 53 - RNA, 23 nt, synthetic construct
  source 1..23: mol_type = other RNA
tacaggatctggattcctgcagt

SEQ ID NO: 54 - RNA, 23 nt, synthetic construct
  source 1..23: mol_type = other RNA
taataaggggctgccacaggatc

SEQ ID NO: 55 - RNA, 23 nt, synthetic construct
  source 1..23: mol_type = other RNA
taacaataaggggctgccacagg

SEQ ID NO: 56 - RNA, 23 nt, synthetic construct
  source 1..23: mol_type = other RNA
tgtataacaataaggggctgcca

SEQ ID NO: 57 - RNA, 23 nt, synthetic construct
  source 1..23: mol_type = other RNA
tcgtataacaataaggggctgcc

SEQ ID NO: 58 - RNA, 23 nt, synthetic construct
  source 1..23: mol_type = other RNA
ttcgtataacaataaggggctgc

SEQ ID NO: 59 - RNA, 23 nt, synthetic construct
  source 1..23: mol_type = other RNA
ttgtgtcaggttgcagtactccc

SEQ ID NO: 60 - RNA, 23 nt, synthetic construct
  source 1..23: mol_type = other RNA
tattgtgtcaggttgcagtactc

SEQ ID NO: 61 - RNA, 23 nt, synthetic construct
  source 1..23: mol_type = other RNA
tgcattgtgtcaggttgcagtac

SEQ ID NO: 62 - RNA, 23 nt, synthetic construct
  source 1..23: mol_type = other RNA
tgagcattgtgtcaggttgcagt

SEQ ID NO: 63 - RNA, 23 nt, synthetic construct
  source 1..23: mol_type = other RNA
ttgagcattgtgtcaggttgcag

SEQ ID NO: 64 - RNA, 23 nt, synthetic construct
  source 1..23: mol_type = other RNA
ttgcgtctgagcattgtgtcagg

SEQ ID NO: 65 - RNA, 23 nt, synthetic construct
  source 1..23: mol_type = other RNA
ttctgcgtctgagcattgtgtca

SEQ ID NO: 66 - RNA, 23 nt, synthetic construct
  source 1..23: mol_type = other RNA
tcttctgcgtctgagcattgtgt

SEQ ID NO: 67 - RNA, 23 nt, synthetic construct
  source 1..23: mol_type = other RNA
tgagcctctaggcttggaatcgg

SEQ ID NO: 68 - RNA, 23 nt, synthetic construct
  source 1..23: mol_type = other RNA
tttcagaaggagcctctaggctt

SEQ ID NO: 69 - RNA, 23 nt, synthetic construct
  source 1..23: mol_type = other RNA
tttgctcagttggtgcttgttca

SEQ ID NO: 70 - RNA, 23 nt, synthetic construct
  source 1..23: mol_type = other RNA
tctttgctcagttggtgcttgtt

SEQ ID NO: 71 - RNA, 23 nt, synthetic construct
  source 1..23: mol_type = other RNA
tgtgccttgataactctgtccat

SEQ ID NO: 72 - RNA, 23 nt, synthetic construct
  source 1..23: mol_type = other RNA
tggatctggatttcggcagtagt

SEQ ID NO: 73 - RNA, 23 nt, synthetic construct
  source 1..23: mol_type = other RNA
ttaacaccaaggggctgccacag

SEQ ID NO: 74 - RNA, 23 nt, synthetic construct
  source 1..23: mol_type = other RNA
tgtataacaccaaggggctgcca

SEQ ID NO: 75 - RNA, 23 nt, synthetic construct
  source 1..23: mol_type = other RNA
tttgtataacaccaaggggctgc

SEQ ID NO: 76 - RNA, 23 nt, synthetic construct
  source 1..23: mol_type = other RNA
tgttgtataacaccaaggggctg

SEQ ID NO: 77 - RNA, 23 nt, synthetic construct
  source 1..23: mol_type = other RNA
ttcgtgtcaggttgcagtactcc

SEQ ID NO: 78 - RNA, 23 nt, synthetic construct
  source 1..23: mol_type = other RNA
tagcatcgtgtcaggttgcagta

SEQ ID NO: 79 - RNA, 23 nt, synthetic construct
  source 1..23: mol_type = other RNA
tgagcatcgtgtcaggttgcagt

SEQ ID NO: 80 - RNA, 23 nt, synthetic construct
  source 1..23: mol_type = other RNA
ttgagcatcgtgtcaggttgcag

SEQ ID NO: 81 - RNA, 23 nt, synthetic construct
  source 1..23: mol_type = other RNA
ttctgagcatcgtgtcaggttgc

SEQ ID NO: 82 - RNA, 23 nt, synthetic construct
  source 1..23: mol_type = other RNA
taaagcctctaggcttggagcca

SEQ ID NO: 83 - RNA, 23 nt, synthetic construct
  source 1..23: mol_type = other RNA
tccataatggtagtagcagtcct

SEQ ID NO: 84 - RNA, 23 nt, synthetic construct
  source 1..23: mol_type = other RNA
ttccataatggtagtagcagtcc

SEQ ID NO: 85 - RNA, 23 nt, synthetic construct
  source 1..23: mol_type = other RNA
tgtccataatggtagtagcagtc

SEQ ID NO: 86 - RNA, 23 nt, synthetic construct
  source 1..23: mol_type = other RNA
ttctgtccataatggtagtagca

SEQ ID NO: 87 - RNA, 23 nt, synthetic construct
  source 1..23: mol_type = other RNA
taactctgtccataatggtagta

SEQ ID NO: 88 - RNA, 23 nt, synthetic construct
  source 1..23: mol_type = other RNA
ttaactctgtccataatggtagt

SEQ ID NO: 89 - RNA, 23 nt, synthetic construct
  source 1..23: mol_type = other RNA
tccaagcttggcaagttcttcct

SEQ ID NO: 90 - RNA, 23 nt, synthetic construct
  source 1..23: mol_type = other RNA
tagatgaccaagcttggcaagtt

SEQ ID NO: 91 - RNA, 23 nt, synthetic construct
  source 1..23: mol_type = other RNA
tatagatgaccaagcttggcaag

SEQ ID NO: 92 - RNA, 23 nt, synthetic construct
  source 1..23: mol_type = other RNA
tcatagatgaccaagcttggcaa

SEQ ID NO: 93 - RNA, 23 nt, synthetic construct
  source 1..23: mol_type = other RNA
tactatgctggtgtggtgtcata

SEQ ID NO: 94 - RNA, 23 nt, synthetic construct
  source 1..23: mol_type = other RNA
tgactatgctggtgtggtgtcat

SEQ ID NO: 95 - RNA, 23 nt, synthetic construct
  source 1..23: mol_type = other RNA
tcgactatgctggtgtggtgtca

SEQ ID NO: 96 - RNA, 23 nt, synthetic construct
  source 1..23: mol_type = other RNA
ttccgactatgctggtgtggtgt

SEQ ID NO: 97 - RNA, 23 nt, synthetic construct
  source 1..23: mol_type = other RNA
tgtccgactatgctggtgtggtg

SEQ ID NO: 98 - RNA, 23 nt, synthetic construct
  source 1..23: mol_type = other RNA
tggtccgactatgctggtgtggt

SEQ ID NO: 99 - RNA, 23 nt, synthetic construct
  source 1..23: mol_type = other RNA
ttttctggggtccgactatgctg

SEQ ID NO: 100 - RNA, 23 nt, synthetic construct
  source 1..23: mol_type = other RNA
tttttctggggtccgactatgct

SEQ ID NO: 101 - RNA, 23 nt, synthetic construct
  source 1..23: mol_type = other RNA
ttagttttctggggtccgactat

SEQ ID NO: 102 - RNA, 23 nt, synthetic construct
  source 1..23: mol_type = other RNA
tatccatggtgtaacaccaaggg

SEQ ID NO: 103 - RNA, 23 nt, synthetic construct
  source 1..23: mol_type = other RNA
tgatccatggtgtaacaccaagg

SEQ ID NO: 104 - RNA, 23 nt, synthetic construct
  source 1..23: mol_type = other RNA
tggatccatggtgtaacaccaag

SEQ ID NO: 105 - RNA, 23 nt, synthetic construct
  source 1..23: mol_type = other RNA
tgggatccatggtgtaacaccaa

SEQ ID NO: 106 - RNA, 23 nt, synthetic construct
  source 1..23: mol_type = other RNA
ttgacactgggatccatggtgta

SEQ ID NO: 107 - RNA, 23 nt, synthetic construct
  source 1..23: mol_type = other RNA
tctgacactgggatccatggtgt

SEQ ID NO: 108 - RNA, 23 nt, synthetic construct
  source 1..23: mol_type = other RNA
tgttgcaaggacacttgattctg

SEQ ID NO: 109 - RNA, 23 nt, synthetic construct
  source 1..23: mol_type = other RNA
tcatggtataacaccaaggacta

SEQ ID NO: 110 - RNA, 23 nt, synthetic construct
  source 1..23: mol_type = other RNA
ttccatggtataacaccaaggac

SEQ ID NO: 111 - RNA, 23 nt, synthetic construct
  source 1..23: mol_type = other RNA
tatccatggtataacaccaagga

SEQ ID NO: 112 - RNA, 23 nt, synthetic construct
  source 1..23: mol_type = other RNA
tgggatccatggtataacaccaa

SEQ ID NO: 113 - RNA, 23 nt, synthetic construct
  source 1..23: mol_type = other RNA
tgacattgtgtcaggttgcagta

SEQ ID NO: 114 - RNA, 23 nt, synthetic construct
  source 1..23: mol_type = other RNA
tggacattgtgtcaggttgcagt

SEQ ID NO: 115 - RNA, 23 nt, synthetic construct
  source 1..23: mol_type = other RNA
ttggacattgtgtcaggttgcag

SEQ ID NO: 116 - RNA, 23 nt, synthetic construct
  source 1..23: mol_type = other RNA
tcactggacattgtgtcaggttg

SEQ ID NO: 117 - RNA, 23 nt, synthetic construct
  source 1..23: mol_type = other RNA
tattctgtcactggacattgtgt

SEQ ID NO: 118 - RNA, 23 nt, synthetic construct
  source 1..23: mol_type = other RNA
tcaccatttgggtagtattctgt

SEQ ID NO: 119 - RNA, 23 nt, synthetic construct
  source 1..23: mol_type = other RNA
tgtataacaccaagggcgaatct

SEQ ID NO: 120 - RNA, 23 nt, synthetic construct
  source 1..23: mol_type = other RNA
tcatggtataacaccaagggcga

SEQ ID NO: 121 - RNA, 23 nt, synthetic construct
  source 1..23: mol_type = other RNA
ttccatggtataacaccaagggc

SEQ ID NO: 122 - RNA, 23 nt, synthetic construct
  source 1..23: mol_type = other RNA
tatccatggtataacaccaaggg

SEQ ID NO: 123 - RNA, 23 nt, synthetic construct
  source 1..23: mol_type = other RNA
ttgacactgggatccatggtata

SEQ ID NO: 124 - RNA, 23 nt, synthetic construct
  source 1..23: mol_type = other RNA
tctgacactgggatccatggtat

SEQ ID NO: 125 - RNA, 23 nt, synthetic construct
  source 1..23: mol_type = other RNA
tggaccaccgtgggagttgtgag

SEQ ID NO: 126 - RNA, 23 nt, synthetic construct
  source 1..23: mol_type = other RNA
tgggaccaccgtgggagttgtga

SEQ ID NO: 127 - RNA, 23 nt, synthetic construct
  source 1..23: mol_type = other RNA
ttgcttggaactgggaccaccgt

SEQ ID NO: 128 - RNA, 23 nt, synthetic construct
  source 1..23: mol_type = other RNA
tattctgtcactggacatcgtgt

SEQ ID NO: 129 - RNA, 23 nt, synthetic construct
  source 1..23: mol_type = other RNA
tgatgccagtgtggtatcataga

SEQ ID NO: 130 - RNA, 23 nt, synthetic construct
  source 1..23: mol_type = other RNA
ttgatgccagtgtggtatcatag

SEQ ID NO: 131 - RNA, 23 nt, synthetic construct
  source 1..23: mol_type = other RNA
tgtgtaacaccagggttgtttcc

SEQ ID NO: 132 - RNA, 23 nt, synthetic construct
  source 1..23: mol_type = other RNA
taggacacctgattctgtttctg

SEQ ID NO: 133 - RNA, 23 nt, synthetic construct
  source 1..23: mol_type = other RNA
ttaggacacctgattctgtttct

SEQ ID NO: 134 - RNA, 23 nt, synthetic construct
  source 1..23: mol_type = other RNA
ttctaggacacctgattctgttt

SEQ ID NO: 135 - RNA, 23 nt, synthetic construct
  source 1..23: mol_type = other RNA
tctctaggacacctgattctgtt

SEQ ID NO: 136 - RNA, 23 nt, synthetic construct
  source 1..23: mol_type = other RNA
tgtctctaggacacctgattctg

SEQ ID NO: 137 - RNA, 23 nt, synthetic construct
  source 1..23: mol_type = other RNA
tgggagtctctaggacacctgat

SEQ ID NO: 138 - RNA, 23 nt, synthetic construct
  source 1..23: mol_type = other RNA
tggaacaacagtgggagtctcta

SEQ ID NO: 139 - RNA, 23 nt, synthetic construct
  source 1..23: mol_type = other RNA
tgaatgtgcctcgataactctgg

SEQ ID NO: 140 - RNA, 23 nt, synthetic construct
  source 1..23: mol_type = other RNA
ttggatgaccaagattgacatgt

SEQ ID NO: 141 - RNA, 23 nt, synthetic construct
  source 1..23: mol_type = other RNA
tatggatgaccaagattgacatg

SEQ ID NO: 142 - RNA, 23 nt, synthetic construct
  source 1..23: mol_type = other RNA
taaaacaccaagggcctgtatcg

SEQ ID NO: 143 - RNA, 23 nt, synthetic construct
  source 1..23: mol_type = other RNA
tgcatcgcgtcaggttgcagtac

SEQ ID NO: 144 - RNA, 23 nt, synthetic construct
  source 1..23: mol_type = other RNA
tagcatcgcgtcaggttgcagta

SEQ ID NO: 145 - RNA, 23 nt, synthetic construct
  source 1..23: mol_type = other RNA
tgagcatcgcgtcaggttgcagt

SEQ ID NO: 146 - RNA, 23 nt, synthetic construct
  source 1..23: mol_type = other RNA
ttgagcatcgcgtcaggttgcag

SEQ ID NO: 147 - RNA, 23 nt, synthetic construct
  source 1..23: mol_type = other RNA
ttctgagcatcgcgtcaggttgc

SEQ ID NO: 148 - RNA, 23 nt, synthetic construct
  source 1..23: mol_type = other RNA
tgtctgagcatcgcgtcaggttg

SEQ ID NO: 149 - RNA, 23 nt, synthetic construct
  source 1..23: mol_type = other RNA
ttgtctgagcatcgcgtcaggtt

SEQ ID NO: 150 - RNA, 23 nt, synthetic construct
  source 1..23: mol_type = other RNA
tgtgtctgagcatcgcgtcaggt

SEQ ID NO: 151 - RNA, 23 nt, synthetic construct
  source 1..23: mol_type = other RNA
ttgtgtctgagcatcgcgtcagg

SEQ ID NO: 152 - RNA, 23 nt, synthetic construct
  source 1..23: mol_type = other RNA
ttctgtgtctgagcatcgcgtca

SEQ ID NO: 153 - RNA, 23 nt, synthetic construct
  source 1..23: mol_type = other RNA
ttacaatgcttccaggacatttc

SEQ ID NO: 154 - RNA, 23 nt, synthetic construct
  source 1..23: mol_type = other RNA
tcagtgatgtaacattcagtcct

SEQ ID NO: 155 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tccaagcttggcaggtccttcct

SEQ ID NO: 156 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
taccaagcttggcaggtccttcc

SEQ ID NO: 157 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tatgaccaagcttggcaggtcct

SEQ ID NO: 158 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tagatgaccaagcttggcaggtc

SEQ ID NO: 159 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tatagatgaccaagcttggcagg

SEQ ID NO: 160 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcatagatgaccaagcttggcag

SEQ ID NO: 161 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tatcaagccagcatttgggtagt

SEQ ID NO: 162 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgtcccttctgcgtctgagcatt

SEQ ID NO: 163 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcggcagtcccttctgcgtctga

SEQ ID NO: 164 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgacggcagtcccttctgcgtct

SEQ ID NO: 165 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcgacggcagtcccttctgcgtc

SEQ ID NO: 166 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgcgacggcagtcccttctgcgt

SEQ ID NO: 167 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgaaccggggtaacagtcggagg

SEQ ID NO: 168 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tggaaccggggtaacagtcggag

SEQ ID NO: 169 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttggaaccggggtaacagtcgga

SEQ ID NO: 170 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgcttggaaccggggtaacagtc

SEQ ID NO: 171 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tggcttggaaccggggtaacagt

SEQ ID NO: 172 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttctaggcttggaaccggggtaa

SEQ ID NO: 173 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tctctaggcttggaaccggggta

SEQ ID NO: 174 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgcctctaggcttggaaccgggg

SEQ ID NO: 175 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tagcctctaggcttggaaccggg

SEQ ID NO: 176 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgagcctctaggcttggaaccgg

SEQ ID NO: 177 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgaaggagcctctaggcttggaa

SEQ ID NO: 178 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcggaaggagcctctaggcttgg

SEQ ID NO: 179 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgttcggaaggagcctctaggct

SEQ ID NO: 180 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgcttgttcggaaggagcctct

SEQ ID NO: 181 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgtgcttgttcggaaggagcctc

SEQ ID NO: 182 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcggtgcttgttcggaaggagcc

SEQ ID NO: 183 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttcggtgcttgttcggaaggagc

SEQ ID NO: 184 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcagtcggtgcttgttcggaagg

SEQ ID NO: 185 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgctcagtcggtgcttgttcgg

SEQ ID NO: 186 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttagcactcctgcaccccaggcc

SEQ ID NO: 187 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgtagcactcctgcaccccaggc

SEQ ID NO: 188 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgataactctgtccattaccatg

SEQ ID NO: 189 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcgataactctgtccattaccat

SEQ ID NO: 190 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttatgtgcctcgataactctgtc

SEQ ID NO: 191 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgtatgtgcctcgataactctgt

SEQ ID NO: 192 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tagtatgtgcctcgataactctg

SEQ ID NO: 193 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tacagtggtggagtatgtgcctc

SEQ ID NO: 194 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
taccaagcttggcaggttcttcc

SEQ ID NO: 195 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tatgaccaagcttggcaggttct

SEQ ID NO: 196 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tagatgaccaagcttggcaggtt

SEQ ID NO: 197 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttaccgtggtagcactcctgcac

SEQ ID NO: 198 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tttaccgtggtagcactcctgca

SEQ ID NO: 199 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcattaccgtggtagcactcctg

SEQ ID NO: 200 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgtccattaccgtggtagcactc

SEQ ID NO: 201 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgtccattaccgtggtagcact

SEQ ID NO: 202 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttctgtccattaccgtggtagca

SEQ ID NO: 203 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tctctgtccattaccgtggtagc

SEQ ID NO: 204 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
taactctgtccattaccgtggta

SEQ ID NO: 205 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgataactctgtccattaccgtg

SEQ ID NO: 206 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcgataactctgtccattaccgt

SEQ ID NO: 207 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tacaggatctggattcctgcagt

SEQ ID NO: 208 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
taataaggggctgccacaggatc

SEQ ID NO: 209 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
taacaataaggggctgccacagg

SEQ ID NO: 210 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgtataacaataaggggctgcca

SEQ ID NO: 211 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcgtataacaataaggggctgcc

SEQ ID NO: 212 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttcgtataacaataaggggctgc

SEQ ID NO: 213 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgtgtcaggttgcagtactccc

SEQ ID NO: 214 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tattgtgtcaggttgcagtactc

SEQ ID NO: 215 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgcattgtgtcaggttgcagtac

SEQ ID NO: 216 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgagcattgtgtcaggttgcagt

SEQ ID NO: 217 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgagcattgtgtcaggttgcag

SEQ ID NO: 218 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgcgtctgagcattgtgtcagg

SEQ ID NO: 219 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttctgcgtctgagcattgtgtca

SEQ ID NO: 220 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcttctgcgtctgagcattgtgt

SEQ ID NO: 221 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgagcctctaggcttggaatcgg

SEQ ID NO: 222 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tttcagaaggagcctctaggctt

SEQ ID NO: 223 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tttgctcagttggtgcttgttca

SEQ ID NO: 224 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tctttgctcagttggtgcttgtt

SEQ ID NO: 225 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgtgccttgataactctgtccat

SEQ ID NO: 226 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tggatctggatttcggcagtagt

SEQ ID NO: 227 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttaacaccaaggggctgccacag

SEQ ID NO: 228 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgtataacaccaaggggctgcca

SEQ ID NO: 229 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tttgtataacaccaaggggctgc

SEQ ID NO: 230 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgttgtataacaccaaggggctg

SEQ ID NO: 231 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttcgtgtcaggttgcagtactcc

SEQ ID NO: 232 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tagcatcgtgtcaggttgcagta

SEQ ID NO: 233 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgagcatcgtgtcaggttgcagt

SEQ ID NO: 234 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgagcatcgtgtcaggttgcag

SEQ ID NO: 235 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttctgagcatcgtgtcaggttgc

SEQ ID NO: 236 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
taaagcctctaggcttggagcca

SEQ ID NO: 237 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tccataatggtagtagcagtcct

SEQ ID NO: 238 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttccataatggtagtagcagtcc

SEQ ID NO: 239 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgtccataatggtagtagcagtc

SEQ ID NO: 240 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttctgtccataatggtagtagca

SEQ ID NO: 241 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
taactctgtccataatggtagta

SEQ ID NO: 242 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttaactctgtccataatggtagt

SEQ ID NO: 243 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tccaagcttggcaagttcttcct

SEQ ID NO: 244 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tagatgaccaagcttggcaagtt

SEQ ID NO: 245 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tatagatgaccaagcttggcaag

SEQ ID NO: 246 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcatagatgaccaagcttggcaa

SEQ ID NO: 247 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tactatgctggtgtggtgtcata

SEQ ID NO: 248 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgactatgctggtgtggtgtcat

SEQ ID NO: 249 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcgactatgctggtgtggtgtca

SEQ ID NO: 250 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttccgactatgctggtgtggtgt

SEQ ID NO: 251 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgtccgactatgctggtgtggtg

SEQ ID NO: 252 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tggtccgactatgctggtgtggt

SEQ ID NO: 253 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttttctggggtccgactatgctg

SEQ ID NO: 254 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tttttctggggtccgactatgct

SEQ ID NO: 255 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttagttttctggggtccgactat

SEQ ID NO: 256 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tatccatggtgtaacaccaaggg

SEQ ID NO: 257 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgatccatggtgtaacaccaagg

SEQ ID NO: 258 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tggatccatggtgtaacaccaag

SEQ ID NO: 259 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgggatccatggtgtaacaccaa

SEQ ID NO: 260 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgacactgggatccatggtgta

SEQ ID NO: 261 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tctgacactgggatccatggtgt

SEQ ID NO: 262 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgttgcaaggacacttgattctg

SEQ ID NO: 263 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcatggtataacaccaaggacta

SEQ ID NO: 264 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttccatggtataacaccaaggac

SEQ ID NO: 265 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tatccatggtataacaccaagga

SEQ ID NO: 266 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgggatccatggtataacaccaa

SEQ ID NO: 267 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgacattgtgtcaggttgcagta

SEQ ID NO: 268 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tggacattgtgtcaggttgcagt

SEQ ID NO: 269 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttggacattgtgtcaggttgcag

SEQ ID NO: 270 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcactggacattgtgtcaggttg

SEQ ID NO: 271 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tattctgtcactggacattgtgt

SEQ ID NO: 272 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcaccatttgggtagtattctgt

SEQ ID NO: 273 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgtataacaccaagggcgaatct

SEQ ID NO: 274 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcatggtataacaccaagggcga

SEQ ID NO: 275 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttccatggtataacaccaagggc

SEQ ID NO: 276 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tatccatggtataacaccaaggg

SEQ ID NO: 277 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgacactgggatccatggtata

SEQ ID NO: 278 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tctgacactgggatccatggtat

SEQ ID NO: 279 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tggaccaccgtgggagttgtgag

SEQ ID NO: 280 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgggaccaccgtgggagttgtga

SEQ ID NO: 281 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgcttggaactgggaccaccgt

SEQ ID NO: 282 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tattctgtcactggacatcgtgt

SEQ ID NO: 283 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgatgccagtgtggtatcataga

SEQ ID NO: 284 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgatgccagtgtggtatcatag

SEQ ID NO: 285 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgtgtaacaccagggttgtttcc

SEQ ID NO: 286 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
taggacacctgattctgtttctg

SEQ ID NO: 287 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttaggacacctgattctgtttct

SEQ ID NO: 288 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttctaggacacctgattctgttt

SEQ ID NO: 289 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tctctaggacacctgattctgtt

SEQ ID NO: 290 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgtctctaggacacctgattctg

SEQ ID NO: 291 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgggagtctctaggacacctgat

SEQ ID NO: 292 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tggaacaacagtgggagtctcta

SEQ ID NO: 293 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgaatgtgcctcgataactctgg

SEQ ID NO: 294 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttggatgaccaagattgacatgt

SEQ ID NO: 295 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tatggatgaccaagattgacatg

SEQ ID NO: 296 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
taaaacaccaagggcctgtatcg

SEQ ID NO: 297 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgcatcgcgtcaggttgcagtac

SEQ ID NO: 298 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tagcatcgcgtcaggttgcagta

SEQ ID NO: 299 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgagcatcgcgtcaggttgcagt

SEQ ID NO: 300 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgagcatcgcgtcaggttgcag

SEQ ID NO: 301 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttctgagcatcgcgtcaggttgc

SEQ ID NO: 302 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgtctgagcatcgcgtcaggttg

SEQ ID NO: 303 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgtctgagcatcgcgtcaggtt

SEQ ID NO: 304 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tgtgtctgagcatcgcgtcaggt

SEQ ID NO: 305 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttgtgtctgagcatcgcgtcagg

SEQ ID NO: 306 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttctgtgtctgagcatcgcgtca

SEQ ID NO: 307 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
ttacaatgcttccaggacatttc

SEQ ID NO: 308 - RNA, 23 nt, synthetic construct
  source 1..23: mol_type = other RNA
  modified_base 1: mod_base = OTHER; note = 2'O-Methyl ribonucleotide
  modified_base 1..3: mod_base = OTHER; note = Phosphorothioate linkage
  modified_base 2: mod_base = OTHER; note = 2'fluoro ribonucleotide
  modified_base 3..6: mod_base = OTHER; note = 2'O-Methyl ribonucleotide
  modified_base 7: mod_base = OTHER; note = 2'fluoro ribonucleotide
  modified_base 8..11: mod_base = OTHER; note = 2'O-Methyl ribonucleotide
  modified_base 12: mod_base = OTHER; note = 2'fluoro ribonucleotide
  modified_base 13: mod_base = OTHER; note = 2'O-Methyl ribonucleotide
  modified_base 14: mod_base = OTHER; note = 2'fluoro ribonucleotide
  modified_base 15: mod_base = OTHER; note = 2'O-Methyl ribonucleotide
  modified_base 16: mod_base = OTHER; note = 2'fluoro ribonucleotide
  modified_base 17..23: mod_base = OTHER; note = 2'O-Methyl ribonucleotide
  modified_base 21..23: mod_base = OTHER; note = Phosphorothioate linkage
tcagtgatgtaacattcagtcct

SEQ ID NO: 309 - RNA, 21 nt, synthetic construct
  source 1..21: mol_type = other RNA
gaaggacctgccaagcttgga

SEQ ID NO: 310 - RNA, 21 nt, synthetic construct
  source 1..21: mol_type = other RNA
aaggacctgccaagcttggta

SEQ ID NO: 311 - RNA, 21 nt, synthetic construct
  source 1..21: mol_type = other RNA
gacctgccaagcttggtcata

SEQ ID NO: 312 - RNA, 21 nt, synthetic construct
  source 1..21: mol_type = other RNA
cctgccaagcttggtcatcta

SEQ ID NO: 313 - RNA, 21 nt, synthetic construct
  source 1..21: mol_type = other RNA
tgccaagcttggtcatctata

SEQ ID NO: 314 - RNA, 21 nt, synthetic construct
  source 1..21: mol_type = other RNA
gccaagcttggtcatctatga

SEQ ID NO: 315 - RNA, 21 nt, synthetic construct
  source 1..21: mol_type = other RNA
tacccaaatgctggcttgata

SEQ ID NO: 316 - RNA, 21 nt, synthetic construct
  source 1..21: mol_type = other RNA
tgctcagacgcagaagggaca

SEQ ID NO: 317 - RNA, 21 nt, synthetic construct
  source 1..21: mol_type = other RNA
agacgcagaagggactgccga

SEQ ID NO: 318 - RNA, 21 nt, synthetic construct
  source 1..21: mol_type = other RNA
acgcagaagggactgccgtca

SEQ ID NO: 319 - RNA, 21 nt, synthetic construct
  source 1..21: mol_type = other RNA
cgcagaagggactgccgtcga

SEQ ID NO: 320 - RNA, 21 nt, synthetic construct
  source 1..21: mol_type = other RNA
gcagaagggactgccgtcgca

SEQ ID NO: 321 - RNA, 21 nt, synthetic construct
  source 1..21: mol_type = other RNA
tccgactgttaccccggttca

SEQ ID NO: 322 - RNA, 21 nt, synthetic construct
  source 1..21: mol_type = other RNA
ccgactgttaccccggttcca

SEQ ID NO: 323 - RNA, 21 nt, synthetic construct
  source 1..21: mol_type = other RNA
cgactgttaccccggttccaa

SEQ ID NO: 324 - RNA, 21 nt, synthetic construct
  source 1..21: mol_type = other RNA
ctgttaccccggttccaagca

SEQ ID NO: 325 - RNA, 21 nt, synthetic construct
  source 1..21: mol_type = other RNA
tgttaccccggttccaagcca

SEQ ID NO: 326 - RNA, 21 nt, synthetic construct
  source 1..21: mol_type = other RNA
accccggttccaagcctagaa

SEQ ID NO: 327 - RNA, 21 nt, synthetic construct
  source 1..21: mol_type = other RNA
ccccggttccaagcctagaga

SEQ ID NO: 328 - RNA, 21 nt, synthetic construct
  source 1..21: mol_type = other RNA
ccggttccaagcctagaggca

SEQ ID NO: 329 - RNA, 21 nt, synthetic construct
  source 1..21: mol_type = other RNA
cggttccaagcctagaggcta

SEQ ID NO: 330 - RNA, 21 nt, synthetic construct
  source 1..21: mol_type = other RNA
ggttccaagcctagaggctca

SEQ ID NO: 331 - RNA, 21 nt, synthetic construct
  source 1..21: mol_type = other RNA
ccaagcctagaggctccttca

SEQ ID NO: 332 - RNA, 21 nt, synthetic construct
  source 1..21: mol_type = other RNA
aagcctagaggctccttccga

SEQ ID NO: 333 - RNA, 21 nt, synthetic construct
  source 1..21: mol_type = other RNA
cctagaggctccttccgaaca

SEQ ID NO: 334 - RNA, 21 nt, synthetic construct
  source 1..21: mol_type = other RNA
aggctccttccgaacaagcaa

SEQ ID NO: 335 - RNA, 21 nt, synthetic construct
  source 1..21: mol_type = other RNA
ggctccttccgaacaagcaca

SEQ ID NO: 336 - RNA, 21 nt, synthetic construct
  source 1..21: mol_type = other RNA
ctccttccgaacaagcaccga

SEQ ID NO: 337 - RNA, 21 nt, synthetic construct
  source 1..21: mol_type = other RNA
tccttccgaacaagcaccgaa

SEQ ID NO: 338 - RNA, 21 nt, synthetic construct
  source 1..21: mol_type = other RNA
ttccgaacaagcaccgactga

SEQ ID NO: 339 - RNA, 21 nt, synthetic construct
  source 1..21: mol_type = other RNA
gaacaagcaccgactgagcaa

SEQ ID NO: 340 - RNA, 21 nt, synthetic construct
  source 1..21: mol_type = other RNA
cctggggtgcaggagtgctaa

SEQ ID NO: 341 - RNA, 21 nt, synthetic construct
  source 1..21: mol_type = other RNA
ctggggtgcaggagtgctaca

SEQ ID NO: 342 - RNA, 21 nt, synthetic construct
  source 1..21: mol_type = other RNA
tggtaatggacagagttatca

SEQ ID NO: 343 - RNA, 21 nt, synthetic construct
  source 1..21: mol_type = other RNA
ggtaatggacagagttatcga

SEQ ID NO: 344 - RNA, 21 nt, synthetic construct
  source 1..21: mol_type = other RNA
cagagttatcgaggcacataa

SEQ ID NO: 345 - RNA, 21 nt, synthetic construct
  source 1..21: mol_type = other RNA
agagttatcgaggcacataca

SEQ ID NO: 346 - RNA, 21 nt, synthetic construct
  source 1..21: mol_type = other RNA
gagttatcgaggcacatacta

SEQ ID NO: 347 - RNA, 21 nt, synthetic construct
  source 1..21: mol_type = other RNA
ggcacatactccaccactgta

SEQ ID NO: 348 - RNA, 21 nt, synthetic construct
  source 1..21: mol_type = other RNA
aagaacctgccaagcttggta

SEQ ID NO: 349 - RNA, 21 nt, synthetic construct
  source 1..21: mol_type = other RNA
aacctgccaagcttggtcata

SEQ ID NO: 350 - RNA, 21 nt, synthetic construct
  source 1..21: mol_type = other RNA
cctgccaagcttggtcatcta

SEQ ID NO: 351 - RNA, 21 nt, synthetic construct
  source 1..21: mol_type = other RNA
gcaggagtgctaccacggtaa

SEQ ID NO: 352 - RNA, 21 nt, synthetic construct
  source 1..21: mol_type = other RNA
caggagtgctaccacggtaaa

SEQ ID NO: 353 - RNA, 21 nt, synthetic construct
  source 1..21: mol_type = other RNA
ggagtgctaccacggtaatga

SEQ ID NO: 354 - RNA, 21 nt, synthetic construct
  source 1..21: mol_type = other RNA
gtgctaccacggtaatggaca

SEQ ID NO: 355 - RNA, 21 nt, synthetic construct
  source 1..21: mol_type = other RNA
tgctaccacggtaatggacaa

SEQ ID NO: 356 - RNA, 21 nt, synthetic construct
  source 1..21: mol_type = other RNA
ctaccacggtaatggacagaa

SEQ ID NO: 357 - RNA, 21 nt, synthetic construct
  source 1..21: mol_type = other RNA
taccacggtaatggacagaga

SEQ ID NO: 358 - RNA, 21 nt, synthetic construct
  source 1..21: mol_type = other RNA
ccacggtaatggacagagtta

SEQ ID NO: 359 - RNA, 21 nt, synthetic construct
  source 1..21: mol_type = other RNA
cggtaatggacagagttatca

SEQ ID NO: 360 - RNA, 21 nt, synthetic construct
  source 1..21: mol_type = other RNA
ggtaatggacagagttatcga

SEQ ID NO: 361 - RNA, 21 nt, synthetic construct
  source 1..21: mol_type = other RNA
tgcaggaatccagatcctgta

SEQ ID NO: 362 - RNA, 21 nt, synthetic construct
  source 1..21: mol_type = other RNA
tcctgtggcagccccttatta

SEQ ID NO: 363 - RNA, 21 nt, synthetic construct
  source 1..21: mol_type = other RNA
tgtggcagccccttattgtta

SEQ ID NO: 364 - RNA, 21 nt, synthetic construct
  source 1..21: mol_type = other RNA
gcagccccttattgttataca

SEQ ID NO: 365 - RNA, 21 nt, synthetic construct
  source 1..21: mol_type = other RNA
cagccccttattgttatacga

SEQ ID NO: 366 - RNA, 21 nt, synthetic construct
  source 1..21: mol_type = other RNA
agccccttattgttatacgaa

SEQ ID NO: 367 - RNA, 21 nt, synthetic construct
  source 1..21: mol_type = other RNA
gagtactgcaacctgacacaa

SEQ ID NO: 368 - RNA, 21 nt, synthetic construct
  source 1..21: mol_type = other RNA
gtactgcaacctgacacaata

SEQ ID NO: 369 - RNA, 21 nt, synthetic construct
  source 1..21: mol_type = other RNA
actgcaacctgacacaatgca

SEQ ID NO: 370 - RNA, 21 nt, synthetic construct
  source 1..21: mol_type = other RNA
tgcaacctgacacaatgctca

SEQ ID NO: 371 - RNA, 21 nt, synthetic construct
  source 1..21: mol_type = other RNA
gcaacctgacacaatgctcaa

SEQ ID NO: 372 - RNA, 21 nt, synthetic construct
  source 1..21: mol_type = other RNA
tgacacaatgctcagacgcaa

SEQ ID NO: 373 - RNA, 21 nt, synthetic construct
  source 1..21: mol_type = other RNA
acacaatgctcagacgcagaa

SEQ ID NO: 374 - RNA, 21 nt, synthetic construct
  source 1..21: mol_type = other RNA
acaatgctcagacgcagaaga

SEQ ID NO: 375 - RNA, 21 nt, synthetic construct
  source 1..21: mol_type = other RNA
gattccaagcctagaggctca

SEQ ID NO: 376 - RNA, 21 nt, synthetic construct
  source 1..21: mol_type = other RNA
gcctagaggctccttctgaaa

SEQ ID NO: 377 - RNA, 21 nt, synthetic construct
  source 1..21: mol_type = other RNA
aacaagcaccaactgagcaaa

SEQ ID NO: 378 - RNA, 21 nt, synthetic construct
  source 1..21: mol_type = other RNA
caagcaccaactgagcaaaga

SEQ ID NO: 379 - RNA, 21 nt, synthetic construct
  source 1..21: mol_type = other RNA
ggacagagttatcaaggcaca

SEQ ID NO: 380 - RNA, 21 nt, synthetic construct
  source 1..21: mol_type = other RNA
tactgccgaaatccagatcca

SEQ ID NO: 381 - RNA, 21 nt, synthetic construct
  source 1..21: mol_type = other RNA
gtggcagccccttggtgttaa

SEQ ID NO: 382 - RNA, 21 nt, synthetic construct
  source 1..21: mol_type = other RNA
gcagccccttggtgttataca

SEQ ID NO: 383 - RNA, 21 nt, synthetic construct
  source 1..21: mol_type = other RNA
agccccttggtgttatacaaa

SEQ ID NO: 384 - RNA, 21 nt, synthetic construct
  source 1..21: mol_type = other RNA
gccccttggtgttatacaaca

SEQ ID NO: 385 - RNA, 21 nt, synthetic construct
  source 1..21: mol_type = other RNA
agtactgcaacctgacacgaa

SEQ ID NO: 386 - RNA, 21 nt, synthetic construct
  source 1..21: mol_type = other RNA
ctgcaacctgacacgatgcta

SEQ ID NO: 387 - RNA, 21 nt, synthetic construct
  source 1..21: mol_type = other RNA
tgcaacctgacacgatgctca

SEQ ID NO: 388 - RNA, 21 nt, synthetic construct
  source 1..21: mol_type = other RNA
gcaacctgacacgatgctcaa

SEQ ID NO: 389 - RNA, 21 nt, synthetic construct
  source 1..21: mol_type = other RNA
aacctgacacgatgctcagaa

SEQ ID NO: 390 - RNA, 21 nt, synthetic construct
  source 1..21: mol_type = other RNA
gctccaagcctagaggcttta

SEQ ID NO: 391 - RNA, 21 nt, synthetic construct
  source 1..21: mol_type = other RNA
gactgctactaccattatgga

SEQ ID NO: 392 - RNA, 21 nt, synthetic construct
  source 1..21: mol_type = other RNA
actgctactaccattatggaa

SEQ ID NO: 393 - RNA, 21 nt, synthetic construct
  source 1..21: mol_type = other RNA
ctgctactaccattatggaca

SEQ ID NO: 394 - RNA, 21 nt, synthetic construct
  source 1..21: mol_type = other RNA
ctactaccattatggacagaa

SEQ ID NO: 395 - RNA, 21 nt, synthetic construct
  source 1..21: mol_type = other RNA
ctaccattatggacagagtta

SEQ ID NO: 396 - RNA, 21 nt, synthetic construct
  source 1..21: mol_type = other RNA
taccattatggacagagttaa

SEQ ID NO: 397 - RNA, 21 nt, synthetic construct
  source 1..21: mol_type = other RNA
gaagaacttgccaagcttgga

SEQ ID NO: 398 - RNA, 21 nt, synthetic construct
  source 1..21: mol_type = other RNA
cttgccaagcttggtcatcta

SEQ ID NO: 399 - RNA, 21 nt, synthetic construct
  source 1..21: mol_type = other RNA
tgccaagcttggtcatctata

SEQ ID NO: 400 - RNA, 21 nt, synthetic construct
  source 1..21: mol_type = other RNA
gccaagcttggtcatctatga

SEQ ID NO: 401 - RNA, 21 nt, synthetic construct
  source 1..21: mol_type = other RNA
tgacaccacaccagcatagta

SEQ ID NO: 402 - RNA, 21 nt, synthetic construct
  source 1..21: mol_type = other RNA
gacaccacaccagcatagtca

SEQ ID NO: 403 - RNA, 21 nt, synthetic construct
  source 1..21: mol_type = other RNA
acaccacaccagcatagtcga

SEQ ID NO: 404 - RNA, 21 nt, synthetic construct
  source 1..21: mol_type = other RNA
accacaccagcatagtcggaa

SEQ ID NO: 405 - RNA, 21 nt, synthetic construct
  source 1..21: mol_type = other RNA
ccacaccagcatagtcggaca

SEQ ID NO: 406 - RNA, 21 nt, synthetic construct
  source 1..21: mol_type = other RNA
cacaccagcatagtcggacca

SEQ ID NO: 407 - RNA, 21 nt, synthetic construct
  source 1..21: mol_type = other RNA
gcatagtcggaccccagaaaa

SEQ ID NO: 408 - RNA, 21 nt, synthetic construct
  source 1..21: mol_type = other RNA
catagtcggaccccagaaaaa

SEQ ID NO: 409 - RNA, 21 nt, synthetic construct
  source 1..21: mol_type = other RNA
agtcggaccccagaaaactaa

SEQ ID NO: 410 - RNA, 21 nt, synthetic construct
  source 1..21: mol_type = other RNA
cttggtgttacaccatggata

SEQ ID NO: 411 - RNA, 21 nt, synthetic construct
  source 1..21: mol_type = other RNA
ttggtgttacaccatggatca

SEQ ID NO: 412 - RNA, 21 nt, synthetic construct
  source 1..21: mol_type = other RNA
tggtgttacaccatggatcca

SEQ ID NO: 413 - RNA, 21 nt, synthetic construct
  source 1..21: mol_type = other RNA
ggtgttacaccatggatccca

SEQ ID NO: 414 - RNA, 21 nt, synthetic construct
  source 1..21: mol_type = other RNA
caccatggatcccagtgtcaa

SEQ ID NO: 415 - RNA, 21 nt, synthetic construct
  source 1..21: mol_type = other RNA
accatggatcccagtgtcaga

SEQ ID NO: 416 - RNA, 21 nt, synthetic construct
  source 1..21: mol_type = other RNA
gaatcaagtgtccttgcaaca

SEQ ID NO: 417 - RNA, 21 nt, synthetic construct
  source 1..21: mol_type = other RNA
gtccttggtgttataccatga

SEQ ID NO: 418 - RNA, 21 nt, synthetic construct
  source 1..21: mol_type = other RNA
ccttggtgttataccatggaa

SEQ ID NO: 419 - RNA, 21 nt, synthetic construct
  source 1..21: mol_type = other RNA
cttggtgttataccatggata

SEQ ID NO: 420 - RNA, 21 nt, synthetic construct
  source 1..21: mol_type = other RNA
ggtgttataccatggatccca

SEQ ID NO: 421 - RNA, 21 nt, synthetic construct
  source 1..21: mol_type = other RNA
ctgcaacctgacacaatgtca

SEQ ID NO: 422 - RNA, 21 nt, synthetic construct
  source 1..21: mol_type = other RNA
tgcaacctgacacaatgtcca

SEQ ID NO: 423 - RNA, 21 nt, synthetic construct
  source 1..21: mol_type = other RNA
gcaacctgacacaatgtccaa

SEQ ID NO: 424 - RNA, 21 nt, synthetic construct
  source 1..21: mol_type = other RNA
acctgacacaatgtccagtga

SEQ ID NO: 425 - RNA, 21 nt, synthetic construct
  source 1..21: mol_type = other RNA
acaatgtccagtgacagaata

SEQ ID NO: 426 - RNA, 21 nt, synthetic construct
  source 1..21: mol_type = other RNA
agaatactacccaaatggtga

SEQ ID NO: 427 - RNA, 21 nt, synthetic construct
  source 1..21: mol_type = other RNA
attcgcccttggtgttataca

SEQ ID NO: 428 - RNA, 21 nt, synthetic construct
  source 1..21: mol_type = other RNA
gcccttggtgttataccatga

SEQ ID NO: 429 - RNA, 21 nt, synthetic construct
  source 1..21: mol_type = other RNA
ccttggtgttataccatggaa

SEQ ID NO: 430 - RNA, 21 nt, synthetic construct
  source 1..21: mol_type = other RNA
cttggtgttataccatggata

SEQ ID NO: 431 - RNA, 21 nt, synthetic construct
  source 1..21: mol_type = other RNA
taccatggatcccagtgtcaa

SEQ ID NO: 432 - RNA, 21 nt, synthetic construct
  source 1..21: mol_type = other RNA
accatggatcccagtgtcaga

SEQ ID NO: 433 - RNA, 21 nt, synthetic construct
  source 1..21: mol_type = other RNA
cacaactcccacggtggtcca

SEQ ID NO: 434 - RNA, 21 nt, synthetic construct
  source 1..21: mol_type = other RNA
acaactcccacggtggtccca

SEQ ID NO: 435 - RNA, 21 nt, synthetic construct
  source 1..21: mol_type = other RNA
ggtggtcccagttccaagcaa

SEQ ID NO: 436 - RNA, 21 nt, synthetic construct
  source 1..21: mol_type = other RNA
acgatgtccagtgacagaata

SEQ ID NO: 437 - RNA, 21 nt, synthetic construct
  source 1..21: mol_type = other RNA
tatgataccacactggcatca

SEQ ID NO: 438 - RNA, 21 nt, synthetic construct
  source 1..21: mol_type = other RNA
atgataccacactggcatcaa

SEQ ID NO: 439 - RNA, 21 nt, synthetic construct
  source 1..21: mol_type = other RNA
aaacaaccctggtgttacaca

SEQ ID NO: 440 - RNA, 21 nt, synthetic construct
  source 1..21: mol_type = other RNA
gaaacagaatcaggtgtccta

SEQ ID NO: 441 - RNA, 21 nt, synthetic construct
  source 1..21: mol_type = other RNA
aaacagaatcaggtgtcctaa

SEQ ID NO: 442 - RNA, 21 nt, synthetic construct
  source 1..21: mol_type = other RNA
acagaatcaggtgtcctagaa

SEQ ID NO: 443 - RNA, 21 nt, synthetic construct
  source 1..21: mol_type = other RNA
cagaatcaggtgtcctagaga

SEQ ID NO: 444 - RNA, 21 nt, synthetic construct
  source 1..21: mol_type = other RNA
gaatcaggtgtcctagagaca

SEQ ID NO: 445 - RNA, 21 nt, synthetic construct
  source 1..21: mol_type = other RNA
caggtgtcctagagactccca

SEQ ID NO: 446 - RNA, 21 nt, synthetic construct
  source 1..21: mol_type = other RNA
gagactcccactgttgttcca

SEQ ID NO: 447 - RNA, 21 nt, synthetic construct
  source 1..21: mol_type = other RNA
agagttatcgaggcacattca

SEQ ID NO: 448 - RNA, 21 nt, synthetic construct
  source 1..21: mol_type = other RNA
atgtcaatcttggtcatccaa

SEQ ID NO: 449 - RNA, 21 nt, synthetic construct
  source 1..21: mol_type = other RNA
tgtcaatcttggtcatccata

SEQ ID NO: 450 - RNA, 21 nt, synthetic construct
  source 1..21: mol_type = other RNA
atacaggcccttggtgtttta

SEQ ID NO: 451 - RNA, 21 nt, synthetic construct
  source 1..21: mol_type = other RNA
actgcaacctgacgcgatgca

SEQ ID NO: 452 - RNA, 21 nt, synthetic construct
  source 1..21: mol_type = other RNA
ctgcaacctgacgcgatgcta

SEQ ID NO: 453 - RNA, 21 nt, synthetic construct
  source 1..21: mol_type = other RNA
tgcaacctgacgcgatgctca

SEQ ID NO: 454 - RNA, 21 nt, synthetic construct
  source 1..21: mol_type = other RNA
gcaacctgacgcgatgctcaa

SEQ ID NO: 455 - RNA, 21 nt, synthetic construct
  source 1..21: mol_type = other RNA
aacctgacgcgatgctcagaa

SEQ ID NO: 456 - RNA, 21 nt, synthetic construct
  source 1..21: mol_type = other RNA
acctgacgcgatgctcagaca

SEQ ID NO: 457 - RNA, 21 nt, synthetic construct
  source 1..21: mol_type = other RNA
cctgacgcgatgctcagacaa

SEQ ID NO: 458 - RNA, 21 nt, synthetic construct
  source 1..21: mol_type = other RNA
ctgacgcgatgctcagacaca

SEQ ID NO: 459 - RNA, 21 nt, synthetic construct
  source 1..21: mol_type = other RNA
tgacgcgatgctcagacacaa

SEQ ID NO: 460 - RNA, 21 nt, synthetic construct
  source 1..21: mol_type = other RNA
acgcgatgctcagacacagaa

SEQ ID NO: 461 - RNA, 21 nt, synthetic construct
  source 1..21: mol_type = other RNA
aatgtcctggaagcattgtaa

SEQ ID NO: 462 - RNA, 21 nt, synthetic construct
  source 1..21: mol_type = other RNA
gactgaatgttacatcactga

SEQ ID NO: 463 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gaaggacctgccaagcttgga

SEQ ID NO: 464 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aaggacctgccaagcttggta

SEQ ID NO: 465 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gacctgccaagcttggtcata

SEQ ID NO: 466 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cctgccaagcttggtcatcta

SEQ ID NO: 467 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgccaagcttggtcatctata

SEQ ID NO: 468 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gccaagcttggtcatctatga

SEQ ID NO: 469 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tacccaaatgctggcttgata

SEQ ID NO: 470 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgctcagacgcagaagggaca

SEQ ID NO: 471 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
agacgcagaagggactgccga

SEQ ID NO: 472 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
acgcagaagggactgccgtca

SEQ ID NO: 473 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cgcagaagggactgccgtcga

SEQ ID NO: 474 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gcagaagggactgccgtcgca

SEQ ID NO: 475 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tccgactgttaccccggttca

SEQ ID NO: 476 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ccgactgttaccccggttcca

SEQ ID NO: 477 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cgactgttaccccggttccaa

SEQ ID NO: 478 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ctgttaccccggttccaagca

SEQ ID NO: 479 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgttaccccggttccaagcca

SEQ ID NO: 480 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
accccggttccaagcctagaa

SEQ ID NO: 481 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ccccggttccaagcctagaga

SEQ ID NO: 482 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ccggttccaagcctagaggca

SEQ ID NO: 483 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cggttccaagcctagaggcta

SEQ ID NO: 484 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ggttccaagcctagaggctca

SEQ ID NO: 485 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ccaagcctagaggctccttca

SEQ ID NO: 486 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aagcctagaggctccttccga

SEQ ID NO: 487 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cctagaggctccttccgaaca

SEQ ID NO: 488 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aggctccttccgaacaagcaa

SEQ ID NO: 489 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ggctccttccgaacaagcaca

SEQ ID NO: 490 - RNA, 21 nt, synthetic construct
  source 1..21: mol_type = other RNA
ctccttccgaacaagcaccga

SEQ ID NO: 491 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tccttccgaacaagcaccgaa

SEQ ID NO: 492 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ttccgaacaagcaccgactga

SEQ ID NO: 493 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gaacaagcaccgactgagcaa

SEQ ID NO: 494 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cctggggtgcaggagtgctaa

SEQ ID NO: 495 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ctggggtgcaggagtgctaca

SEQ ID NO: 496 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tggtaatggacagagttatca

SEQ ID NO: 497 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ggtaatggacagagttatcga

SEQ ID NO: 498 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cagagttatcgaggcacataa

SEQ ID NO: 499 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
agagttatcgaggcacataca

SEQ ID NO: 500 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gagttatcgaggcacatacta

SEQ ID NO: 501 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ggcacatactccaccactgta

SEQ ID NO: 502 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aagaacctgccaagcttggta

SEQ ID NO: 503 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aacctgccaagcttggtcata

SEQ ID NO: 504 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cctgccaagcttggtcatcta

SEQ ID NO: 505 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gcaggagtgctaccacggtaa

SEQ ID NO: 506 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
caggagtgctaccacggtaaa

SEQ ID NO: 507 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ggagtgctaccacggtaatga

SEQ ID NO: 508 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gtgctaccacggtaatggaca

SEQ ID NO: 509 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgctaccacggtaatggacaa

SEQ ID NO: 510 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ctaccacggtaatggacagaa

SEQ ID NO: 511 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
taccacggtaatggacagaga

SEQ ID NO: 512 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ccacggtaatggacagagtta

SEQ ID NO: 513 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cggtaatggacagagttatca

SEQ ID NO: 514 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ggtaatggacagagttatcga

SEQ ID NO: 515 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgcaggaatccagatcctgta

SEQ ID NO: 516 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tcctgtggcagccccttatta

SEQ ID NO: 517 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgtggcagccccttattgtta

SEQ ID NO: 518 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gcagccccttattgttataca

SEQ ID NO: 519 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cagccccttattgttatacga

SEQ ID NO: 520 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
agccccttattgttatacgaa

SEQ ID NO: 521 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gagtactgcaacctgacacaa

SEQ ID NO: 522 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gtactgcaacctgacacaata

SEQ ID NO: 523 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
actgcaacctgacacaatgca

SEQ ID NO: 524 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgcaacctgacacaatgctca

SEQ ID NO: 525 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gcaacctgacacaatgctcaa

SEQ ID NO: 526 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgacacaatgctcagacgcaa

SEQ ID NO: 527 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
acacaatgctcagacgcagaa

SEQ ID NO: 528 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
acaatgctcagacgcagaaga

SEQ ID NO: 529 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gattccaagcctagaggctca

SEQ ID NO: 530 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gcctagaggctccttctgaaa

SEQ ID NO: 531 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aacaagcaccaactgagcaaa

SEQ ID NO: 532 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
caagcaccaactgagcaaaga

SEQ ID NO: 533 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ggacagagttatcaaggcaca

SEQ ID NO: 534 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tactgccgaaatccagatcca

SEQ ID NO: 535 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gtggcagccccttggtgttaa

SEQ ID NO: 536 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gcagccccttggtgttataca

SEQ ID NO: 537 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
agccccttggtgttatacaaa

SEQ ID NO: 538 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gccccttggtgttatacaaca

SEQ ID NO: 539 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
agtactgcaacctgacacgaa

SEQ ID NO: 540 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ctgcaacctgacacgatgcta

SEQ ID NO: 541 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgcaacctgacacgatgctca

SEQ ID NO: 542 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gcaacctgacacgatgctcaa

SEQ ID NO: 543 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aacctgacacgatgctcagaa

SEQ ID NO: 544 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gctccaagcctagaggcttta

SEQ ID NO: 545 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gactgctactaccattatgga

SEQ ID NO: 546 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
actgctactaccattatggaa

SEQ ID NO: 547 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ctgctactaccattatggaca

SEQ ID NO: 548 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ctactaccattatggacagaa

SEQ ID NO: 549 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ctaccattatggacagagtta

SEQ ID NO: 550 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
taccattatggacagagttaa

SEQ ID NO: 551 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gaagaacttgccaagcttgga

SEQ ID NO: 552 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cttgccaagcttggtcatcta

SEQ ID NO: 553 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgccaagcttggtcatctata

SEQ ID NO: 554 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gccaagcttggtcatctatga

SEQ ID NO: 555 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgacaccacaccagcatagta

SEQ ID NO: 556 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gacaccacaccagcatagtca

SEQ ID NO: 557 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
acaccacaccagcatagtcga

SEQ ID NO: 558 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
accacaccagcatagtcggaa

SEQ ID NO: 559 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ccacaccagcatagtcggaca

SEQ ID NO: 560 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cacaccagcatagtcggacca

SEQ ID NO: 561 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gcatagtcggaccccagaaaa

SEQ ID NO: 562 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
catagtcggaccccagaaaaa

SEQ ID NO: 563 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
agtcggaccccagaaaactaa

SEQ ID NO: 564 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cttggtgttacaccatggata

SEQ ID NO: 565 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ttggtgttacaccatggatca

SEQ ID NO: 566 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tggtgttacaccatggatcca

SEQ ID NO: 567 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ggtgttacaccatggatccca

SEQ ID NO: 568 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
caccatggatcccagtgtcaa

SEQ ID NO: 569 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
accatggatcccagtgtcaga

SEQ ID NO: 570 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gaatcaagtgtccttgcaaca

SEQ ID NO: 571 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gtccttggtgttataccatga

SEQ ID NO: 572 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ccttggtgttataccatggaa

SEQ ID NO: 573 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cttggtgttataccatggata

SEQ ID NO: 574 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ggtgttataccatggatccca

SEQ ID NO: 575 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ctgcaacctgacacaatgtca

SEQ ID NO: 576 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgcaacctgacacaatgtcca

SEQ ID NO: 577 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gcaacctgacacaatgtccaa

SEQ ID NO: 578 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
acctgacacaatgtccagtga

SEQ ID NO: 579 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
acaatgtccagtgacagaata

SEQ ID NO: 580 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
agaatactacccaaatggtga

SEQ ID NO: 581 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
attcgcccttggtgttataca

SEQ ID NO: 582 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gcccttggtgttataccatga

SEQ ID NO: 583 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ccttggtgttataccatggaa

SEQ ID NO: 584 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cttggtgttataccatggata

SEQ ID NO: 585 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
taccatggatcccagtgtcaa

SEQ ID NO: 586 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
accatggatcccagtgtcaga

SEQ ID NO: 587 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cacaactcccacggtggtcca

SEQ ID NO: 588 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
acaactcccacggtggtccca

SEQ ID NO: 589 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ggtggtcccagttccaagcaa

SEQ ID NO: 590 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
acgatgtccagtgacagaata

SEQ ID NO: 591 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tatgataccacactggcatca

SEQ ID NO: 592 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
atgataccacactggcatcaa

SEQ ID NO: 593 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aaacaaccctggtgttacaca

SEQ ID NO: 594 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gaaacagaatcaggtgtccta

SEQ ID NO: 595 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aaacagaatcaggtgtcctaa

SEQ ID NO: 596 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
acagaatcaggtgtcctagaa

SEQ ID NO: 597 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cagaatcaggtgtcctagaga

SEQ ID NO: 598 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gaatcaggtgtcctagagaca

SEQ ID NO: 599 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
caggtgtcctagagactccca

SEQ ID NO: 600 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gagactcccactgttgttcca

SEQ ID NO: 601 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
agagttatcgaggcacattca

SEQ ID NO: 602 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
atgtcaatcttggtcatccaa

SEQ ID NO: 603 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgtcaatcttggtcatccata

SEQ ID NO: 604 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
atacaggcccttggtgtttta

SEQ ID NO: 605 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
actgcaacctgacgcgatgca

SEQ ID NO: 606 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ctgcaacctgacgcgatgcta

SEQ ID NO: 607 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgcaacctgacgcgatgctca

SEQ ID NO: 608 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gcaacctgacgcgatgctcaa

SEQ ID NO: 609 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aacctgacgcgatgctcagaa

SEQ ID NO: 610 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
acctgacgcgatgctcagaca

SEQ ID NO: 611 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
cctgacgcgatgctcagacaa

SEQ ID NO: 612 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
ctgacgcgatgctcagacaca

SEQ ID NO: 613 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
tgacgcgatgctcagacacaa

SEQ ID NO: 614 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
acgcgatgctcagacacagaa

SEQ ID NO: 615 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
aatgtcctggaagcattgtaa

SEQ ID NO: 616 - RNA, 21 nt, synthetic construct
  source 1..21: mol_type = other RNA
  modified_base 1..6: mod_base = OTHER; note = 2'O-Methyl ribonucleotide
  modified_base 1..3: mod_base = OTHER; note = Phosphorothioate linkage
  modified_base 7: mod_base = OTHER; note = 2'fluoro ribonucleotide
  modified_base 8: mod_base = OTHER; note = 2'O-Methyl ribonucleotide
  modified_base 9: mod_base = OTHER; note = 2'fluoro ribonucleotide
  modified_base 10: mod_base = OTHER; note = 2'O-Methyl ribonucleotide
  modified_base 11: mod_base = OTHER; note = 2'fluoro ribonucleotide
  modified_base 12..21: mod_base = OTHER; note = 2'O-Methyl ribonucleotide
gactgaatgttacatcactga

SEQ ID NO: 617 - protein, 5 aa, synthetic construct
YIGSR

SEQ ID NO: 618 - protein, 4 aa, synthetic construct
  SITE 1: note = N-formyl-methionyl
  SITE 2: note = leucyl
MLFK